(19) United States
(12) Patent Application Publication
(10) Pub. No.: US 2009/0136945 A1
(43) Pub. Date: May 28, 2009

(54) COMPOSITIONS AND METHODS FOR ASSESSING DISORDERS

(75) Inventors: Robert D. Loberg, Saline, MI (US); Kenneth Pienta, Ann Arbor, MI (US)

Correspondence Address: Casimir Jones, S.C., 440 Science Drive, Suite 203, Madison, WI, 53711, US

(73) Assignee: The Regents of the University of Michigan, Ann Arbor, MI (US)

(21) Appl. No.: 12/249,730

(22) Filed: Oct. 10, 2008

Related U.S. Application Data
(60) Provisional application No. 60/978,998, filed on Oct. 10, 2007.

(51) Int. Cl. C12Q 1/68; C12Q 1/02

(52) U.S. Cl. 435/6; 435/29

(57) ABSTRACT

The present invention relates to compositions and methods for disorder (e.g., hyperproliferative disorders, inflammatory disorders) research, diagnosis, and treatment, including but not limited to, bio-markers specific for a particular disorder (e.g., cancer, systemic lupus erythematosus). In particular, the present invention relates to peripheral blood mononuclear cells (PBMCs) as bio-markers specific for particular disorders.

### CROSS-REFERENCE TO RELATED APPLICATIONS

This application claims priority to U.S. Provisional Application Ser. No. 60/978,998, filed Oct. 10, 2007, which is herein incorporated by reference in its entirety.

### STATEMENT REGARDING FEDERALLY SPONSORED RESEARCH OR DEVELOPMENT

This invention was made with government support under Grant No. CA069568 awarded by the National Institutes of Health. The government has certain rights in the invention.

### FIELD OF THE INVENTION

The present invention relates to compositions and methods for disorder (e.g., hyperproliferative disorders, inflammatory disorders) research, diagnosis, and treatment, including but not limited to, bio-markers specific for a particular disorder (e.g., cancer, systemic lupus erythematosus). In particular, the present invention relates to peripheral blood mononuclear cells (PBMCs) as bio-markers specific for particular disorders.

### BACKGROUND OF THE INVENTION

Prostate cancer is a disease in which cancer develops in the prostate, a gland in the male reproductive system. Cancer occurs when cells of the prostate mutate and begin to multiply out of control. These cells may spread (metastasize) from the prostate to other parts of the body, especially the bones and lymph nodes. Prostate cancer may cause pain, difficulty in urinating, erectile dysfunction and other symptoms.

Rates of prostate cancer vary widely across the world. It is least common in South and East Asia, more common in Europe—though the rates vary widely between countries—and most common in the United States. According to the American Cancer Society, prostate cancer is least common among Asian men and most common among black men with figures for European men in between. However, these high rates may be affected by increasing rates of detection.

Prostate cancer develops most frequently in men over fifty. This cancer can only occur in men; the prostate is exclusively of the male reproductive tract. It is the second most common type of cancer in men in the United States, where it is responsible for more male deaths than any other cancer except lung cancer. However, many men who develop prostate cancer never have symptoms, undergo no therapy, and eventually die of other causes. Many factors, including genetics and diet, have been implicated in the development of prostate cancer.

Prostate cancer is most often discovered by physical examination or by screening blood tests, such as the PSA (prostate specific antigen) test. There is some current concern about the accuracy of the PSA test and its usefulness. Suspected prostate cancer is typically confirmed by removing a piece of the prostate (biopsy) and examining it under a microscope. Further tests, such as X-rays and bone scans, may be performed to determine whether prostate cancer has spread.

Prostate cancer is typically diagnosed with a digital rectal exam and/or prostate specific antigen (PSA) screening. An elevated PSA level can indicate the presence of PCA. PSA is used as a marker for prostate cancer because it is essentially restricted to prostate cells. A healthy prostate will produce a stable amount—typically below 4 nanograms per milliliter, or a PSA reading of “4” or less—whereas cancer cells produce escalating amounts that correspond with the severity of the cancer. A level between 4 and 10 may raise a doctor's suspicion that a patient has prostate cancer, while amounts above 50 may show that the tumor has spread elsewhere in the body.

The development of additional methods for characterizing prostate cancer is needed to supplement currently available screening methods.

### SUMMARY OF THE INVENTION

A cell's behavior is dictated by a complex dynamic system of genetic interactions. A central role in the understanding of a multi-cellular system, the stability in a changing environment, and how such systems change with the presence of cancer, is highly dependent upon the process of differentiation. Myeloid progenitor cells from the bone marrow compartment mature and differentiate into promonoblasts and monoblasts (where they acquire CD11b expression) where they are released into the blood stream and further differentiate into monocytes. Circulating monocytes receive molecular signals from surrounding tissues and migrate in response to stimuli which are classically in response to infection or inflammation. Monocytes terminally differentiate into tissue resident macrophages in response to these molecular stimuli and comprise a major component of the innate immune system.

The population of circulating CD11b+ cells are myeloid progenitors which arise from the bone marrow compartment and undergo differentiation upon stimulus to terminally differentiated effector cells (e.g. tissue macrophages) (see, e.g., Nat Rev Cancer. 2008 August; 8(8):618-31; herein incorporated by reference in its entirety). In the presence of cancer, a subset of CD11b+ cells, the monocytes, are recruited to tumors and differentiate into tumor associated macrophages (TAMs). TAMs promote tumorigenesis through a variety of mechanisms, including the secretion of VEGF and matrix MMPs (see, e.g., Sica, et al., Eur J Cancer, 42: 717-727, 2006; Nature. 2008 Jul. 24; 454(7203): 436-44; Lewis, et al., Cancer Res, 66: 605-612, 2006; Mantovani, et al., Immunol Today, 13: 265-270, 1992; each herein incorporated by reference in their entireties).

Experiments conducted during the development of embodiments for the present invention demonstrated that the circulating CD11b+ cells are “educated” prior to terminal differentiation and infiltration into the tumor and can act as detectors of inflammatory disorders (e.g., systemic lupus erythematosus) and hyperproliferative disorders (e.g., cancer). For example, different classes of cancer generate a unique expression signature that reflects the presence of that type of cancer. Thus, the cells provide a marker of cancer type, stage, etc. These cells are educated by, for example, circulating chemokines/cytokines released by the tumor environment and/or by circulation through the tumor and release into the circulation. Different types of cancer secrete, for example, a unique signature of soluble factors or have a unique set of insoluble factors detected by the circulating monocytes that is below the ability of detection by other mechanisms. The present invention utilizes the cells of the body to act as detectors of disorders (e.g., inflammatory disorders, hyperproliferative disorders). For example, experiments conducted during the course of development of embodiments for the present invention demonstrated that prostate cancer has the ability to change the characteristics of circulating peripheral blood mononuclear cells (e.g., CD11b+ PBMCs) and that this change can be used for research, diagnostic, and therapeutic uses.

For example, it was shown that subjects (e.g., human patients) diagnosed with prostate cancer have different genetic expression within CD11b+ PBMCs (e.g., down-regulated MSRA, ZFAND6, THADA, FYN, RABGAP1L, IMMP2L, RICTOR, JMJD2C, NPTN, and/or VTI1A gene expression) (see, altered gene expression in any one or more (e.g., 1, 5, 10, 15, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 5A) in comparison with subjects not diagnosed with prostate cancer. It was shown that subjects diagnosed with advanced prostate cancer have different genetic expression within CD11b+ PBMCs (e.g., ERG, CHIT1, FAM20A, CRISP3, CEACAM6, MS4A1, FLJ22795, GLDN, CEACAM8, and ARG1) in comparison with control subjects (see, altered gene expression in any one or more (e.g., 1, 5, 10, 15, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 5B). It was shown that subjects diagnosed with advanced prostate cancer have different genetic expression within CD11b+ PBMCs (e.g., ANXA1, KLF4, NR4A2, FOSB, JUN, VIM, FOS, JUN, PPP1R15A, and SOS1) in comparison with subjects diagnosed with early prostate cancer (see, altered gene expression in any one or more (e.g., 1, 5, 10, 15, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 5C).

However, the present invention is not limited by the nature of CD11b+ characteristic used to assess a patient sample. The present invention identifies that CD11b+ cells have informative value in assessing prostate cancer status and risk. Any method or approach that assesses CD11b+ characteristics associated with disease status may be used, including, but not limited to, gene expression analysis, proteome analysis, cell morphology, and the like. Exemplary embodiments are described in more detail below to illustrate aspects of embodiments of the invention. In particular, gene expression analysis is focused on to illustrate embodiments of the invention. As such, experiments conducted during the course of development of embodiments for the present invention demonstrated that prostate cancer has the ability to change the genetic expression of circulating peripheral blood mononuclear cells (e.g., CD11b+ PBMCs) and that this genetic change can be exploited for research and disease characterization uses and diagnostic uses.

In some embodiments, the present invention provide methods for assessing a disorder, comprising: identifying a characteristic associated with the disorder of a CD11b+ peripheral blood mononuclear cell obtained from a subject sample. The methods are not limited to particular disorders. In some embodiments, the disorder is a hyperproliferative disorder such as, for example, cancer (e.g., lung cancer, colon cancer, prostate cancer, pancreatic cancer, breast cancer). In some embodiments, the disorder is an inflammatory disorder such as, for example, systemic lupus erythematosus.

In some embodiments, the characteristic of the CD11b+ peripheral blood mononuclear cell associated with the disorder is altered gene expression (e.g., over-expression and/or under-expression) (e.g., compared to a control sample). For example, in some embodiments wherein the disorder is prostate cancer, the altered gene expression is from one or more of the genes: MSRA, ZFAND6, THADA, FYN, RABGAP1L, IMMP2L, RICTOR, JMJD2C, NPTN, and VTI1A. In some embodiments where the disorder is prostate cancer, the altered gene expression is from one or more of the genes found in FIG. 5A. In some embodiments, altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 5B (e.g., ERG, CHIT1, FAM20A, CRISP3, CEACAM6, MS4A1, FLJ22795, GLDN, CEACAM8, and ARG1) distinguishes patients having advanced prostate cancer from patients not having prostate cancer. In some embodiments, altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 5C (e.g., ANXA1, KLF4, NR4A2, FOSB, JUN, VIM, FOS, JUN, PPP1R15A, and SOS1) distinguishes patients having advanced prostate cancer from patients having early prostate cancer. In some embodiments, altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 7C (e.g., FCRL5, TMEM156, OASL, PPP1R9A, COL4A4, BTLA, FAM110B, TPD52, MGC39900, KIAA0125) distinguishes patients having breast cancer from patients not having breast cancer. In some embodiments, altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 8C (e.g., METTL7B, GATA2, CHRM3, SPRYD5, ENPP3, FLVCR2, SEPT1, NLRC3, PHC2, FAM84B) distinguishes patients having lung cancer from patients not having lung cancer. In some embodiments, altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 9B (e.g., LGR4, C1QC, FAM20A, FMNL2, C1QA, TCF7L2, C1QB, METTL7B, EZR, CACNA2D3) distinguishes patients having pancreatic cancer from patients not having pancreatic cancer. In some embodiments, altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 10B (e.g., FAM20A, FLVCR2, METTL7B, CNTNAP2, WASF1, TCF7L2, ATXN3, ME1, CCR7, GAS6) distinguishes patients having colon cancer from patients not having colon cancer. In some embodiments, altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 11B (e.g., FAM20A, FLVCR2, METTL7B, CNTNAP2, WASF1, TCF7L2, ATXN3, ME1, CCR7, GAS6) distinguishes patients having active systemic lupus erythematosus from patients not having active systemic lupus erythematosus. In some embodiments, altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 11D (e.g., PPBP, SDPR, SPARC, PF4, GNG11, STAT1, PATL1, TBXA2R, PPCSK6, ITGB3) distinguishes patients having inactive systemic lupus erythematosus from patients not having active systemic lupus erythematosus. In some embodiments, altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 11F (e.g., PTBP1, ZNF274, RHEB, LARP5, KIAA1128, C10orf46, SMAD7, NARG1, FAM123B, CYP4V2) distinguishes patients having active systemic lupus erythematosus from patients having inactive systemic lupus erythematosus. Markers useful for analyzing other disorders or conditions may be selected using the approaches described herein.

The present invention is not limited by the nature of the sample used to assess CD11b+ cells. In some embodiments, the sample is a biopsy sample. In some embodiments, the sample is a blood, serum, or plasma sample.

In some embodiments, information obtained from the method is used for research (e.g., drug screening or analysis), diagnostic, or therapeutic purposes. For example, in some embodiments, information about a subject is provided to a treating physician. The treating physician may use the information, alone or in combination with other information, to select an appropriate treatment course of action for the subject (e.g., watchful waiting, surgery, pharmaceutical intervention, etc.). In some embodiments, treatment is carried out on the subject. An exemplary research method involves testing a drug candidate or other medical intervention on a patient and monitoring CD11b+ characteristics to assess the impact of the drug or medical intervention on the patient.

The characteristics of the CD11b+ cells may be analyzed to see if they are similar to or different from characteristics of known sample types. In some embodiments, the known sample type may be analyzed side-by-side with an experimental sample. However, in other embodiments, a database, look-up table, or the like, containing characteristics of a previously tested known sample or samples, is used to identify the nature of the experimental sample. Such analyses find use, for example, in assessing whether the experimental sample is associated with the presence or absence of a particular disorder (e.g., systemic lupus erythematosus, breast cancer, pancreatic cancer, prostate cancer) with, for example, a low risk for developing the disorder, with a medium risk for developing the disorder, with a high risk for developing the disorder, or, for example, with remission from a particular cancer (e.g., breast cancer, pancreatic cancer, prostate cancer), with recurrence from a particular cancer (e.g., breast cancer, pancreatic cancer, prostate cancer), with active cancer (e.g., breast cancer, pancreatic cancer, prostate cancer), with cell proliferation (e.g., prostate epithelial cell proliferation), with cellular metastasis (e.g., prostate epithelial cell metastasis), and etc.

The present invention also provides compositions (e.g., reagents, reaction mixtures, etc.) and kits associated with the methods. For example, kits may comprise one or more components (e.g., reagents, software, devices, etc.) necessary, sufficient, or useful for isolating samples, preparing samples for testing, testing samples (e.g., for gene expression of one or more genes), collecting data, analyzing data, and reporting data.

The methods of the present invention are not limited to assessing a specific type of disorder. In some embodiments, the methods are used for assessing disorders including, but not limited to, immune disorders, hyperproliferative disorders, and inflammatory disorders. With regard to immune disorders, the methods can be used to assess, for example, graft versus host disease, rheumatoid arthritis, and systemic lupus erythematosus. With regard to hyperproliferative disorders, the methods can be used to assess, for example, cancer, which can be either malignant or benign. Exemplary cancers that can be assessed include, for example, adenomas, adenocarcinomas, carcinomas, leukemias, lymphomas, melanomas, myelomas, sarcomas, and teratomas. In addition, the methods can be used to assess cancers of the bladder and the renal system, brain, breast, cervix, thyroid, skin, pancrease, colon, lung, ovaries, prostate, rectum. With regard to inflammatory disorders, the methods can be used to assess, for example, asthma and psoriasis.

### DESCRIPTION OF THE FIGURES

FIG. 1 shows in vivo data demonstrating altered gene expression in CD11b+ PBMCs from mice pre- and post-tumor challenge. CD11b+ PBMCs were collected from male SCID mice prior to PC-3 xenograft implantation. Further, CD11b+ PBMCs were collected from the same mice 3 weeks post-xenograft implantation and gene profiles were compared by cDNA microarray. The top 20 statistically changed genes were identified and verified by QPCR. Approximately 5000 genes were differentially expressed in the CD11b+ PBMCs following xenograft implantation compared to prexenograft samples.

FIG. 2 shows a list of genes intersecting between TAM and cancer present monocytes.

FIG. 3 shows gene expression profiles for CD11b+ cells isolated from patients with advanced hormone refractory prostate cancer (AC=active cancer by clinical definitions including rising PSA not improving with therapy) compared to age-matched normal controls (N). cDNA microarray analysis revealed >5000 differentially expressed genes in AC samples vs. N. Displayed are the top 400 differentially expressed genes based on a weighted sum of t-statistic and fold change expression values.

FIG. 4 shows that gene expression changes in CD11b+ cells can detect prostate cancer in patients versus age-matched controls. Data from a study of five men with advanced prostate cancer and five age matched controls demonstrated that 902 genes were significantly different between prostate cancer patients and the normal controls (>2 fold expression; p<0.01) (FIG. 4A). In a second set of experiments, 17 men with castration resistant prostate cancer and 10 age-matched controls were compared. In addition, 5 men with castration sensitive prostate cancer (men with no evidence of disease, e.g., undetectable PSA, but on castration therapy) were compared. Principal component analysis revealed separation of these groups (FIG. 4A). Generation of probe sets using differentially expressed genes generated ROC curves with high sensitivity and specificity (FIG. 4B). The advanced prostate cancer patients were further compared to another set of controls of 10 age—matched men undergoing prostate cancer biopsy for an elevated PSA who were not diagnosed with cancer (FIG. 4C).

FIG. 5A shows genes up-regulated and down-regulated in CD11b+ cells isolated from patients with advanced hormone refractory prostate cancer (AC=active cancer by clinical definitions including rising PSA not improving with therapy) compared to age-matched normal controls (N). FIG. 5B shows genes up-regulated and down-regulated in CD11b+ cells isolated from patients with advanced prostate cancer compared to control patients. FIG. 5C shows genes up-regulated and down-regulated in CD11b+ cells isolated from patients with advanced prostate cancer compared to patients with early prostate cancer.

FIGS. 6A and 6B show mouse and human CD11b+ PBMCs exposed to prostate cancer cells in vivo share similar gene expression profiles.

FIGS. 7A and 7B show that PBMCs from women with active breast cancer can be differentiated from women with no active cancer. Blood was collected from seven women with metastatic breast cancer who had failed hormonal therapy. Age matched controls consisted of 10 women with no history of cancer and 12 women with a history of breast cancer on hormonal therapy with no evidence of disease for a minimum of two years. Analysis revealed that CD11b+ cells from women could be differentiated from women without evidence of cancer. Women on hormonal therapy with no evidence of cancer could be differentiated from women with cancer. FIG. 7C shows genes up-regulated and down-regulated in CD11b+ cells isolated from patients with active breast cancer compared to women with no breast cancer.

FIG. 8 show that PBMCs from patients with active lung cancer can be differentiated from controls with no active cancer. A lung cancer specific gene expression set was generated from 3 men and 5 women non-small cell lung cancer as well as 1 man and 2 women with small cell lung cancer. All patients were undergoing or eligible for chemotherapy. Controls were derived from the common set of 10 age-matched men and 10 age-matched women with no history of cancer by verbal report (FIG. 8A). The compound covariate was lung cancer specific. Separation by sex improved the power to differentiate between patients and controls (FIG. 8B). FIG. 8C shows genes up-regulated and down-regulated in CD11b+ cells isolated from patients with lung cancer compared to controls with no lung cancer. FIG. 8D shows genes up-regulated and down-regulated in CD11b+ cells isolated from female patients with lung cancer compared to female patients with no lung cancer.

FIG. 9A shows that PBMCs from patients with active pancreatic cancer can be differentiated from controls with no active cancer. Similar results were found for 5 men and 8 women with advanced pancreatic cancer. All patients were undergoing or eligible for chemotherapy. Controls were derived from the common set of 10 age-matched men and 10 age-matched women with no history of cancer by verbal report. The compound covariate was pancreatic cancer specific. Separation by sex improved the power to differentiate between patients and controls. FIG. 9B shows genes up-regulated and down-regulated in CD11b+ cells isolated from patients with pancreatic cancer compared to controls with no pancreatic cancer. FIG. 9C shows genes up-regulated and down-regulated in CD11b+ cells isolated from female patients with pancreatic cancer compared to female patients with no pancreatic cancer.

FIG. 10A shows that PBMCs from patients with active colon cancer can be differentiated from controls with no active cancer. Similar results were found for patients advanced colon cancer. All patients were undergoing or eligible for chemotherapy. Controls were derived from the common set of 10 age-matched men and 10 age-matched women with no history of cancer by verbal report. The compound covariate was colon cancer specific. FIG. 10B shows genes up-regulated and down-regulated in CD11b+ cells isolated from patients with colon cancer compared to patients with no colon cancer.

FIG. 11 shows that PBMCs from patients with active systemic lupus erythematosus can be differentiated from controls with no active disease. Controls were derived from a common set of 10 age-matched women with no history of systemic lupus erythematosus by verbal report. Analysis revealed a unique signature that identified these patients from a control pool of ten age and sex matched controls (FIG. 11A). FIG. 11B shows genes up-regulated and down-regulated in CD11b+ cells isolated from patients with active systemic lupus erythematosus compared to patients with no systemic lupus erythematosus. This signature was also different from the cancer signatures. In addition, patients with inactive systemic lupus erythematosus could also be differentiated from the control population (FIG. 11C). FIG. 11D shows genes up-regulated and down-regulated in CD11b+ cells isolated from patients with inactive systemic lupus erythematosus compared to patients with no systemic lupus erythematosus. Patients with active systemic lupus erythematosus could also be differentiated from patients with inactive systemic lupus erythematosus (FIG. 11E). FIG. 11F shows genes up-regulated and down-regulated in CD11b+ cells isolated from patients with active systemic lupus erythematosus compared to patients with inactive systemic lupus erythematosus.

### DEFINITIONS

To facilitate an understanding of the present invention, a number of terms and phrases are defined below:

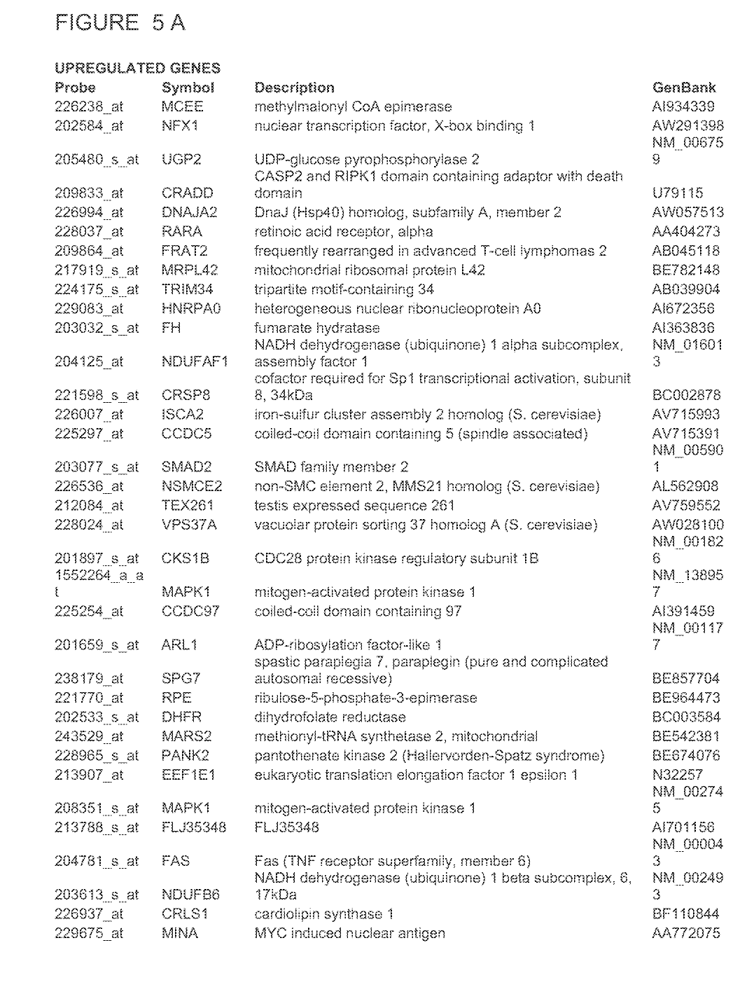

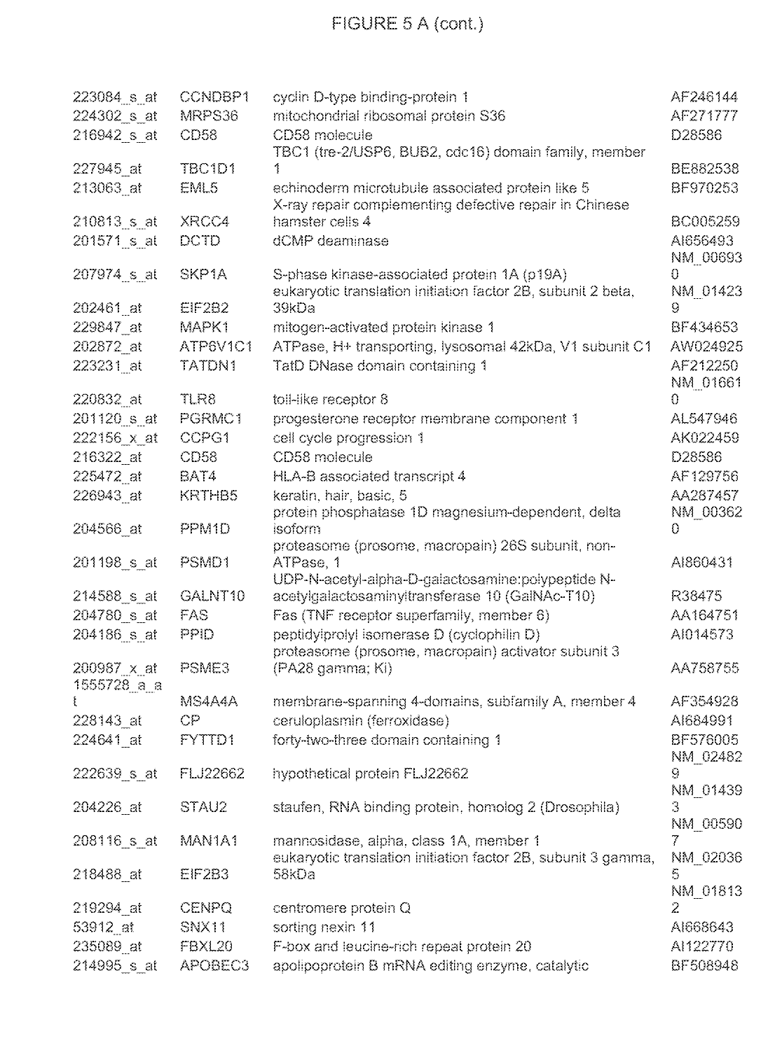

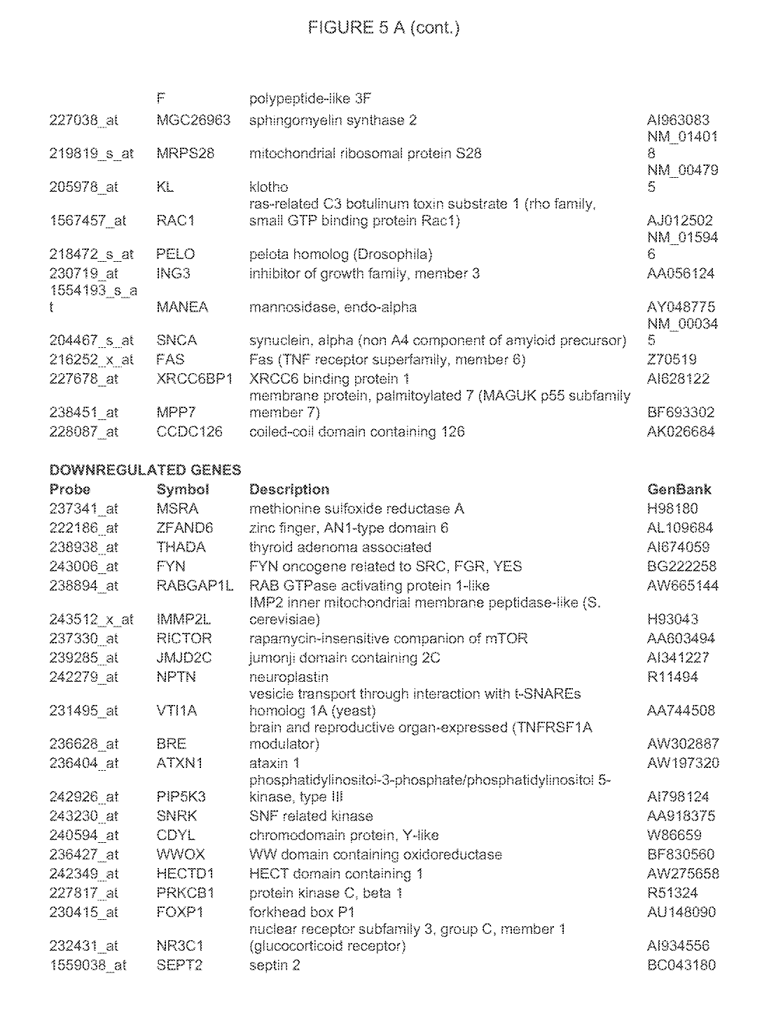

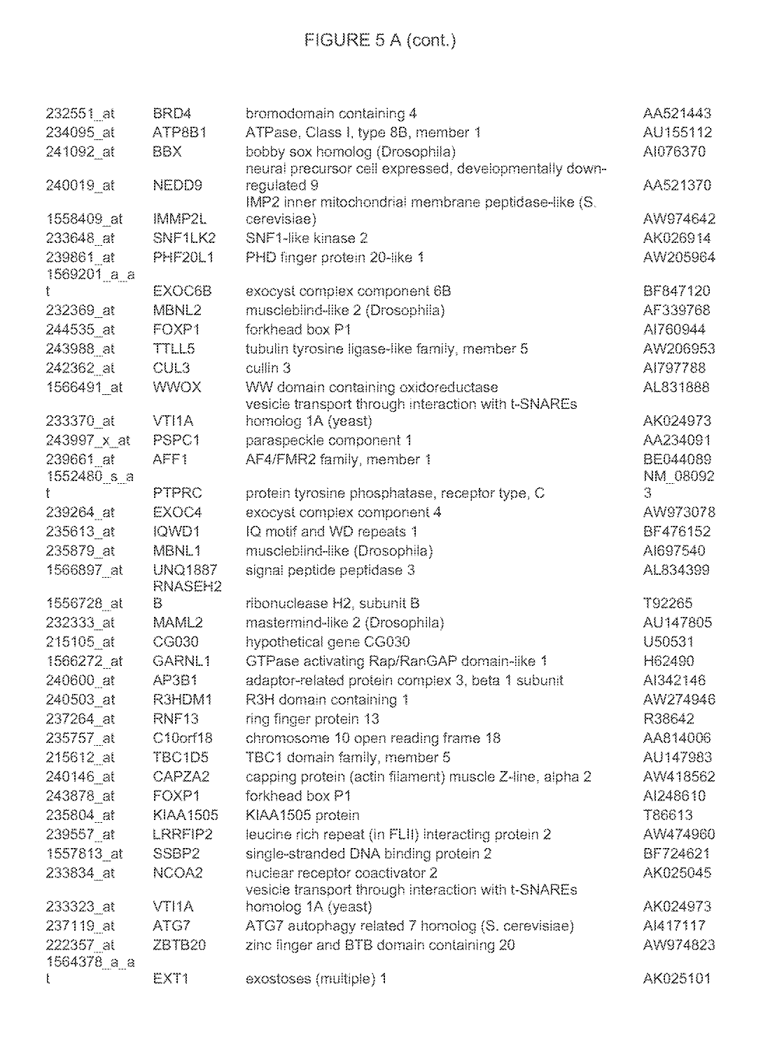

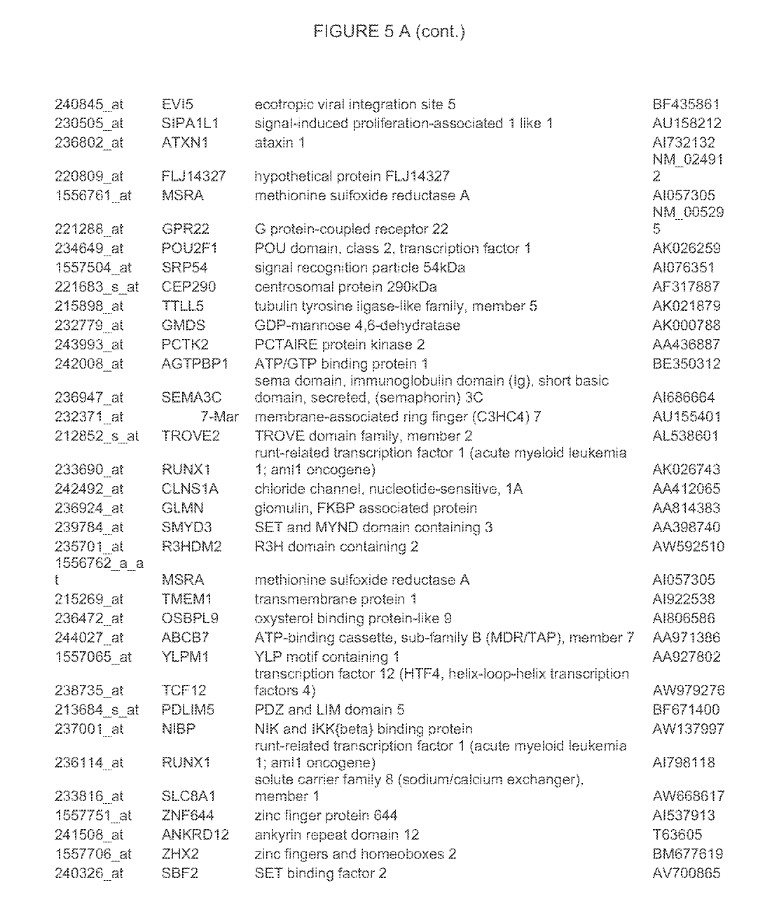

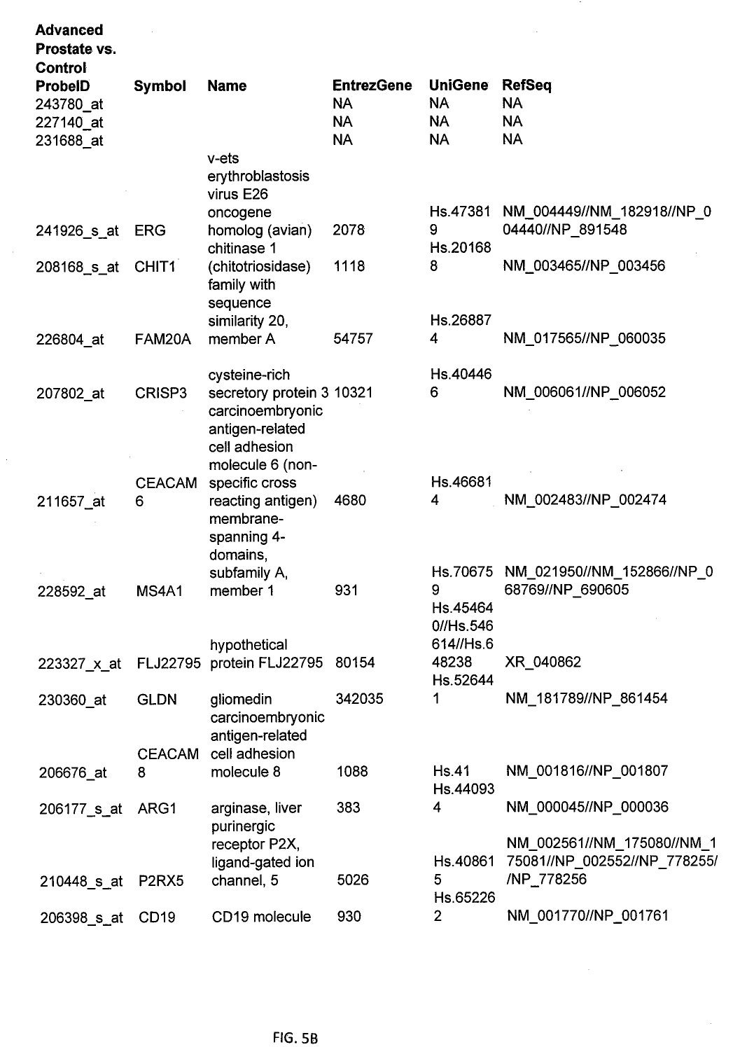

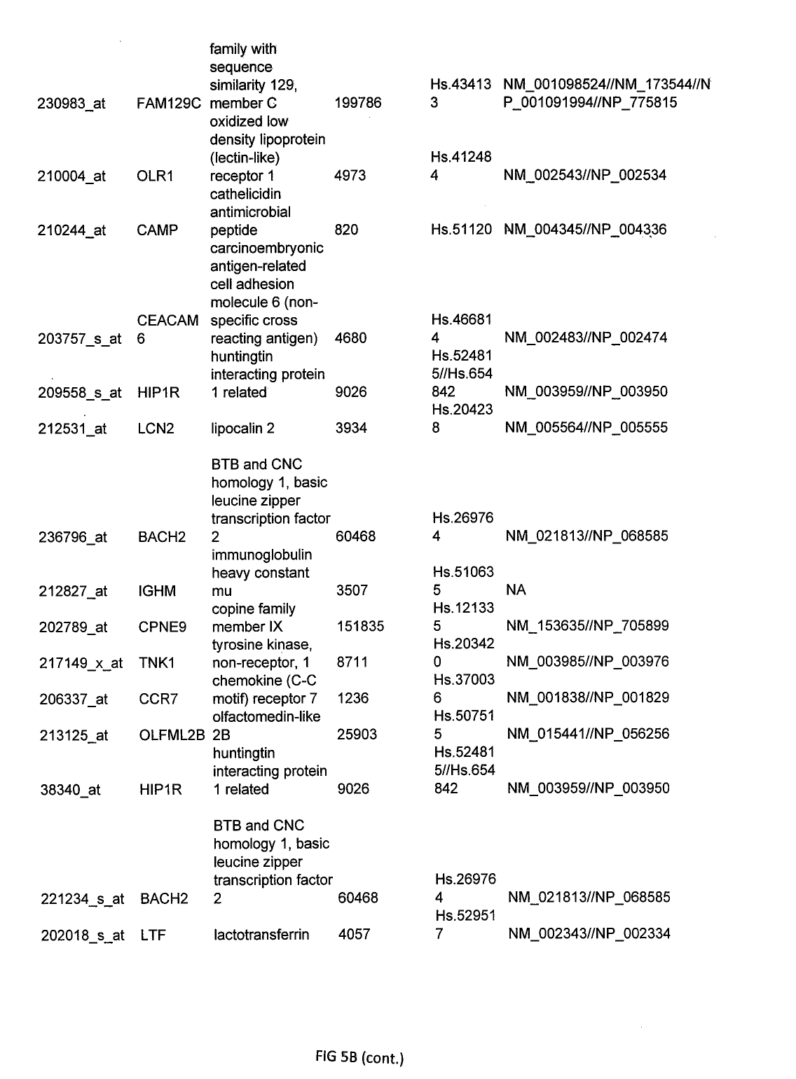

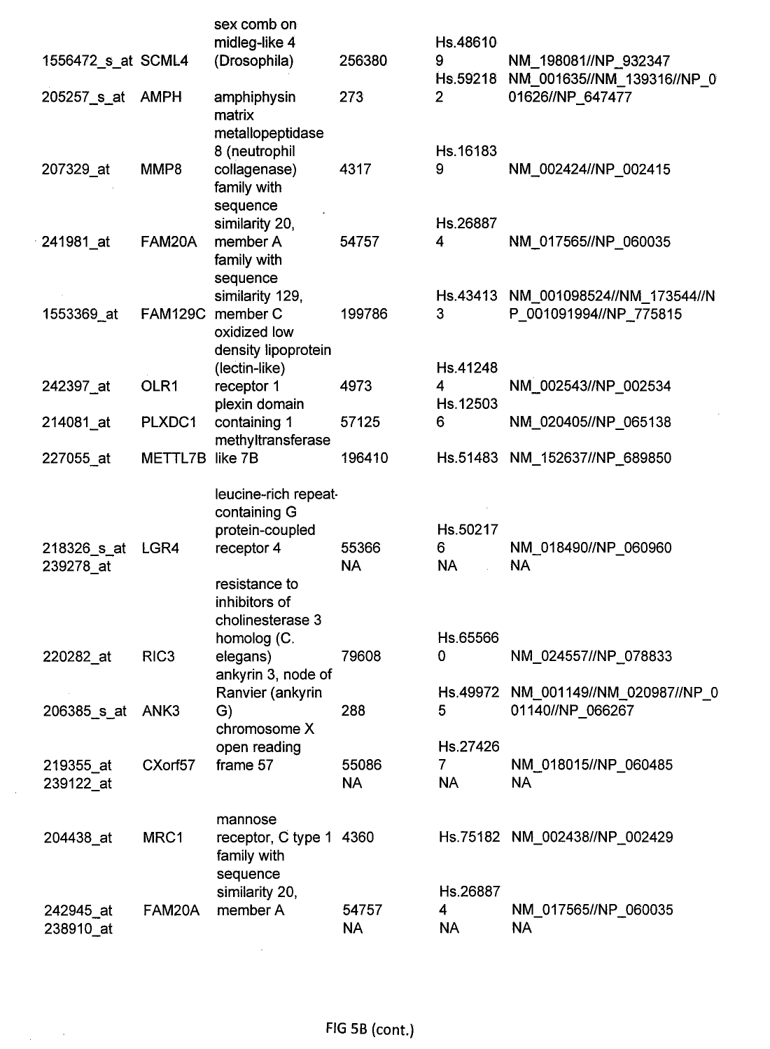

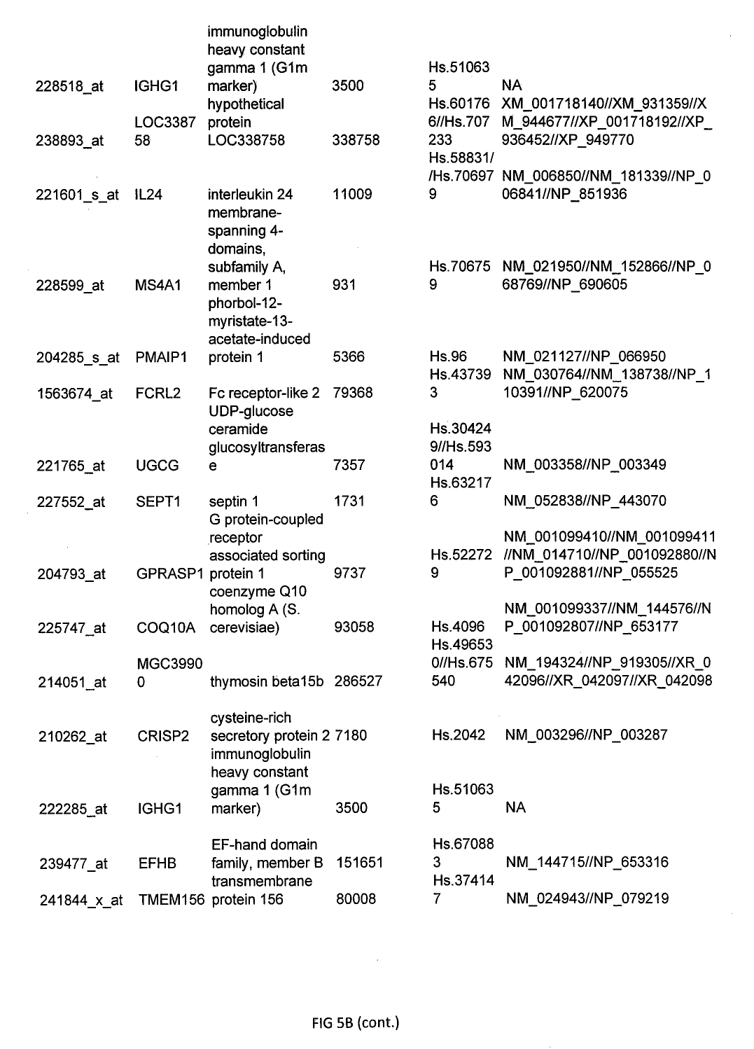

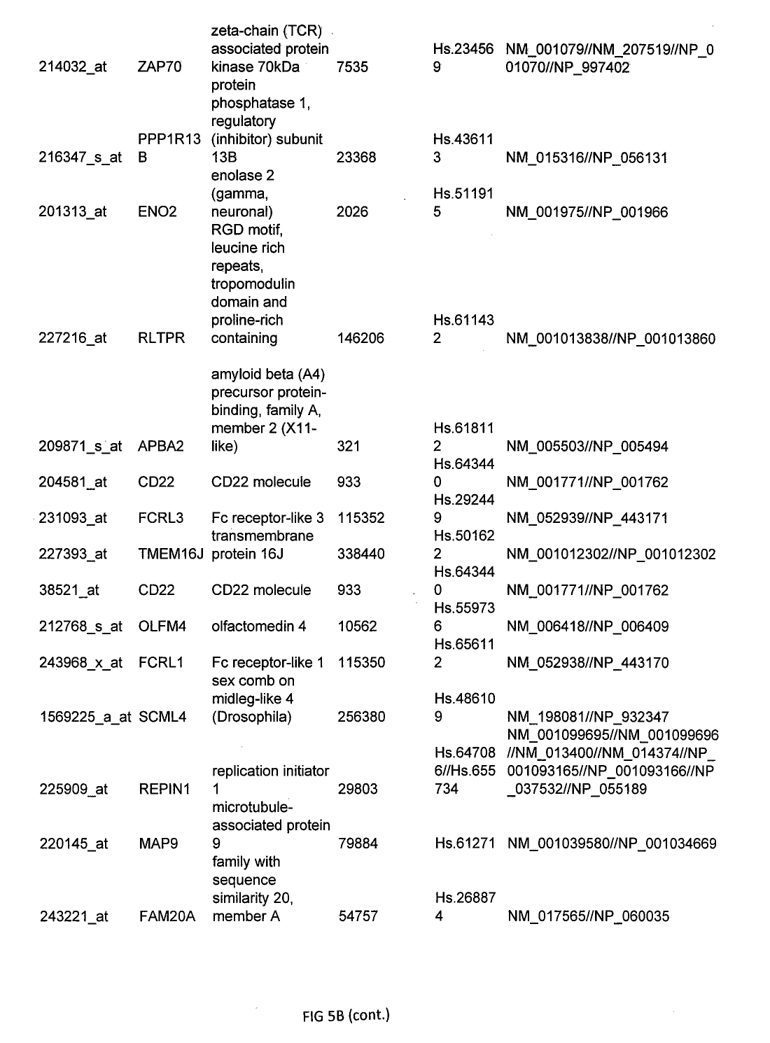

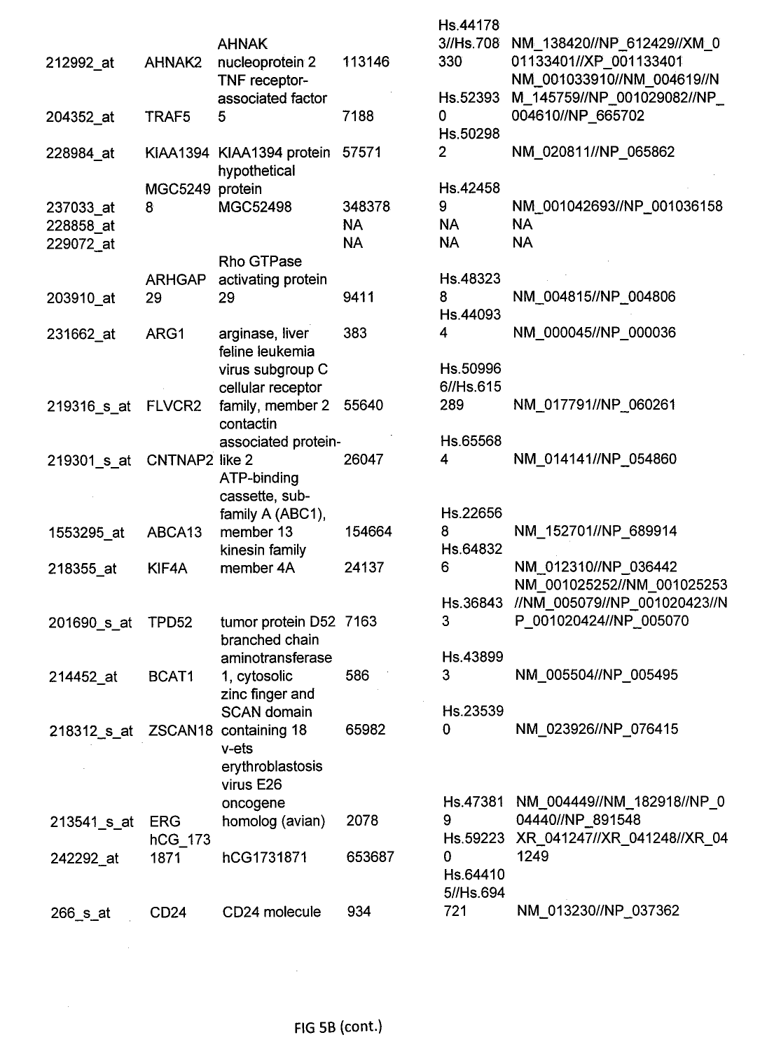

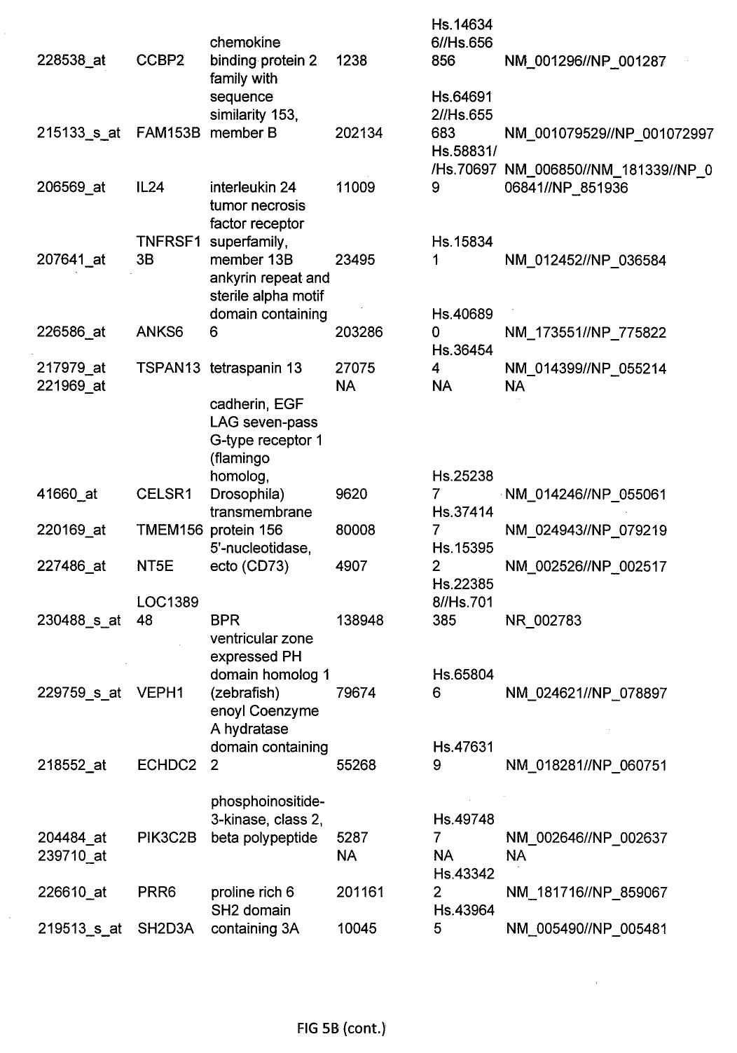

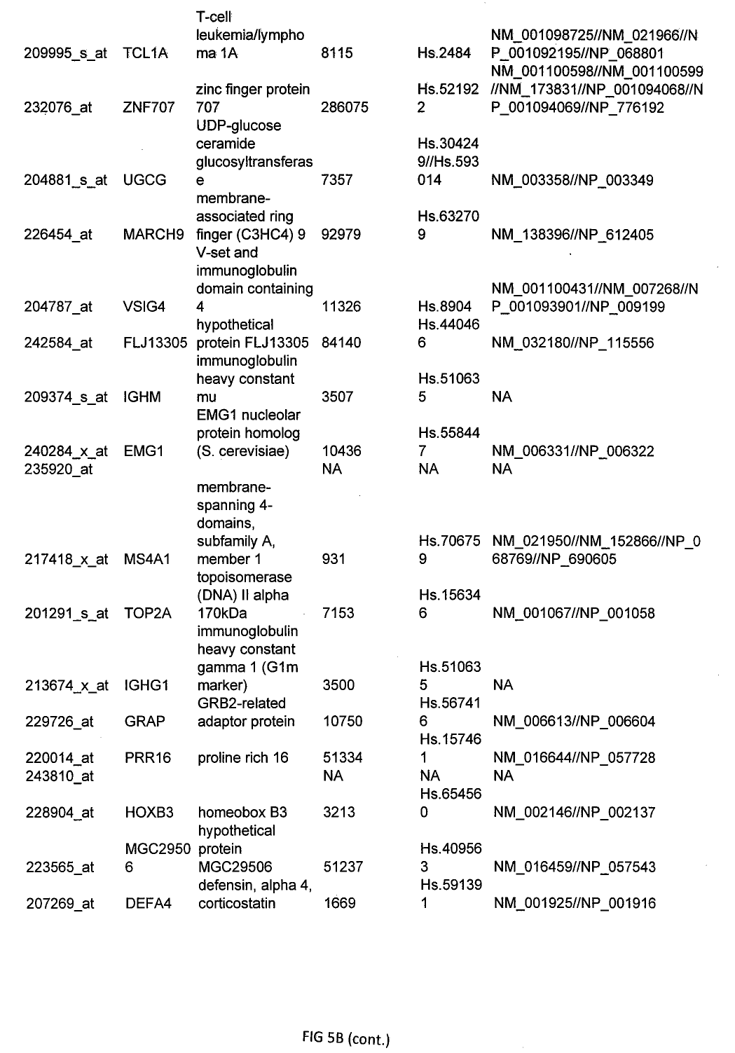

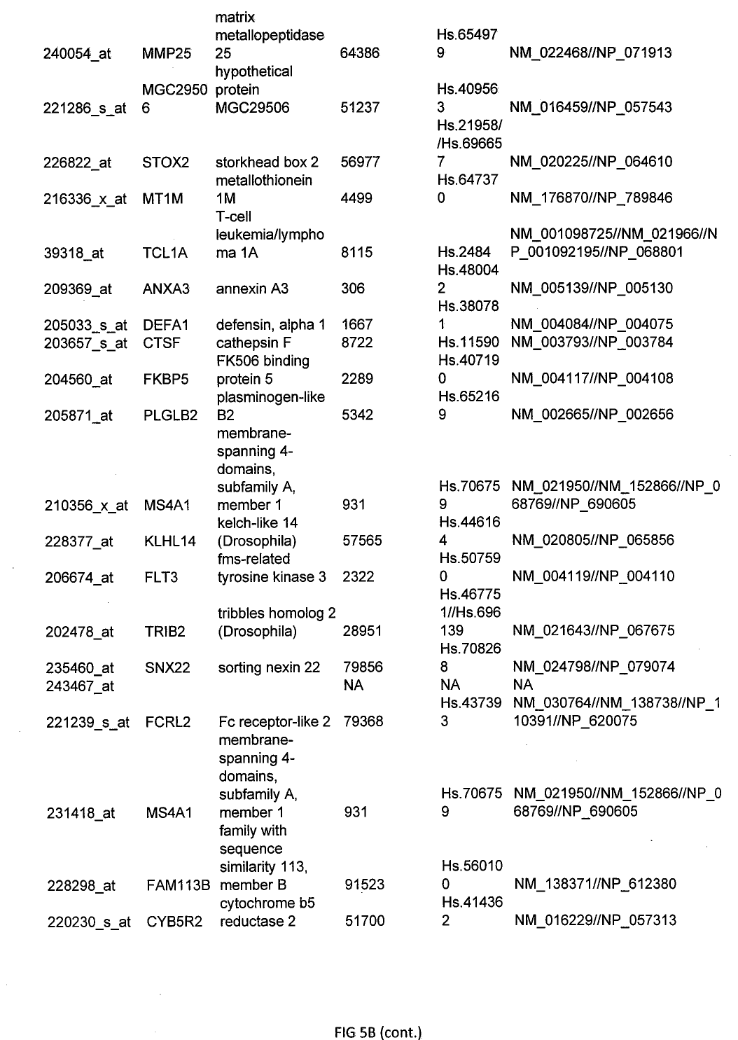

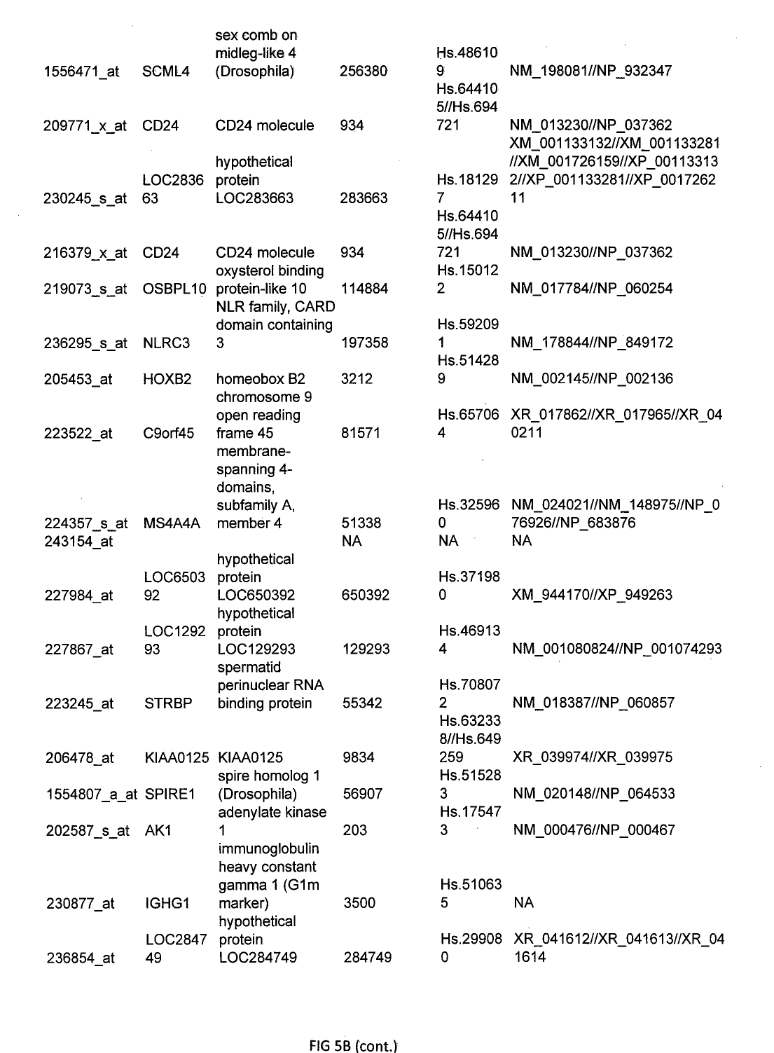

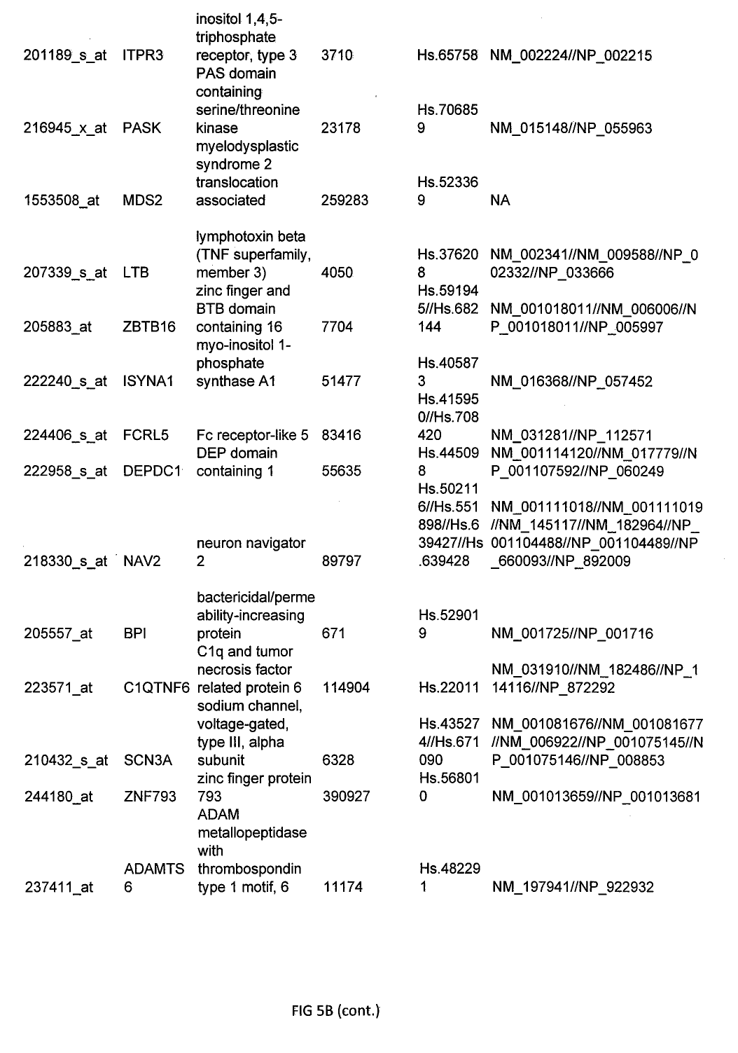

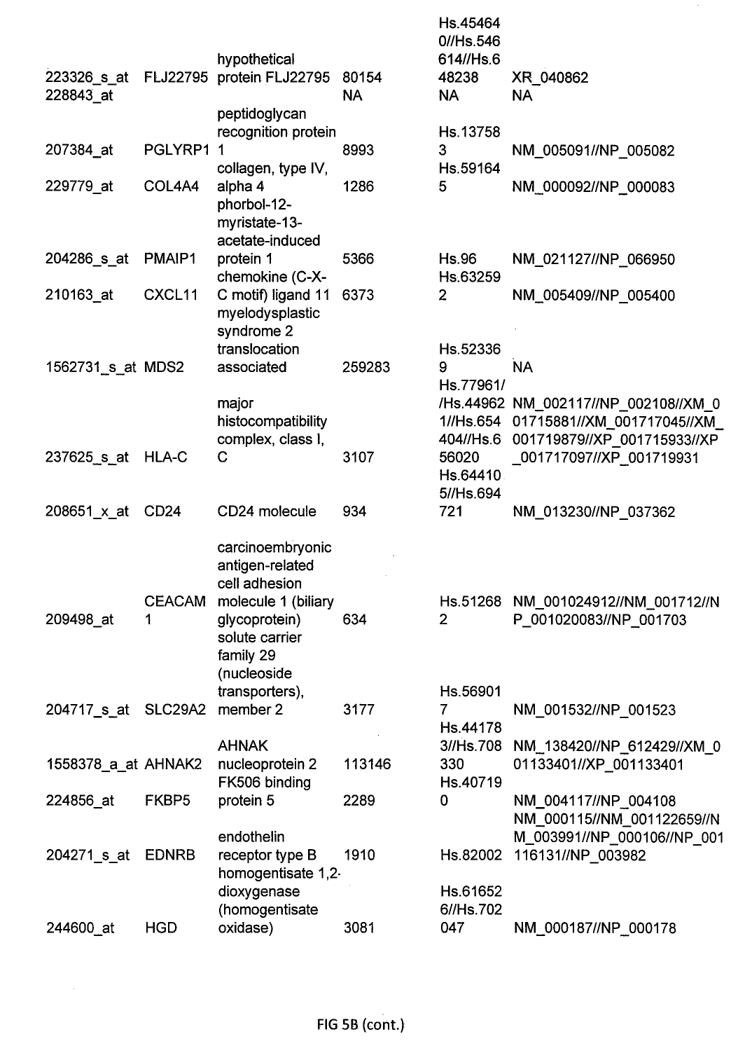

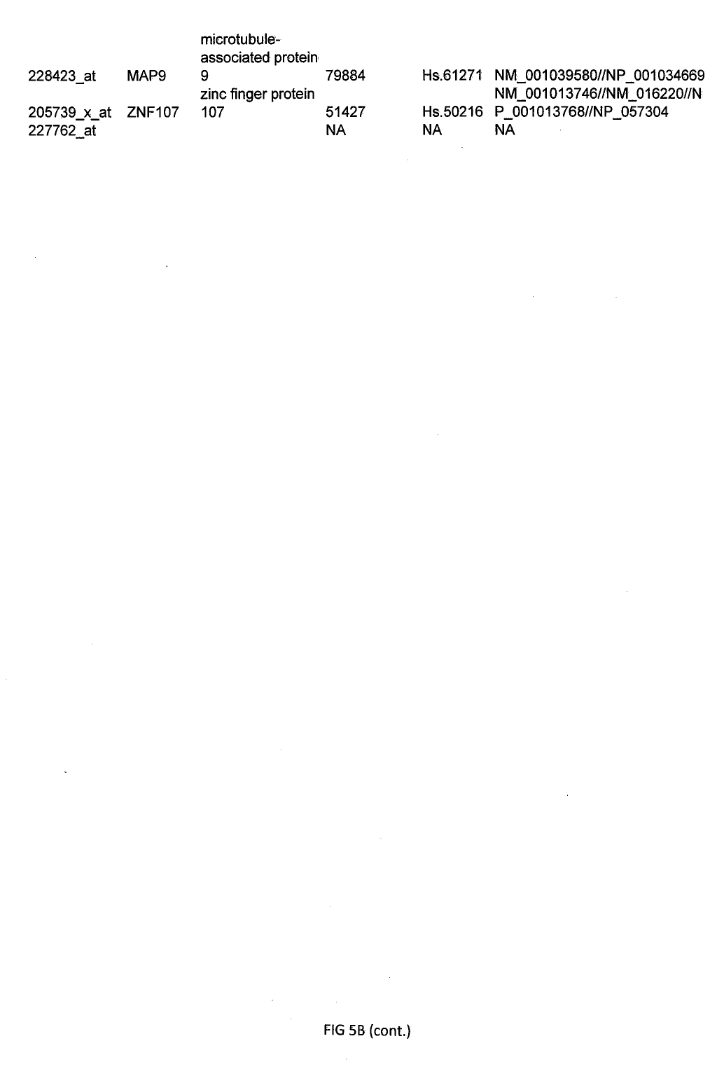

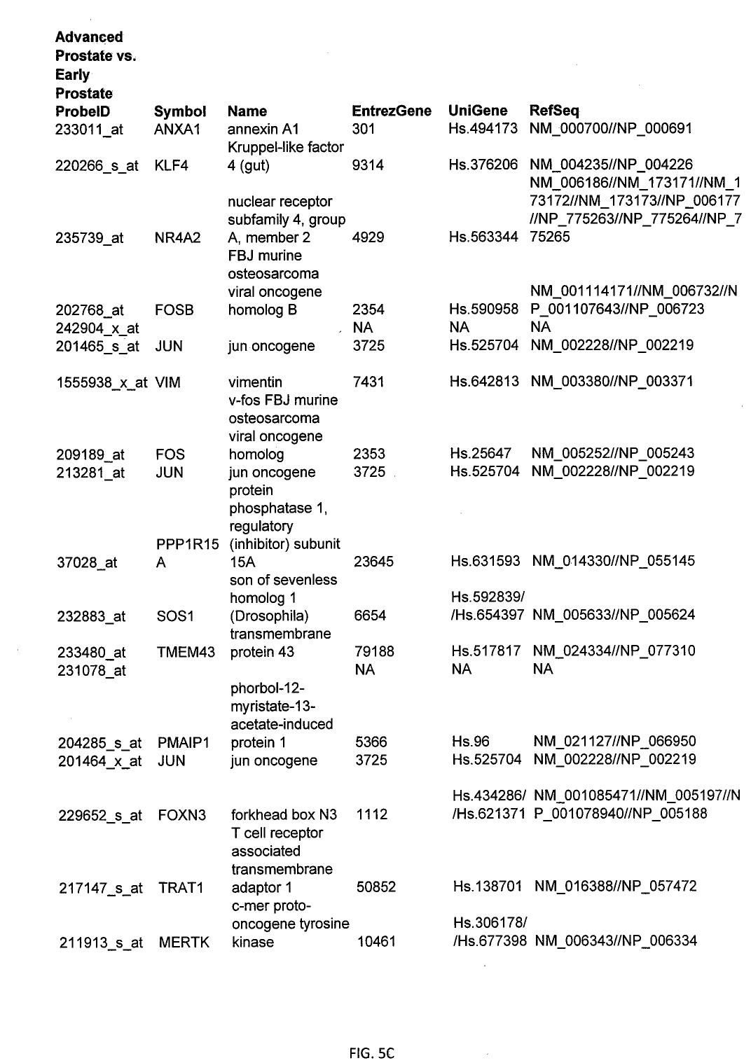

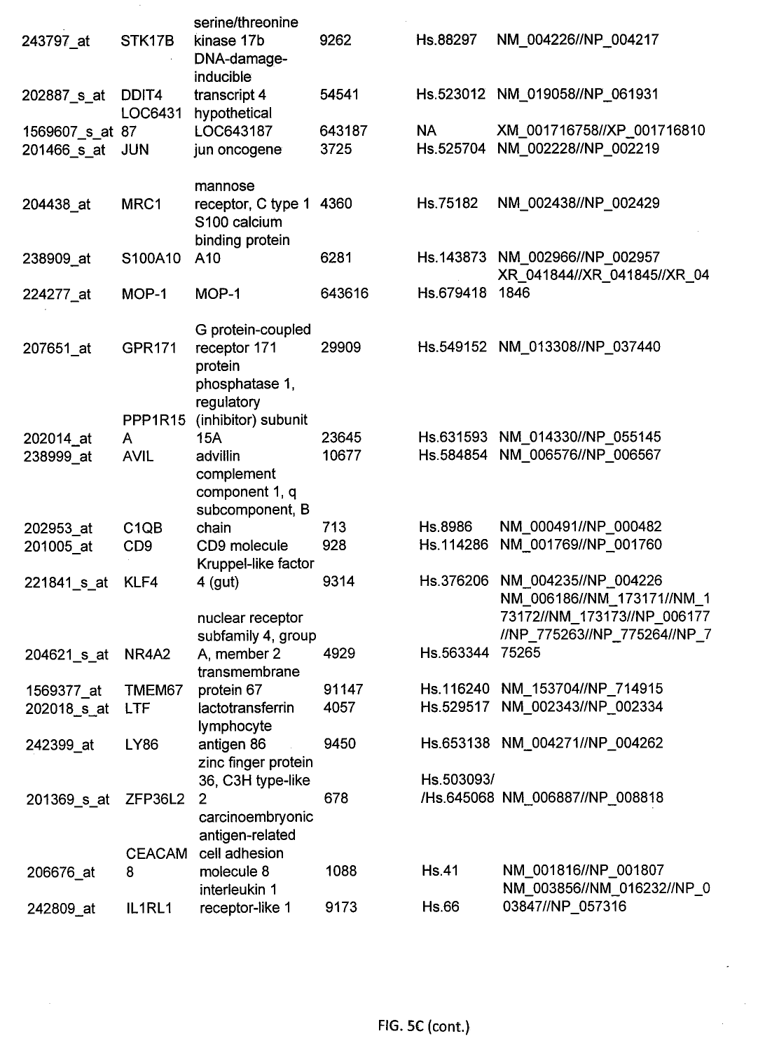

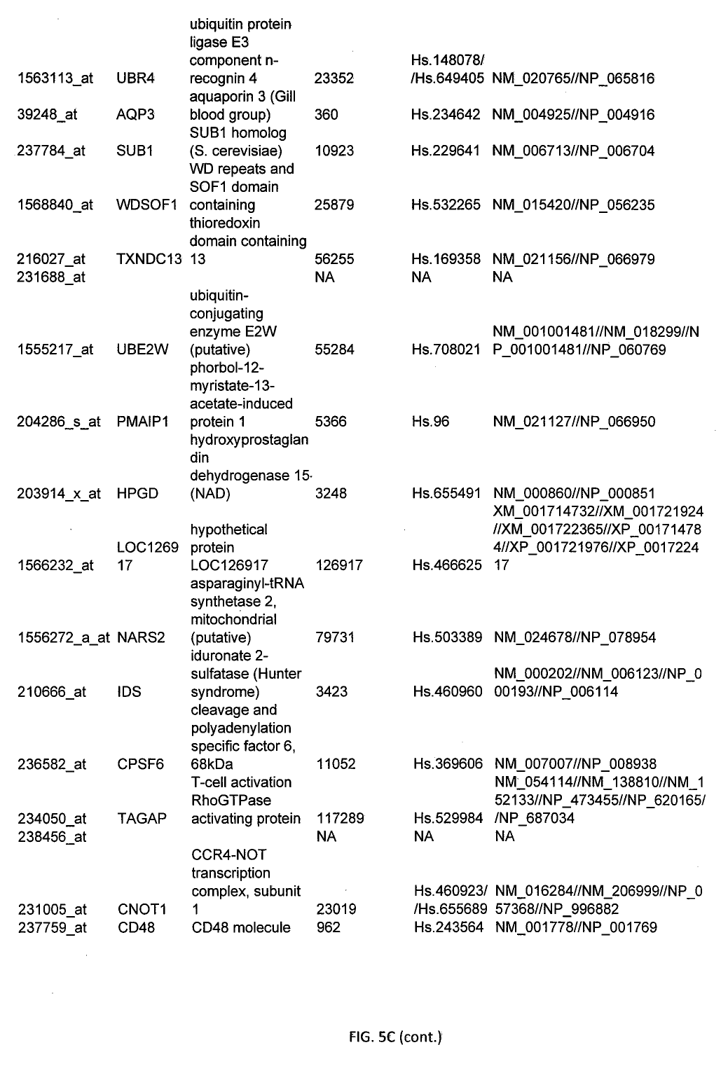

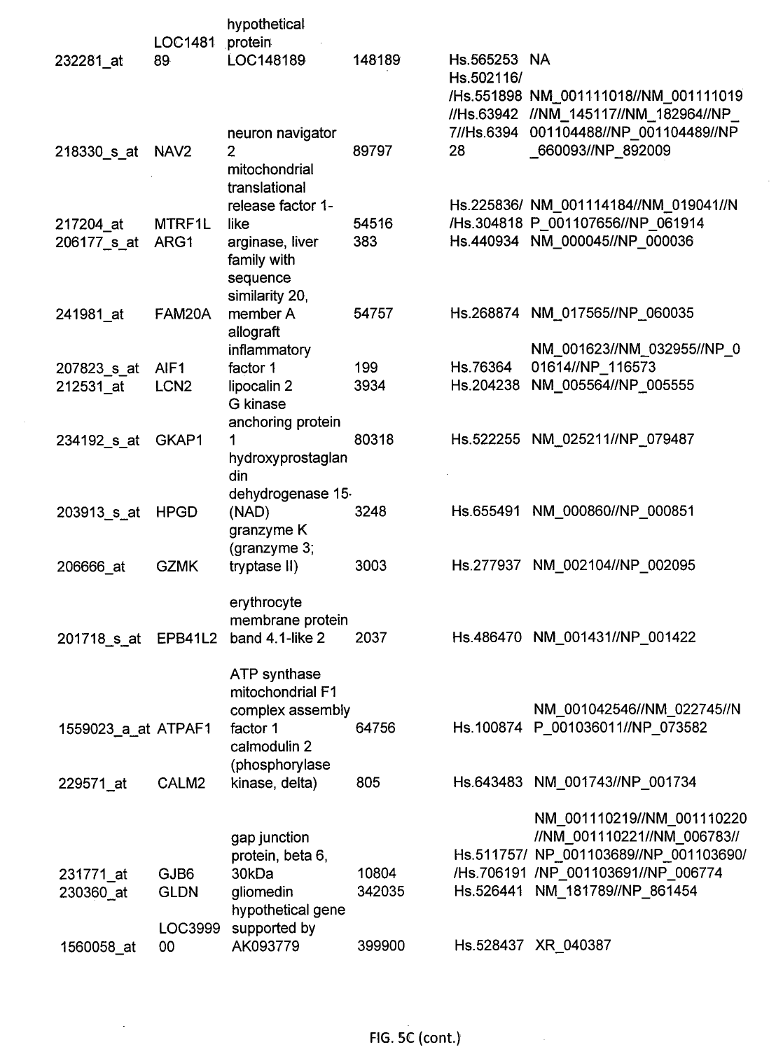

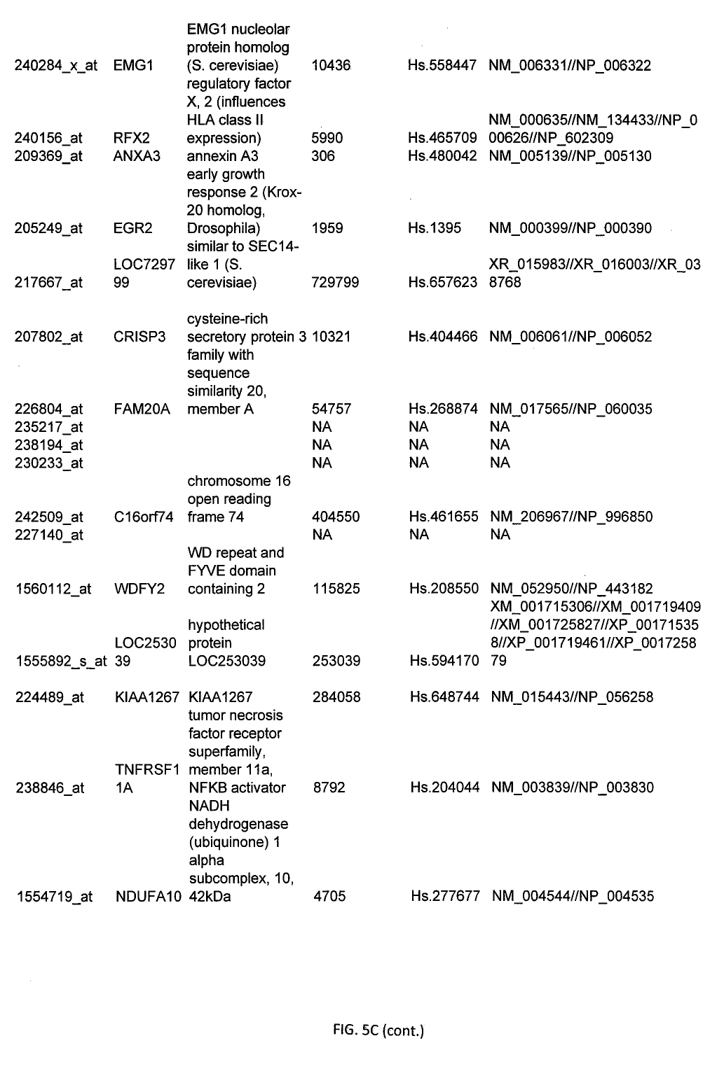

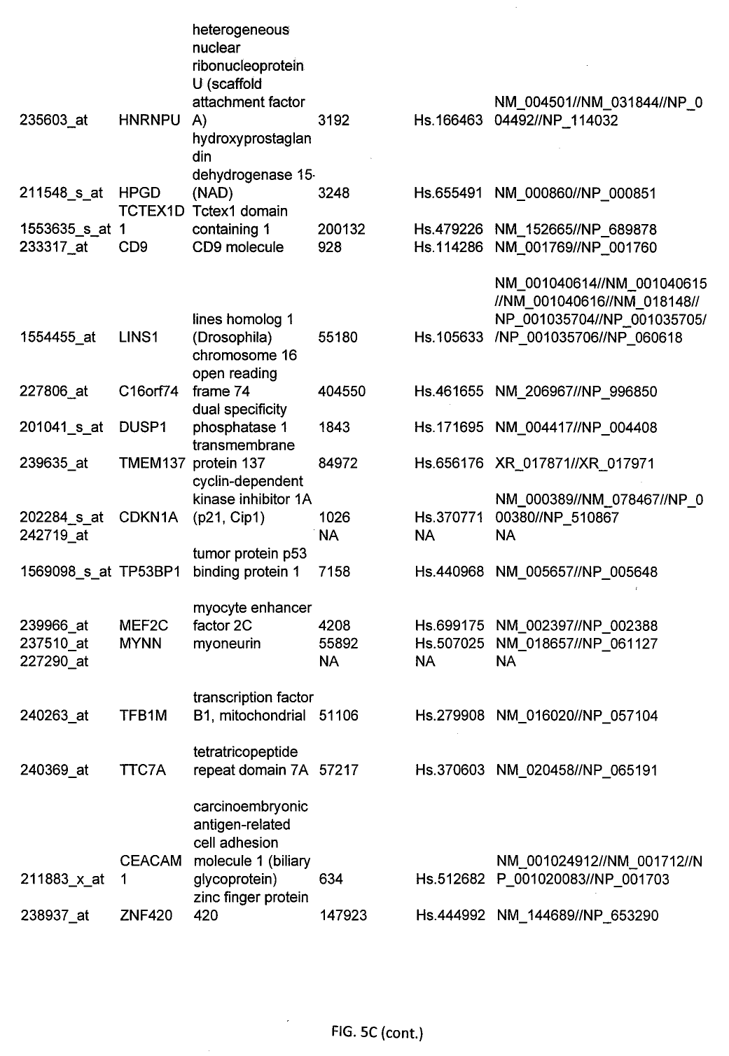

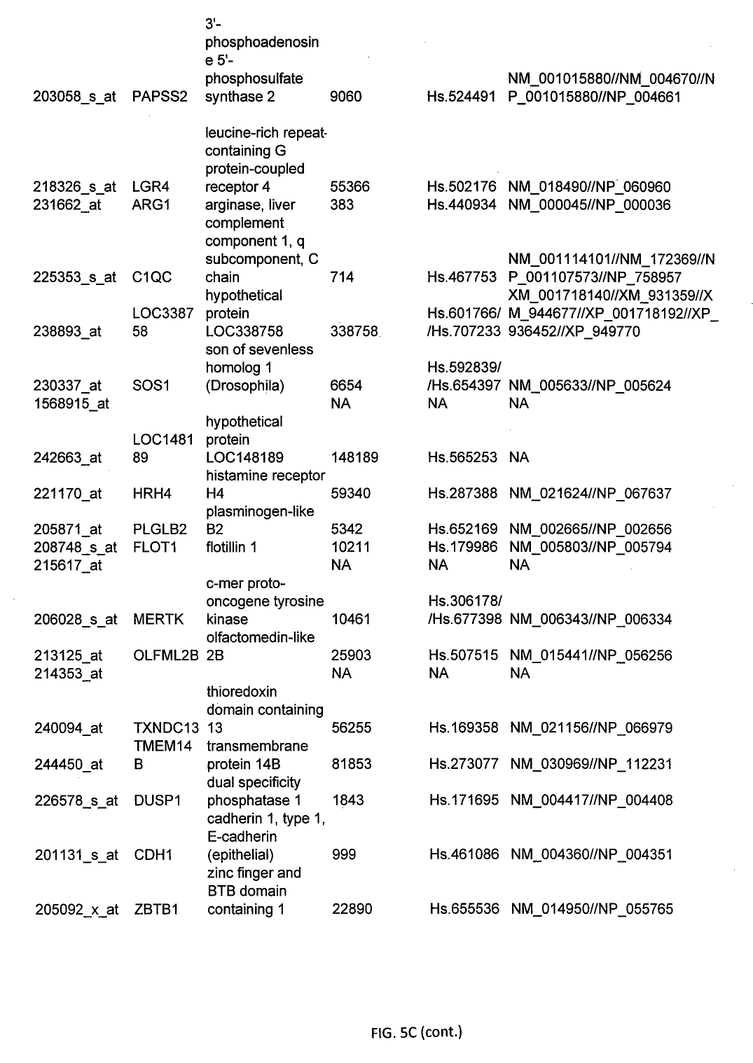

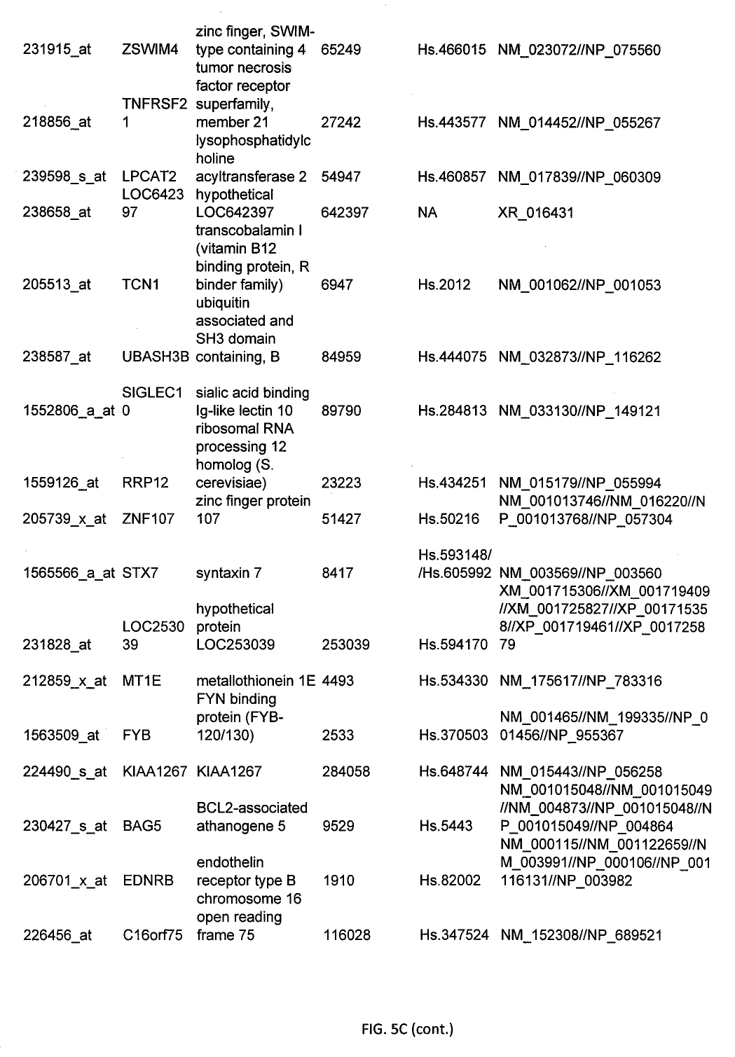

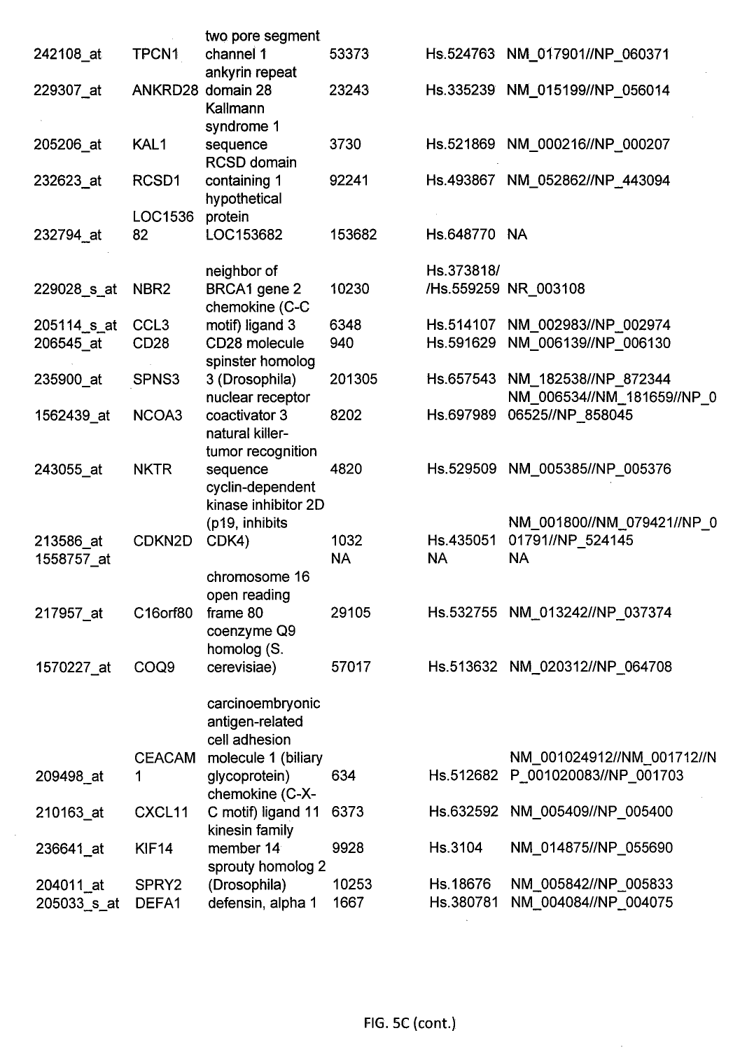

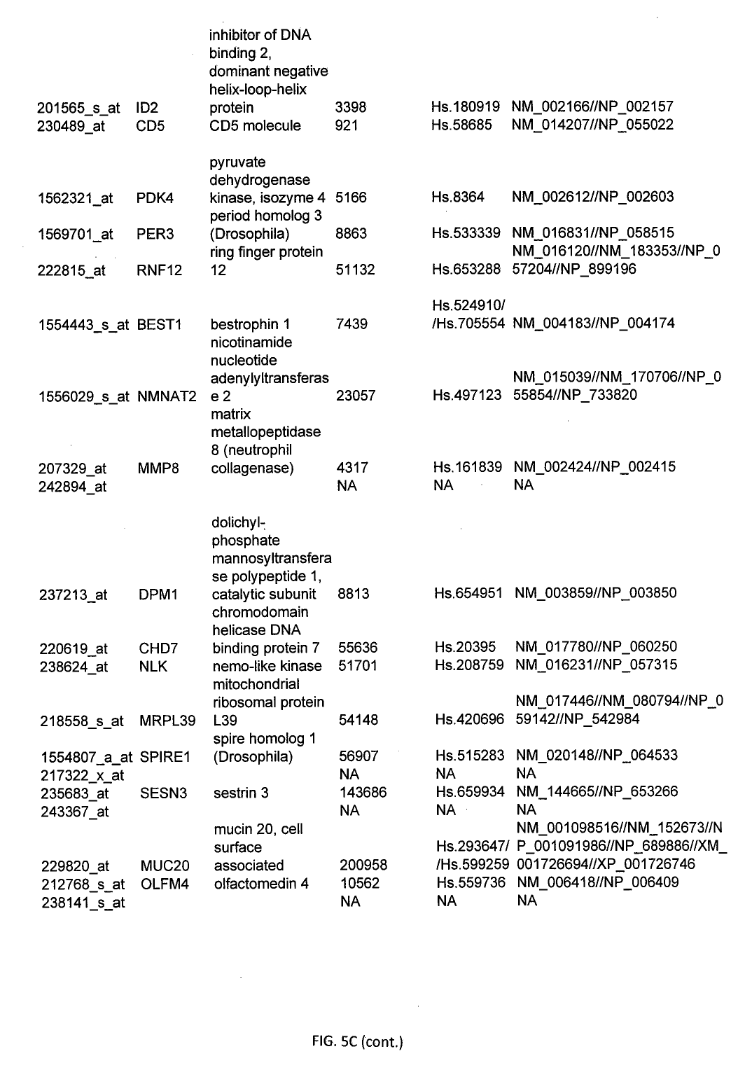

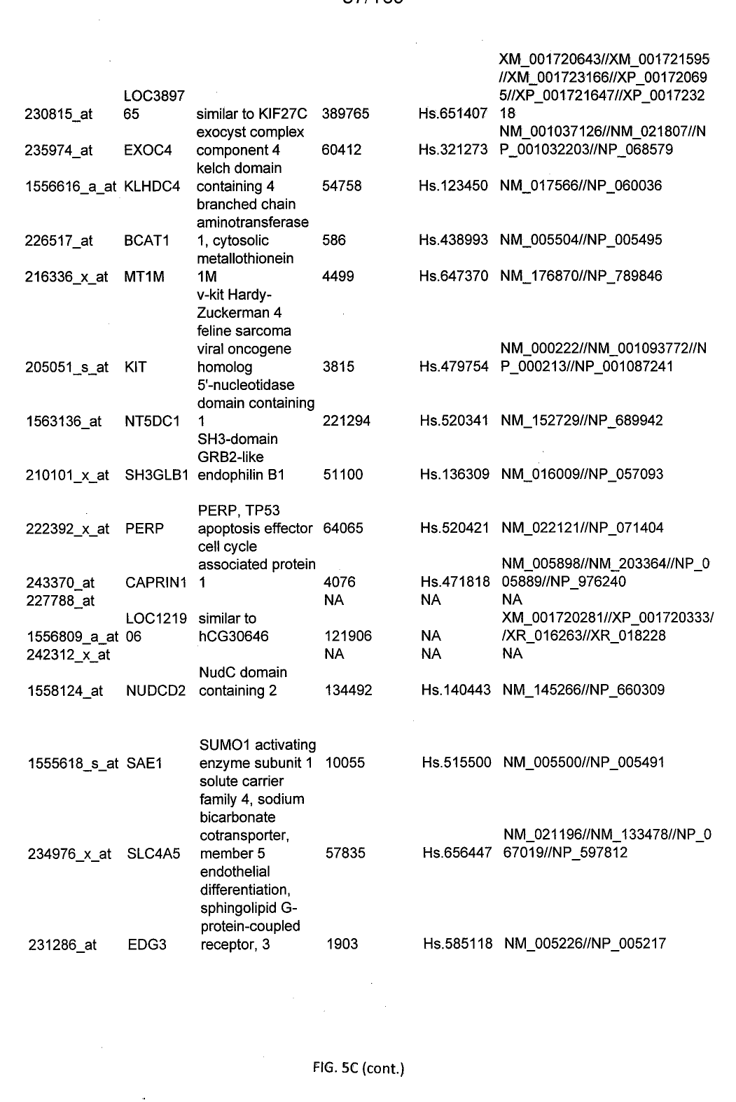

As used herein, the term “under-expression of genes within CD11b+ PBMCs” refers to a lower level of expression of specific genes within CD11b+ PBMCs (see, e.g., FIGS. 5, and 7-11) (e.g., mRNA or genomic DNA) or related protein relative to the level normally found. In some embodiments, expression is decreased at least 10%, preferably at least 20%, even more preferably at least 50%, yet more preferably at least 75%, still more preferably at least 90%, and most preferably at least 100% relative the level of expression normally found (e.g., in non-cancerous tissue). Expression levels may be determined using any suitable method, including, but not limited to, those disclosed herein.

As used herein, the term “over-expression of genes within CD11b+ PBMCs” refers to a higher level of expression of specific genes within PBMCs (see, e.g., FIGS. 5, and 7-11) (e.g., mRNA or genomic DNA) or related protein relative to the level normally found. In some embodiments, expression is increased at least 10%, preferably at least 20%, even more preferably at least 50%, yet more preferably at least 75%, still more preferably at least 90%, and most preferably at least 100% relative the level of expression normally found (e.g., in non-cancerous tissue). Expression levels may be determined using any suitable method, including, but not limited to, those disclosed herein.

A “hyperproliferative disorder,” as used herein refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo, invasion or metastasis and malignant if it does either of these. A metastatic cell or tissue means that the cell can invade and destroy neighboring body structures. Hyperplasia is a form of cell proliferation involving an increase in cell number in a tissue or organ, without significant alteration in structure or function. Metaplasia is a form of controlled cell growth in which one type of fully differentiated cell substitutes for another type of differentiated cell. Metaplasia can occur in epithelial or connective tissue cells. A typical metaplasia involves a somewhat disorderly metaplastic epithelium.

As used herein, the term “immune disorder” refers to any condition in which an organism produces antibodies or immune cells which recognize the organism's own molecules, cells or tissues. Non-limiting examples of autoimmune disorders include autoimmune hemolytic anemia, autoimmune hepatitis, Berger's disease or IgA nephropathy, Celiac Sprue, chronic fatigue syndrome, Crohn's disease, dermatomyositis, fibromyalgia, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjorgren syndrome, systemic lupus erythematosus, type 1 diabetes, ulcerative colitis, vitiligo, tuberculosis, and the like. Graft versus host disease can result from an immune response to transplanted tissues, organs and the like (e.g., bone marrow, solid organ, skin, etc.).

As used herein, the term “inflammatory disorder” refers to a condition wherein the organism's immune cells are activated. Such a condition is characterized by a persistent inflammatory response with pathologic sequelae. This state is characterized by infiltration of mononuclear cells, proliferation of fibroblasts and small blood vessels, increased connective tissue, and tissue destruction. Examples of inflammatory disorders include, but are not limited to, Crohn's disease, psoriasis, chronic obstructive pulmonary disease, inflammatory bowel disease, multiple sclerosis, and asthma. Immune diseases such as rheumatoid arthritis and systemic lupus erythematosus can also result in an inflammatory state.

As used herein, the term “subject” refers to organisms to be assessed by the methods of the present invention. Such organisms preferably include, but are not limited to, mammals (e.g., murines, simians, equines, bovines, porcines, canines, felines, and the like), and most preferably includes humans.

As used herein, the term “subject is suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention. A “preliminary diagnosis” is one based only on visual (e.g., CT scan or the presence of a lump) and antigen tests.

As used herein, the term “initial diagnosis” refers to a test result of initial cancer diagnosis that reveals the presence or absence of cancerous cells (e.g., using a biopsy and histology). An initial diagnosis does not include information about the stage of the cancer or the risk of metastasis.

As used herein, the term “post-surgical tissue” refers to tissue that has been removed from a subject during a surgical procedure. Examples include, but are not limited to, biopsy samples, excised organs, and excised portions of organs.

As used herein, the term “biopsy” refers to a tissue sample excised from a subject. Tissue samples may be obtained using any suitable method, including, but not limited to, needle biopsies, aspiration, scraping, excision using surgical equipment, etc.

As used herein, the terms “detect”, “detecting”, or “detection” may describe either the general act of discovering or discerning or the specific observation of a detectably labeled composition.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

### DETAILED DESCRIPTION OF THE INVENTION

The American Cancer Society estimates that over 1,437,000 people will be diagnosed with cancer in the United States in 2008 and that 565,650 will die of the collection of diseases that we term cancer (see, e.g., CA Cancer J Clin. 2008 March-April; 58(2):71-96; each herein incorporated by reference in its entirety). The National Institutes of Health estimate overall costs of cancer in 2007 at $219.2 billion: $89.0 billion for direct medical costs (total of all health expenditures); $18.2 billion for indirect morbidity costs (cost of lost productivity due to illness); and $112.0 billion for indirect mortality costs (cost of lost productivity due to premature death) (see, e.g., CA Cancer J Clin. 2008 March-April; 58(2):71-96; each herein incorporated by reference in its entirety). Although inroads have been made in diagnosis and treatment, cancer remains a major health burden in the United States and the world.

It is widely recognized that cancer becomes lethal because it spreads from the primary organ of origin and metastasizes to sites around the body (see, e.g., CA Cancer J Clin. 2007 July-August; 57(4):225-41; Proc Natl Acad Sci USA. 2003 Feb. 4; 100(3):776-81; Cell. 2000 Jan. 7; 100(1):57-70; each herein incorporated by reference in their entireties). Metastatic cancer, except for a few exceptions, is not curable and leads to the death of the patient. It is generally accepted that if cancer could be diagnosed and treated before it had the time and opportunity to metastasize, it would be more frequently cured. The fact that the 5-year survival rate for cancer has increased from 50% in 1975 to 66% in 2003 is explained by improvements in early diagnosis combined with effective treatments with surgery and/or radiation. The need for new biomarkers for cancer detection, therefore, is clear.

Many malignant tumors are associated with a leukocytic infiltrate that consists mainly of macrophages (often called tumor-associated macrophages, TAMs), which in some instances, comprise up to 70% of the cell tumor mass (see, e.g., Kelly, et al., Br J Cancer, 57: 174-177, 1988; herein incorporated by reference in its entirety). These cells are an essential cellular component of the innate immune system and are derived from myeloid progenitor cells in the bone marrow compartment. These progenitor cells develop into pro-monocytes and are released into the circulation where they undergo differentiation into monocytes. Monocytes then migrate into tissues where they differentiate into resident tissue macrophages and help to protect these sites from infection and injury (or in the case of cancer monocytes differentiate into TAMs which are capable of promoting tumor growth and metastasis). In addition to their role in innate immunity, recent evidence suggests that macrophages also play an important role in the regulation of angiogenesis in both normal and diseased tissues, including malignant tumors (see, e.g., Crowther, et al., J Leukoc Biol, 70: 478-490, 2001; Craig, et al., J Cell Biochem. 2008 Jan. 1; 103(1):1-8; each herein incorporated by reference in their entireties). The propensity of infiltrating TAMs to promote tumor growth and metastasis is becoming clear (see, e.g., Kelly, et al., Br J Cancer, 57: 174-177, 1988; Crowther, et al., J Leukoc Biol, 70: 478-490, 2001; each herein incorporated by reference in their entireties). Some evidence suggests that the origins of the infiltrating TAMs result from the phenotypic differentiation of resident macrophages that are already in the healthy tissue before tumor develops/metastasizes (see, e.g., Stephens, et al., Br J Cancer, 38: 573-582, 1978; herein incorporated by reference in its entirety). Alternative evidence suggests that macrophage precursor cells (monocytes) are recruited from the circulation are induced to differentiate by the tumor mileu upon recruitment (see, e.g., Mantovani, et al., Immunol Today, 13: 265-270, 1992; herein incorporated by reference in its entirety). A growing body of evidence suggests that cancer can alter the bone marrow microenvironment prior to metastatic disease (see, e.g., Shiozawa, et al., J Cell Biochem. 2008 Oct. 1; 105(2):370-80; Havens, et al., Neoplasia. 2008 April; 10(4):371-80; each herein incorporated by reference in their entireties). Elevated expression of a number of monocyte chemoattractants, including CCL2, CCL3, CCL4, CCL8 and CCL5 (RANTES) by both tumor and stromal cells within tumors have been shown to positively correlate with increased TAM numbers in many human tumors (see, e.g., Murdoch, et al., Blood, 104: 2224-2234, 2004; Milliken, et al., Clin Cancer Res, 8: 1108-1114, 2002; Azenshtein, et al., Cancer Res, 62: 1093-1102, 2002; each herein incorporated by reference in their entireties). When associated with tumors, macrophages demonstrate what has been described as a “polarization” towards one of two phenotypically different subsets of macrophages: M1 macrophages (classically activated) or M2 macrophages (alternatively activated) (see, e.g., Sica, et al., Eur J Cancer, 42: 717-727, 2006; Nature. 2008 Jul. 24; 454(7203):436-44; each herein incorporated by reference in their entireties). M1 macrophages are known to produce pro-inflammatory cytokines and play an active role in cell destruction while M2 macrophages primarily scavenge debris and promote angiogenesis and wound repair (see, e.g., Sica, et al., Eur J Cancer, 42: 717-727, 2006; Nature. 2008 Jul. 24; 454(7203):436-44; Lewis, et al., Cancer Res, 66: 605-612, 2006; Mantovani, et al., Immunol Today, 13: 265-270, 1992; each herein incorporated by reference in their entireties). The M2 macrophage population is phenotypically similar to the tumor-associated macrophage population that promotes tumor growth and development (see, e.g., Sica, et al., Eur J Cancer, 42: 717-727, 2006; Nature. 2008 Jul. 24; 454(7203):436-44; Lewis, et al., Cancer Res, 66: 605-612, 2006; Mantovani, et al., Immunol Today, 13: 265-270, 1992; each herein incorporated by reference in their entireties).

Given the recruitment of monocytes to the tumor, and the subsequent importance of TAMs to tumorigenesis, experiments conducted during the development of embodiments for the present invention demonstrated that the presence of cancer and other disorders and conditions (e.g., other hyperproliferative disorders, inflammatory disorders, immune disorders) in the body can be detected by circulating myeloid cells and that these cells undergo genetic changes to respond to the presence of a particular disorder (e.g., inflammatory disorder (e.g., systemic lupus erythematosus), hyperproliferative disorder (e.g., cancer)). It was demonstrated these changes can be detected in gene expression within the circulating myeloid cells and that these changes serve as a biomarker to detect the presence of a particular disorder (e.g., systemic lupus erythematosus, cancer (e.g., lung cancer, colon cancer, breast cancer, prostate cancer, pancreatic cancer)). It was shown that the ability of a xenograft tumor of a human prostate cancer murine model dictates the molecular profile in cells of the monocyte lineage and identifies a genetic signature in CD11b+ PBMCs that reflects the presence of cancer. It was shown, for example, that the presence of a prostate tumor changes the genetic profile of circulating CD11b+ PBMCs compared to autologous monocytes to a genotype/phenotype that promotes tumorigenesis and metastases. It was shown that a gene signature from CD11b+ PBMCs can be detected in men with prostate cancer that differentiates them from age-matched control men with no clinical evidence of prostate cancer. The CD11b+ signature is similar in prostate cancer mouse models as well as in human disease. In addition, it was demonstrated that unique CD11b+ gene signatures can be detected for men and women with a variety of cancer types (e.g., lung cancer, colon cancer, pancreatic cancer, breast cancer, prostate cancer, and colon cancer) in a tumor specific fashion, and in inflammatory disorders (e.g., systemic lupus erythematosus).

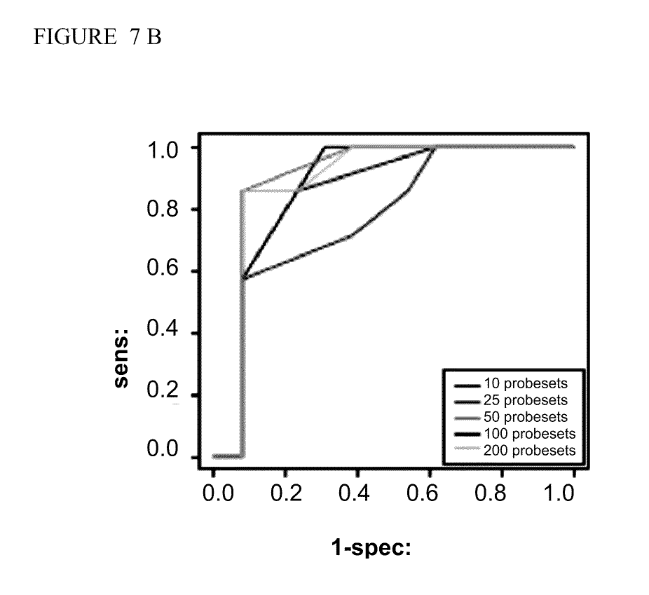

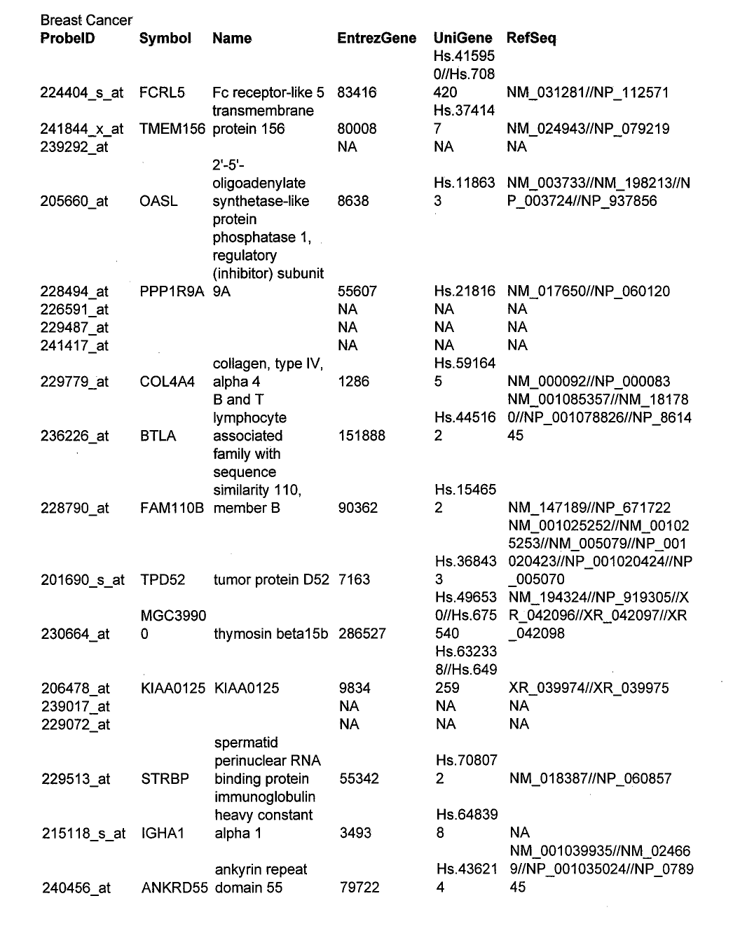

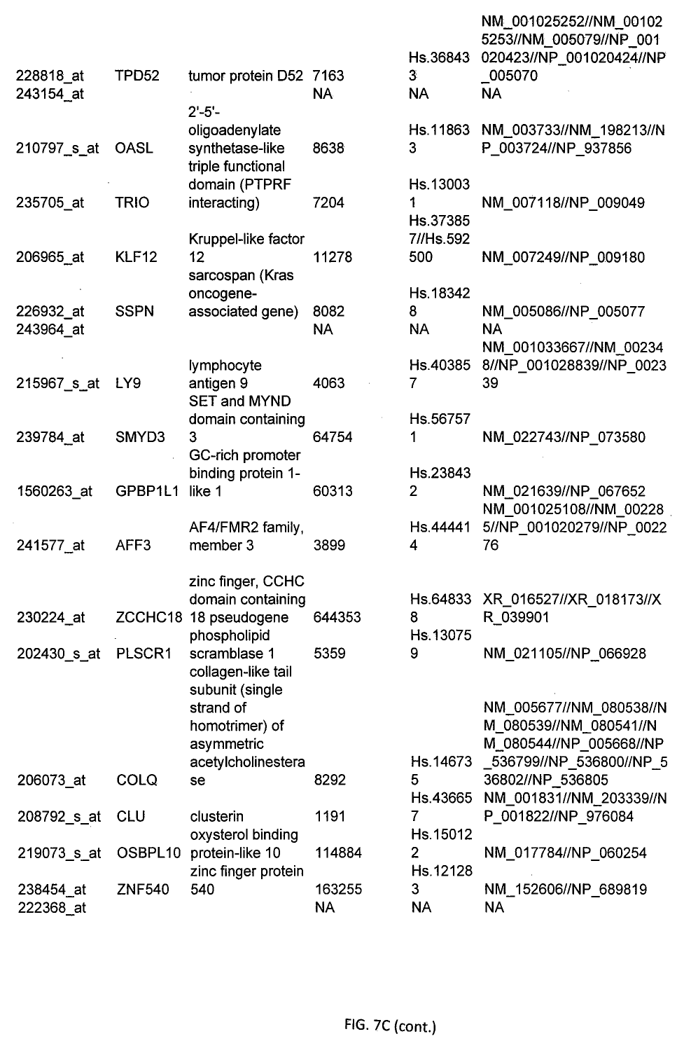

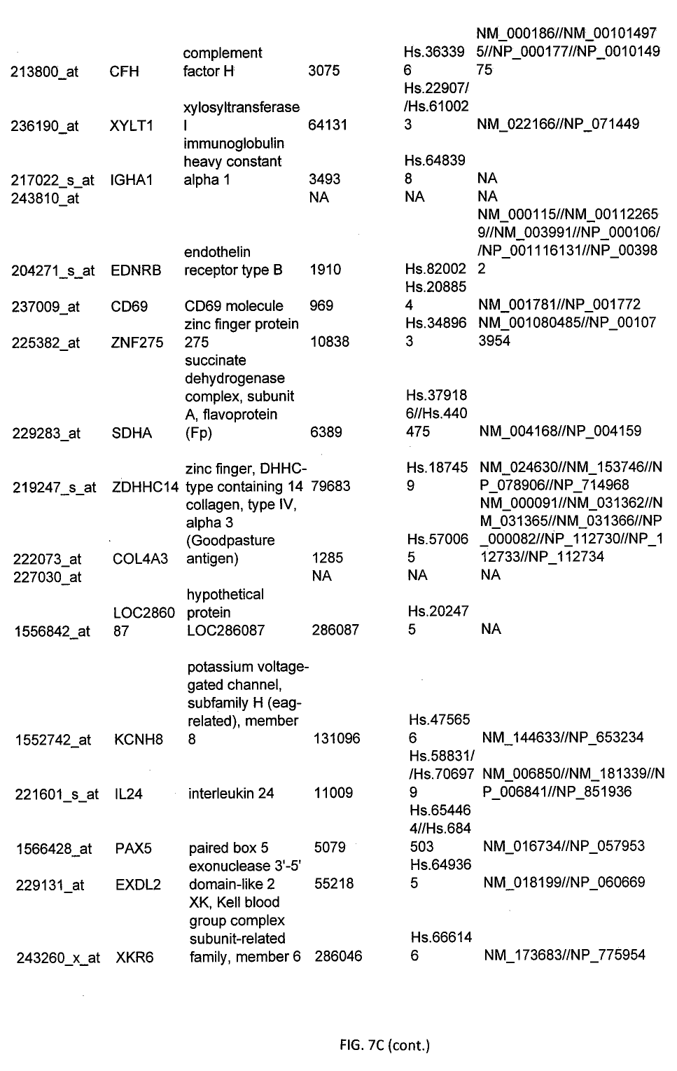

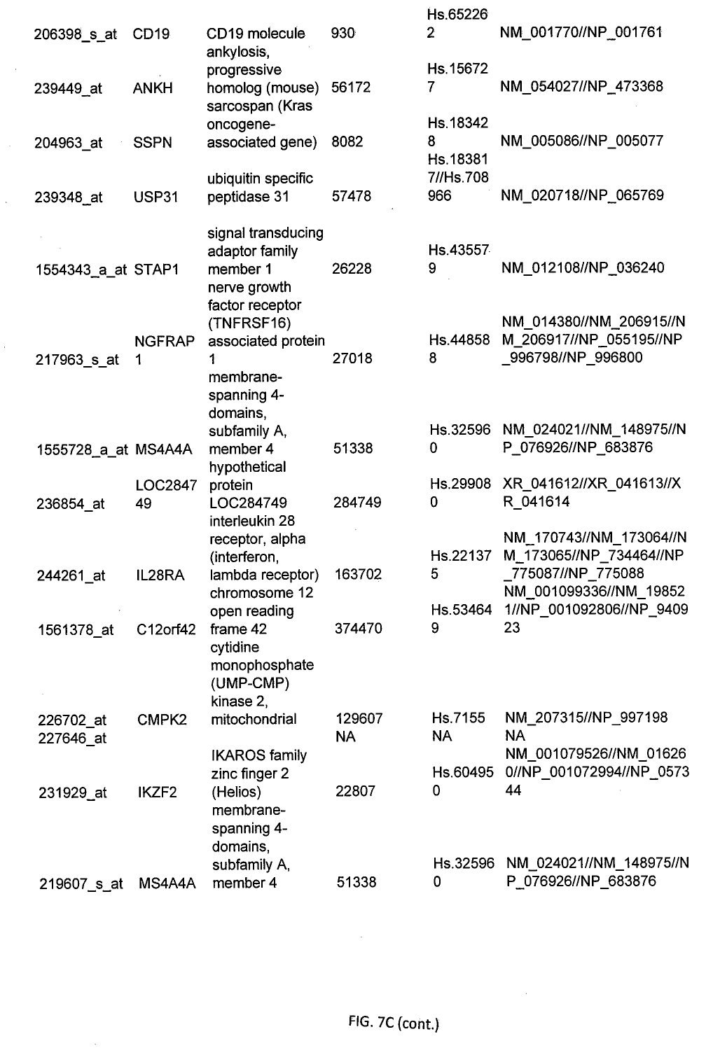

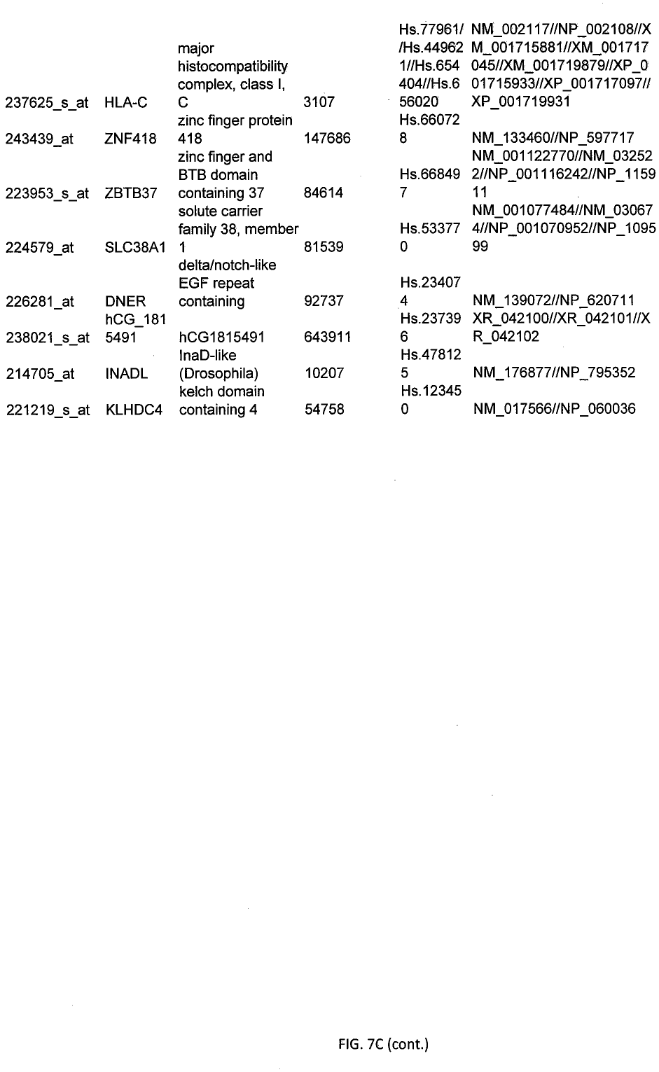

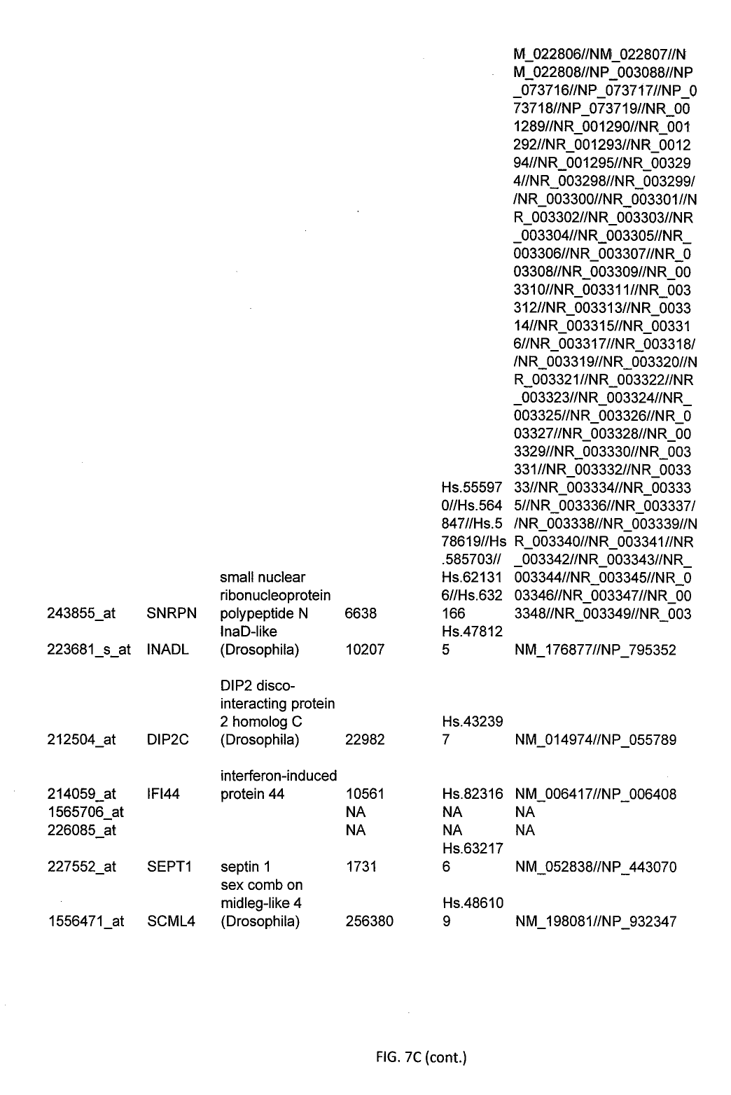

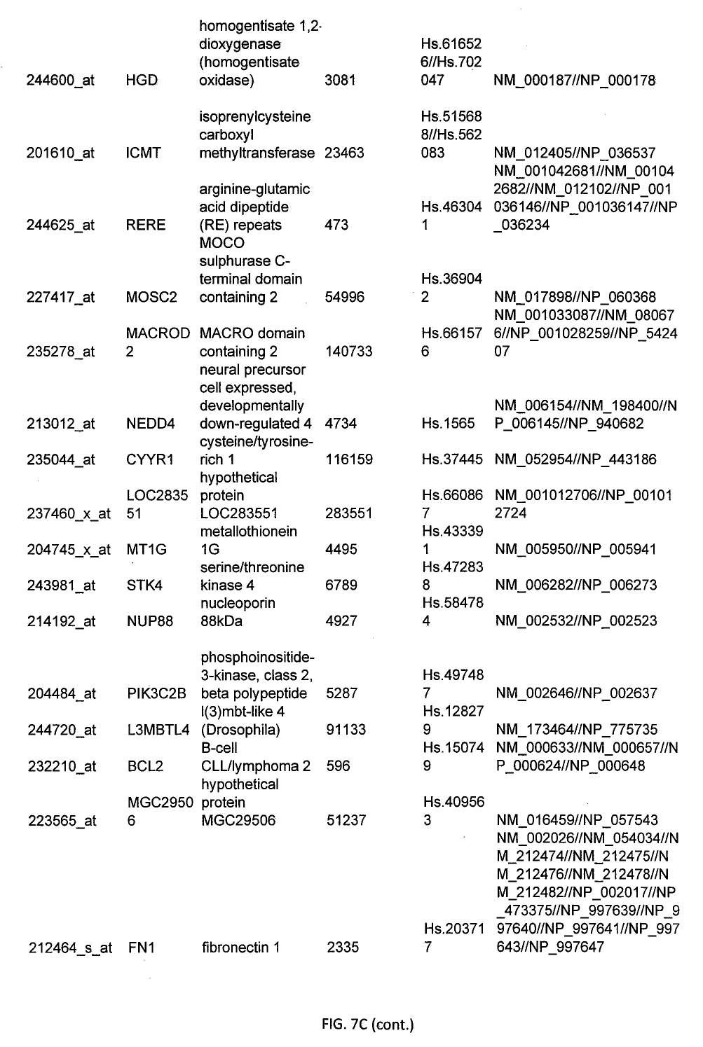

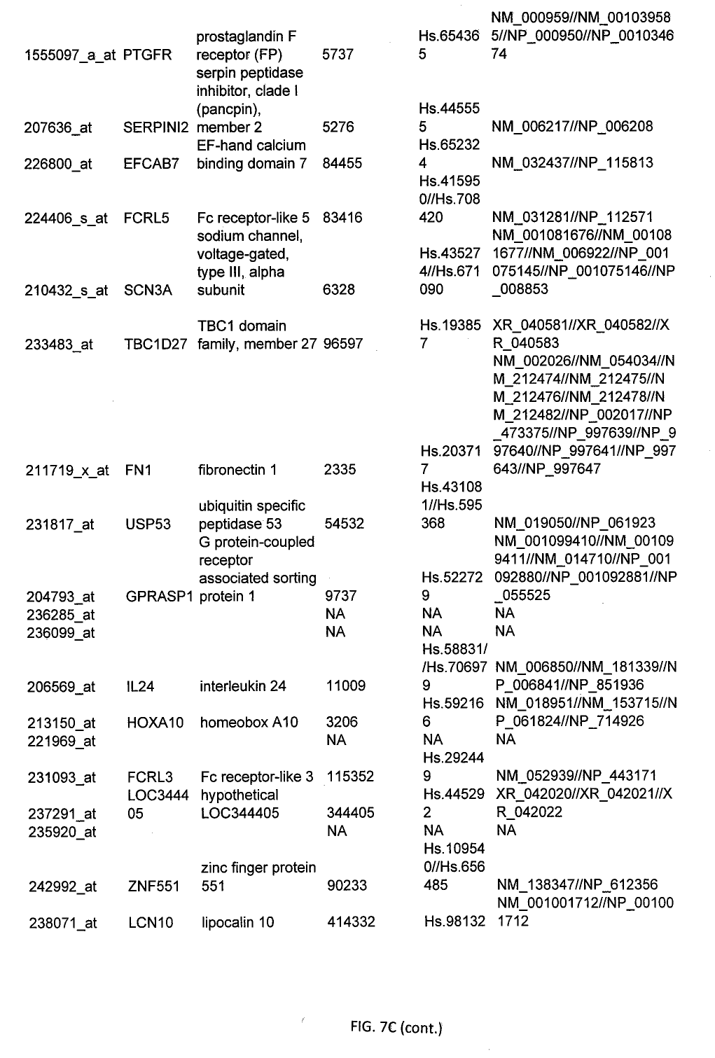

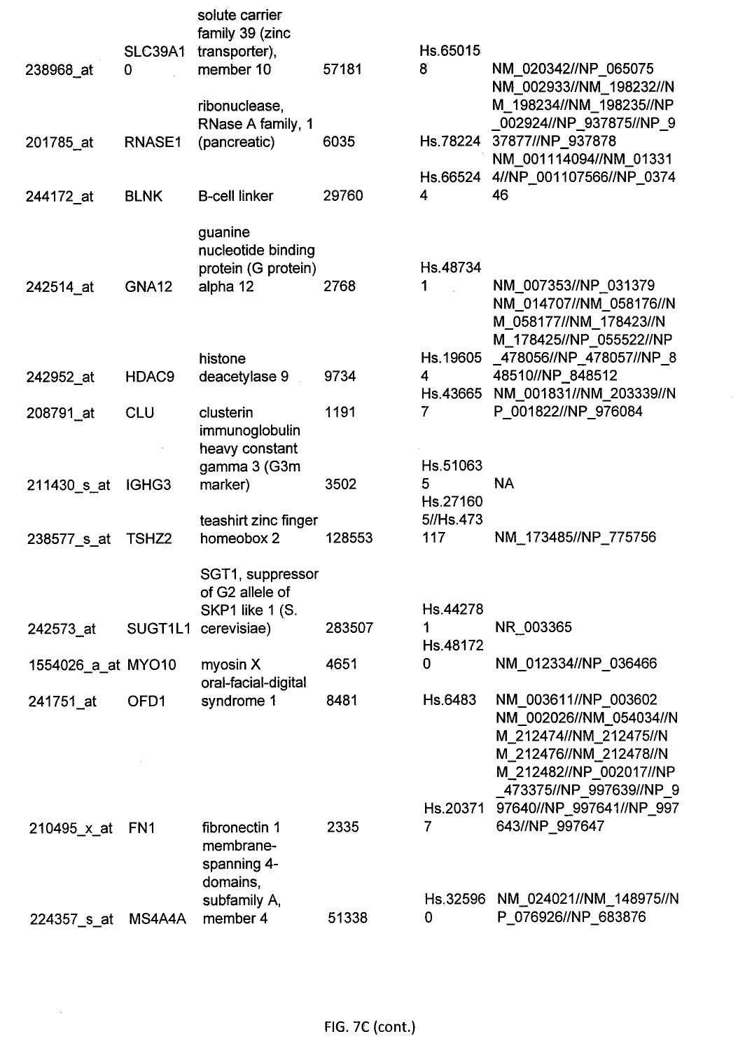

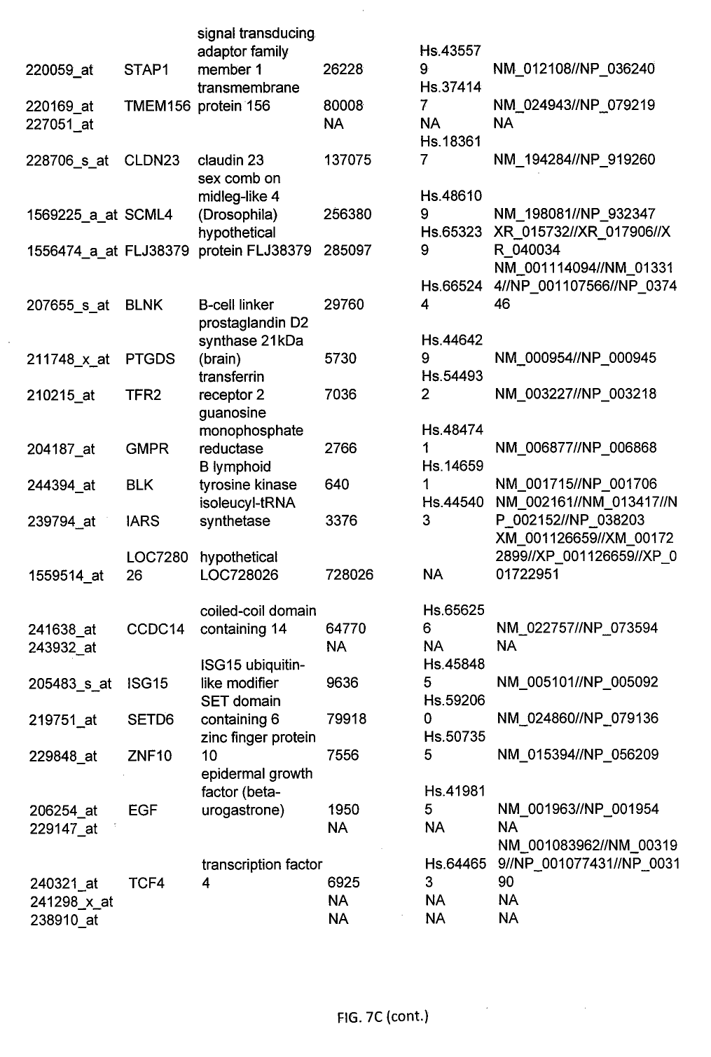

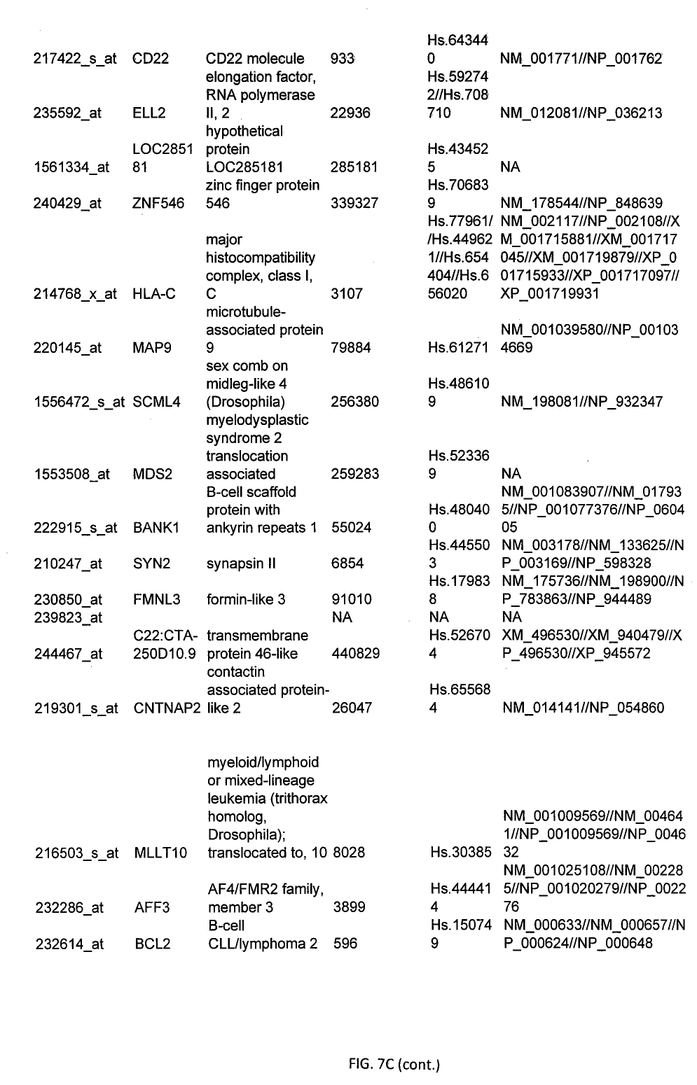

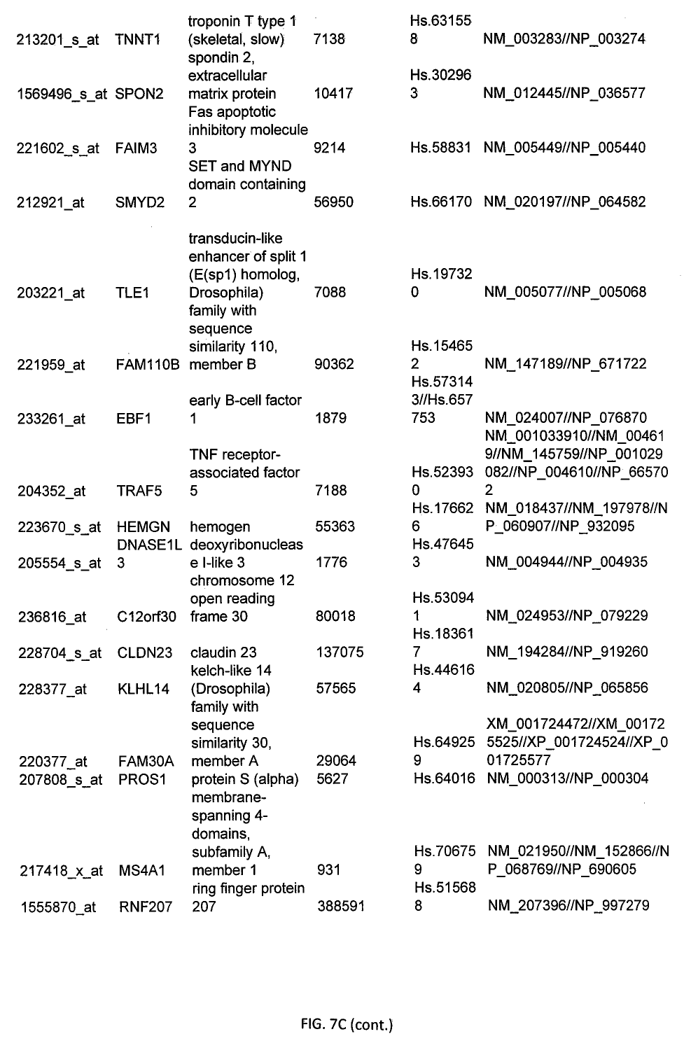

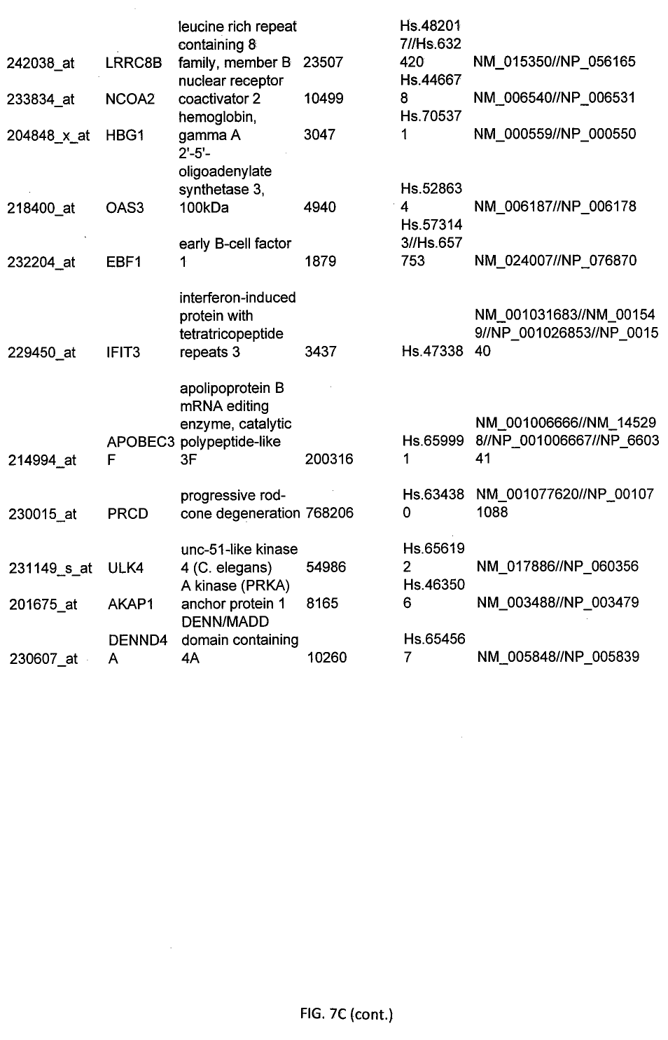

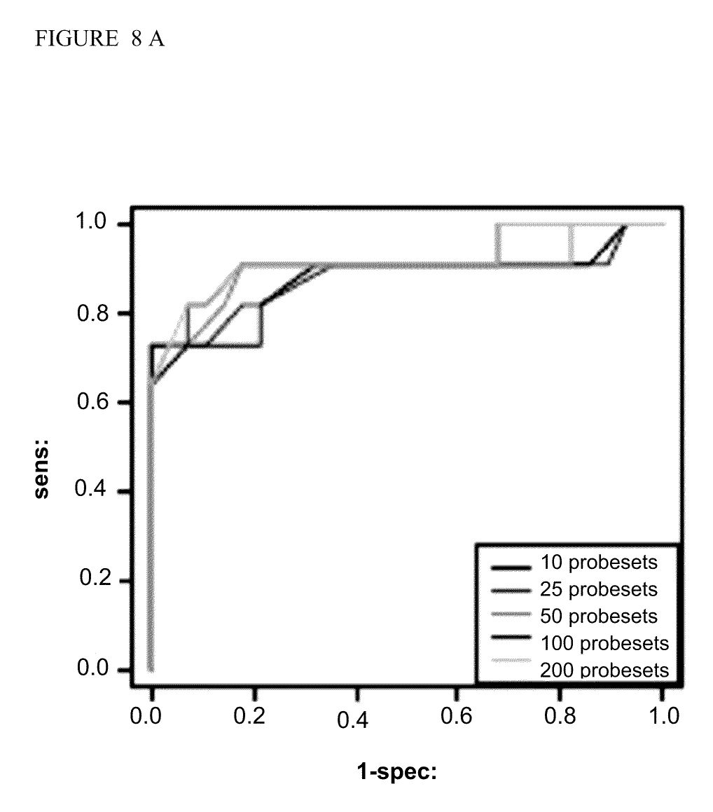

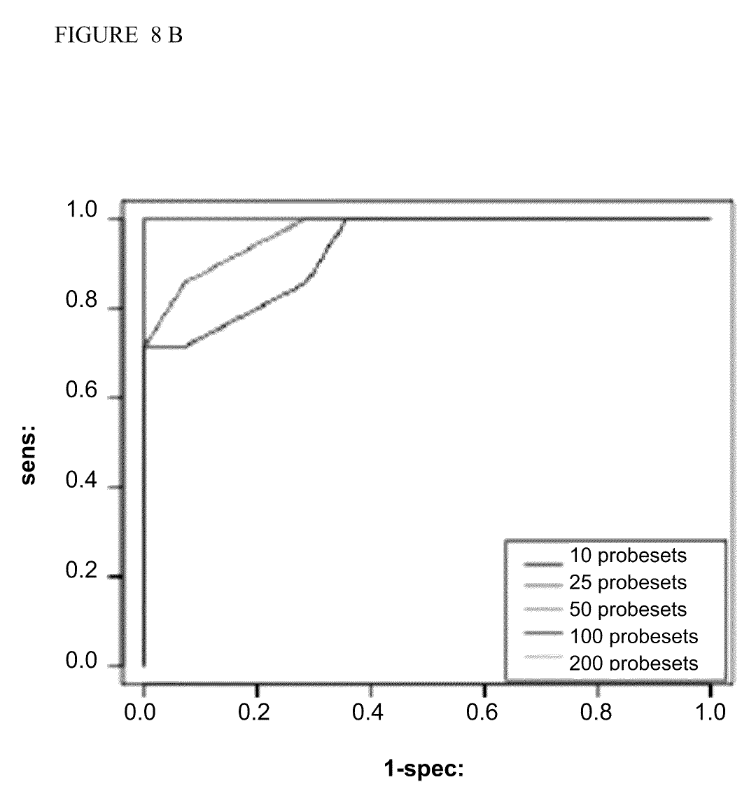

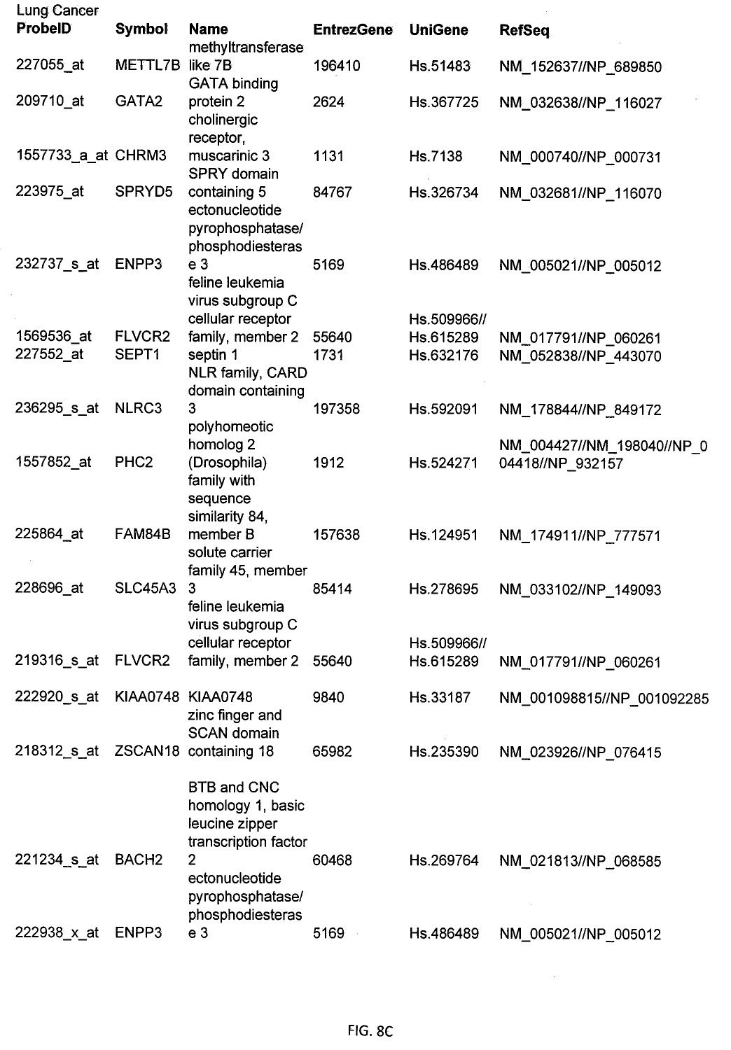

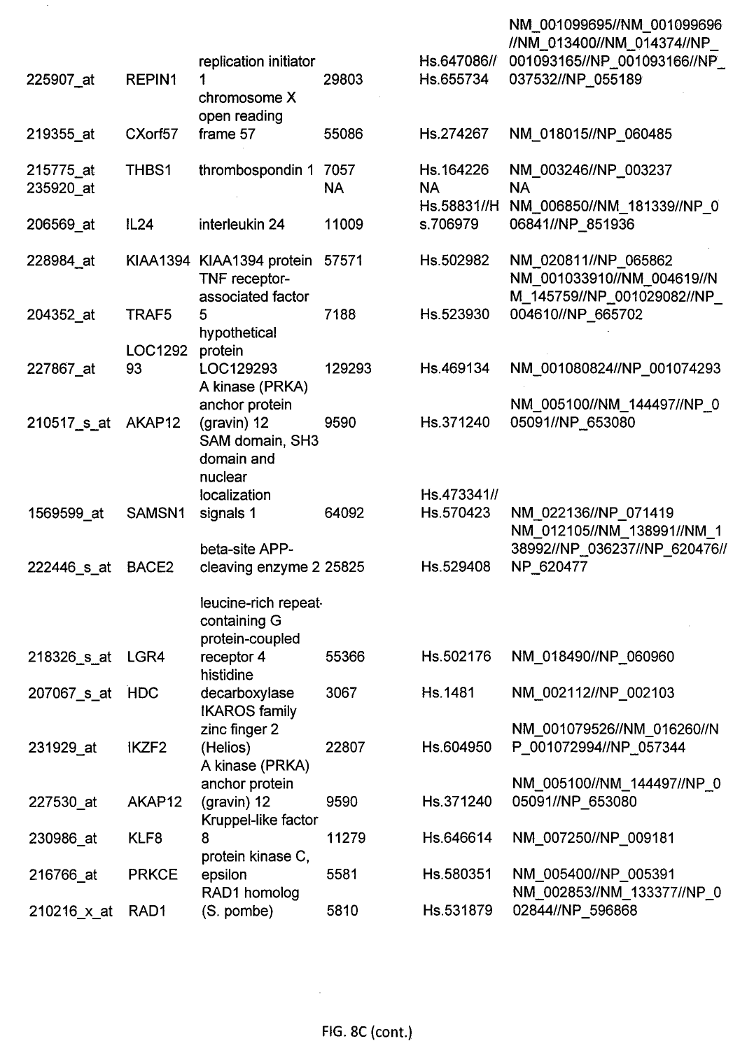

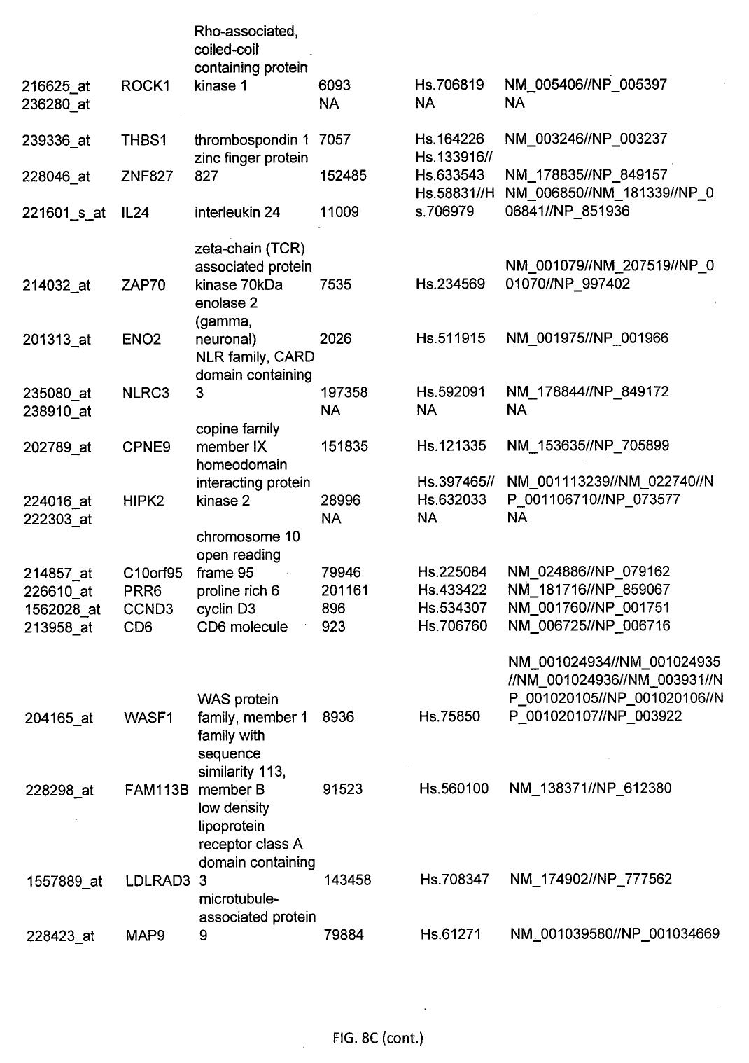

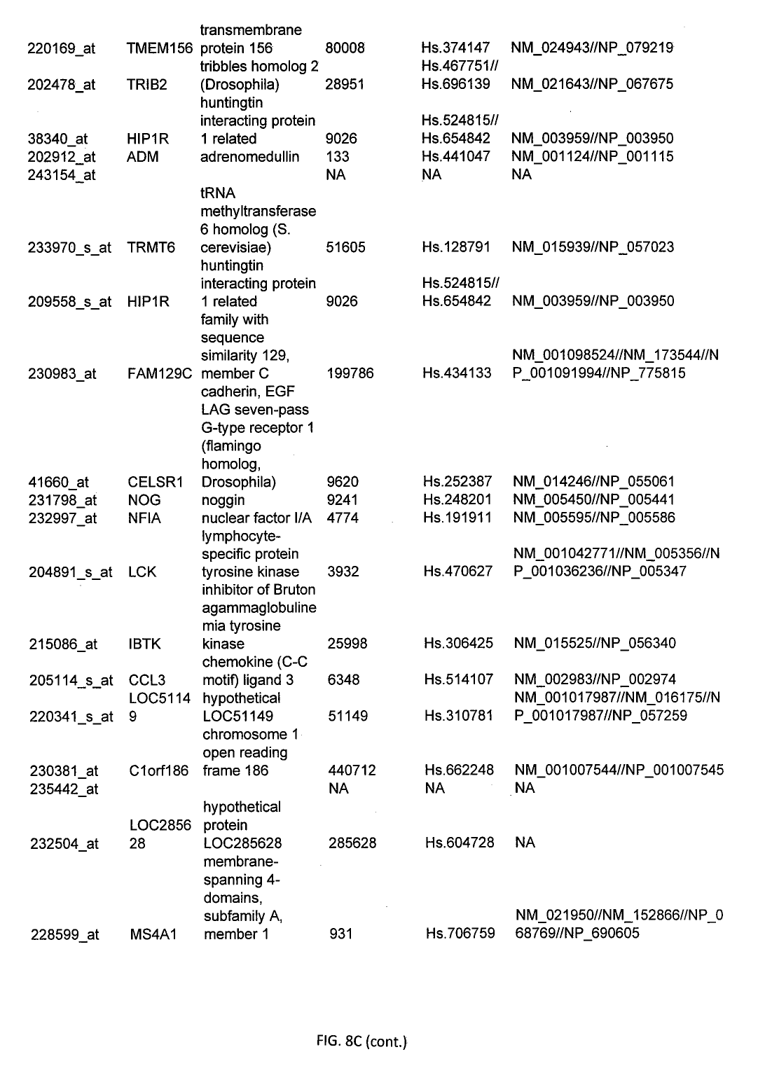

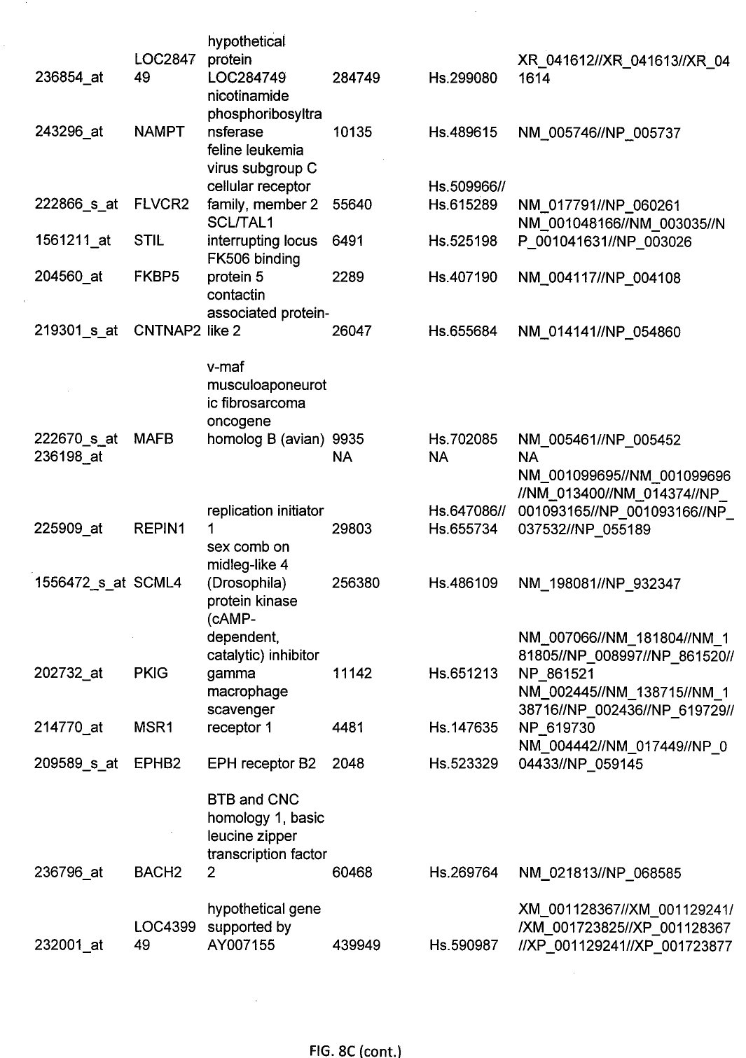

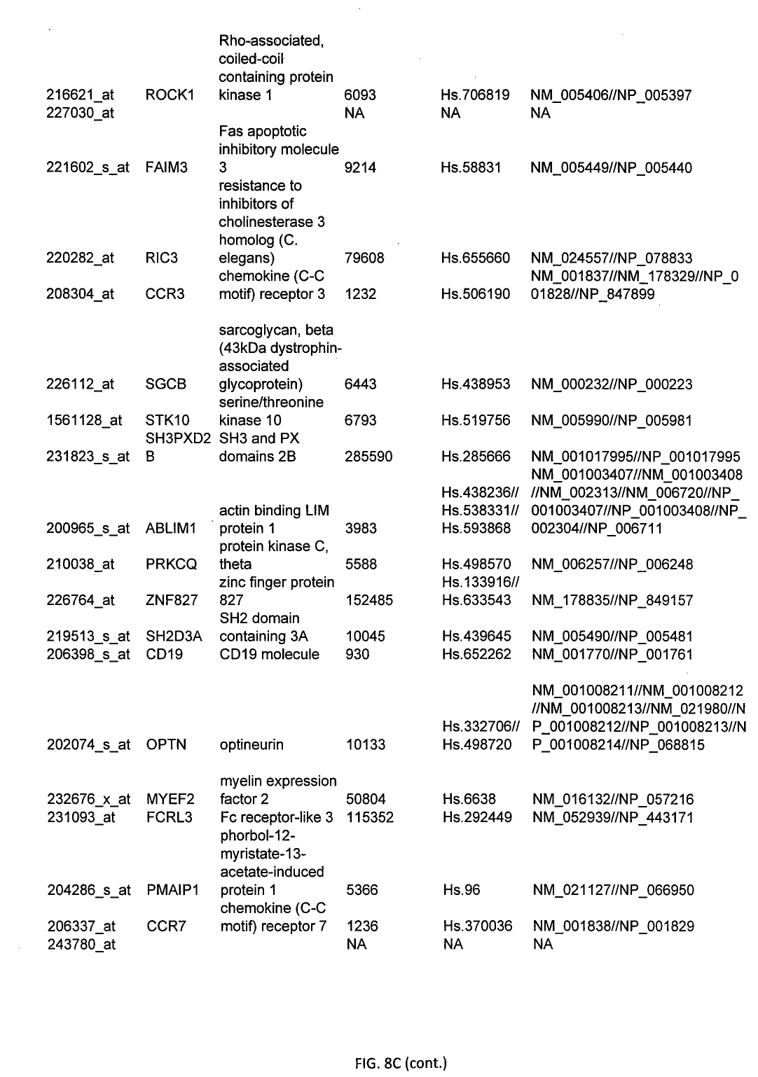

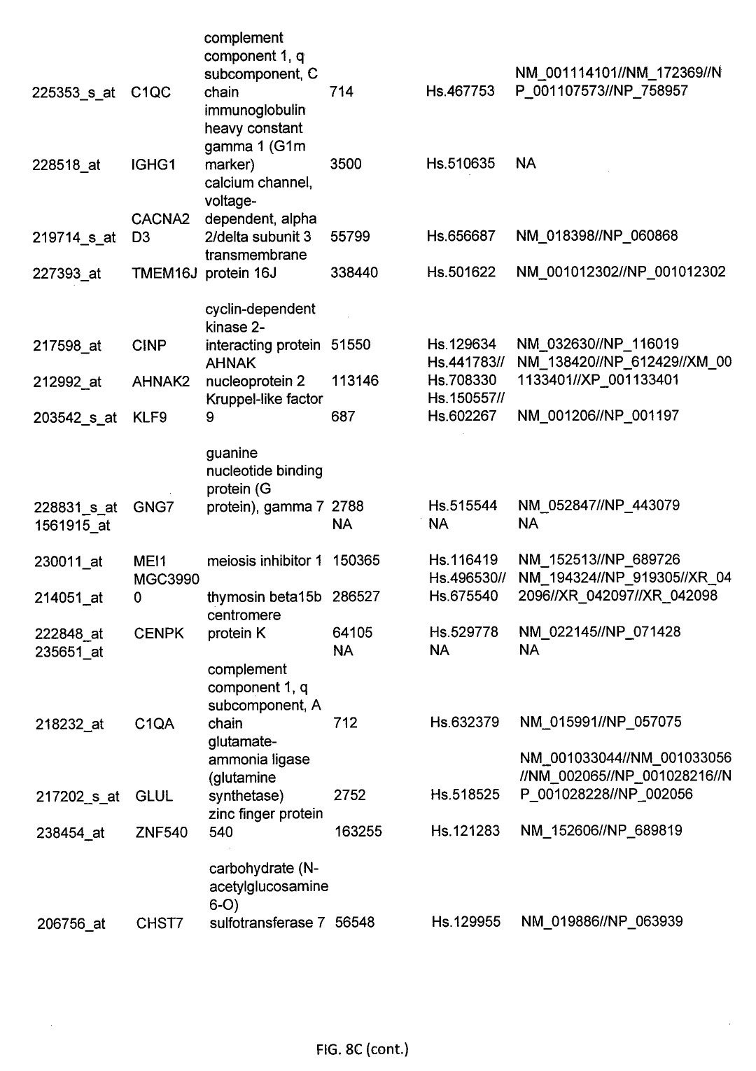

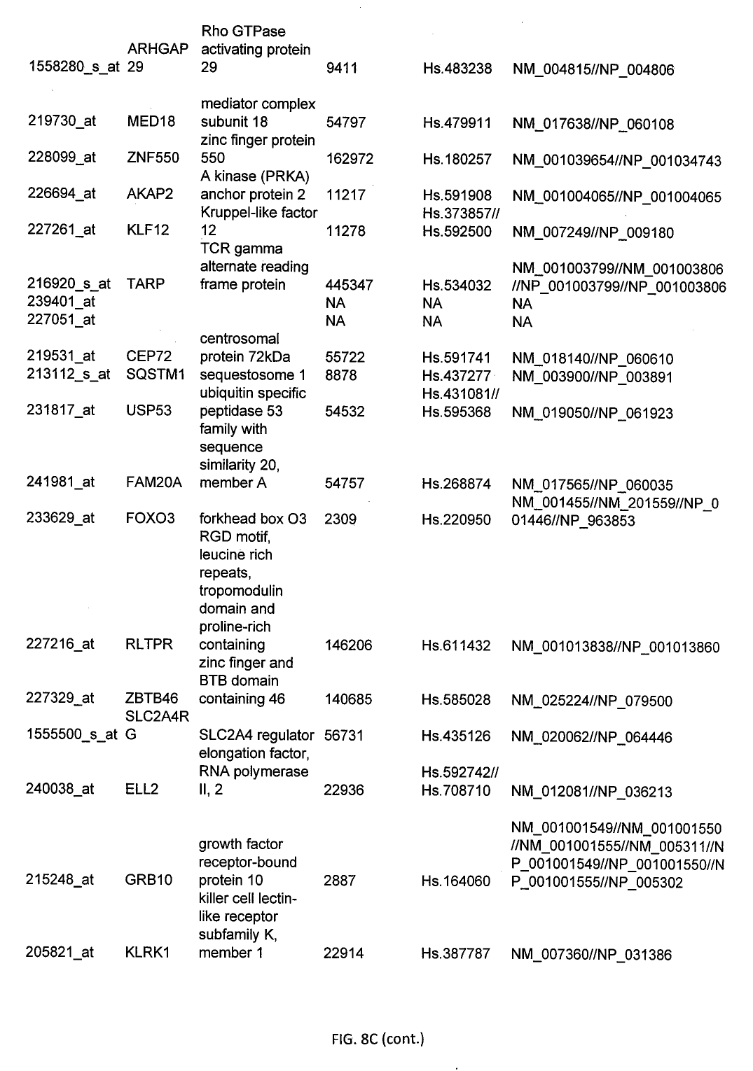

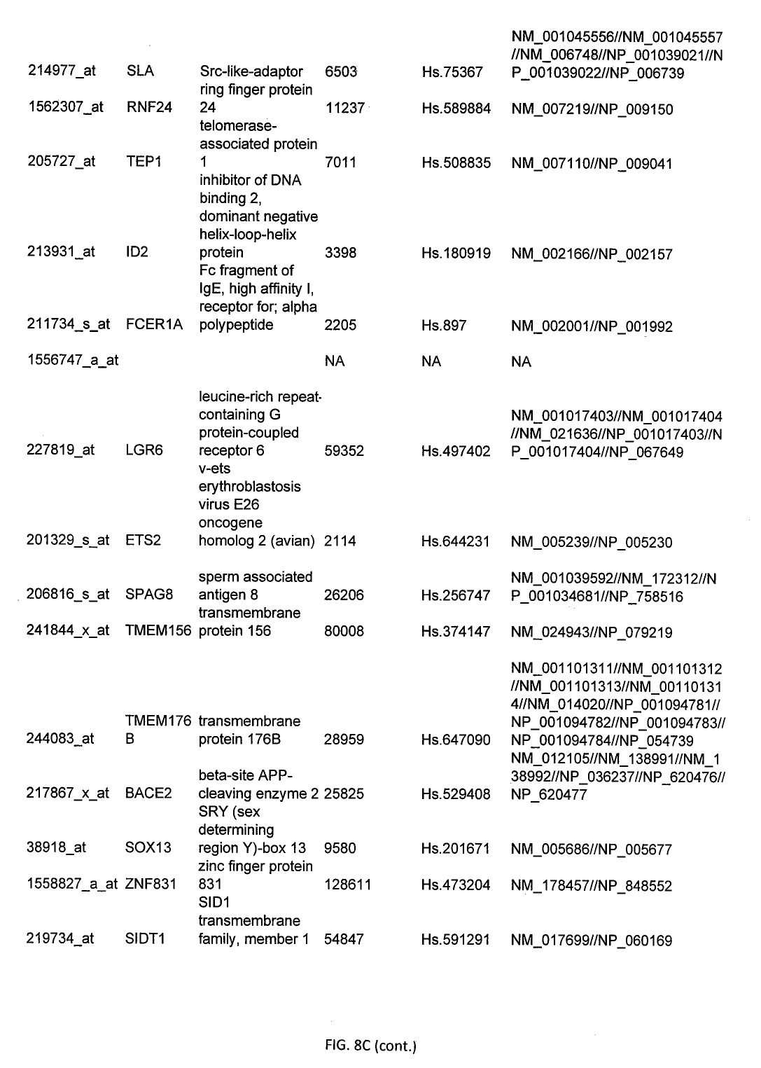

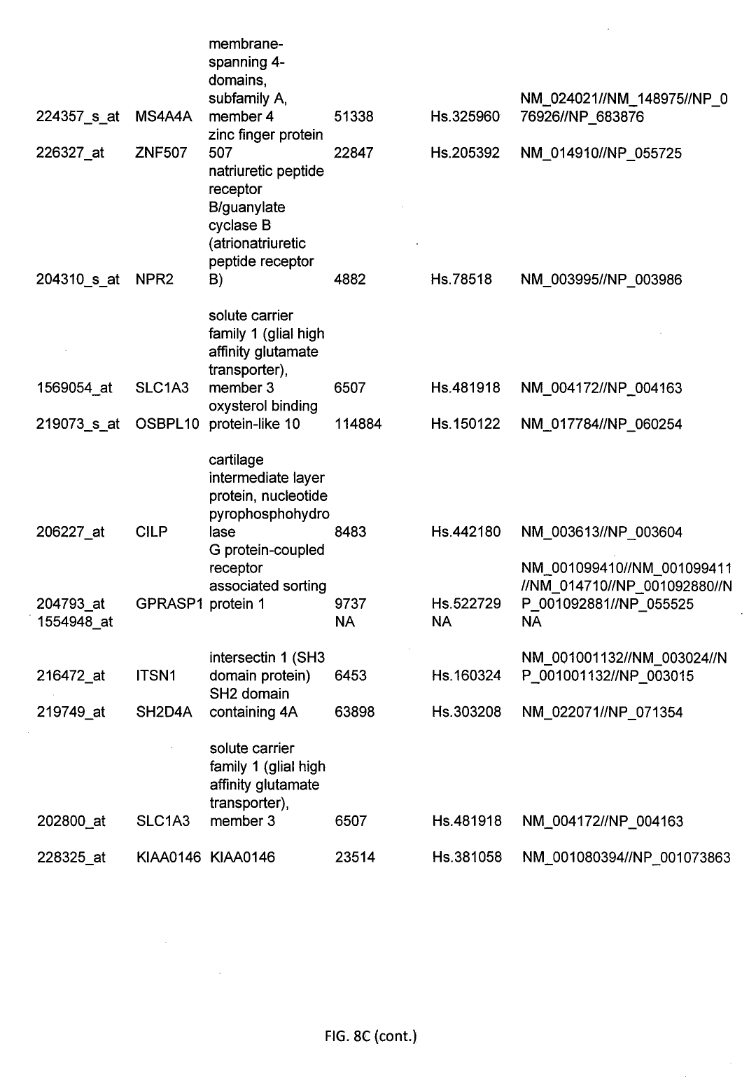

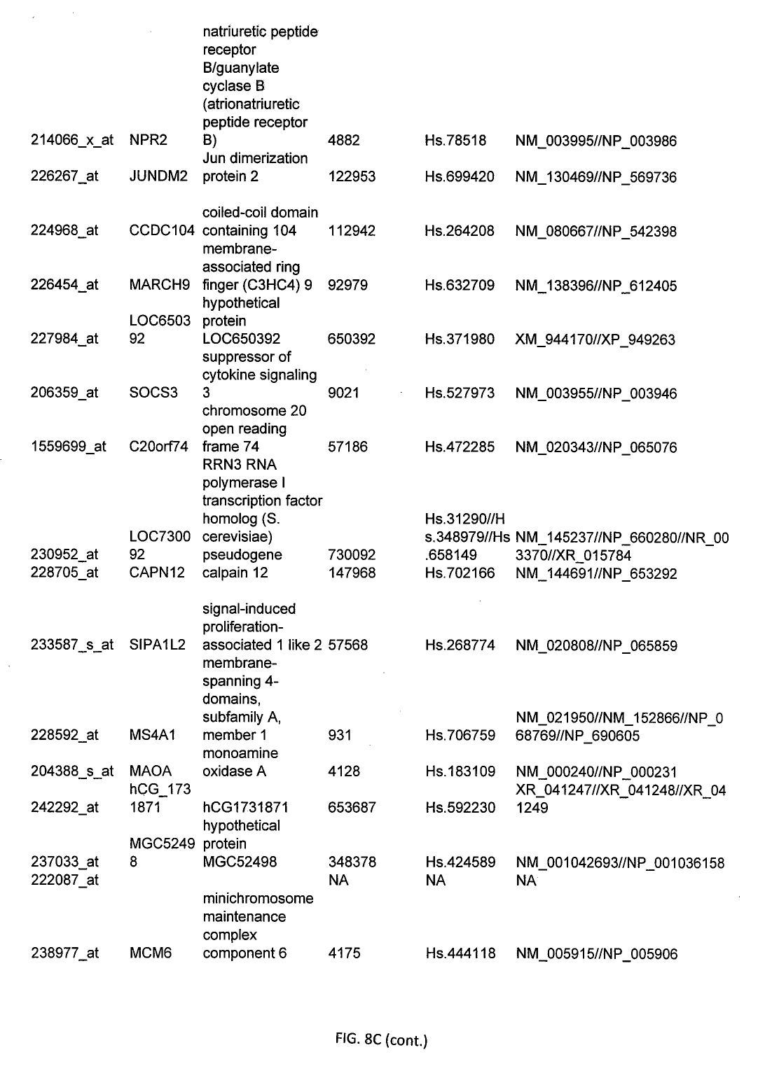

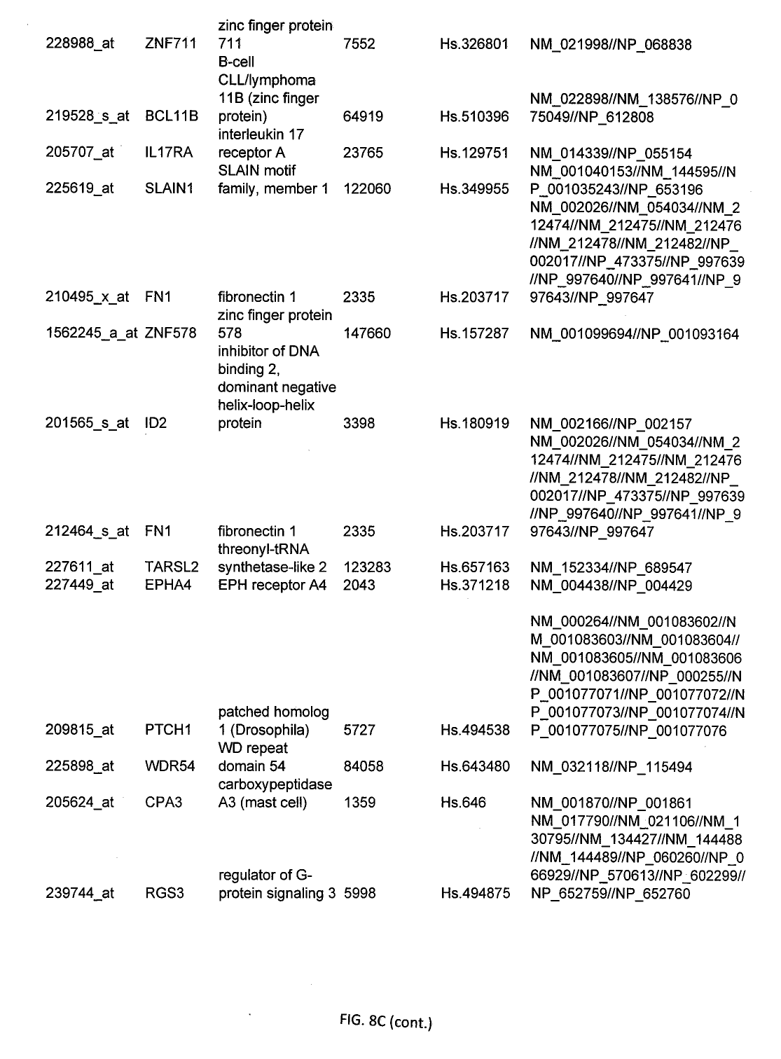

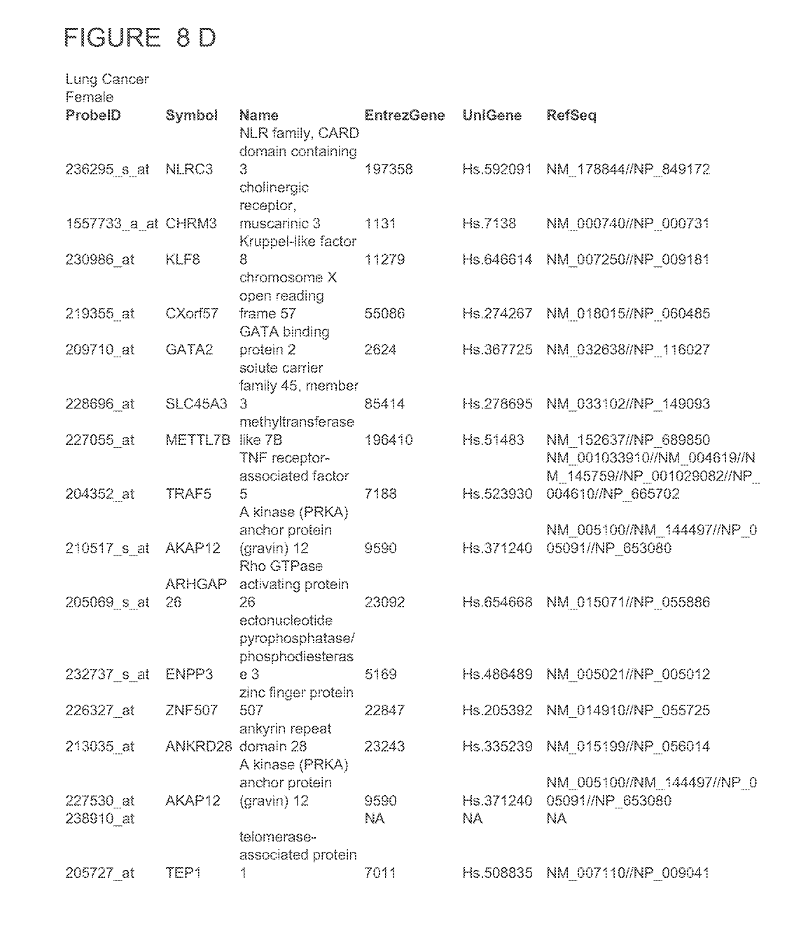

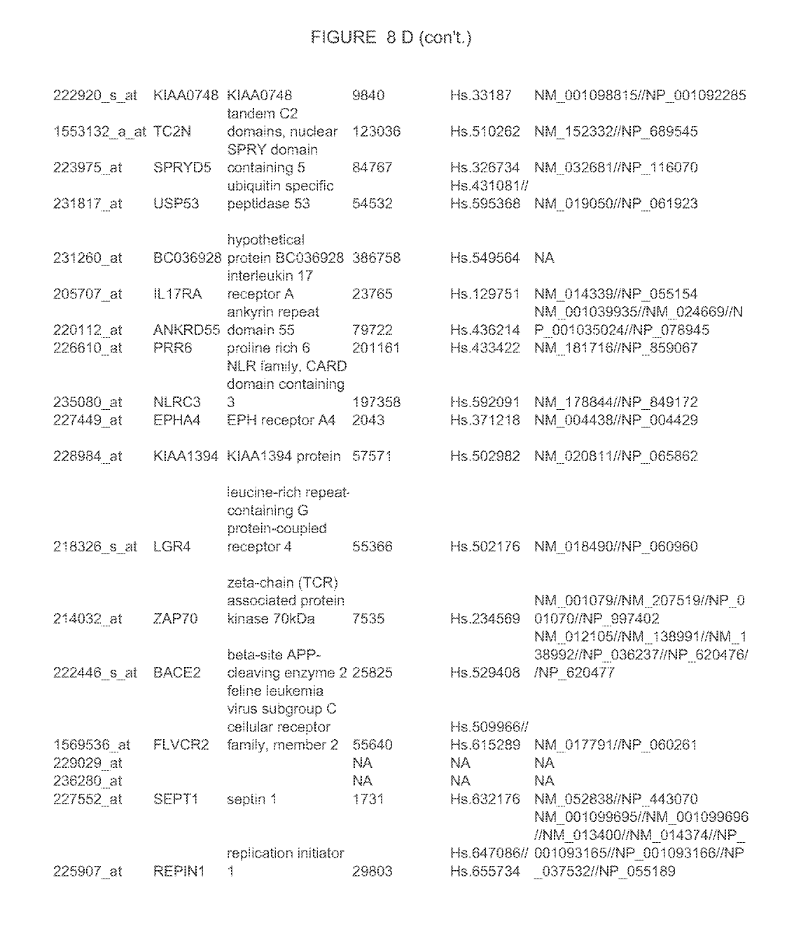

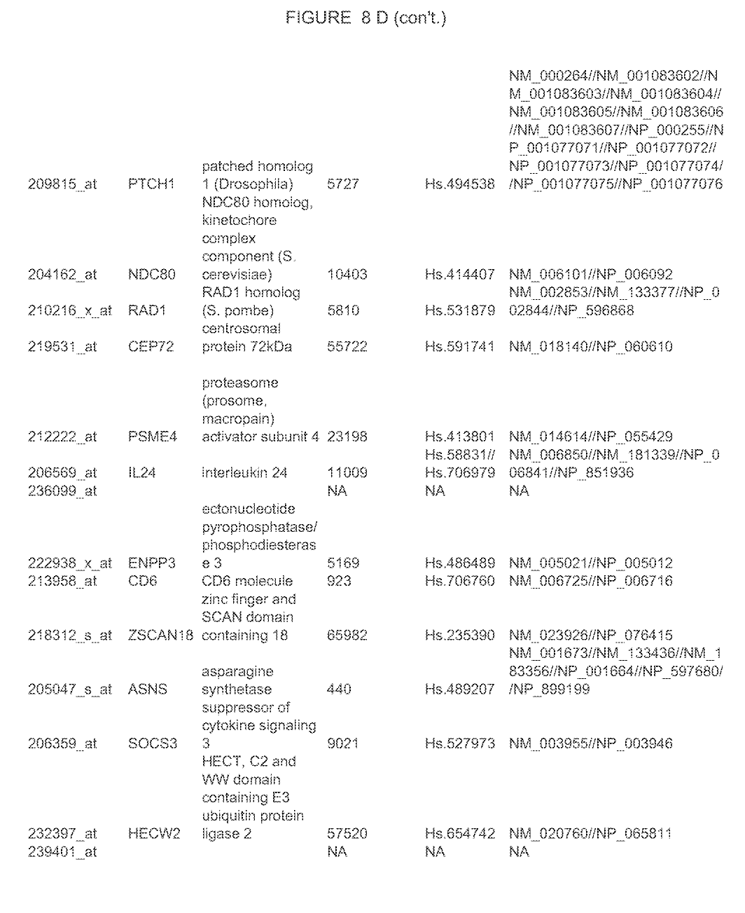

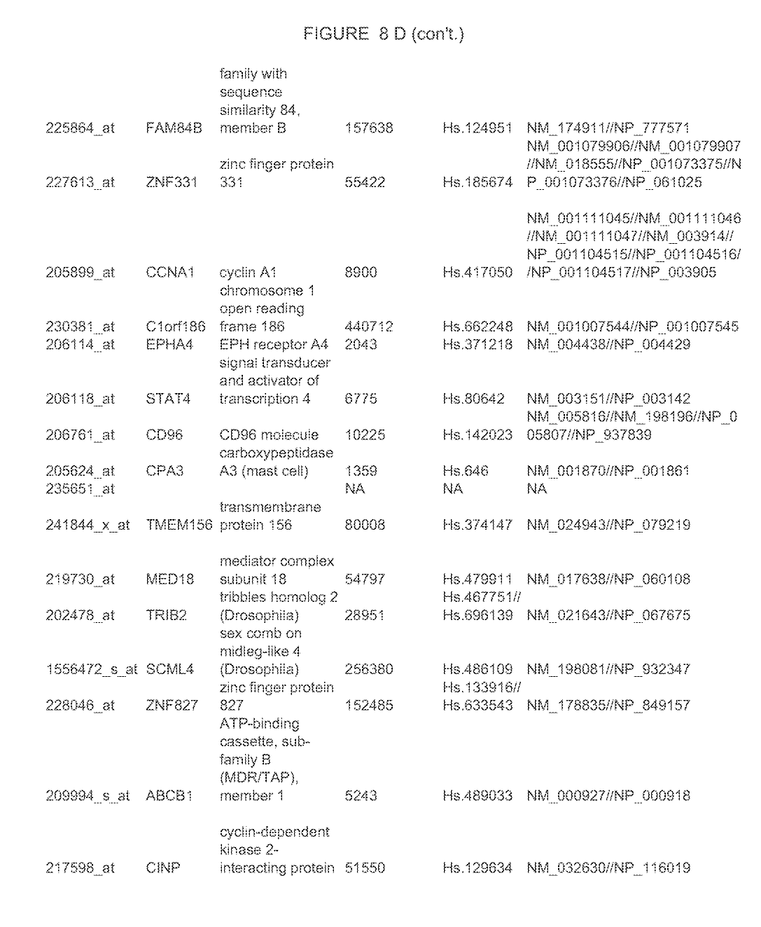

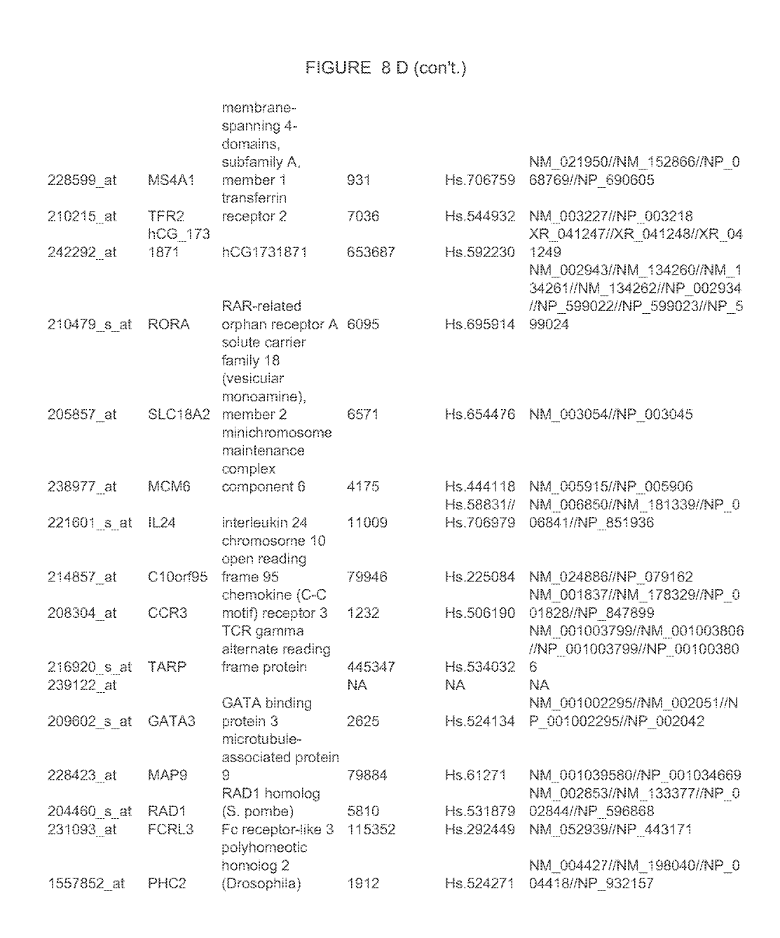

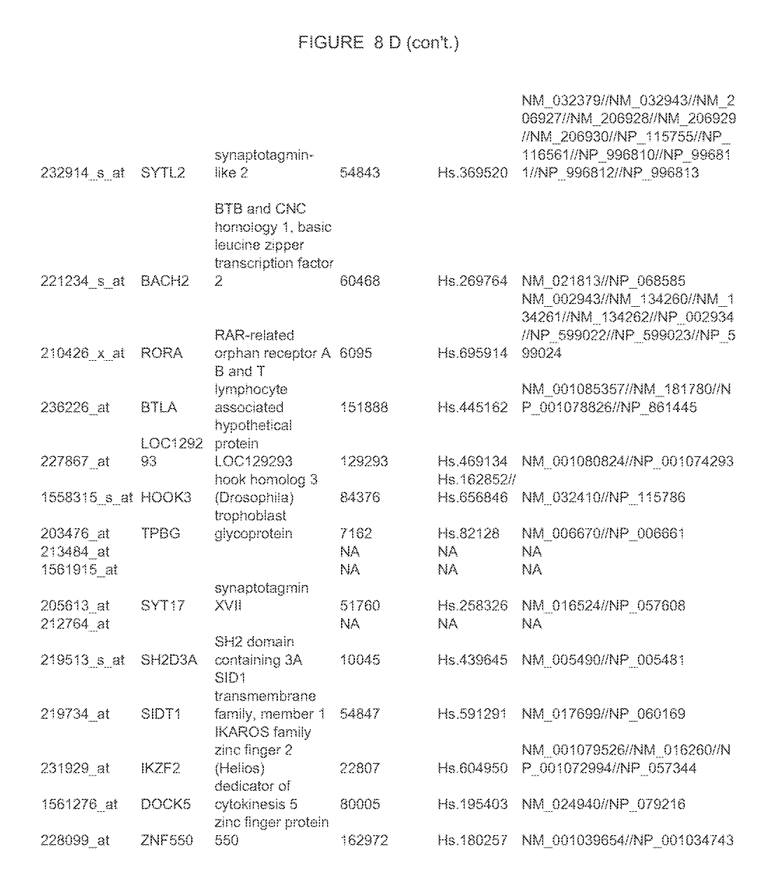

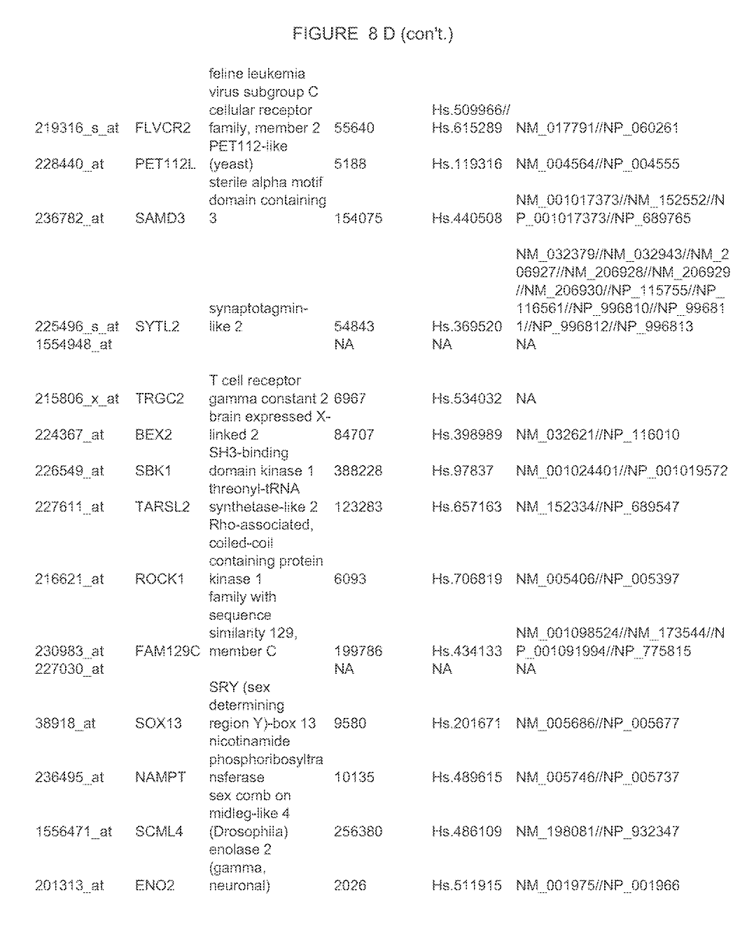

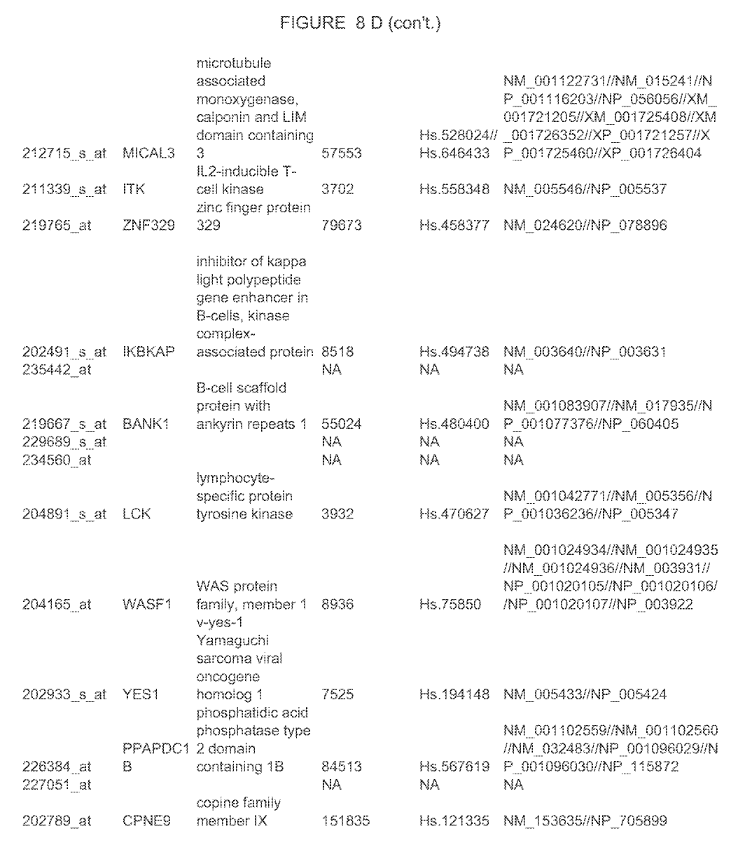

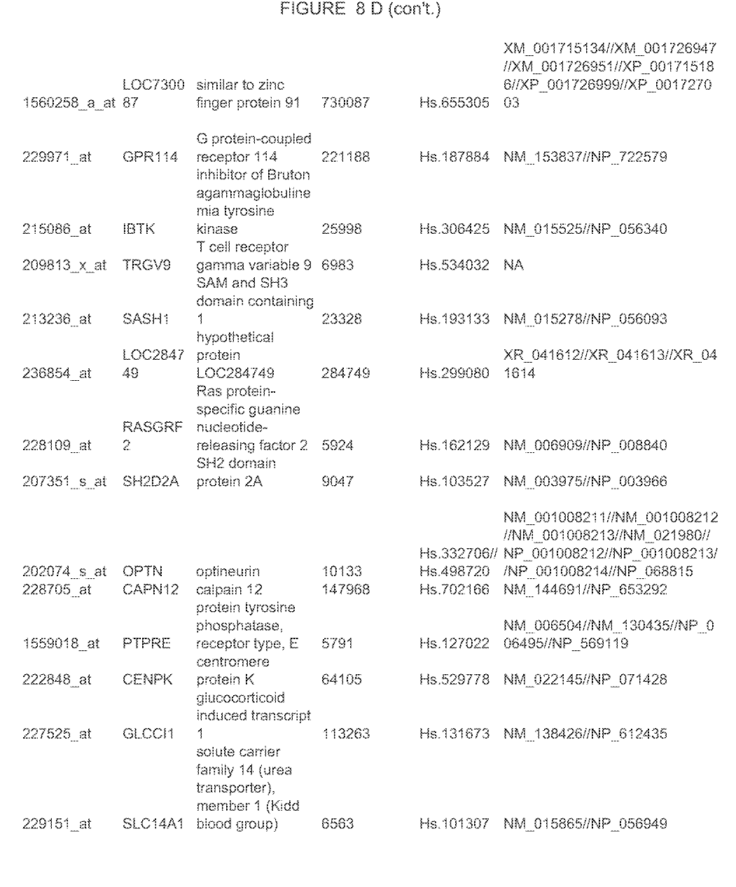

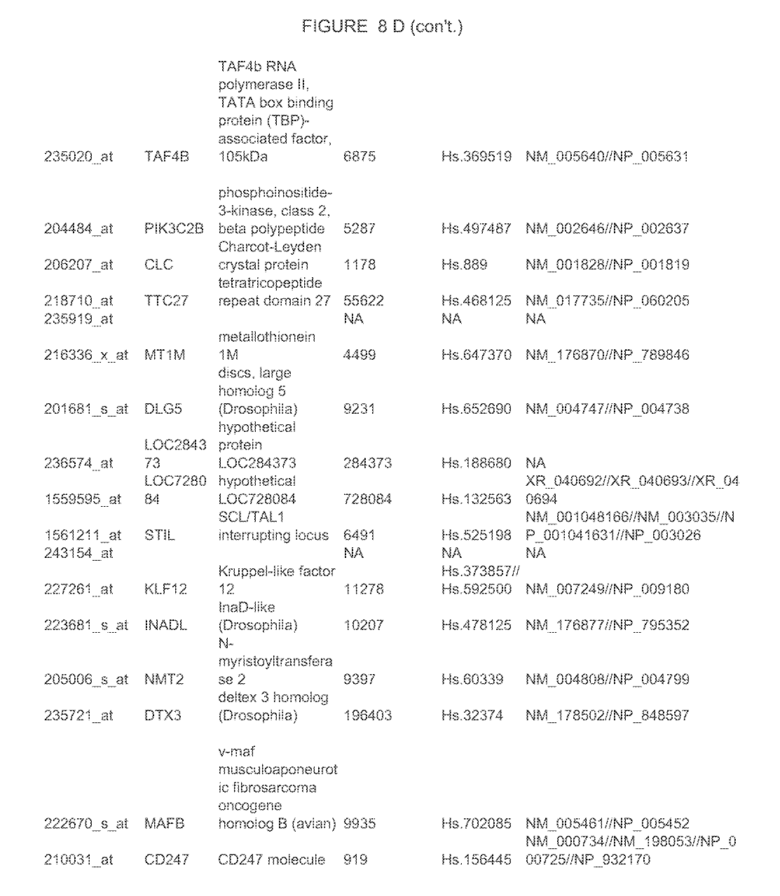

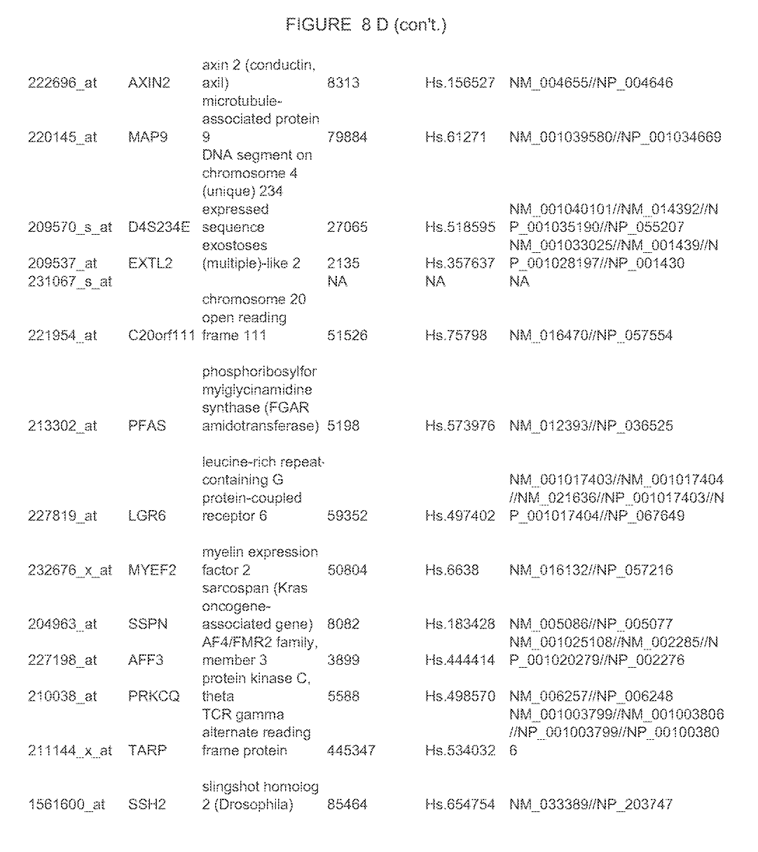

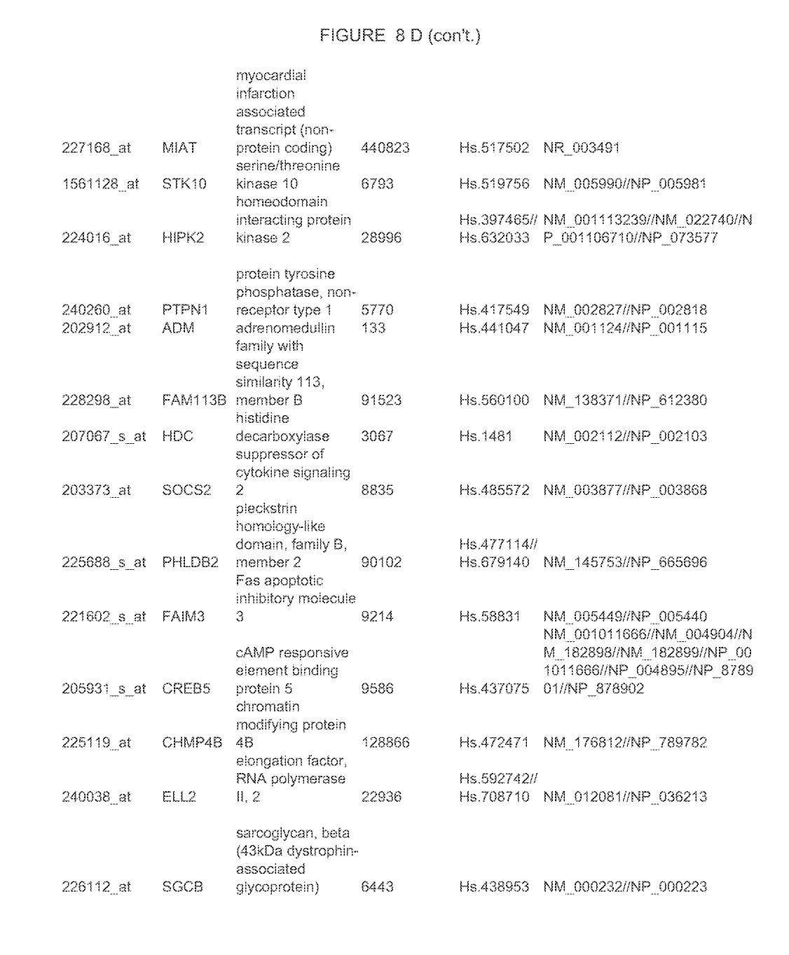

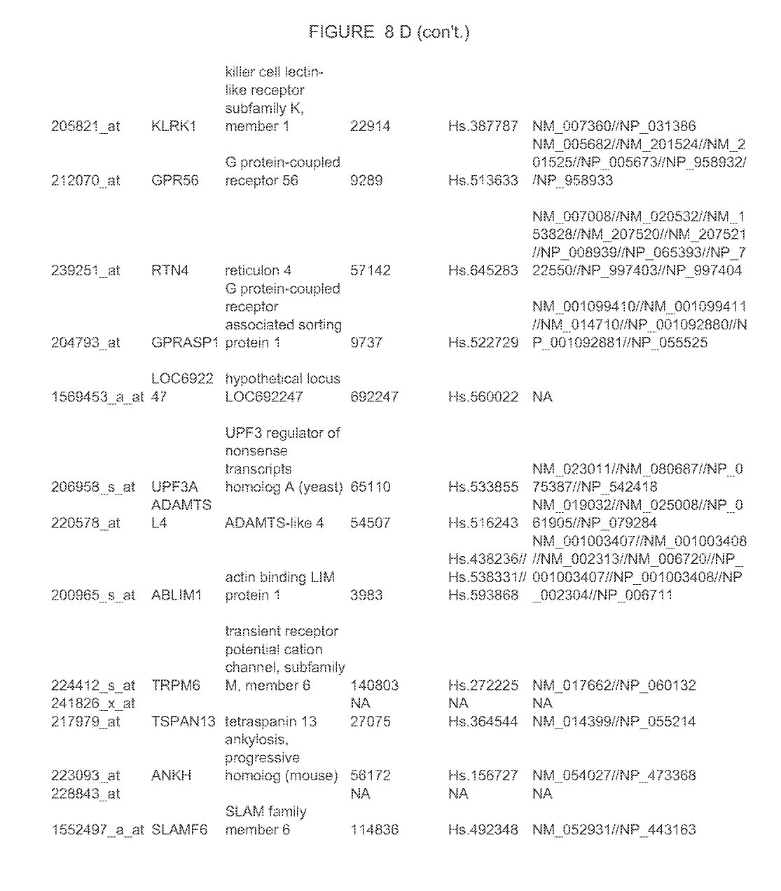

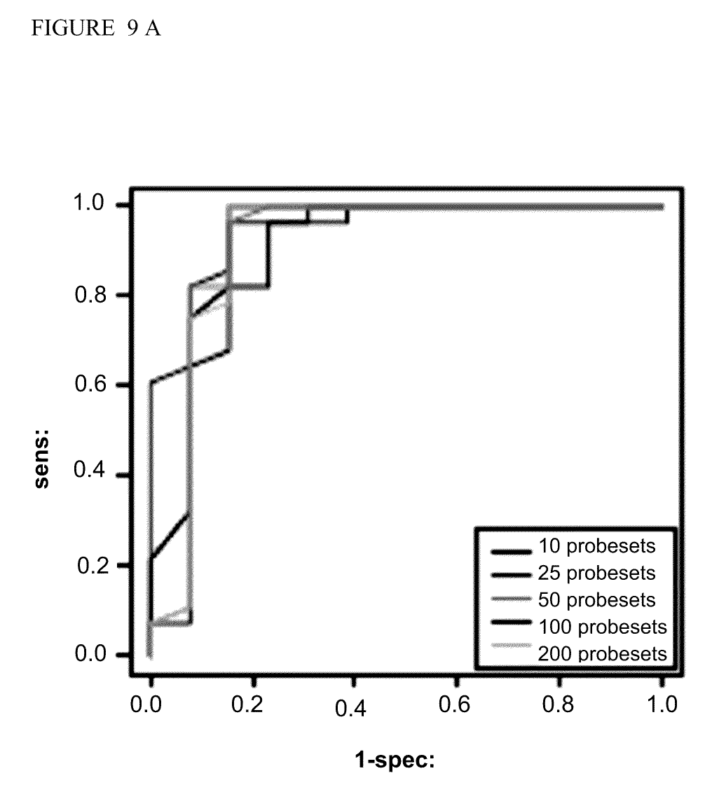

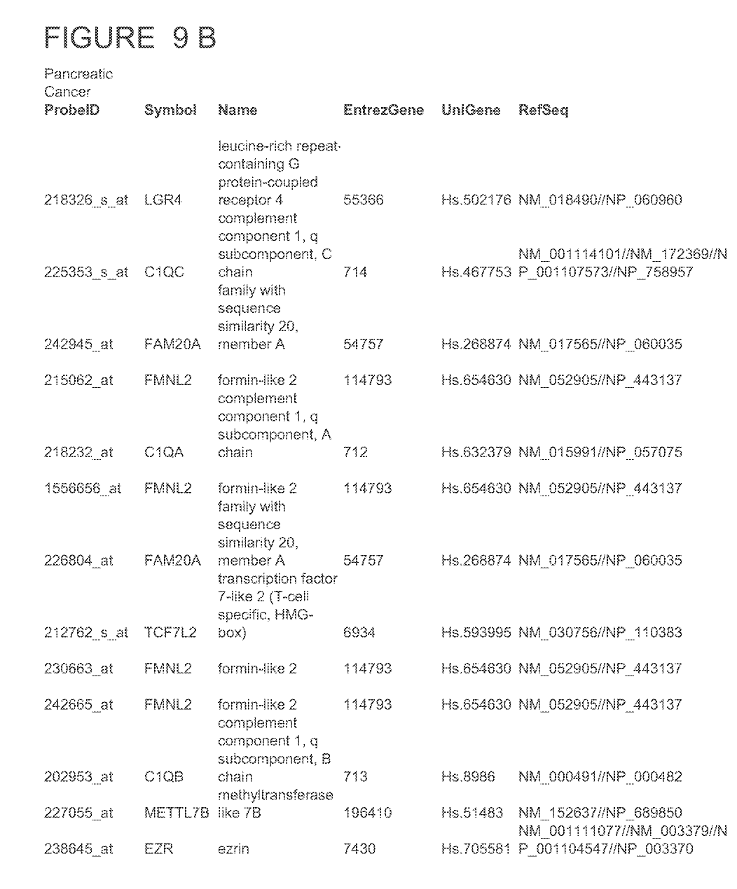

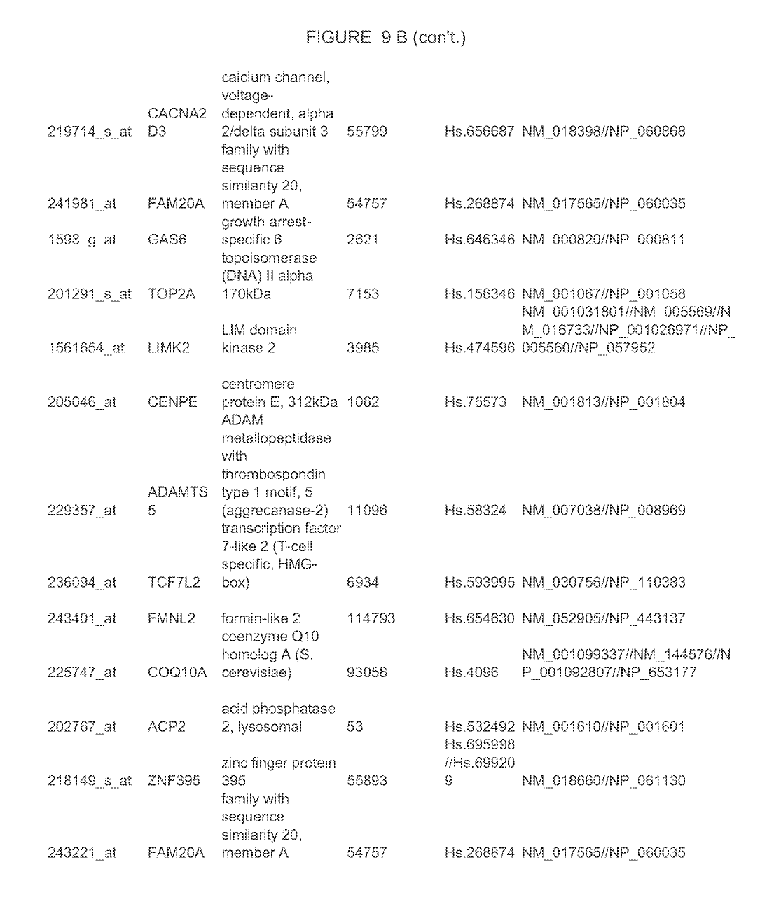

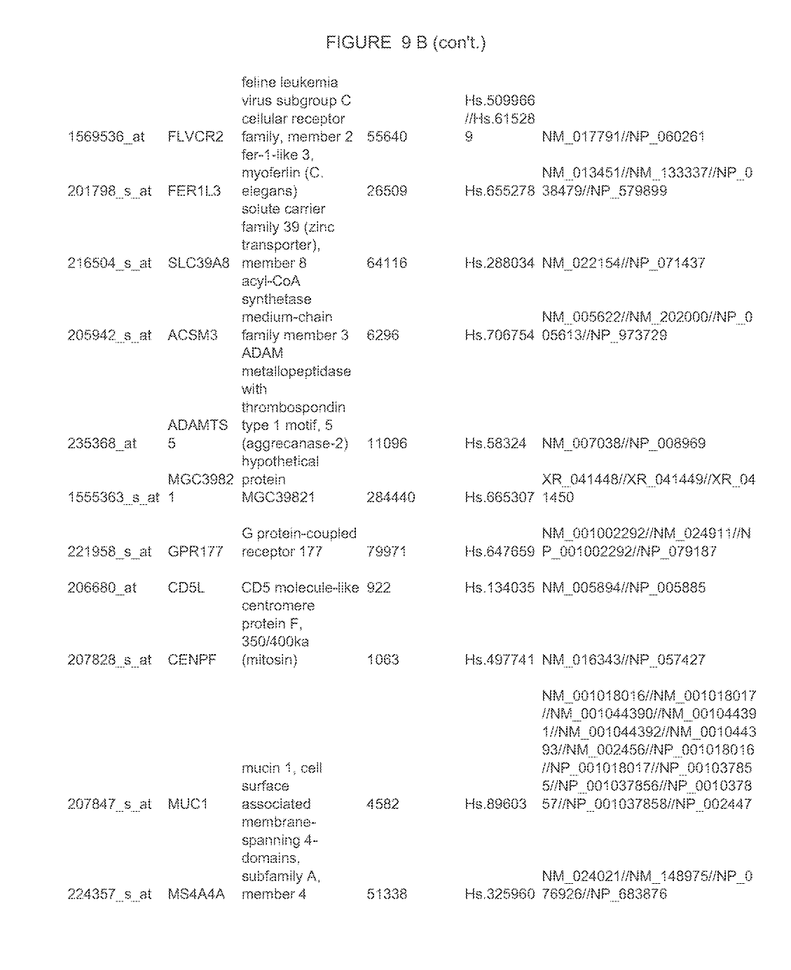

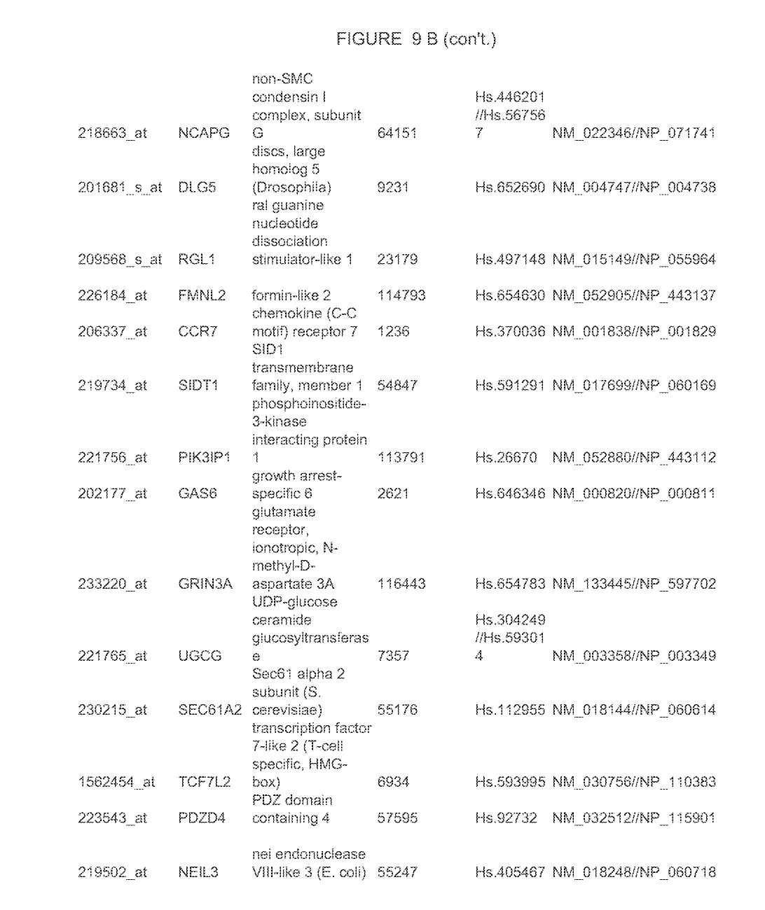

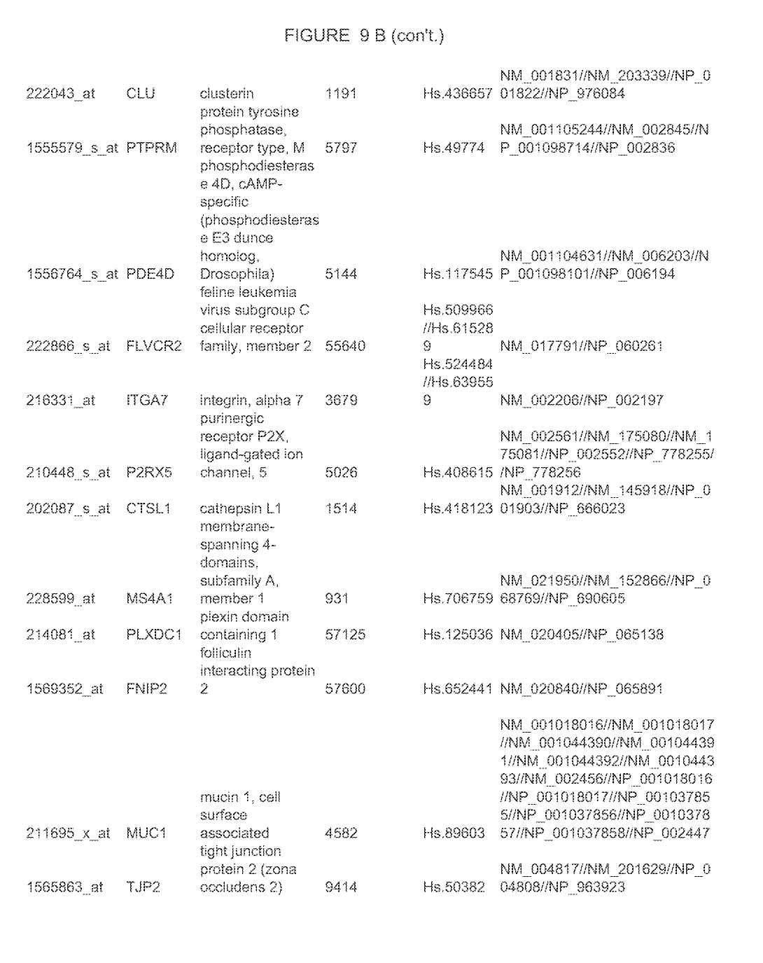

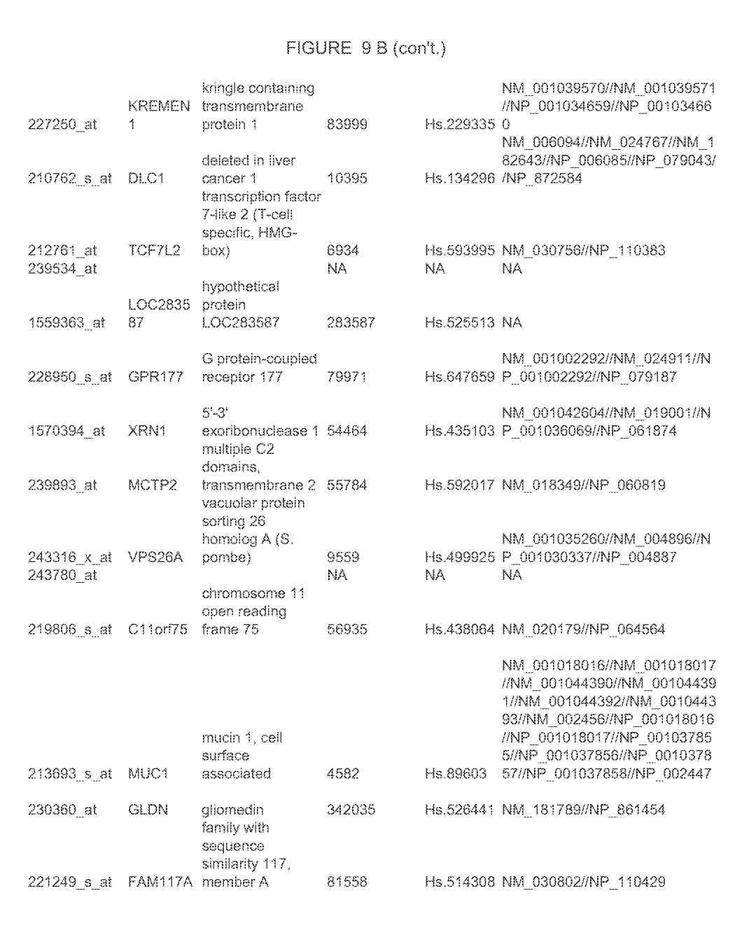

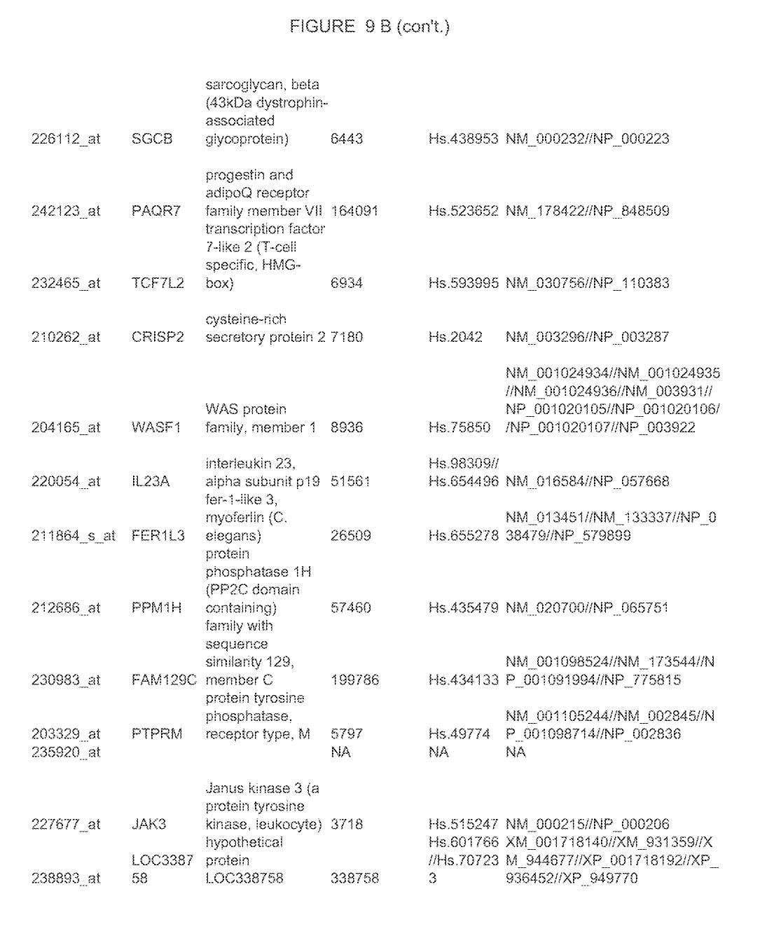

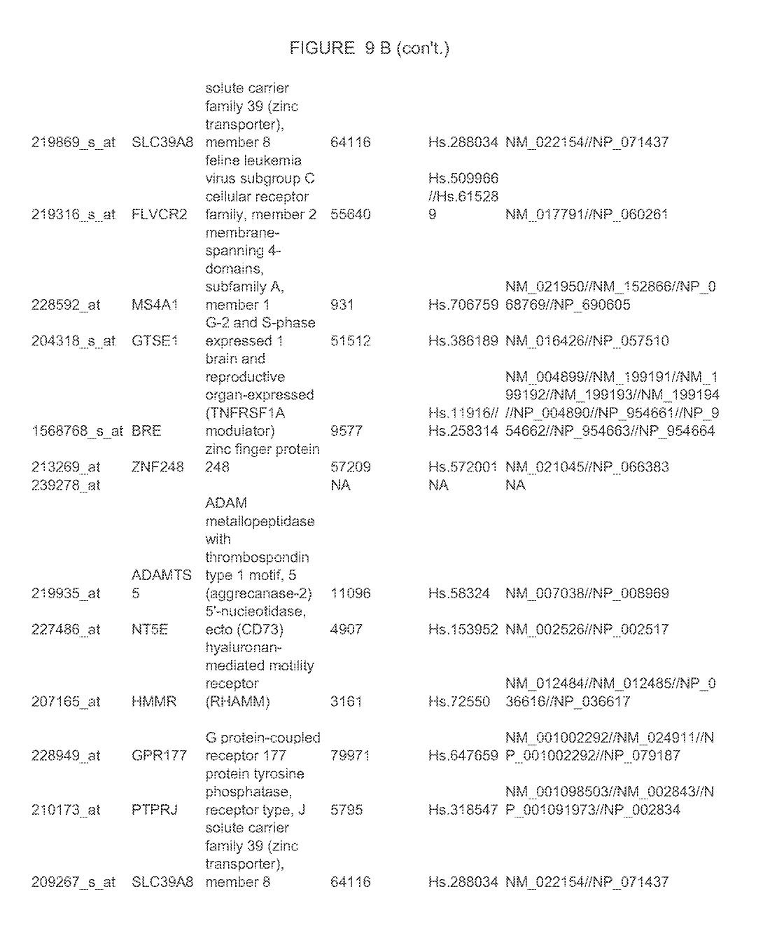

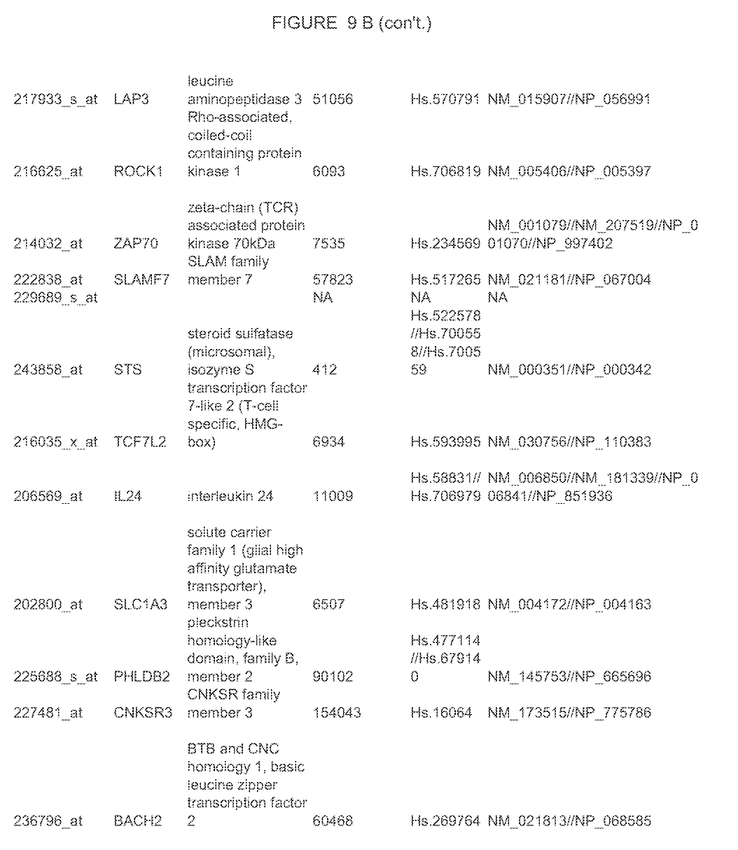

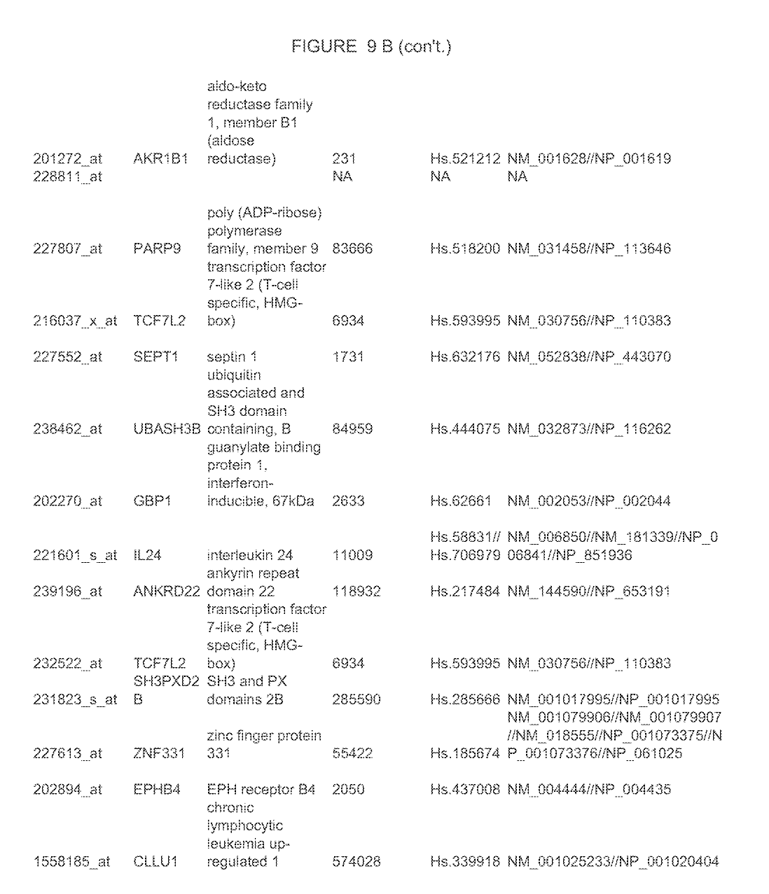

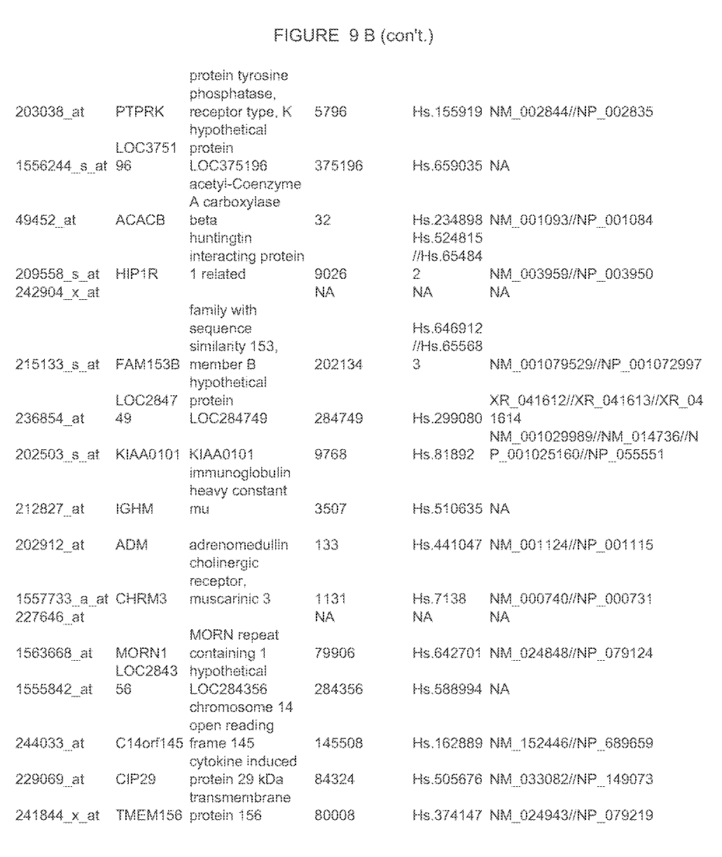

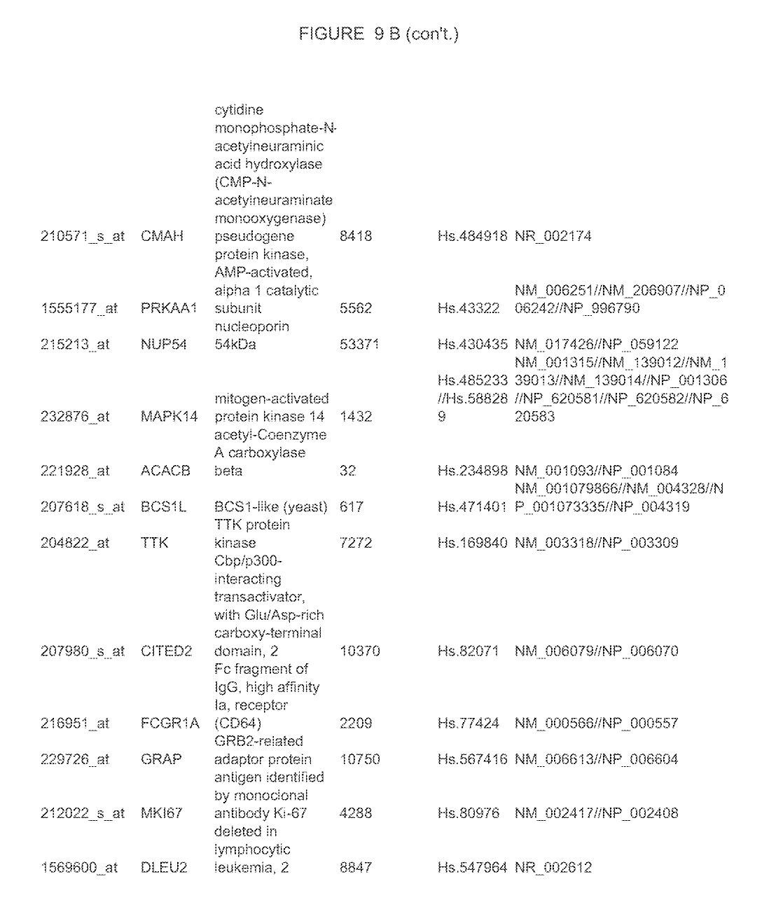

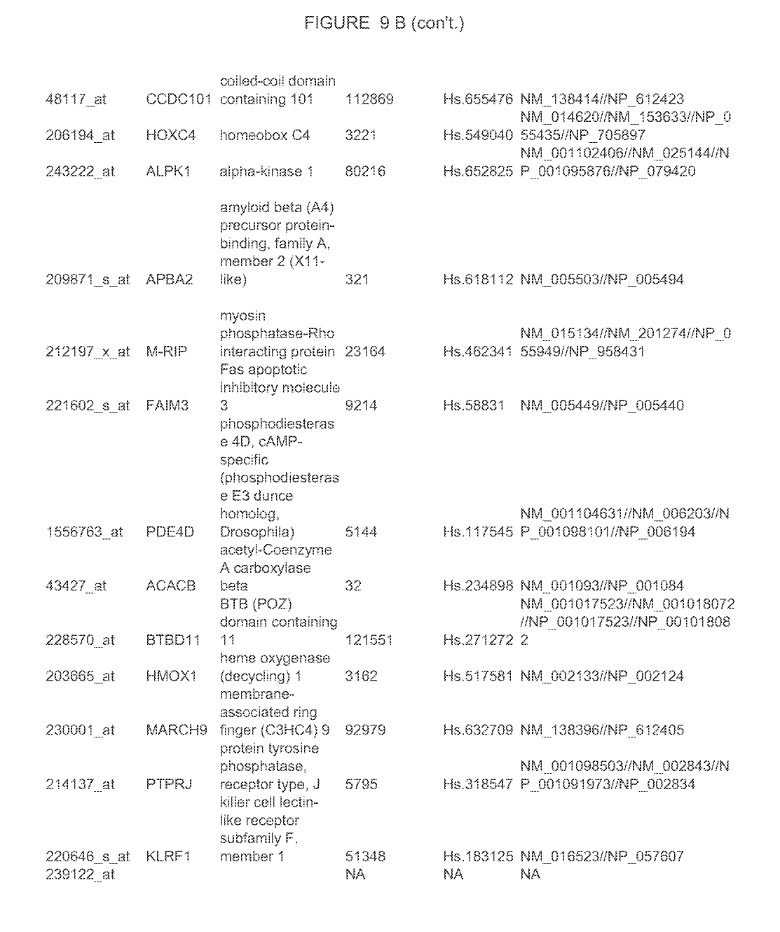

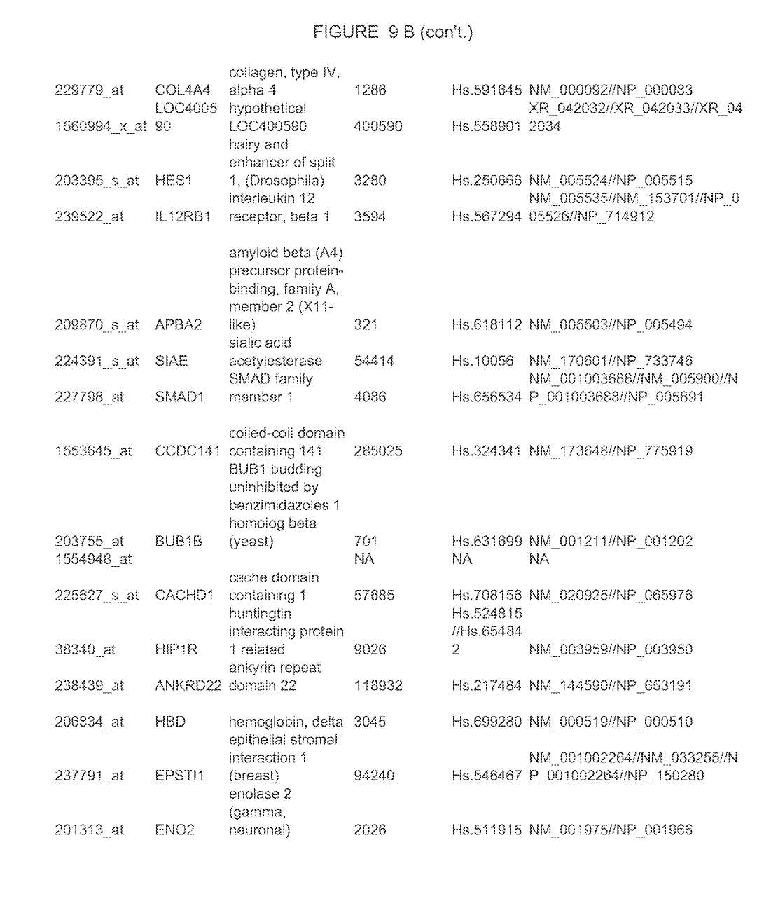

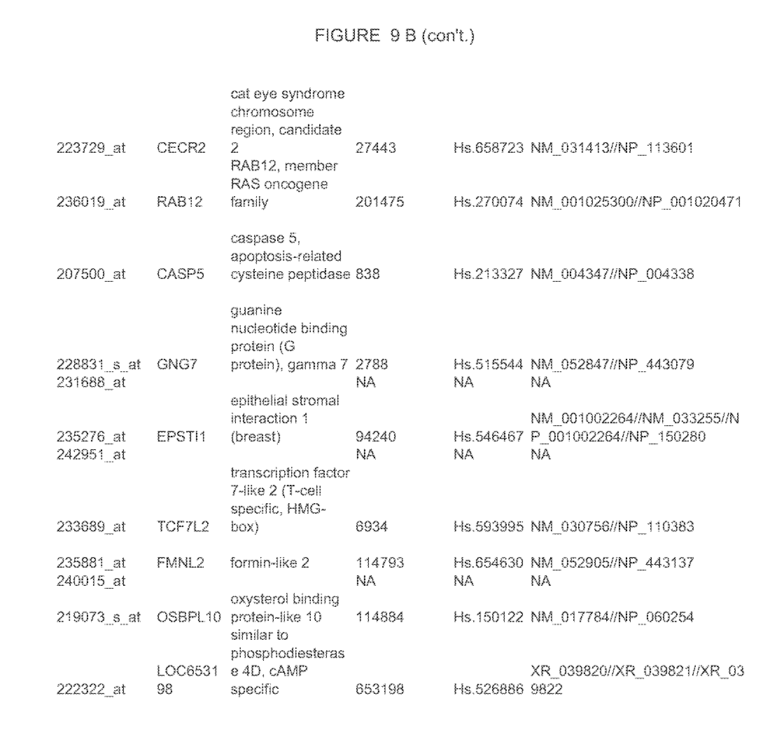

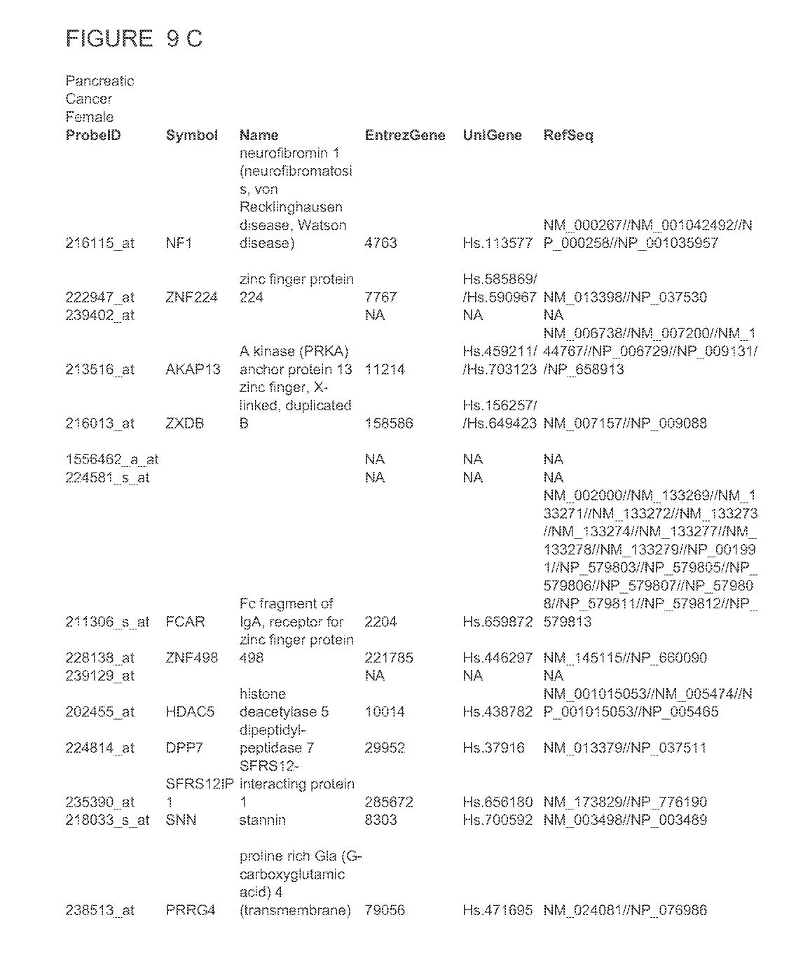

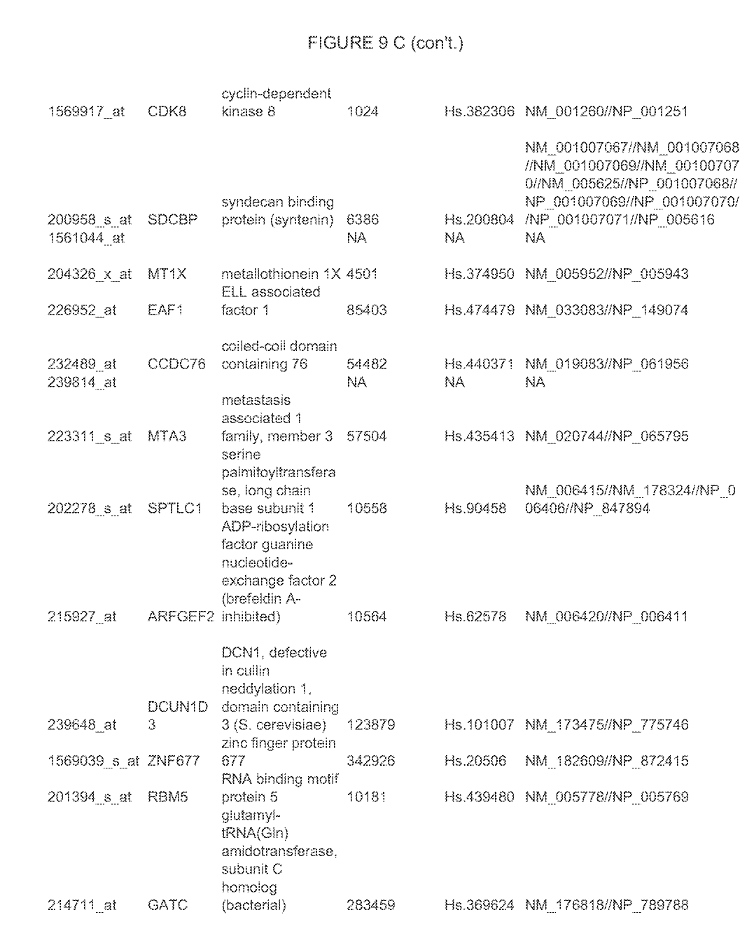

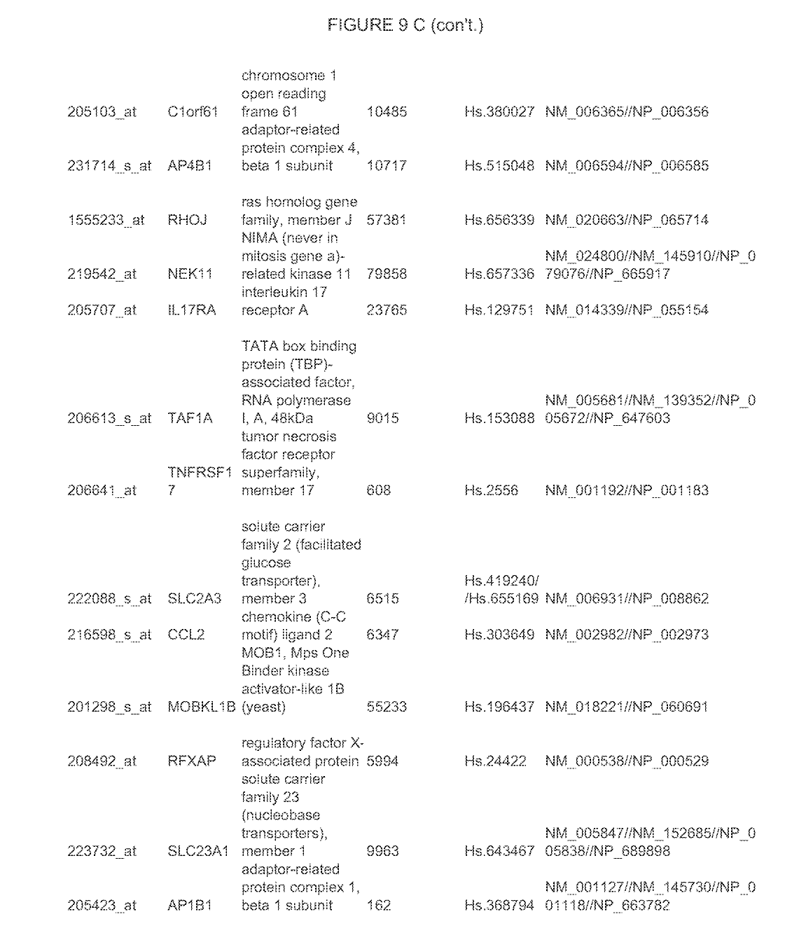

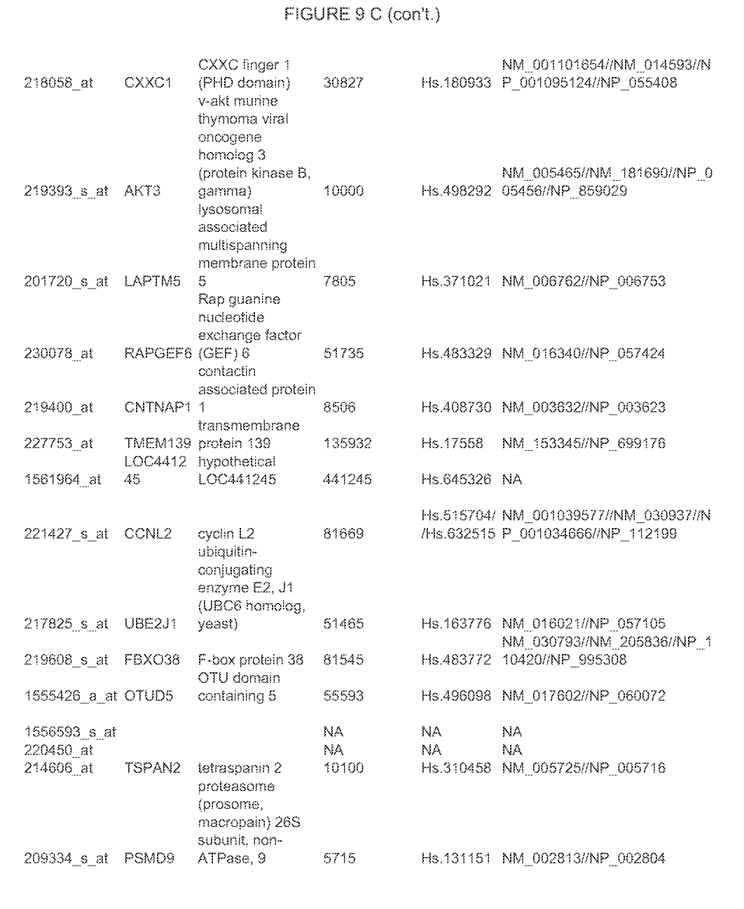

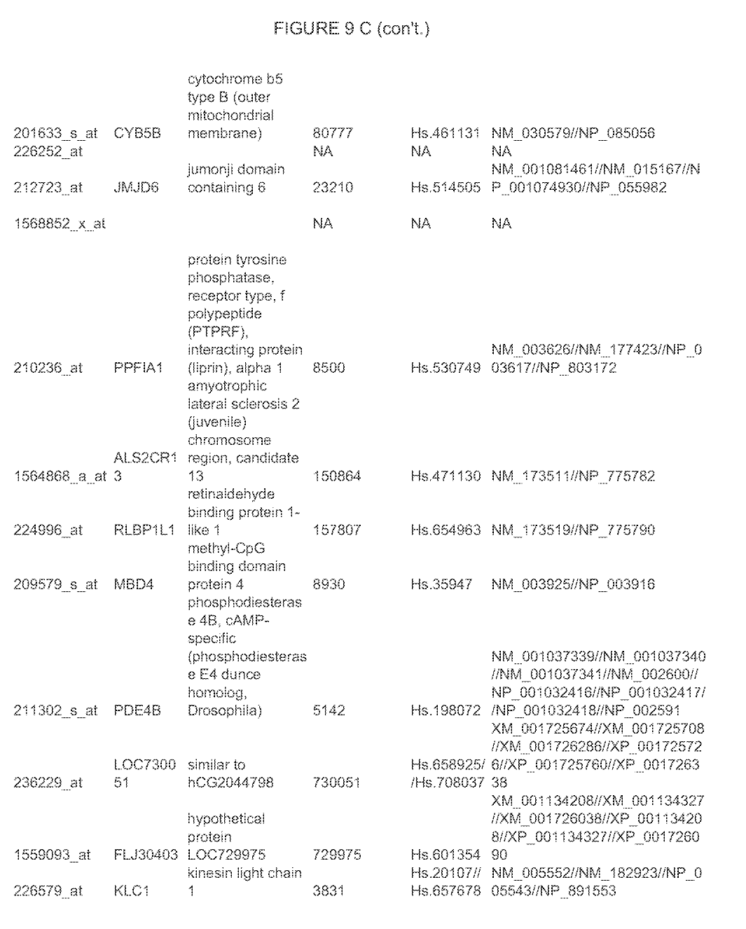

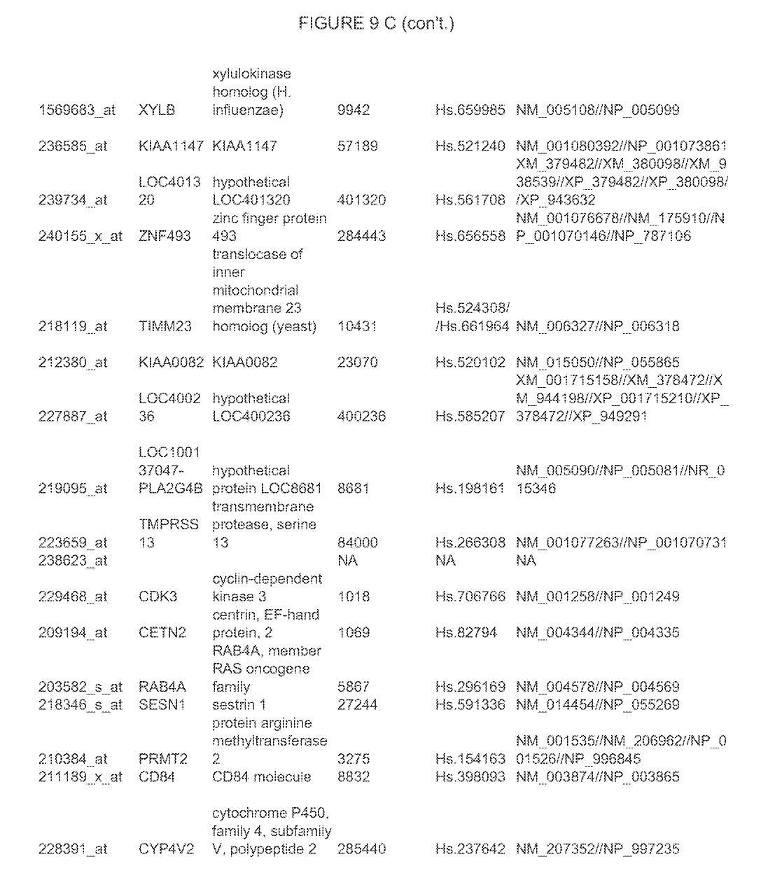

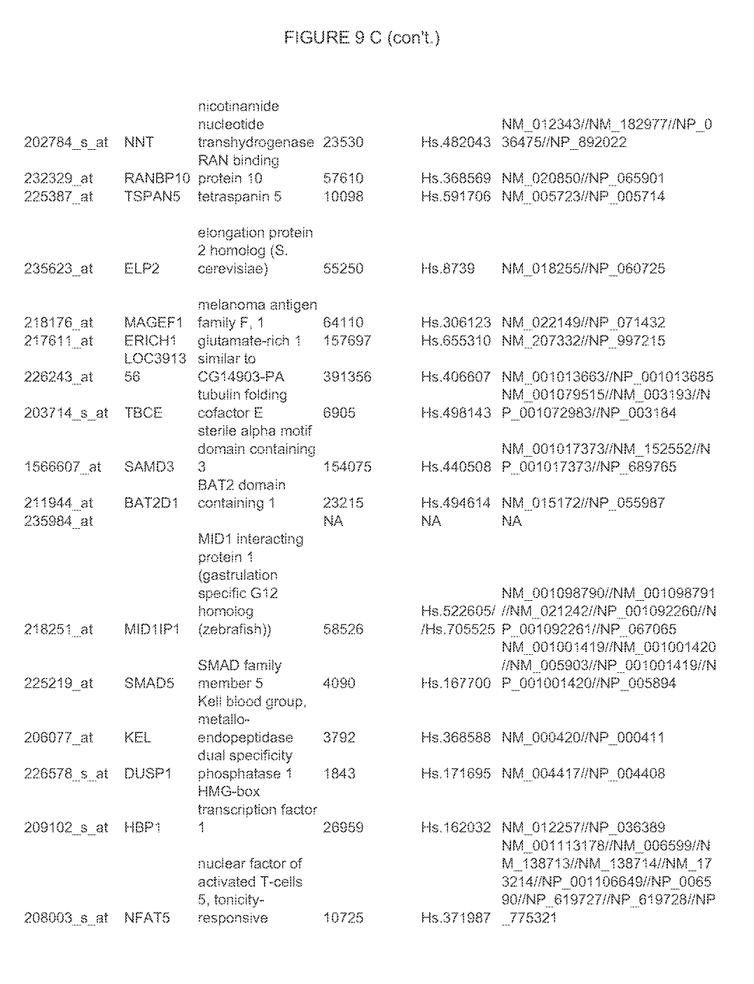

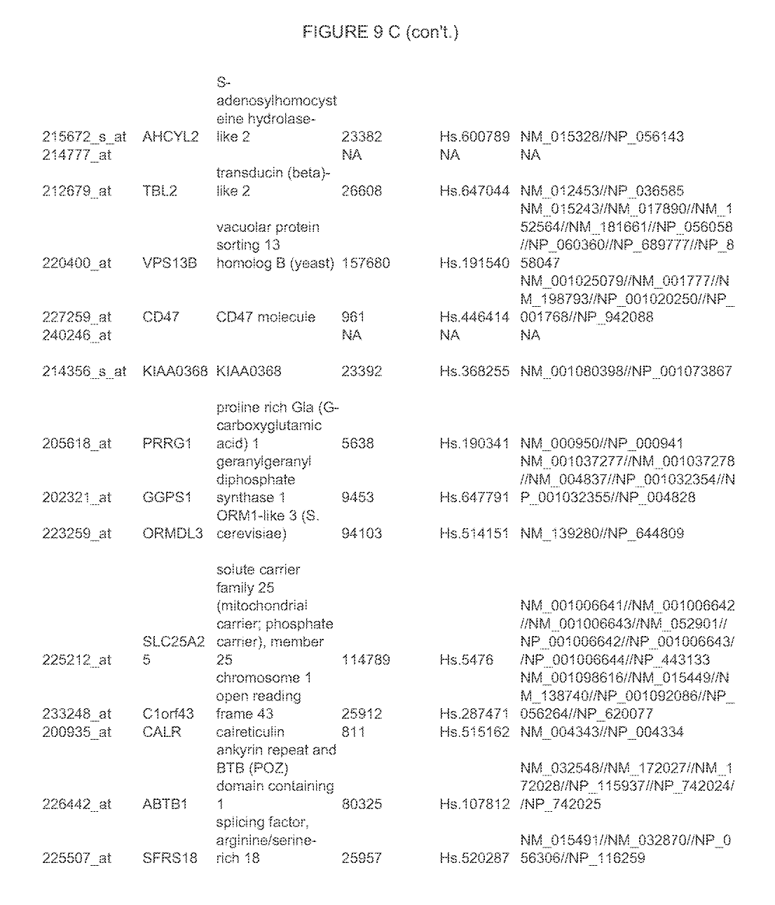

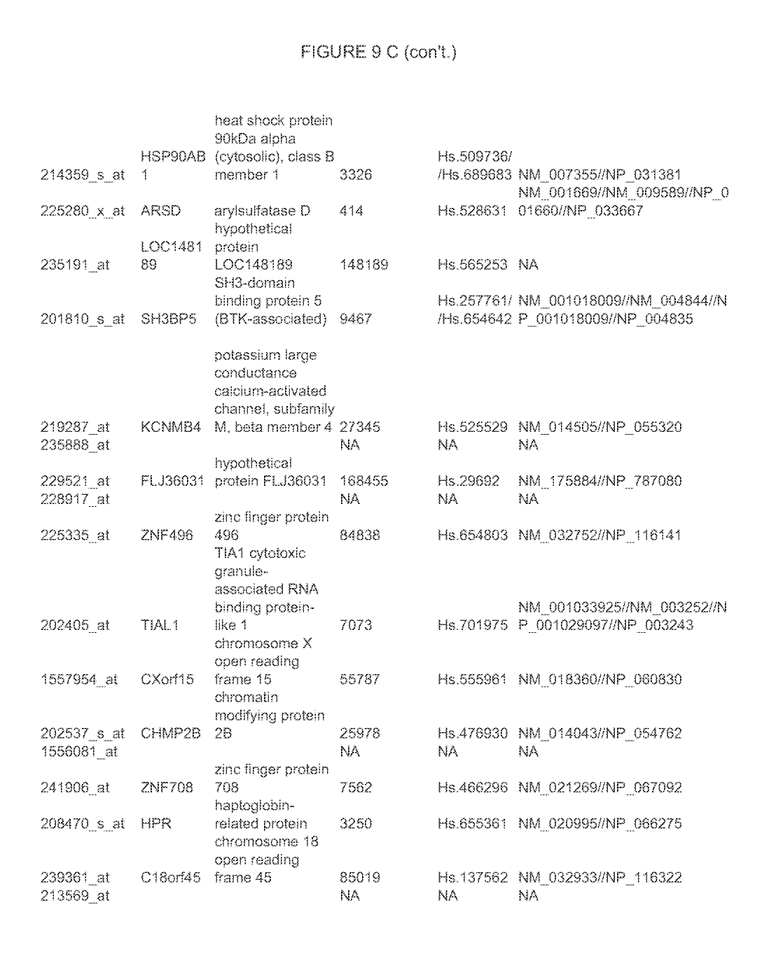

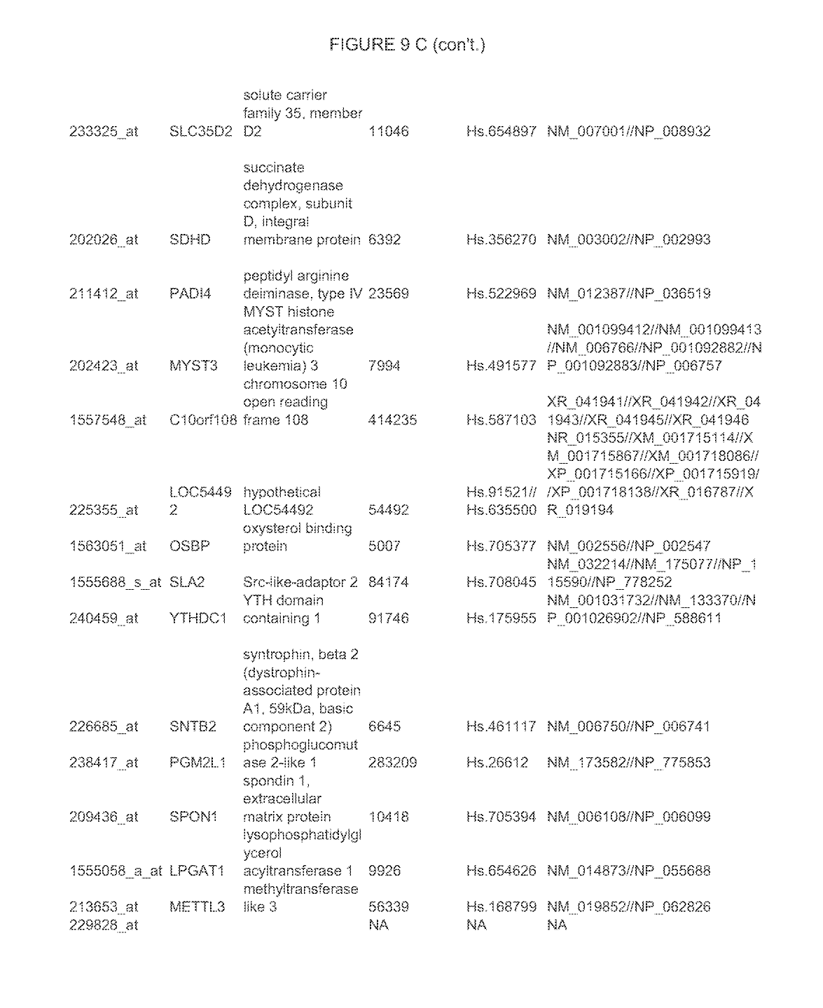

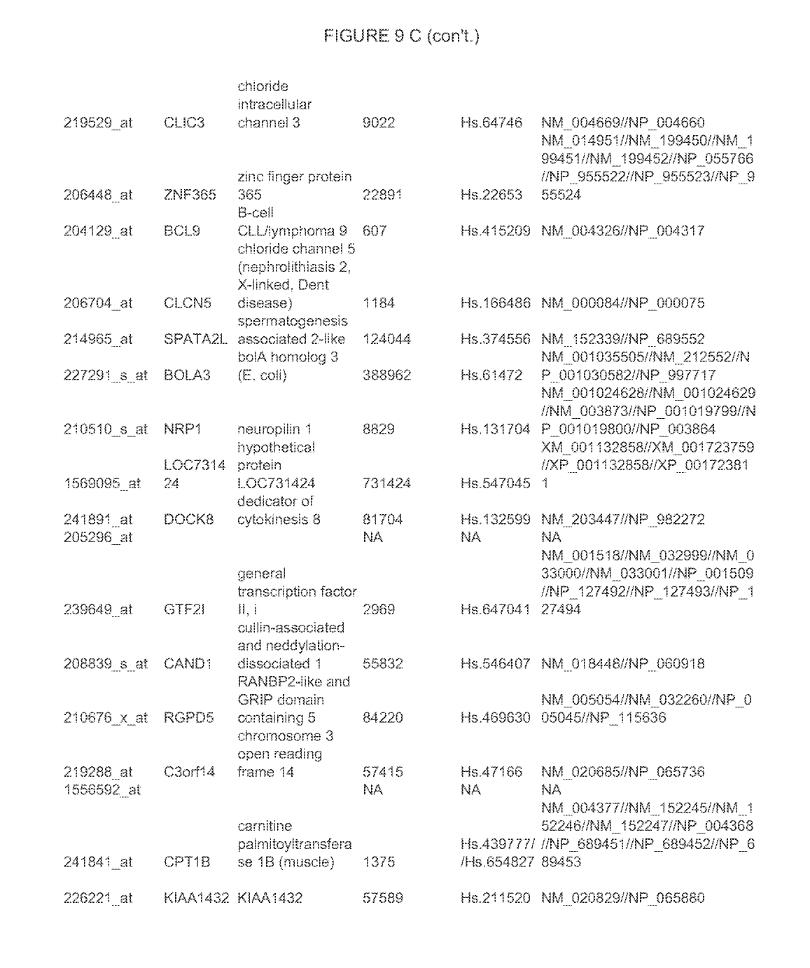

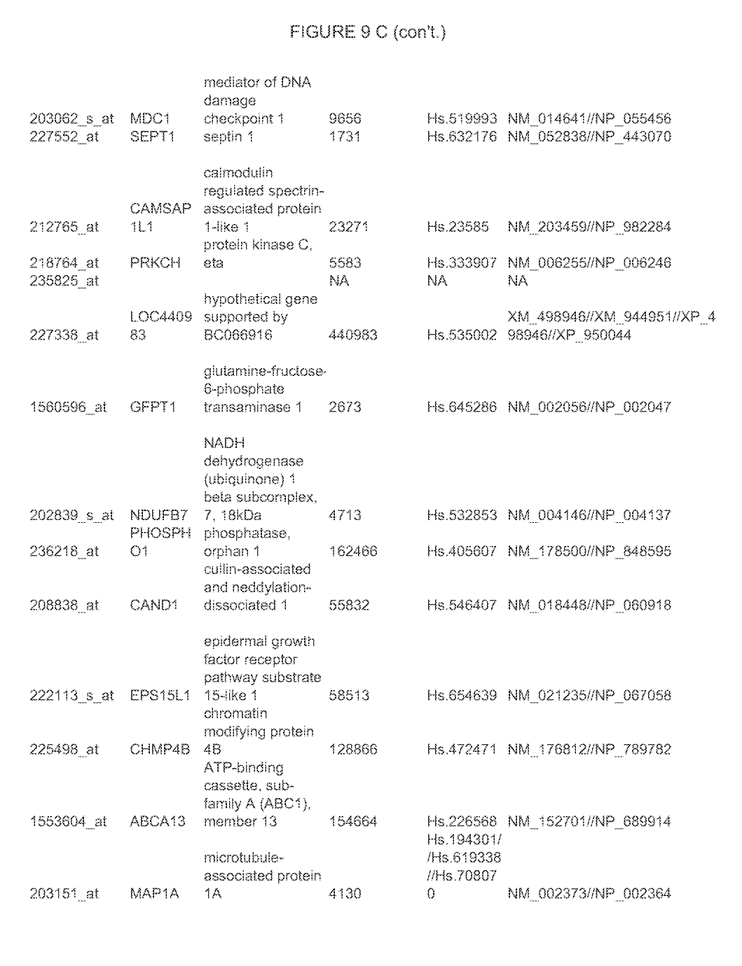

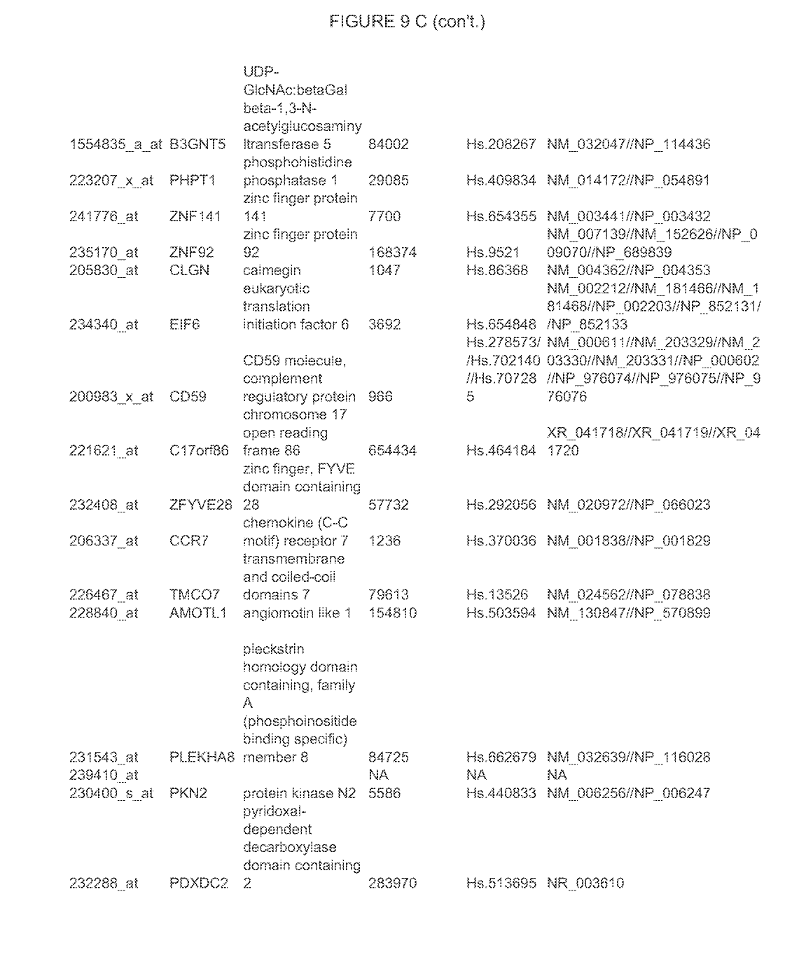

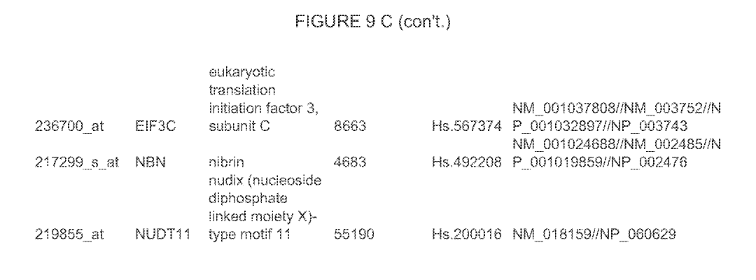

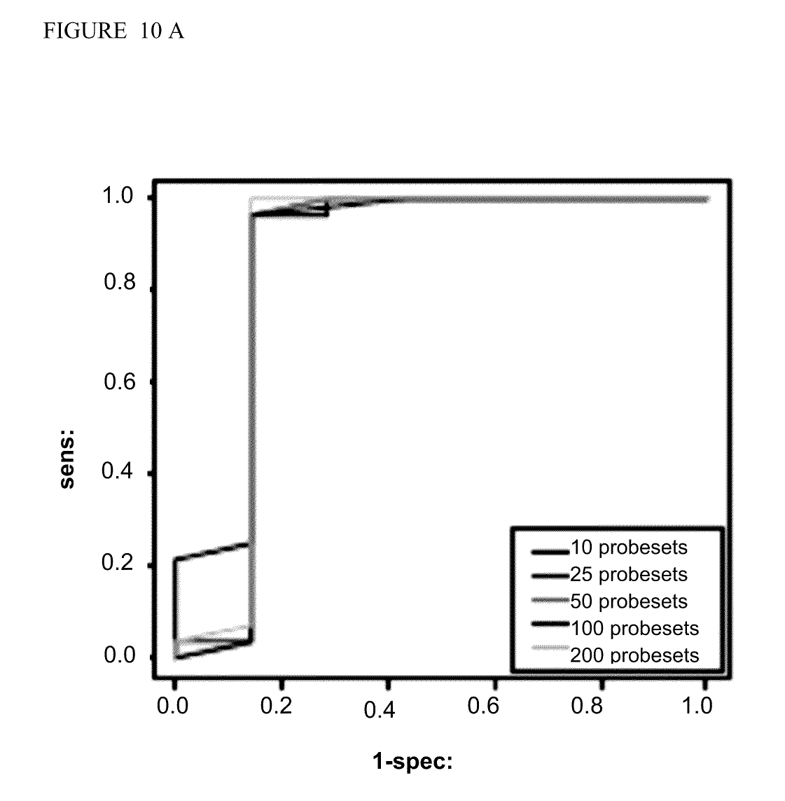

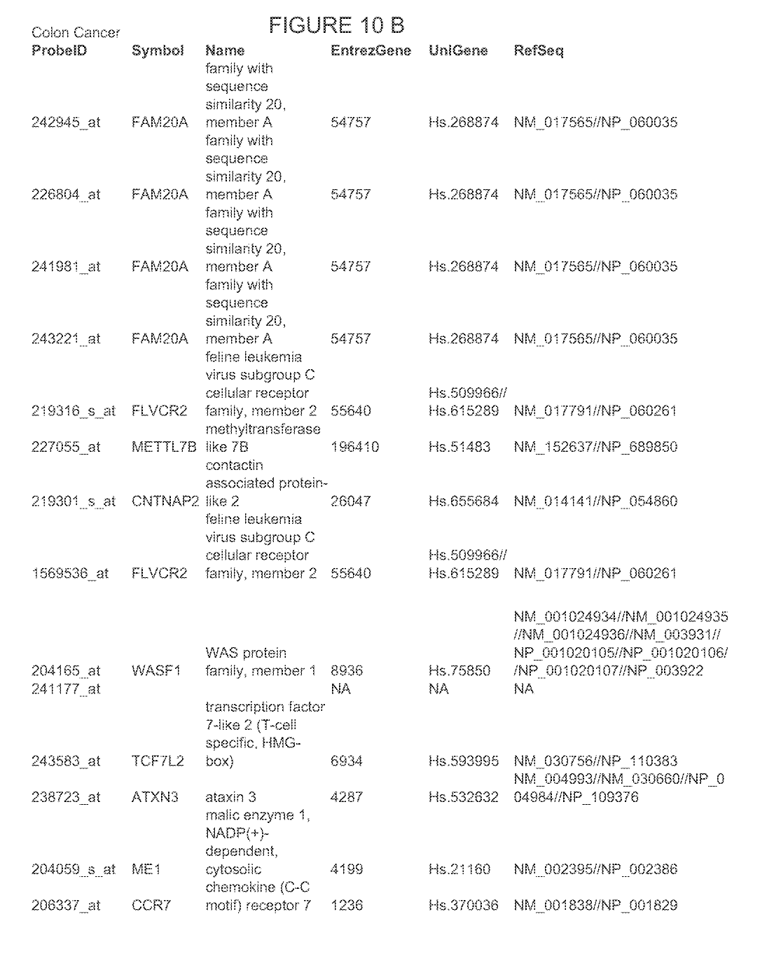

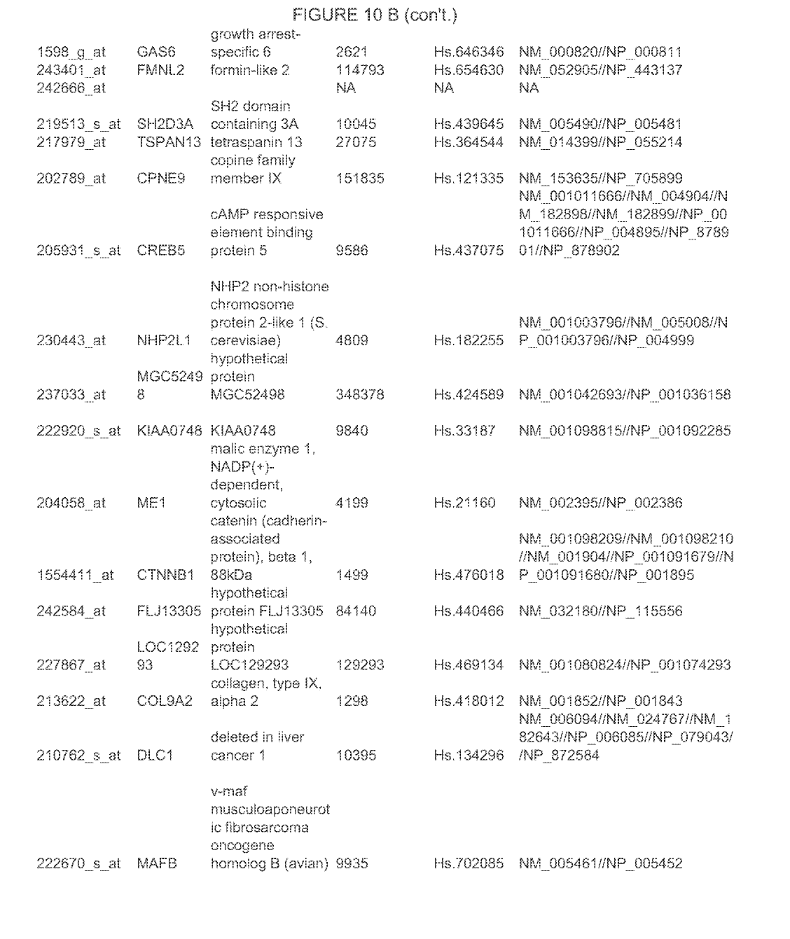

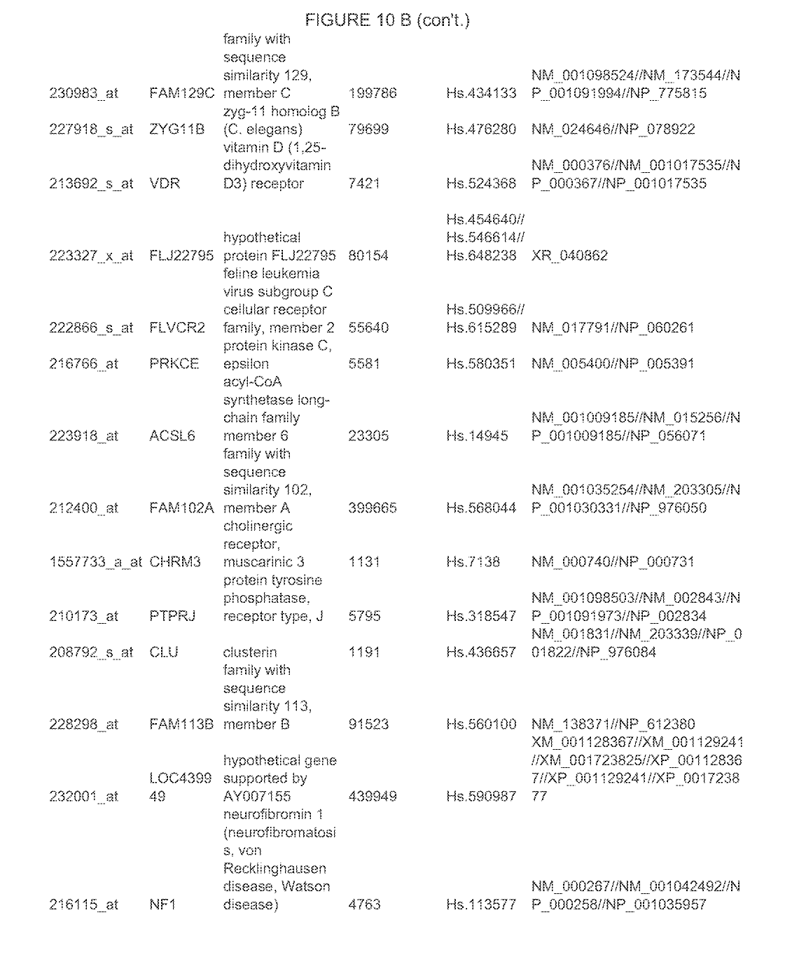

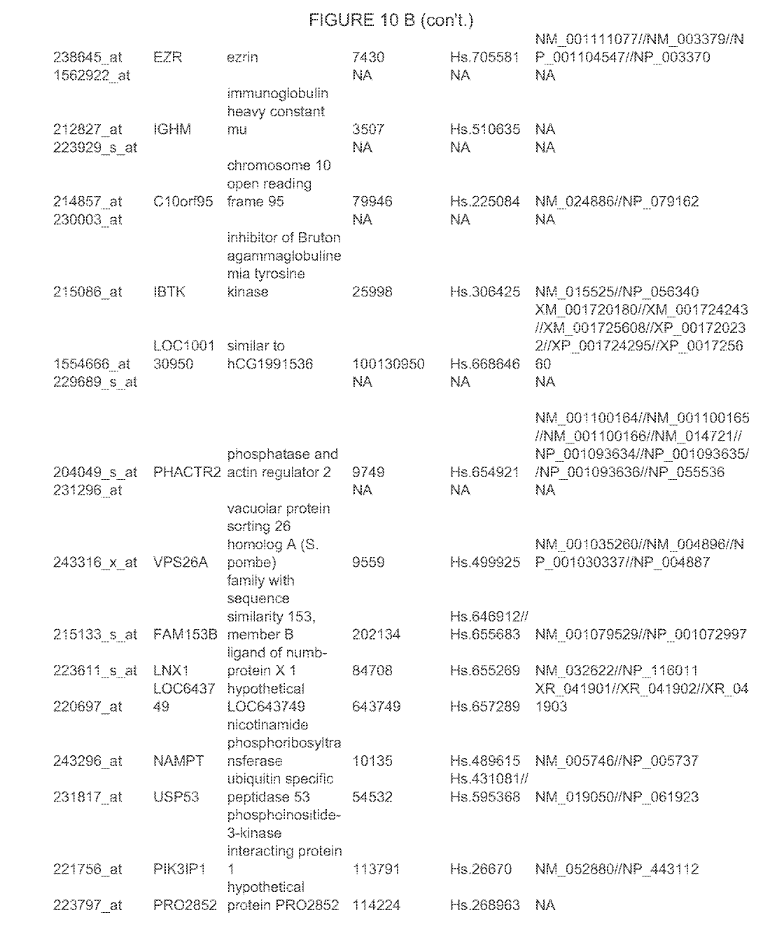

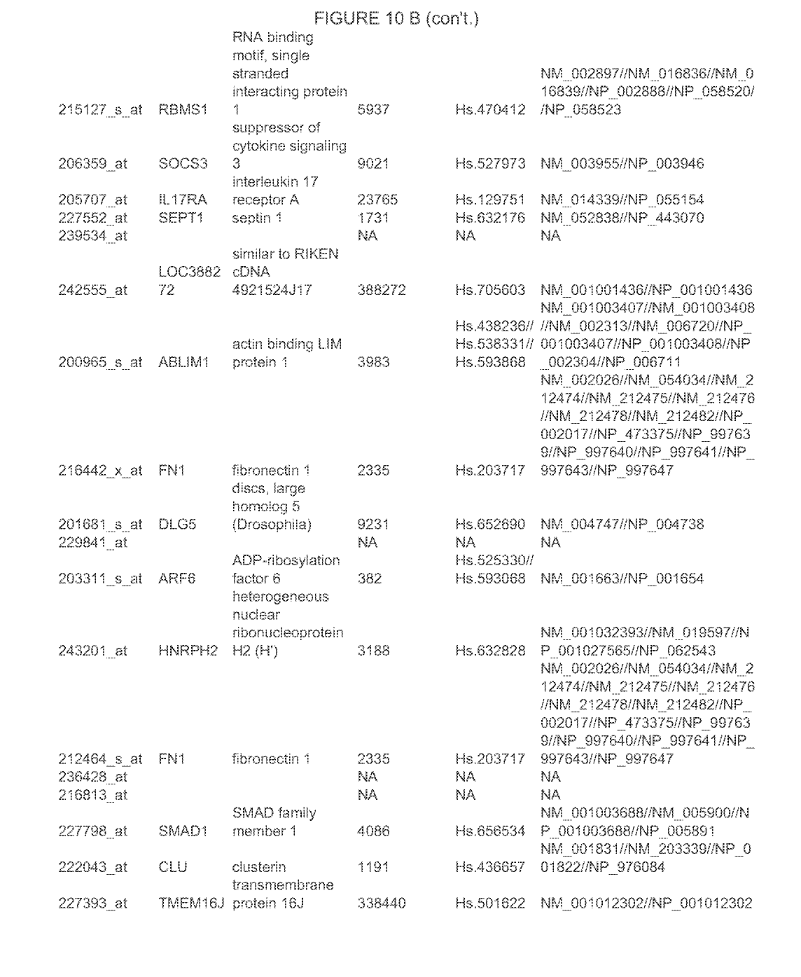

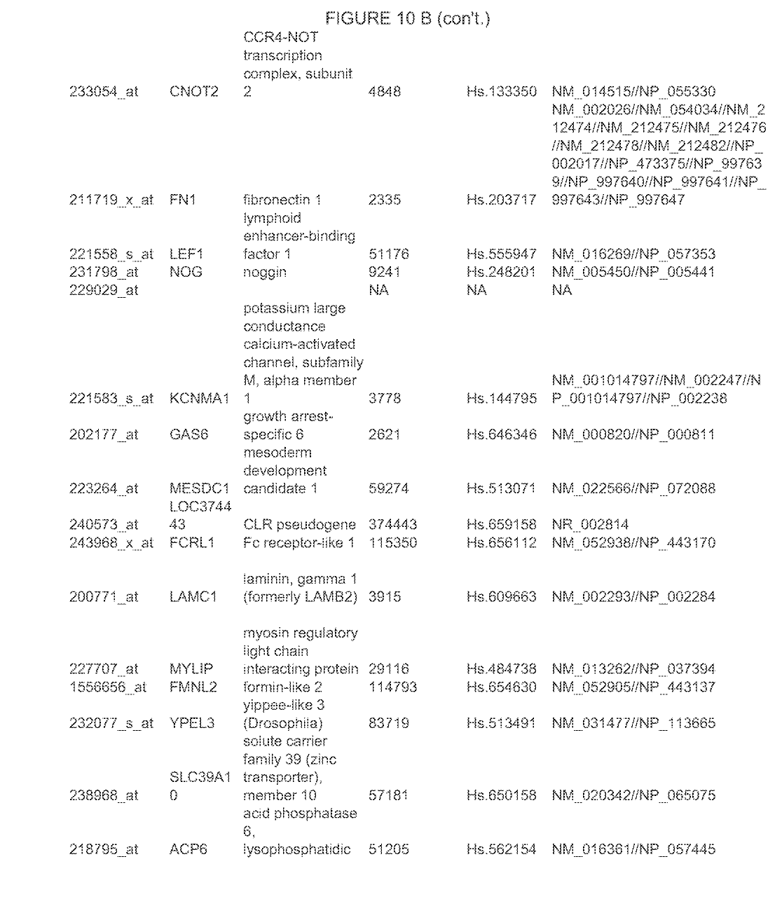

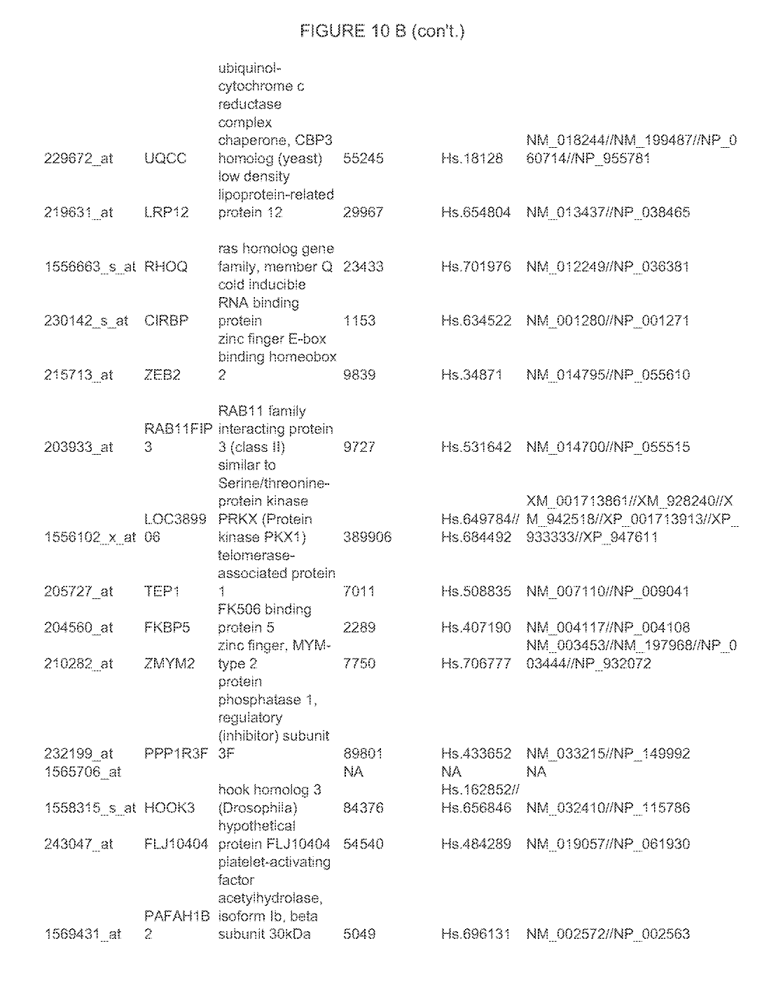

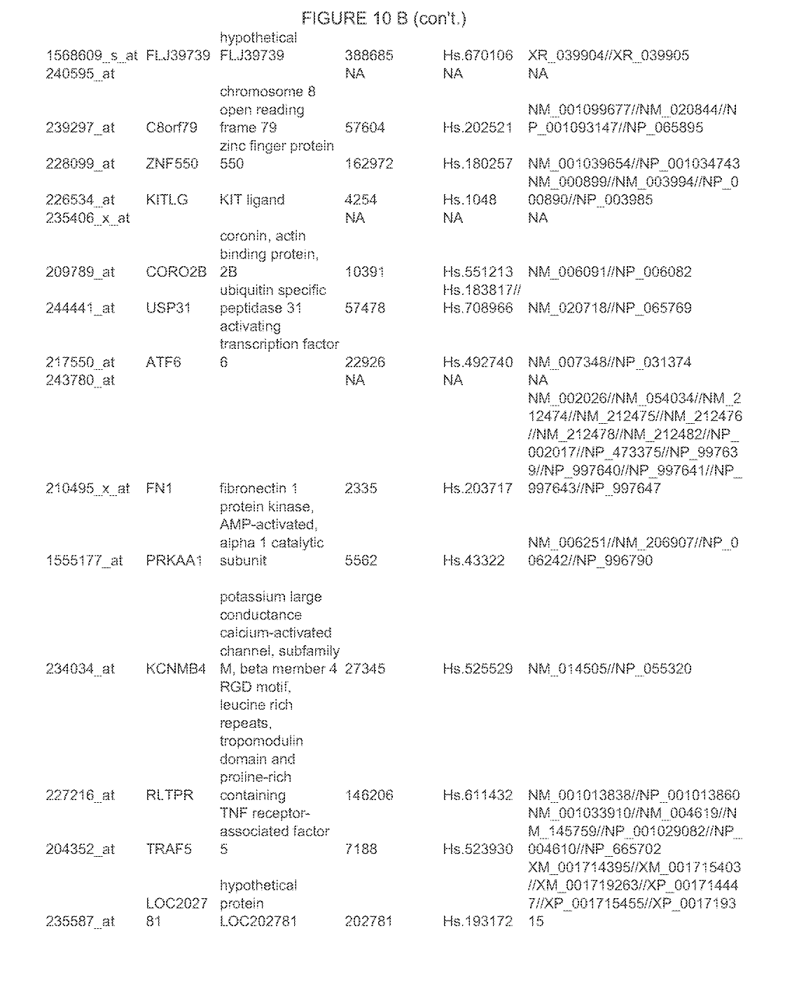

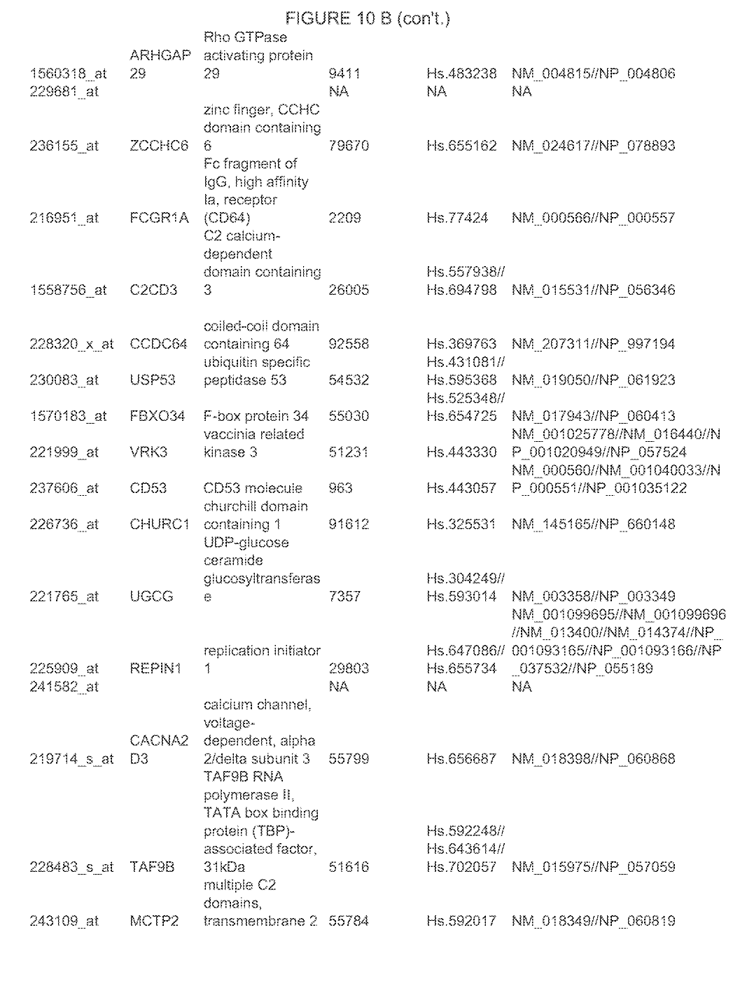

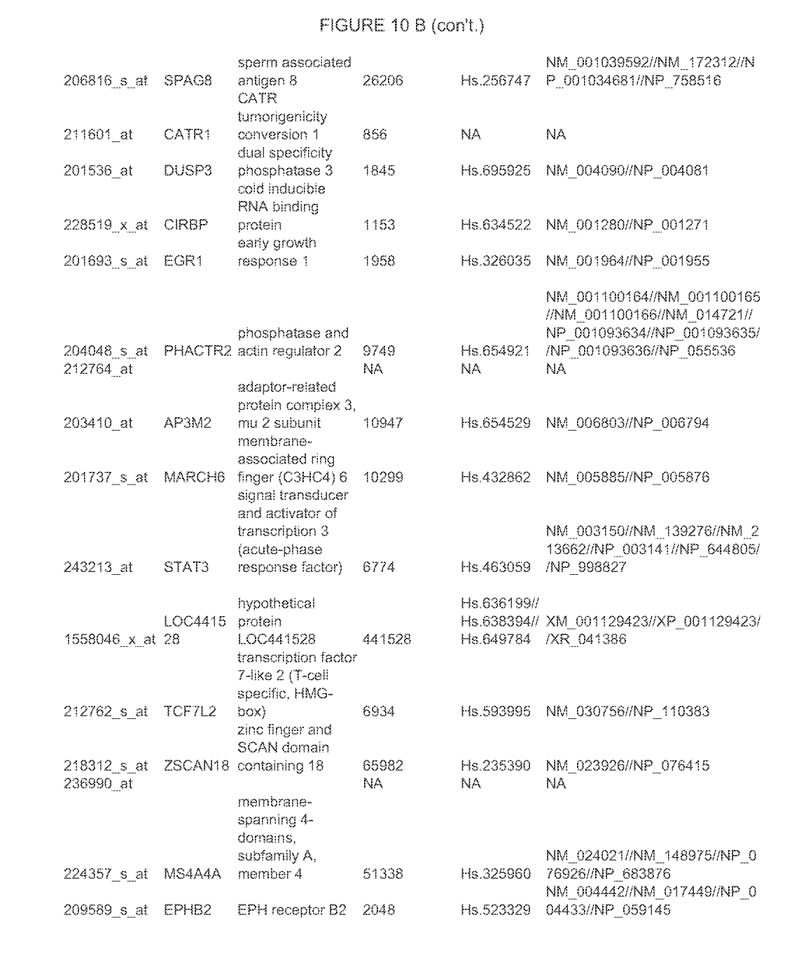

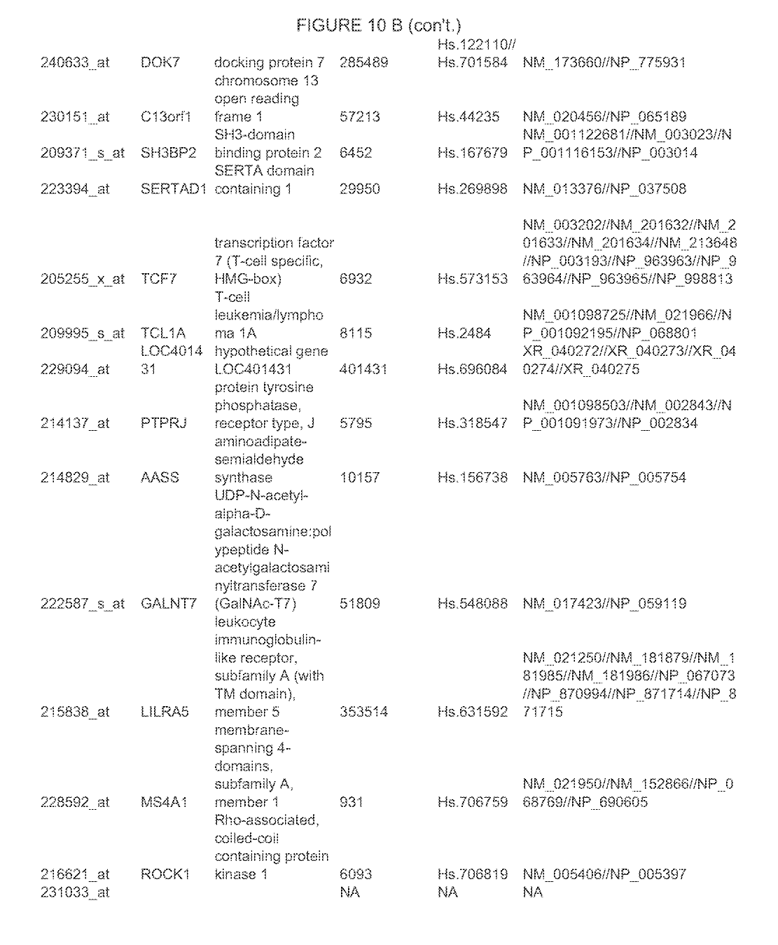

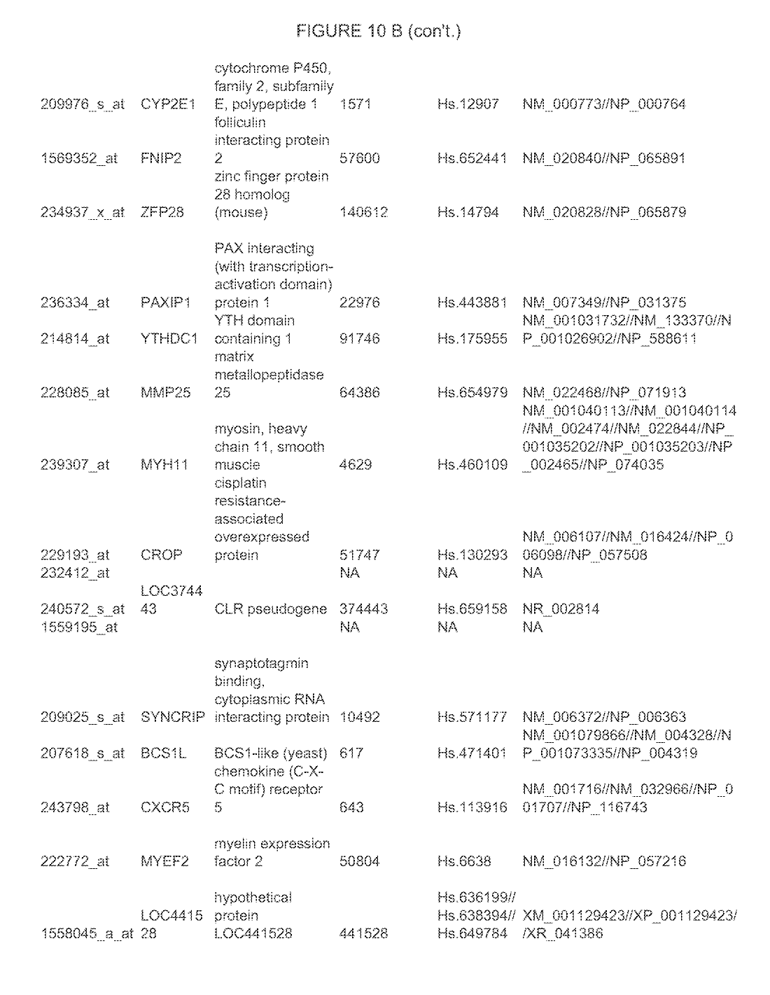

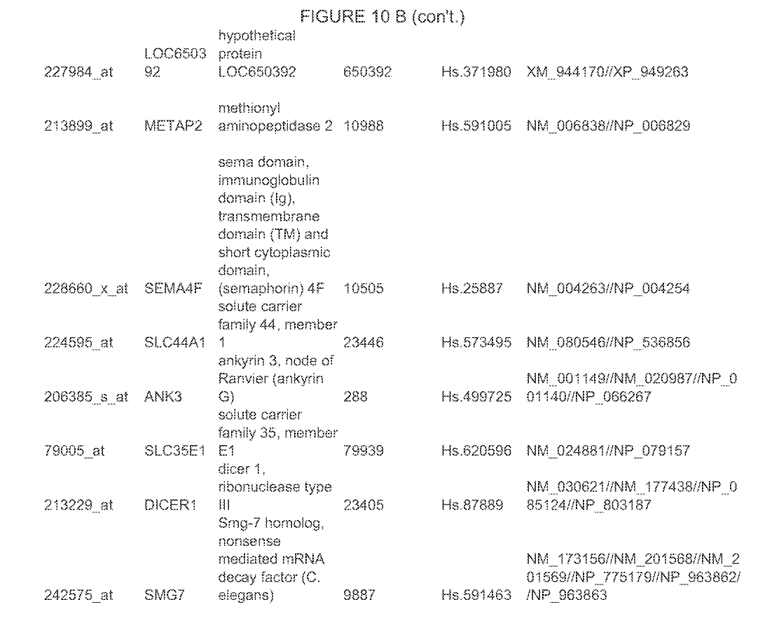

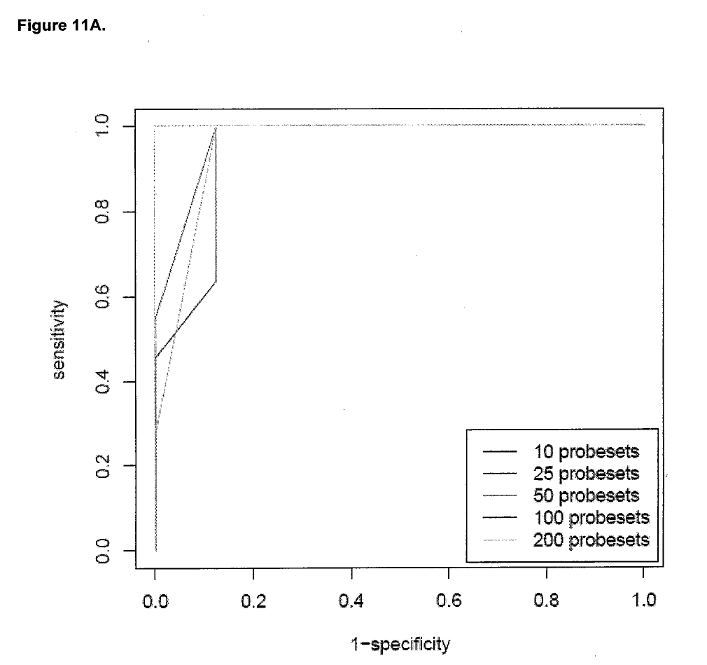

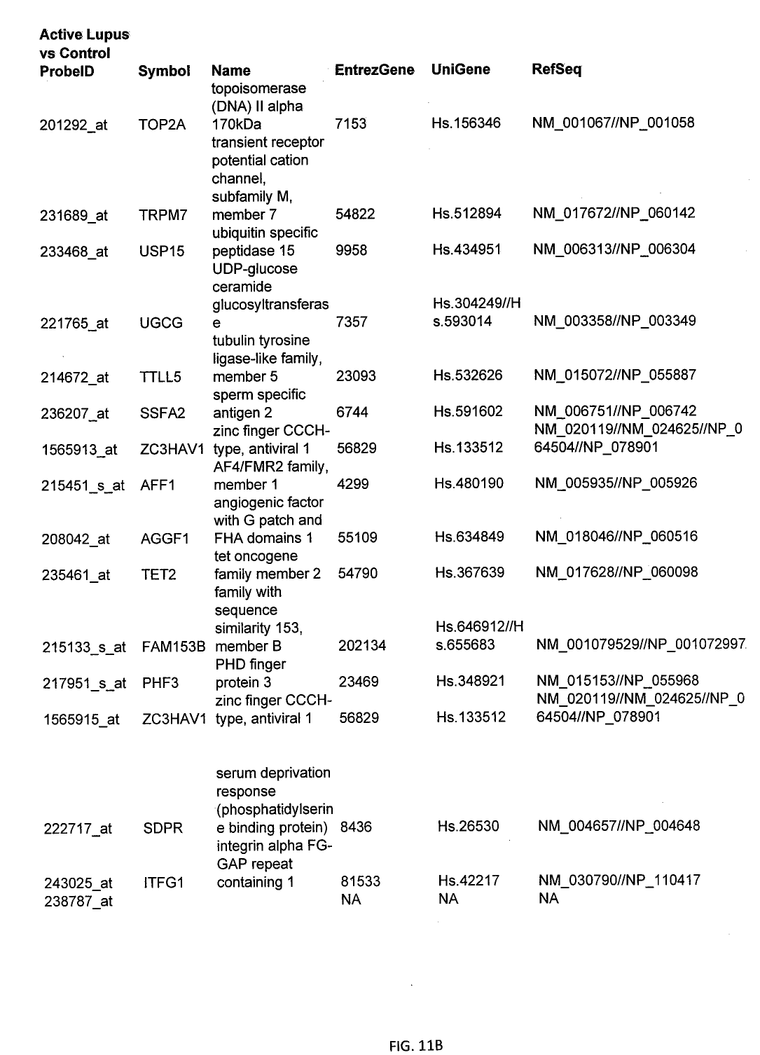

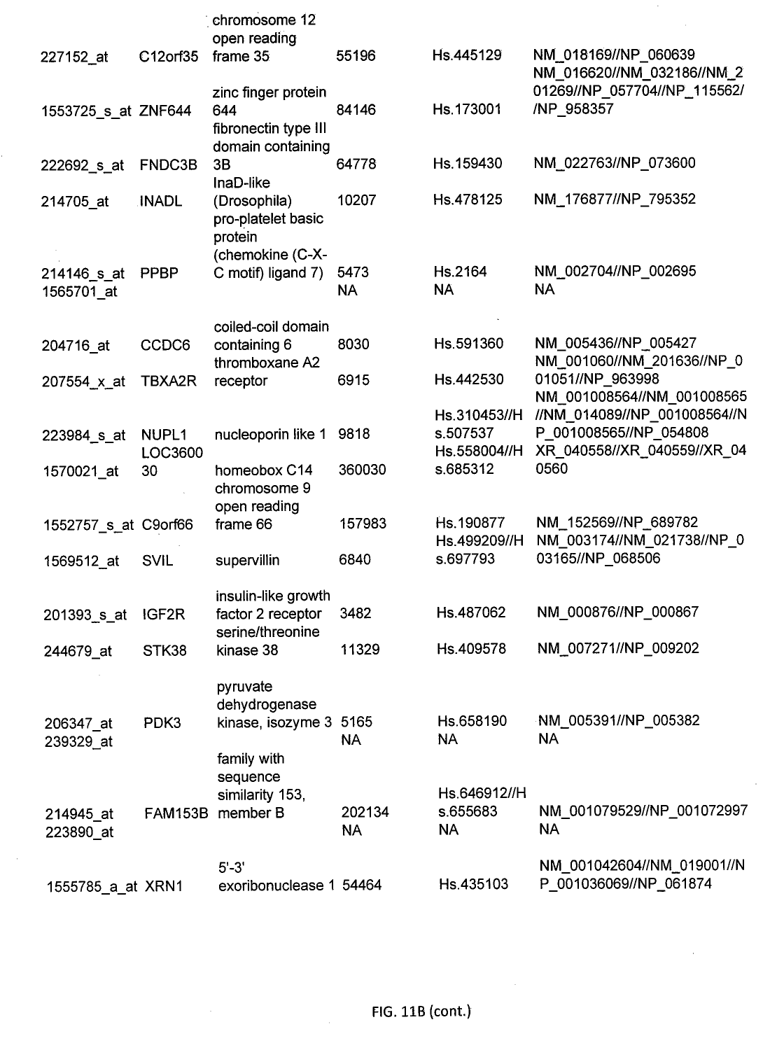

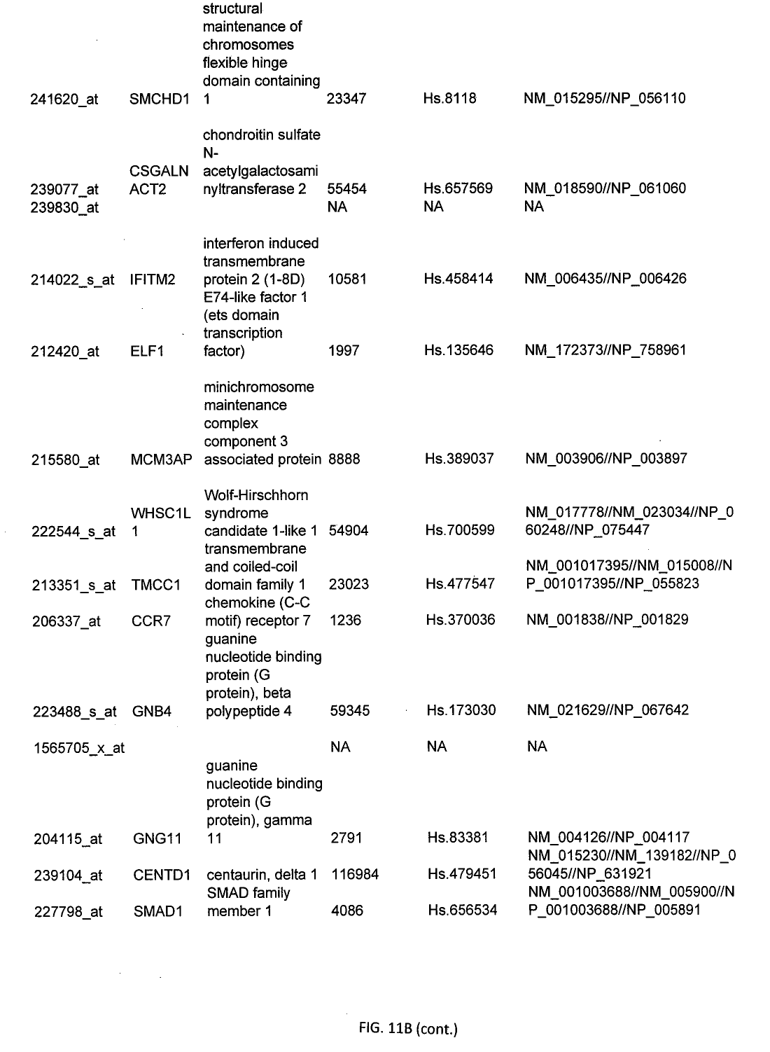

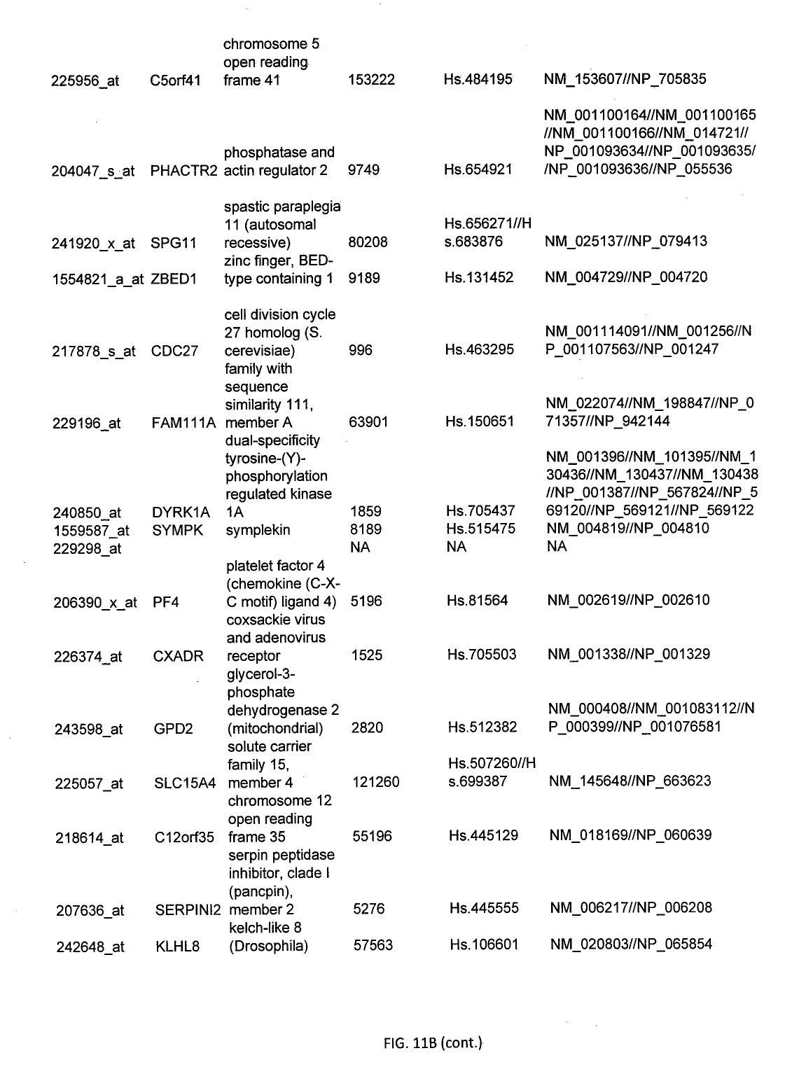

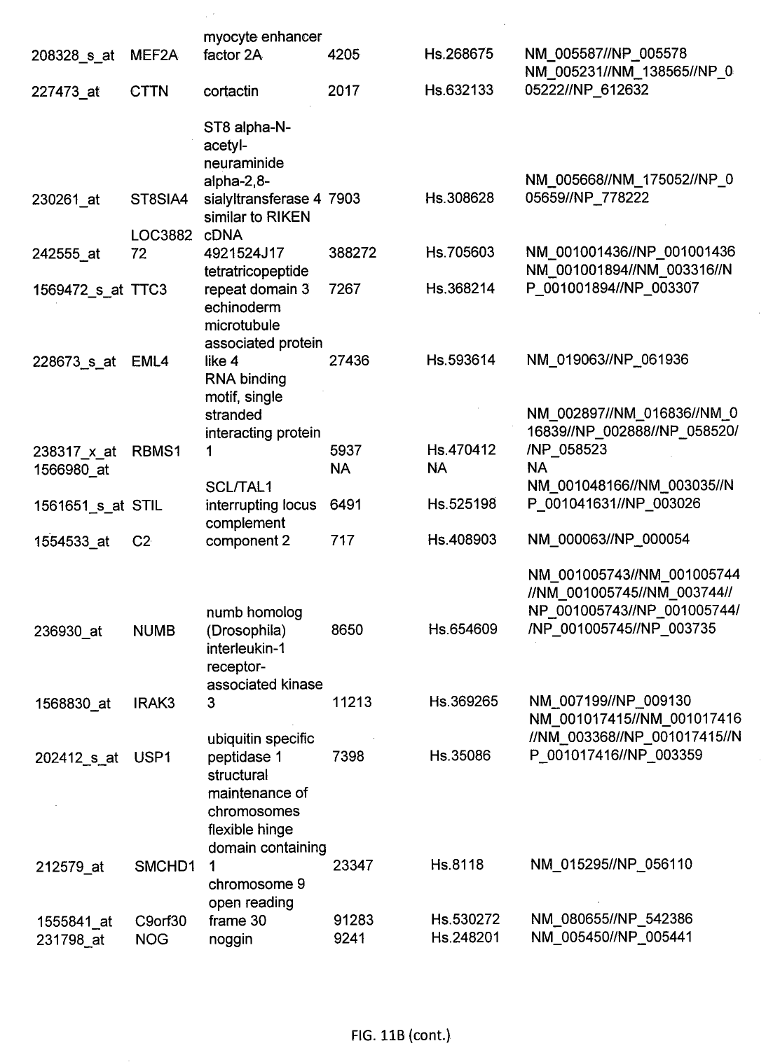

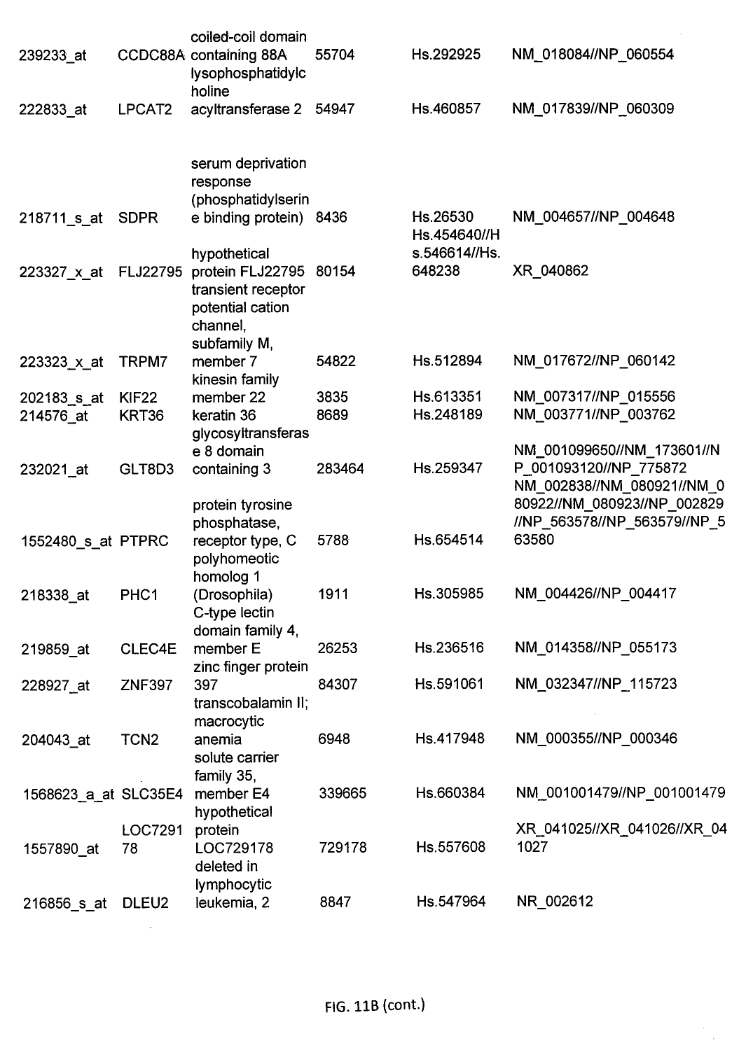

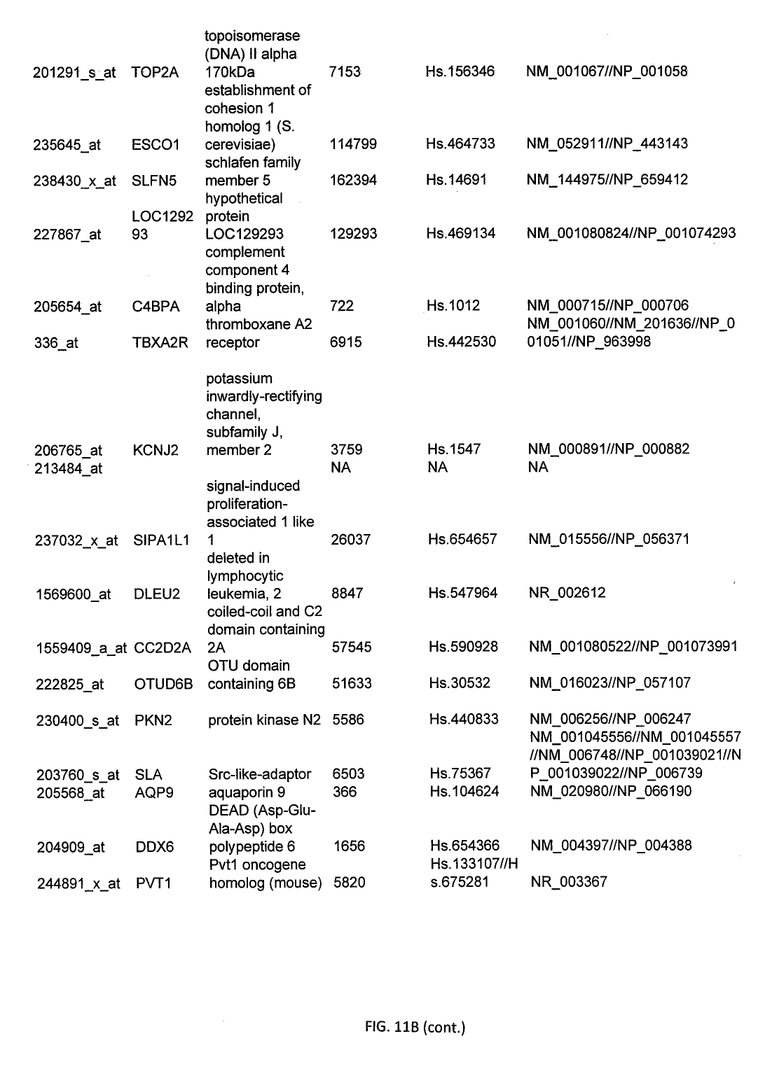

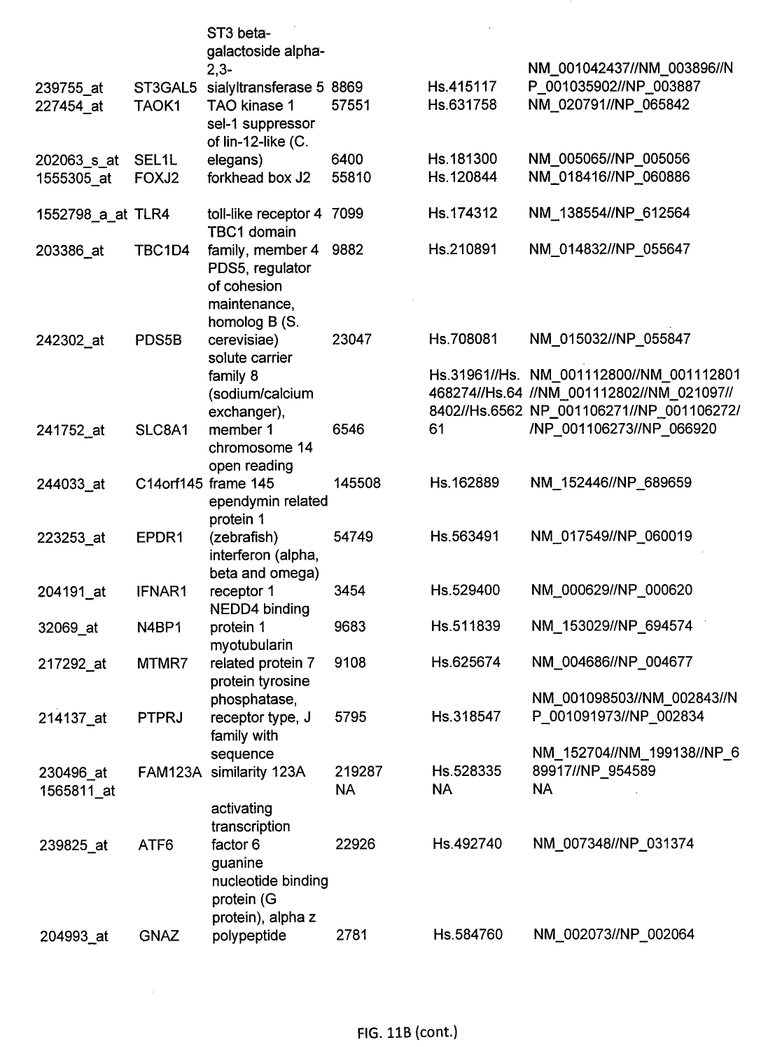

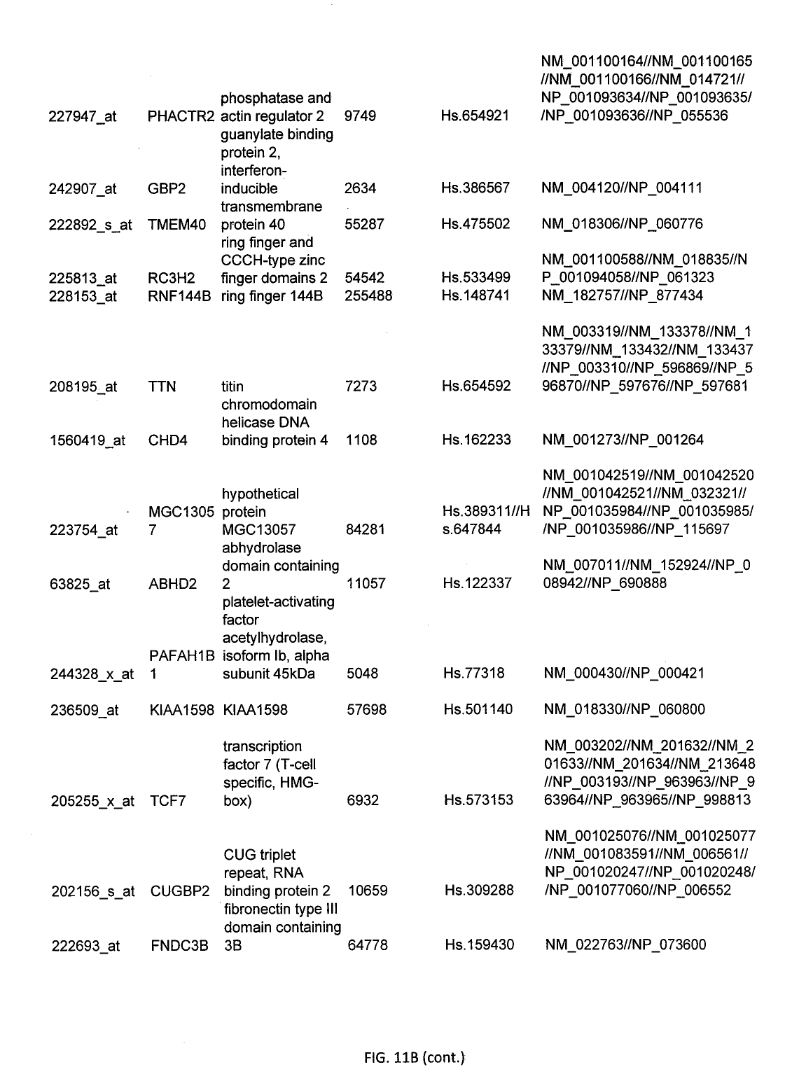

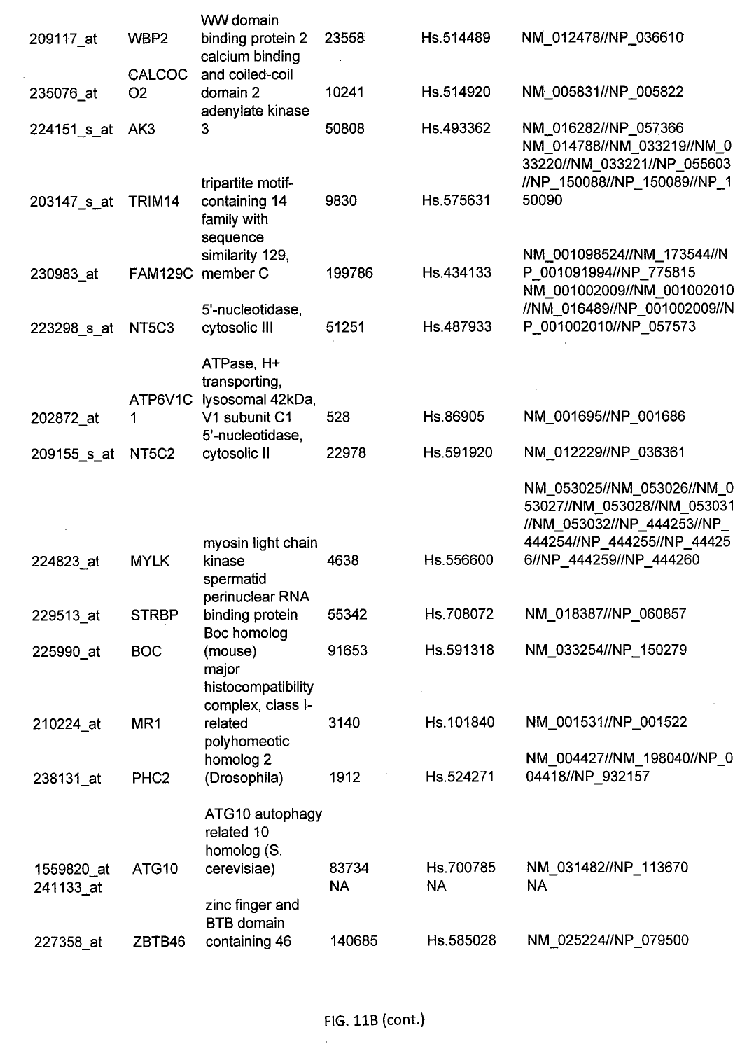

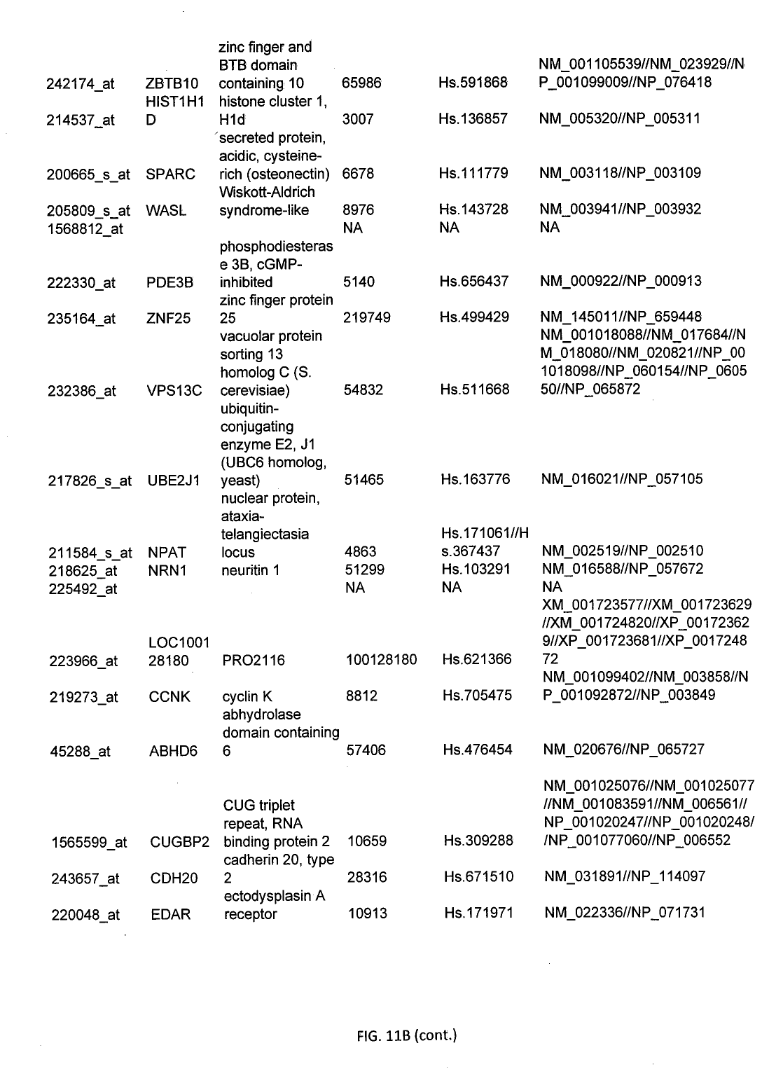

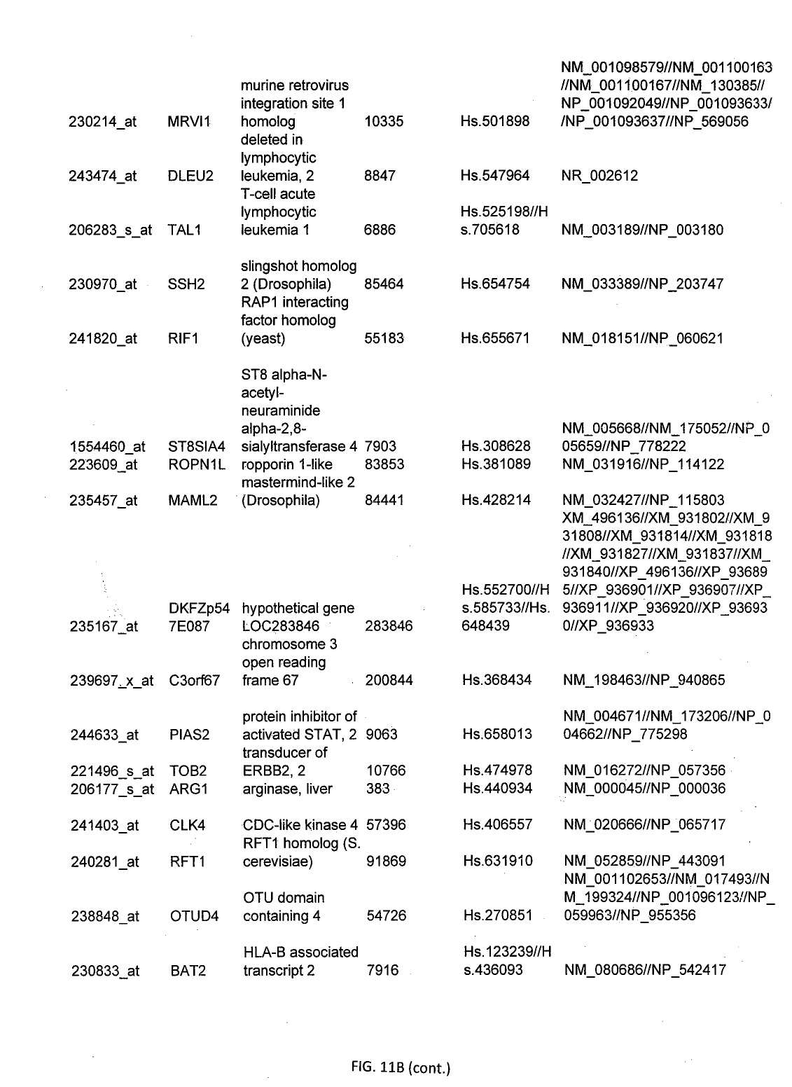

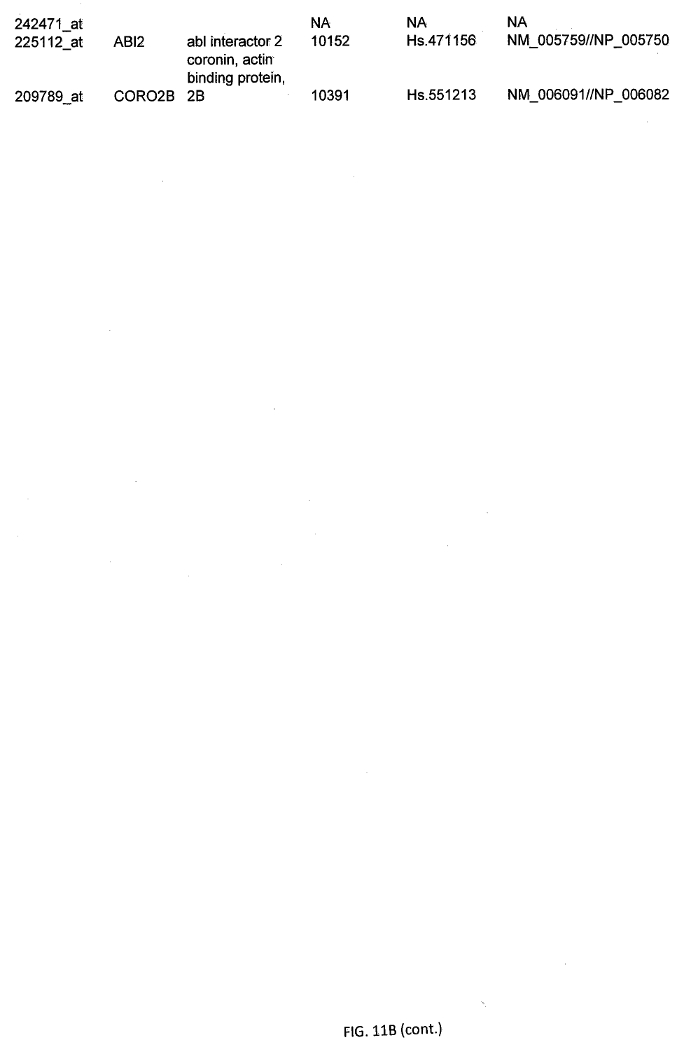

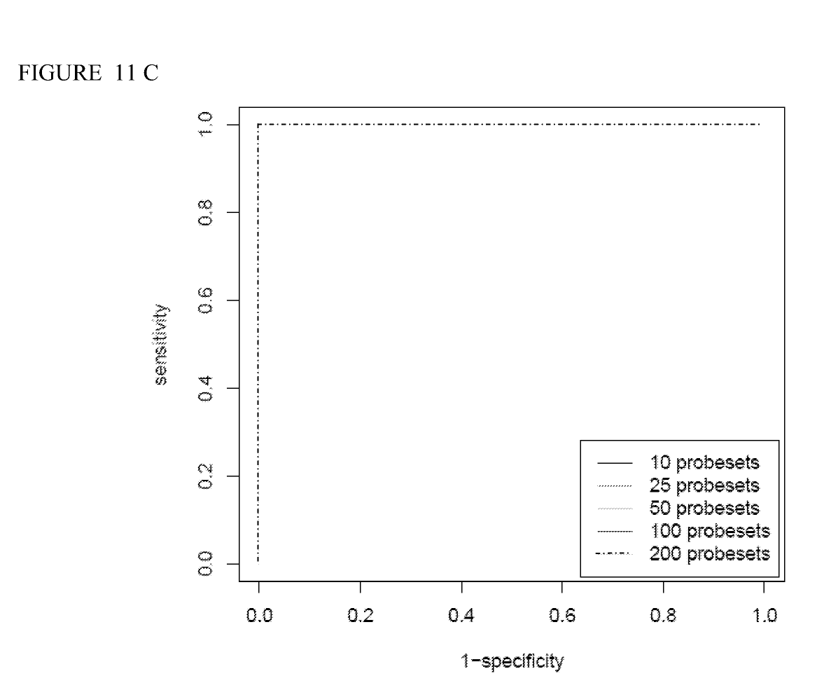

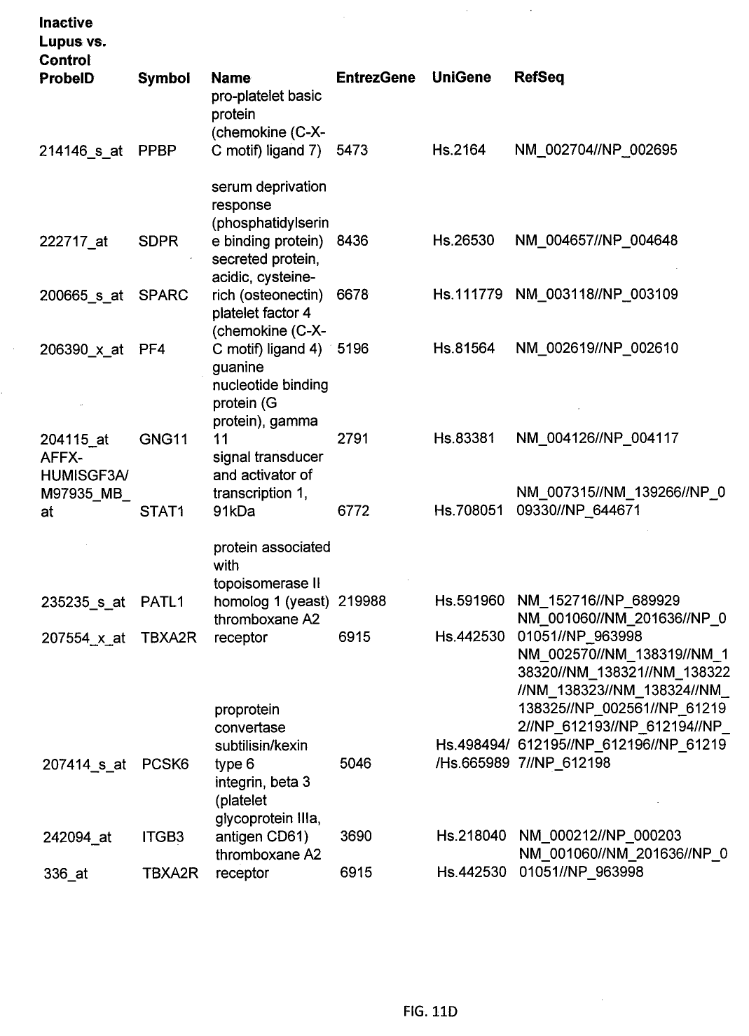

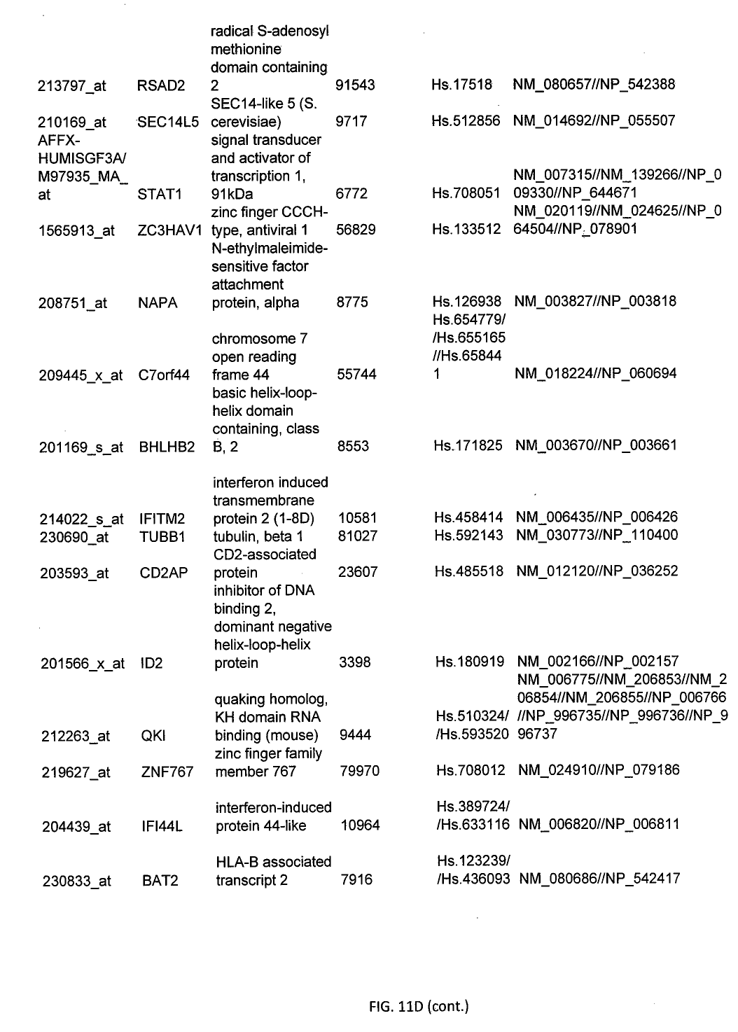

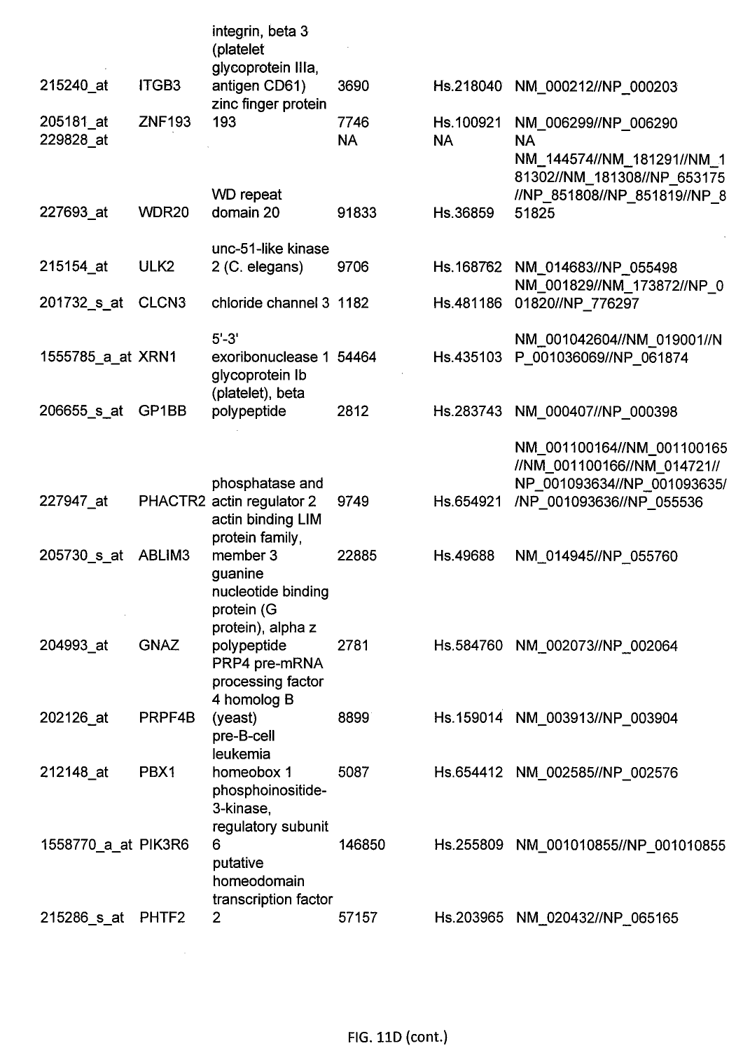

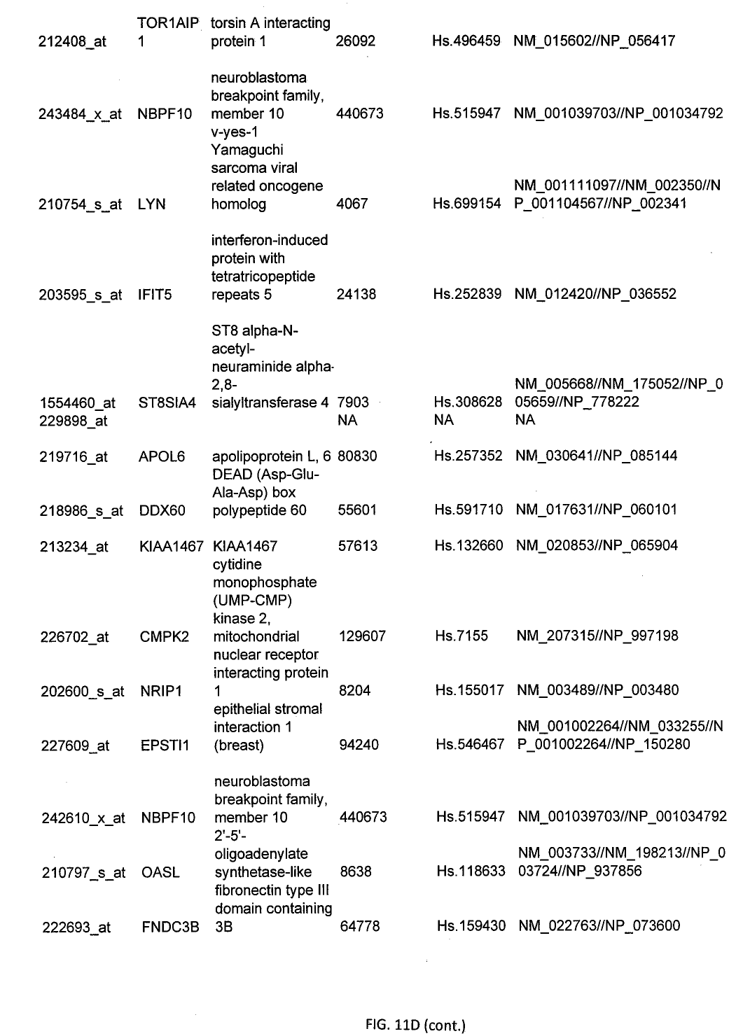

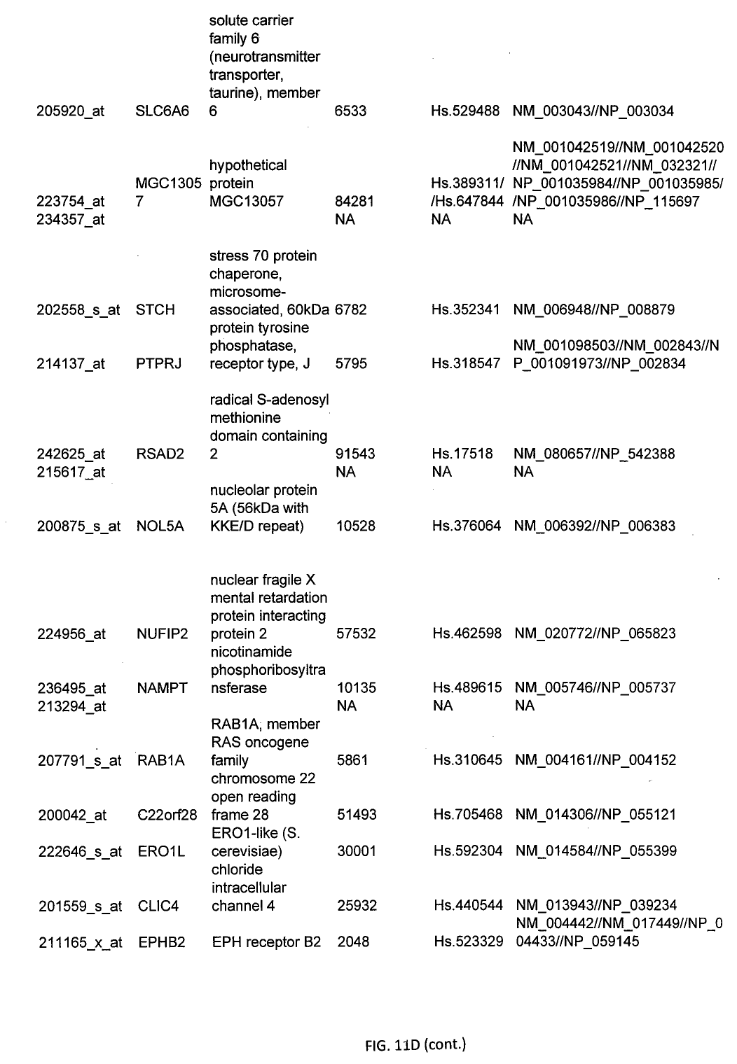

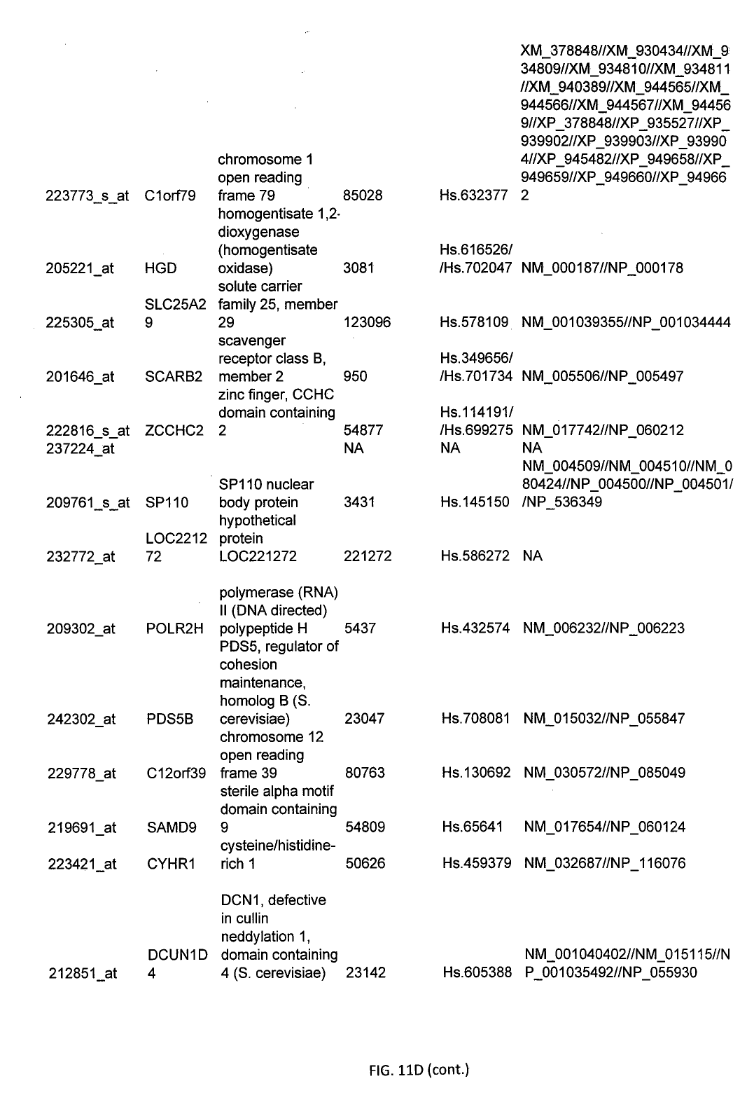

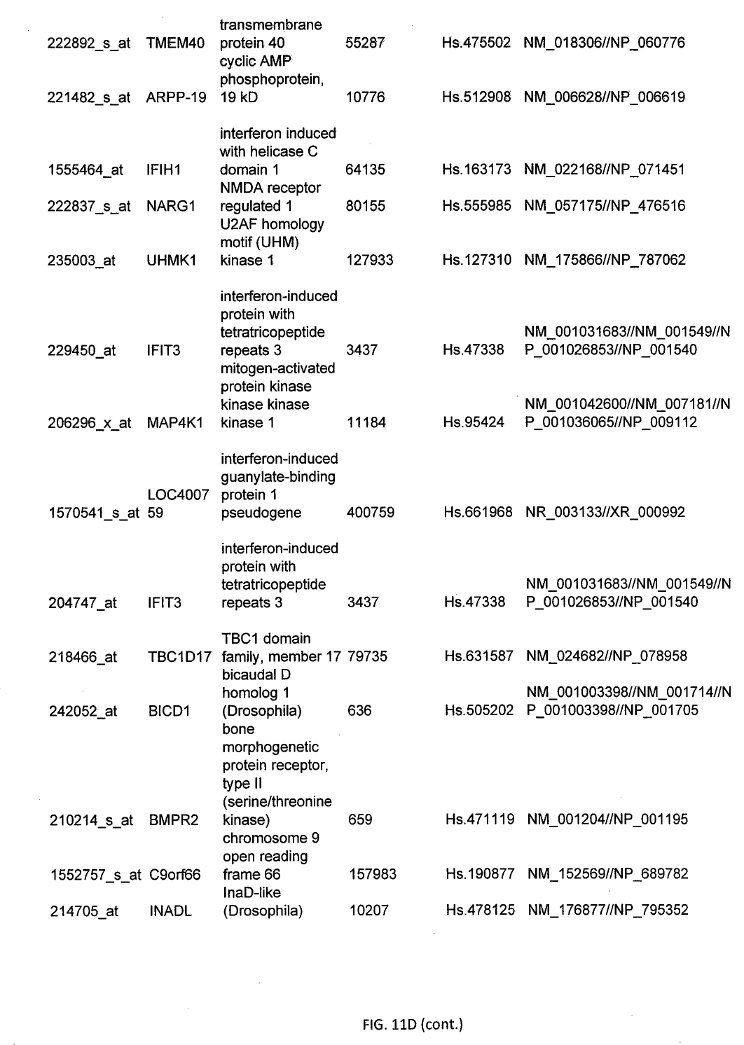

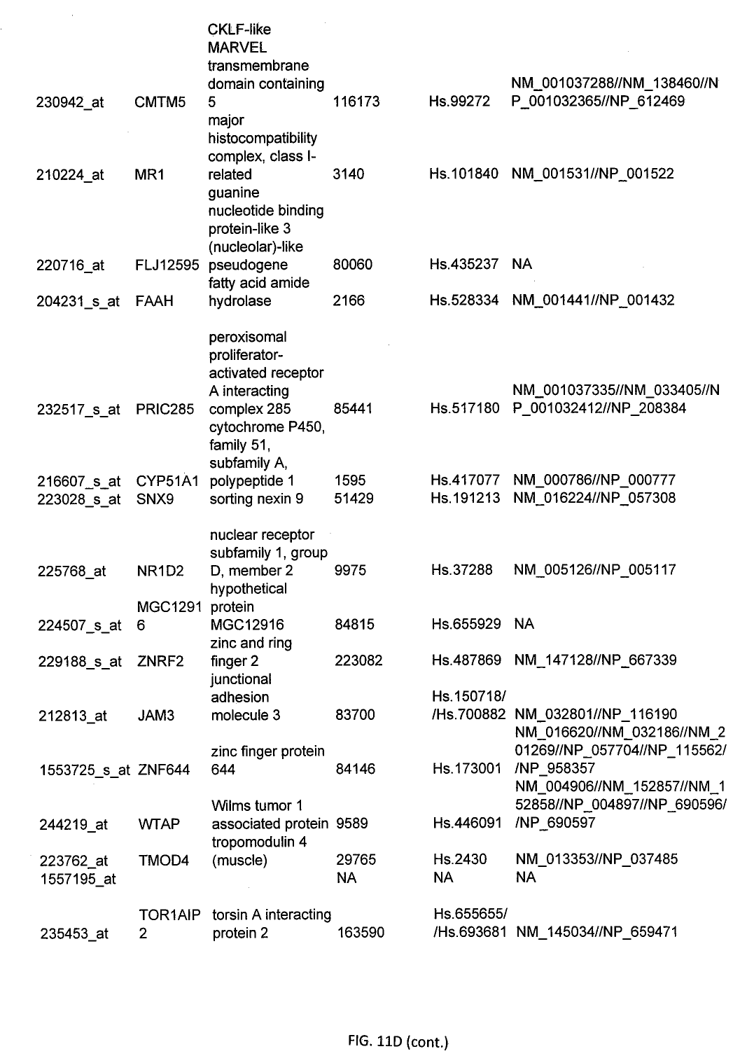

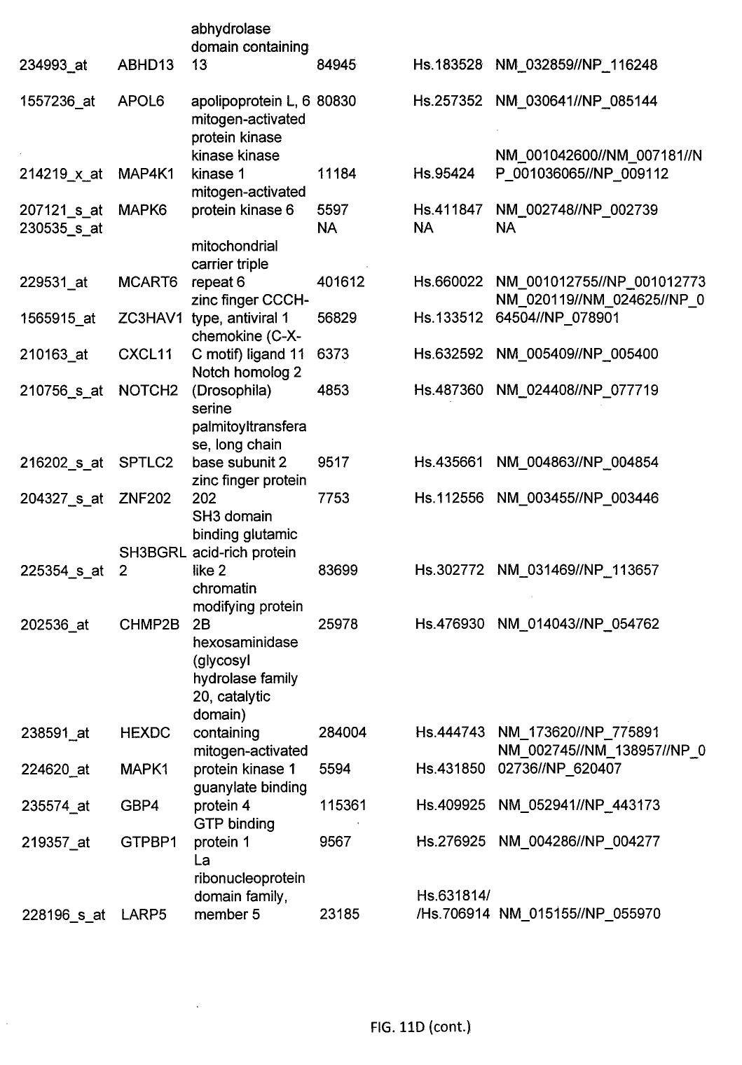

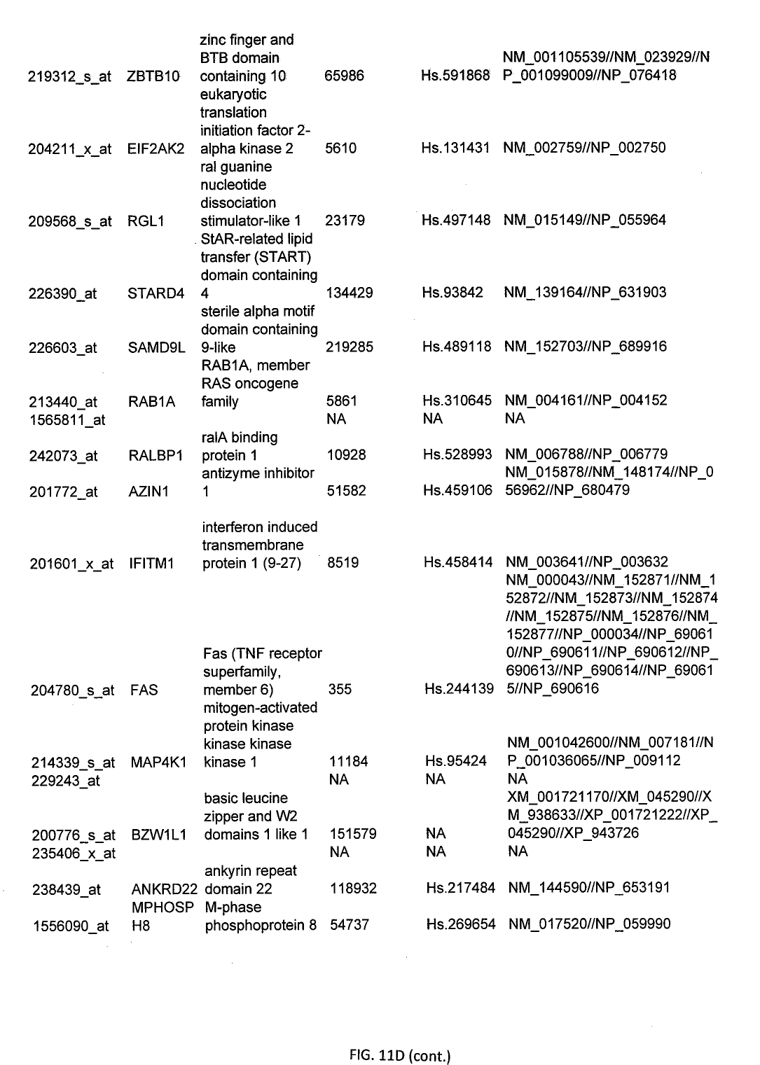

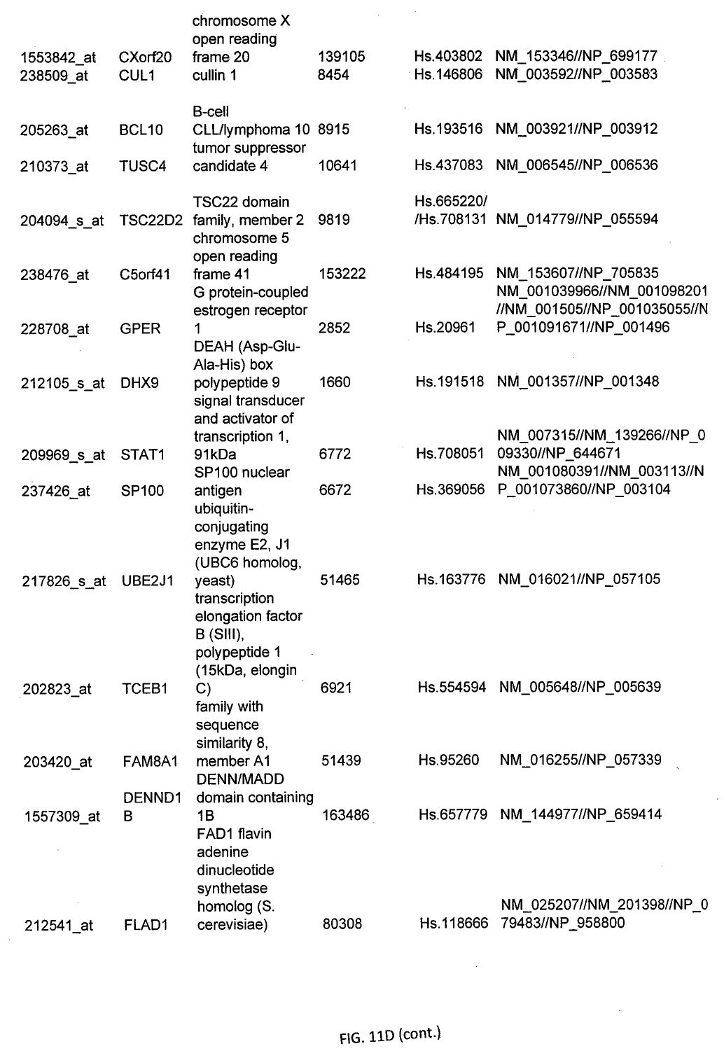

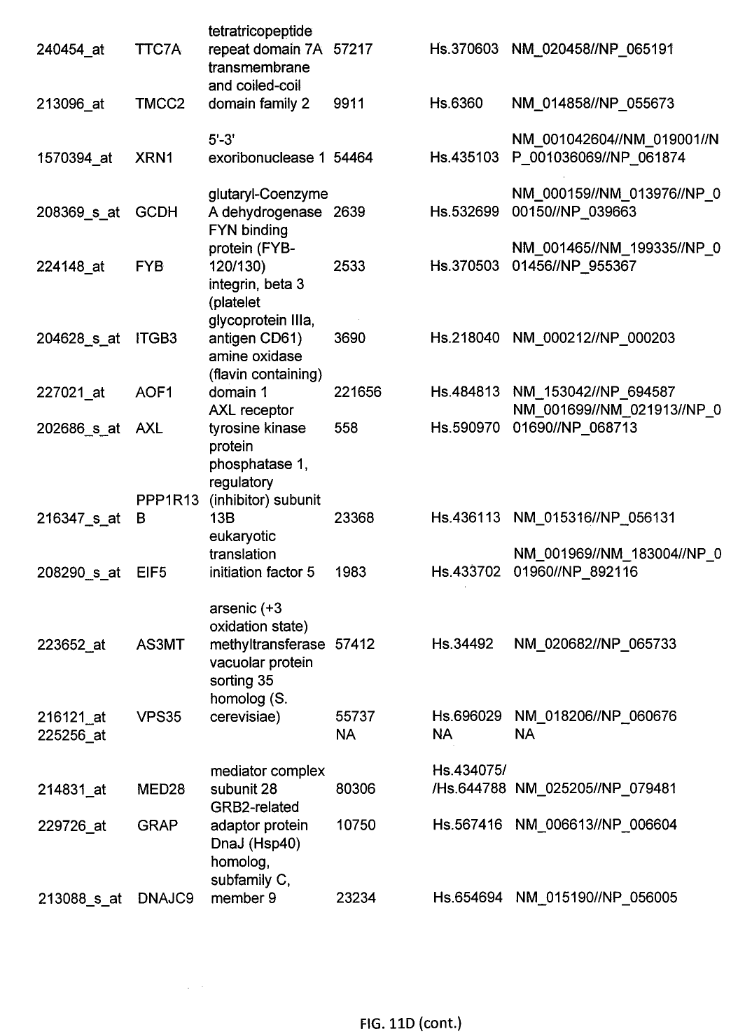

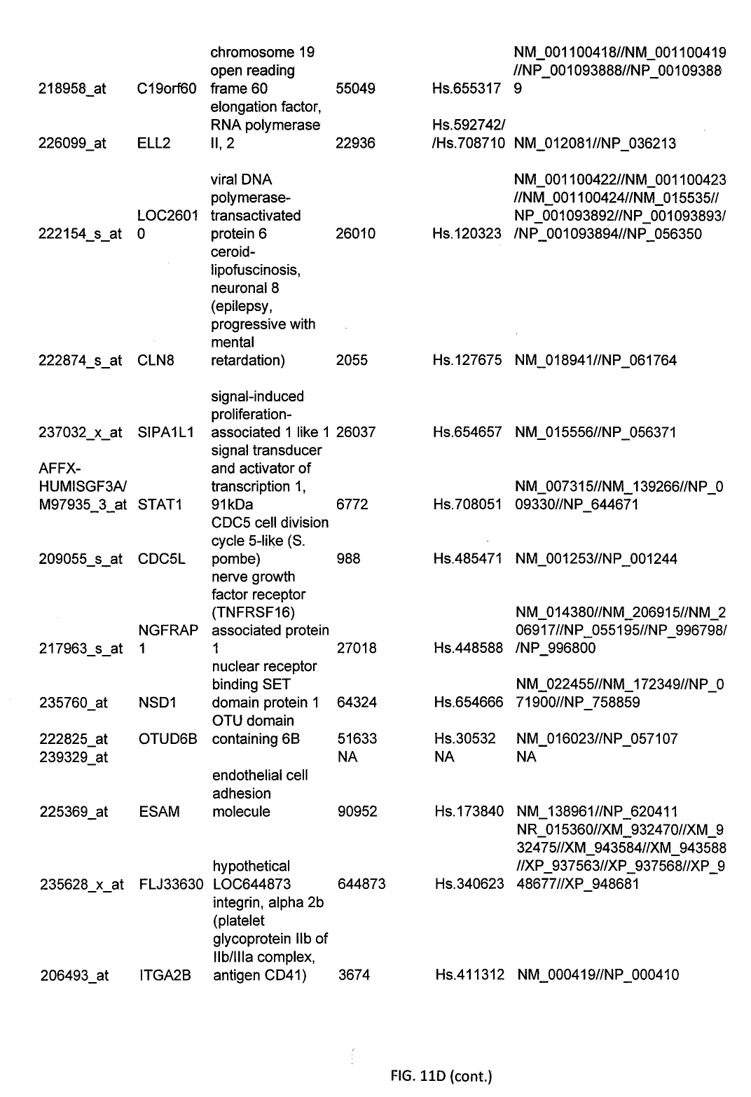

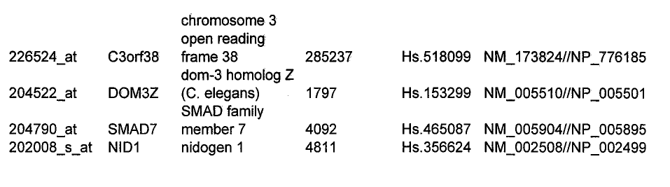

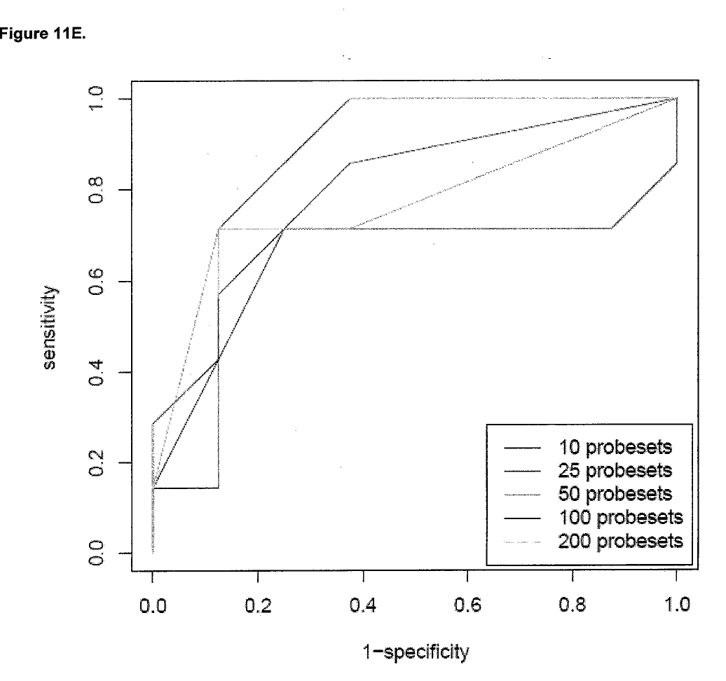

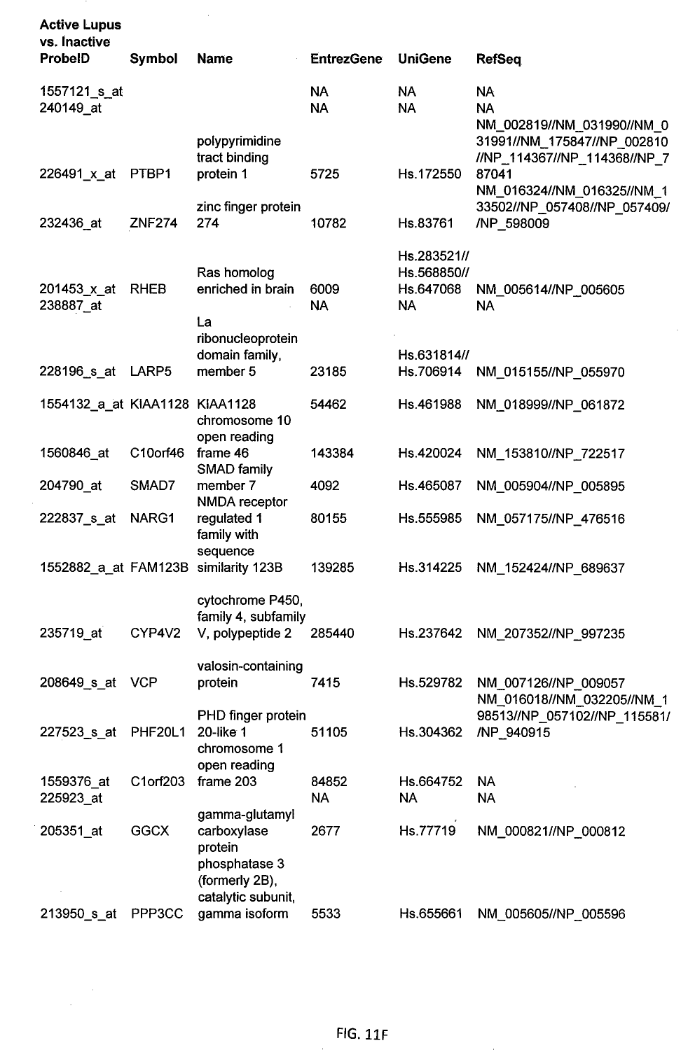

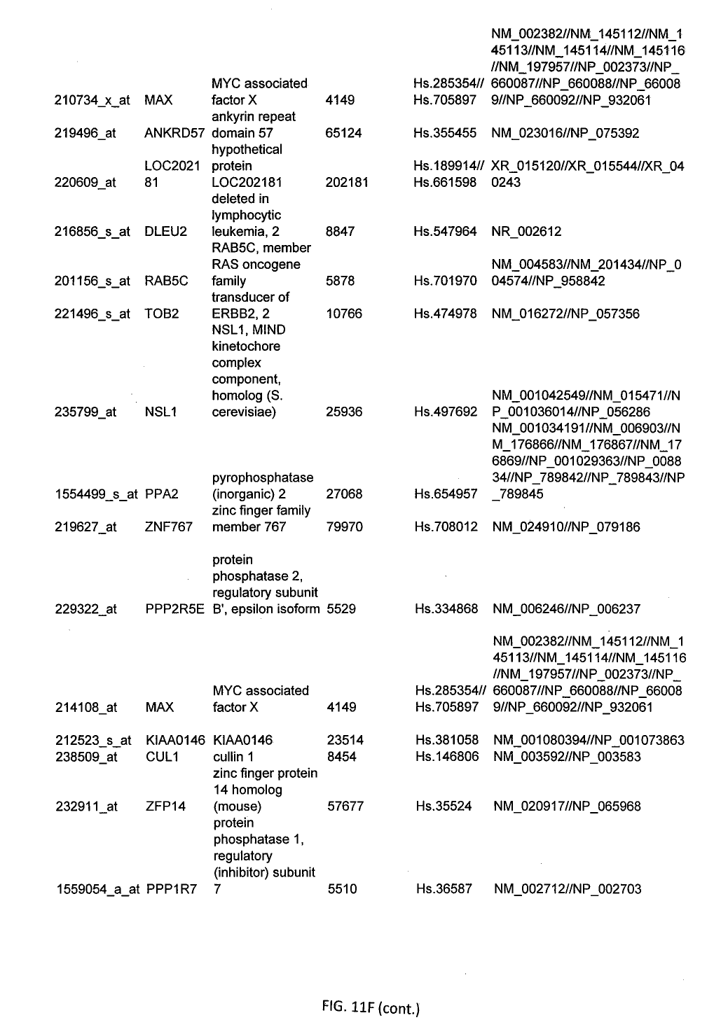

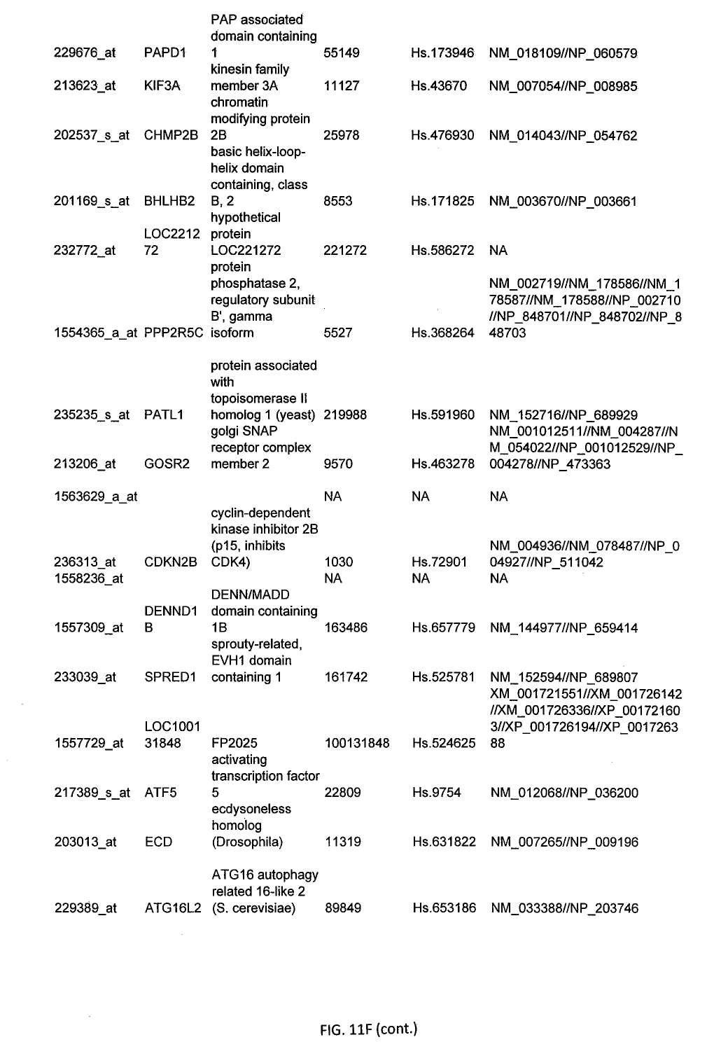

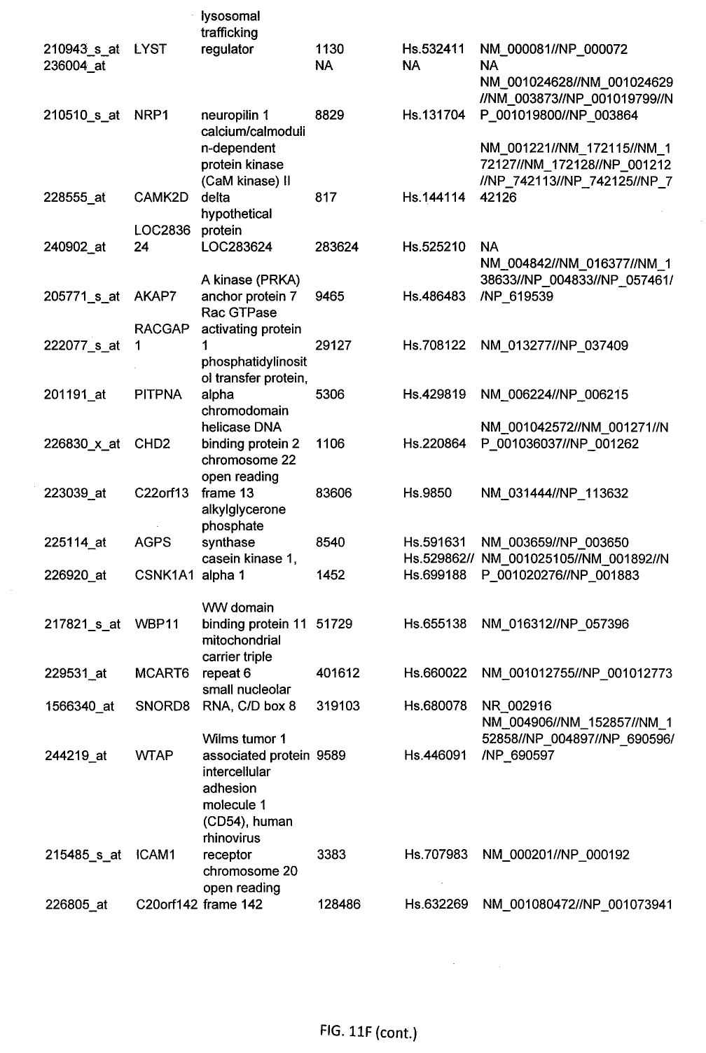

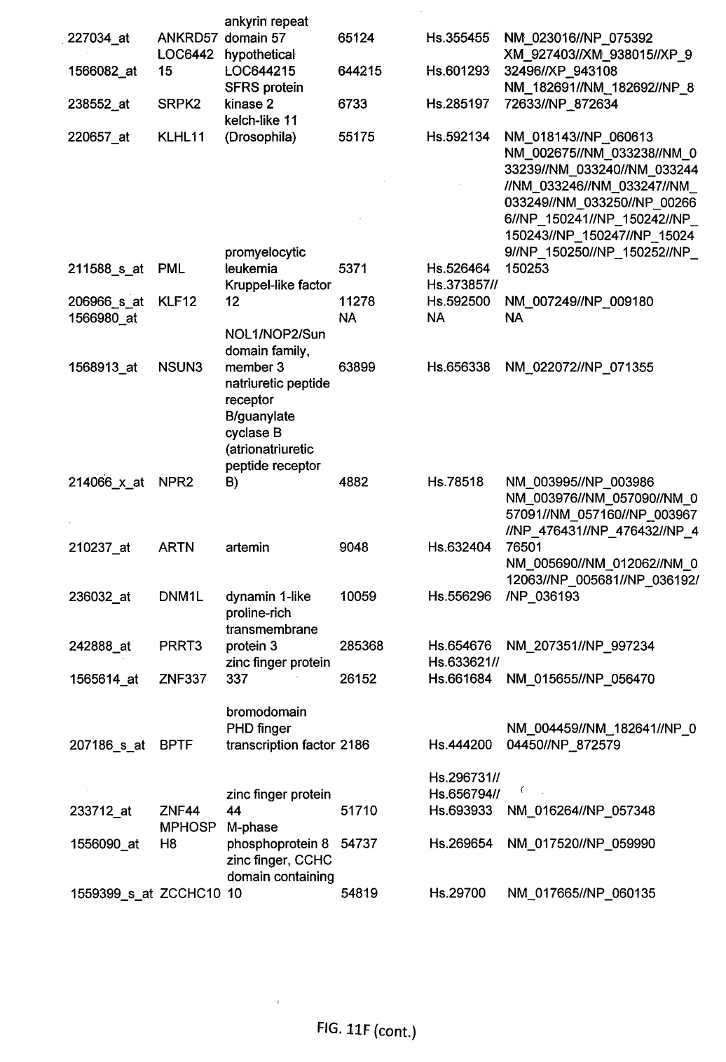

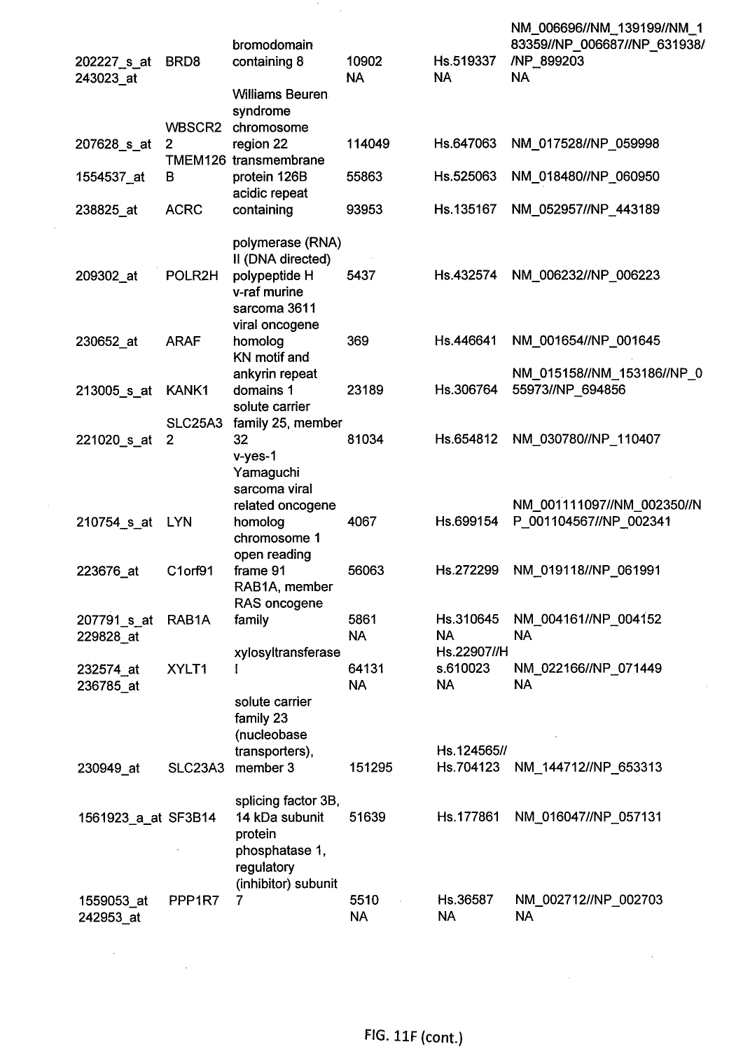

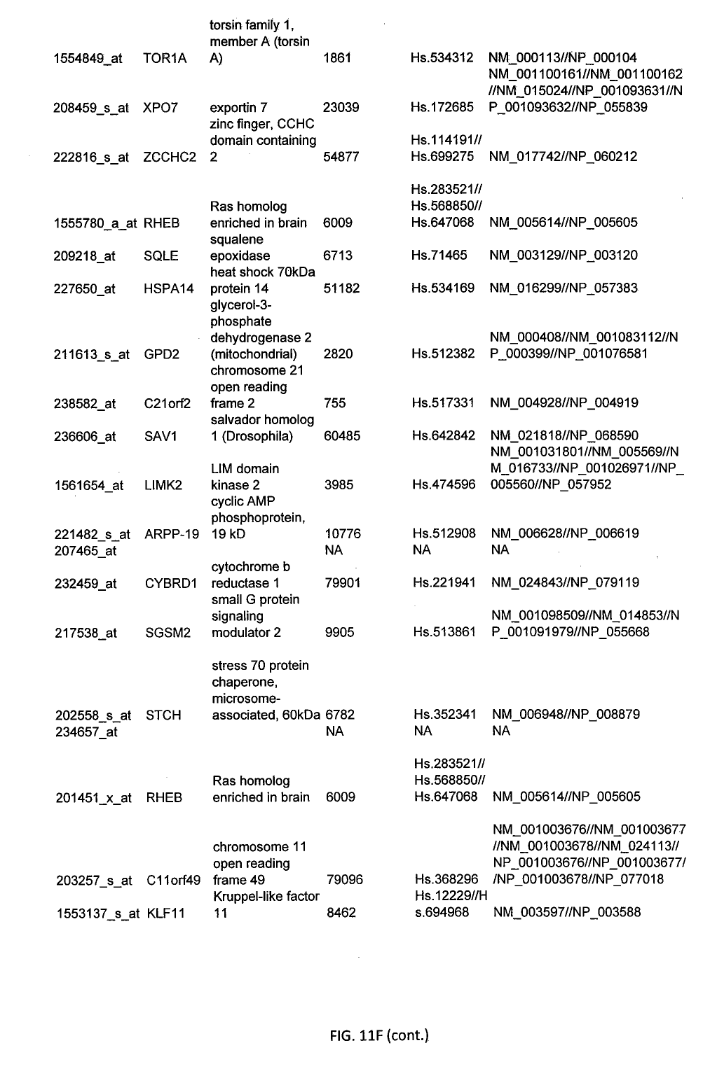

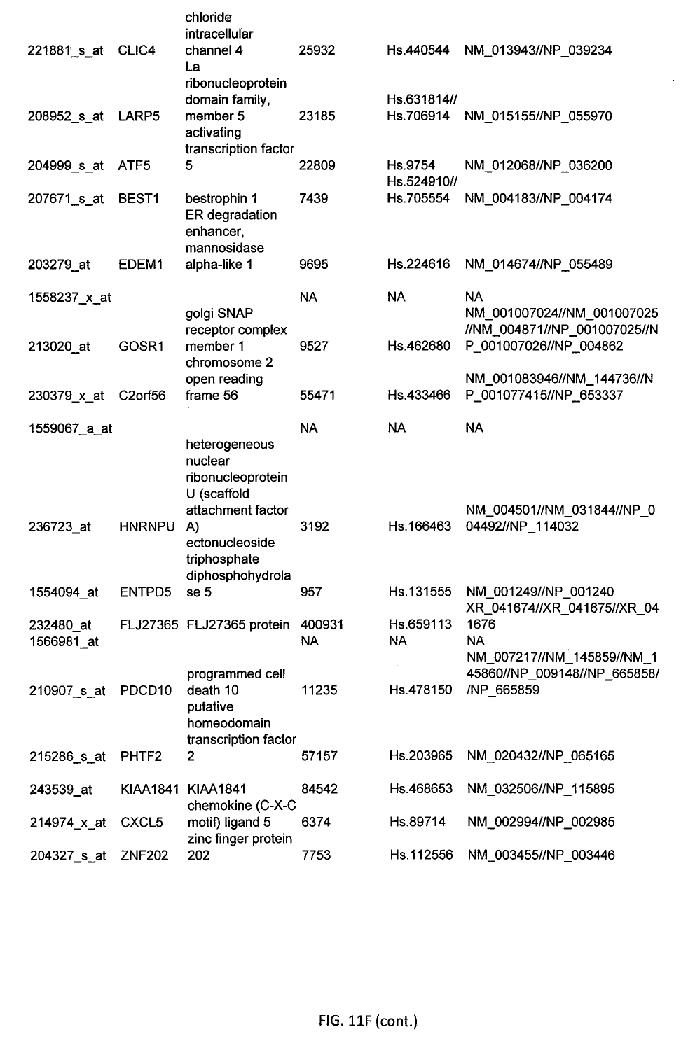

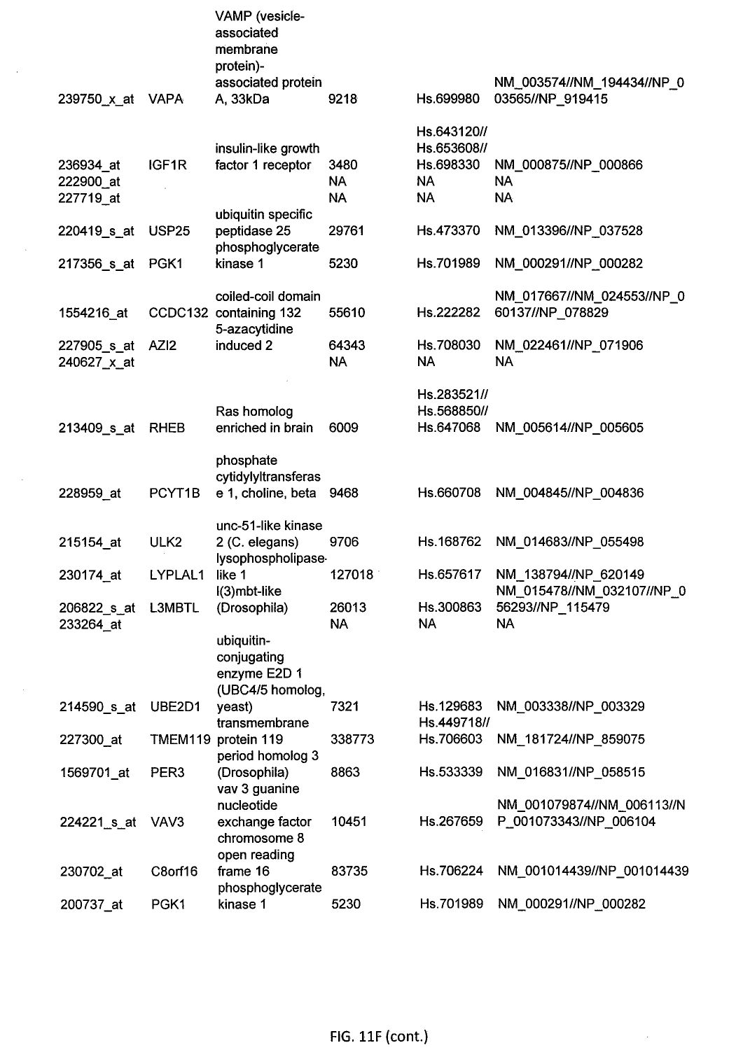

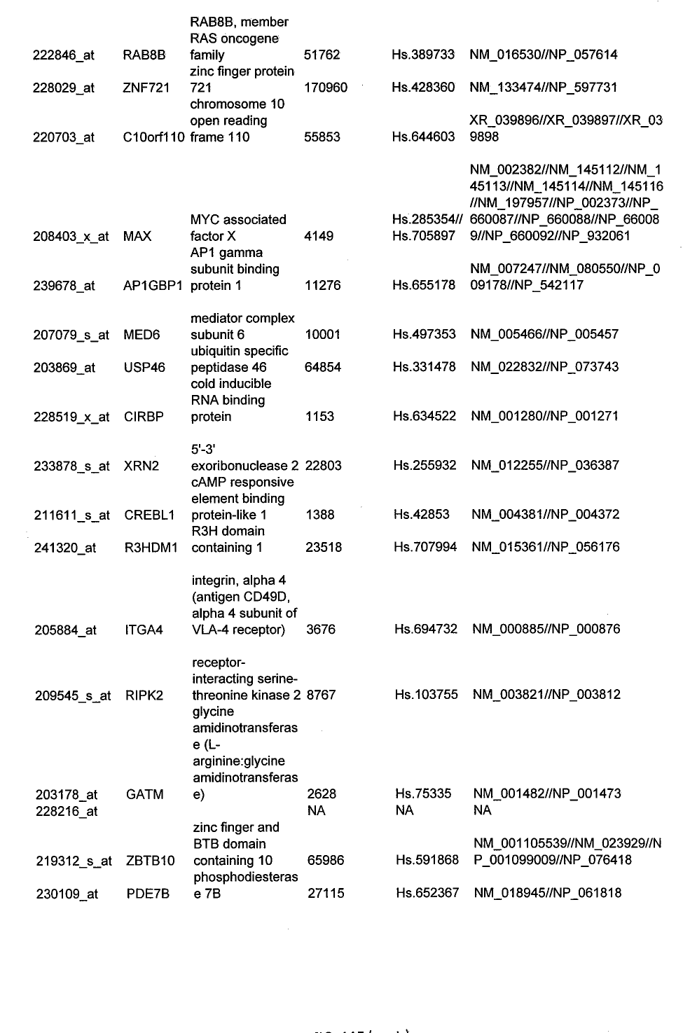

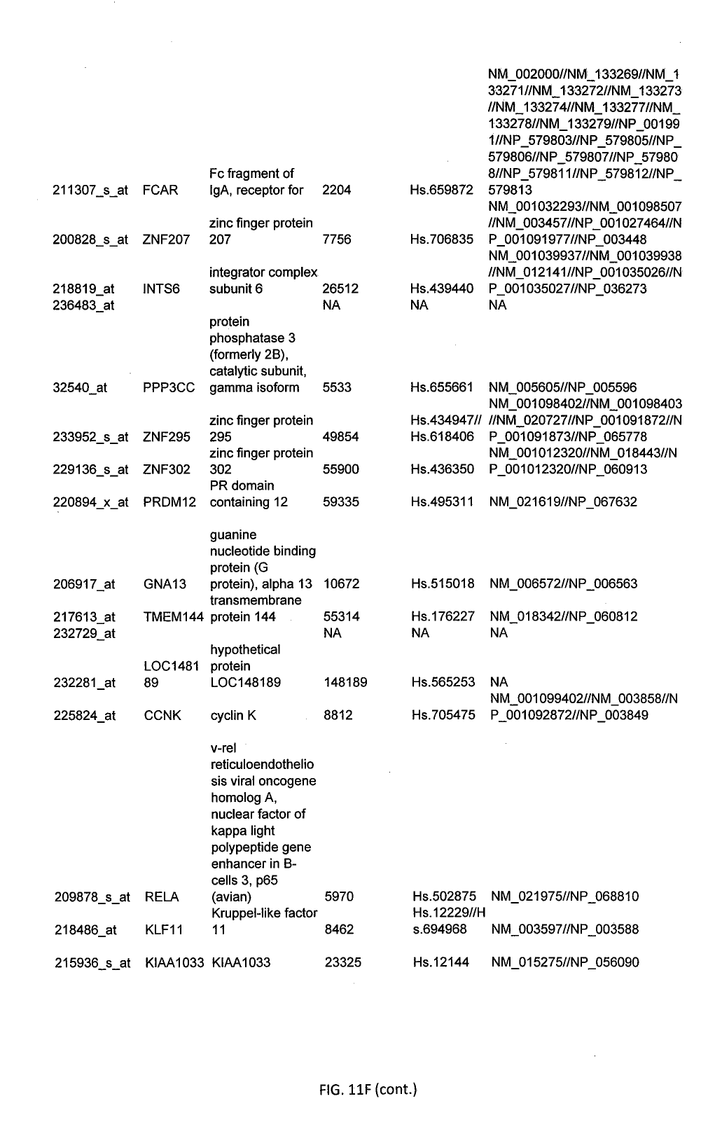

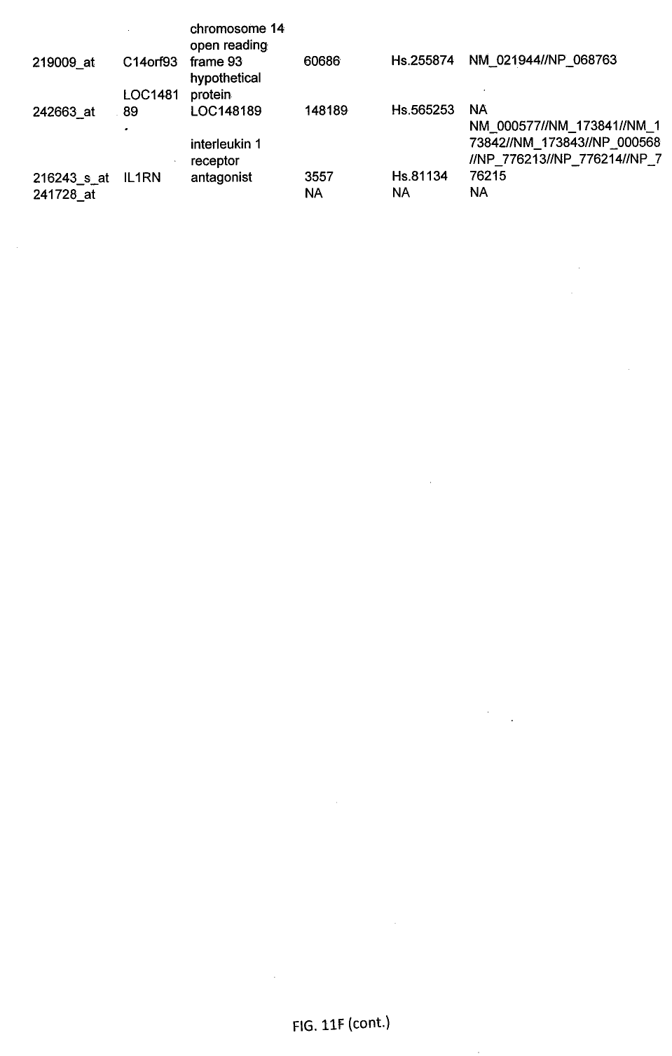

For example, it was shown that where the disorder is prostate cancer, altered gene expression (e.g., over-expression and/or under-expression) from one or more of the genes shown in FIG. 5A (e.g., MSRA, ZFAND6, THADA, FYN, RABGAP1L, IMMP2L, RICTOR, JMJD2C, NPTN, and VTI1A) distinguishes patients having prostate cancer from patients not having prostate cancer. It was shown that altered gene expression (e.g., over-expression and/or under-expression) in any one or more of the genes shown in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 5B (e.g., ERG, CHIT1, FAM20A, CRISP3, CEACAM6, MS4A1, FLJ22795, GLDN, CEACAM8, and ARG1) distinguishes patients having advanced prostate cancer from patients not having prostate cancer. It was shown that altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 5C (e.g., ANXA1, KLF4, NR4A2, FOSB, JUN, VIM, FOS, JUN, PPP1R15A, and SOS1) distinguishes patients having advanced prostate cancer from patients having early prostate cancer. It was shown that altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 7C (e.g., FCRL5, TMEM156, OASL, PPP1R9A, COL4A4, BTLA, FAM110B, TPD52, MGC39900, KIAA0125) distinguishes patients having breast cancer from patients not having breast cancer. It was shown that altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 8C (e.g., METTL7B, GATA2, CHRM3, SPRYD5, ENPP3, FLVCR2, SEPT1, NLRC3, PHC2, FAM84B) distinguishes patients having lung cancer from patients not having lung cancer. It was shown that altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 9B (e.g., LGR4, C1QC, FAM20A, FMNL2, C1QA, TCF7L2, C1QB, METTL7B, EZR, CACNA2D3) distinguishes patients having pancreatic cancer from patients not having pancreatic cancer. It was shown that altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 10B (e.g., FAM20A, FLVCR2, METTL7B, CNTNAP2, WASF1, TCF7L2, ATXN3, ME1, CCR7, GAS6) distinguishes patients having colon cancer from patients not having colon cancer. It was shown that altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 11B (e.g., FAM20A, FLVCR2, METTL7B, CNTNAP2, WASF1, TCF7L2, ATXN3, ME1, CCR7, GAS6) distinguishes patients having active systemic lupus erythematosus from patients not having active systemic lupus erythematosus. It was shown that altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 11D (e.g., PPBP, SDPR, SPARC, PF4, GNG1, STAT1, PATL1, TBXA2R, PPCSK6, ITGB3) distinguishes patients having inactive systemic lupus erythematosus from patients not having active systemic lupus erythematosus. It was shown that altered gene expression (e.g., over-expression and/or under-expression) in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 11F (e.g., PTBP1, ZNF274, RHEB, LARP5, KIAA1128, C10orf46, SMAD7, NARG1, FAM123B, CYP4V2) distinguishes patients having active systemic lupus erythematosus from patients having inactive systemic lupus erythematosus.

The present invention identifies that CD11b+ cells have informative value in assessing disorder (e.g., hyperproliferative, inflammatory) status and risk. Any method or approach that assesses CD11b+ characteristics associated with disease status may be used, including, but not limited to, gene expression analysis, proteome analysis, cell morphology, and the like.

Accordingly, the present invention provides methods utilizing circulating CD11b+ cells as biomarkers for detecting particular disorders (e.g., inflammatory disorder and/or immune disorder (e.g., systemic lupus erythematosus), hyperproliferative disorder (e.g., cancer (e.g., lung cancer, colon cancer, prostate cancer, pancreatic cancer, breast cancer))). Exemplary embodiments are described in more detail below to illustrate aspects of embodiments of the invention. In particular, gene expression analysis is focused on to illustrate embodiments of the invention.

### I. Systems and Methods for Characterizing Disorders Via Gene Expression Analysis

Embodiments of the present invention provide methods of characterizing disorders in a subject through detection of genetic expression profiles within PBMCs (e.g., CD11b+ PBMCs). The present invention is not limited to characterizing particular disorders through detection of genetic expression profiles within PBMCs. In some embodiments, the disorders include, but are not limited to, immune disorders, inflammatory disorders, and hyperproliferative disorders. With regard to immune disorders, the methods can be used to assess, for example, graft versus host disease, rheumatoid arthritis, and systemic lupus erythematosus. With regard to hyperproliferative disorders, the methods can be used to assess, for example, cancer, which can be either malignant or benign. Exemplary cancers that can be assessed include, for example, adenomas, adenocarcinomas, carcinomas, leukemias, lymphomas, melanomas, myelomas, sarcomas, and teratomas. In addition, the methods can be used to assess cancers of the bladder and the renal system, brain, breast, cervix, thyroid, skin, pancreas, colon, lung, ovaries, prostate, rectum. With regard to inflammatory disorders, the methods can be used to assess, for example, asthma and psoriasis.

The present invention provides comprehensive systems and methods for the detection of genetic expression profiles within PBMCs (e.g., CD11b+ PBMCs). Experiments conducted during the course of the development of embodiments for the present invention determined that certain genes are either over-expressed or under-expressed (e.g., altered gene expression) in certain disorders (e.g., cancer, systemic lupus erythematosus). For example, experiments determined that within prostate cancer certain genes are over-expressed (e.g., up-regulated) or under-expressed (e.g., down-regulated) (e.g., down-regulated MSRA, ZFAND6, THADA, FYN, RABGAP1L, IMMP2L, RICTOR, JMJD2C, NPTN, and/or VT1A gene expression) (see, also, any one or more of the genes shown in FIG. 5A) in CD11b+ PBMCs for patients diagnosed with prostate cancer versus patients not diagnosed with prostate cancer. In addition, it was shown that altered gene expression in any one or more of the genes shown in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 5B (e.g., ERG, CHIT1, FAM20A, CRISP3, CEACAM6, MS4A1, FLJ22795, GLDN, CEACAM8, and ARG1) distinguishes patients having advanced prostate cancer from patients not having prostate cancer. It was shown that altered gene expression in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 5C (e.g., ANXA1, KLF4, NR4A2, FOSB, JUN, VIM, FOS, JUN, PPP1R15A, and SOS1) distinguishes patients having advanced prostate cancer from patients having early prostate cancer. It was shown that altered gene expression in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 7C (e.g., FCRL5, TMEM156, OASL, PPP1R9A, COL4A4, BTLA, FAM110B, TPD52, MGC39900, KIAA0125) distinguishes female patients having breast cancer from female patients not having breast cancer. It was shown that altered gene expression in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 8C (e.g., METTL7B, GATA2, CHRM3, SPRYD5, ENPP3, FLVCR2, SEPT1, NLRC3, PHC2, FAM84B) distinguishes patients having lung cancer from patients not having lung cancer. It was shown that altered gene expression in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 9B (e.g., LGR4, C1QC, FAM20A, FMNL2, C1QA, TCF7L2, C1QB, METTL7B, EZR, CACNA2D3) distinguishes patients having pancreatic cancer from patients not having pancreatic cancer. It was shown that altered gene expression in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 10B (e.g., FAM20A, FLVCR2, METTL7B, CNTNAP2, WASF1, TCF7L2, ATXN3, ME1, CCR7, GAS6) distinguishes patients having colon cancer from patients not having colon cancer. It was shown that altered gene expression in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 11B (e.g., FAM20A, FLVCR2, METTL7B, CNTNAP2, WASF1, TCF7L2, ATXN3, ME1, CCR7, GAS6) distinguishes patients having active systemic lupus erythematosus from patients not having active systemic lupus erythematosus. It was shown that altered gene expression in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 11D (e.g., PPBP, SDPR, SPARC, PF4, GNG11, STAT 1, PATL1, TBXA2R, PPCSK6, ITGB3) distinguishes patients having inactive systemic lupus erythematosus from patients not having active systemic lupus erythematosus. It was shown that altered gene expression in any one or more (e.g., 1, 5, 10, 25, 50, 100, all) (e.g., altered gene expression in 1% of the genes, 2%, 5%, 7%, 10%, 15%, 25%, 50%, 70%, 80%, 85%, 99%, etc.)) of the genes shown in FIG. 11F (e.g., PTBP1, ZNF274, RHEB, LARP5, KIAA1128, C10orf46, SMAD7, NARG1, FAM123B, CYP4V2) distinguishes patients having active systemic lupus erythematosus from patients having inactive systemic lupus erythematosus. Accordingly, in some embodiments, the present invention provides systems and methods for detecting the genetic expression profiles for specific genes within PBMCs (e.g., CD11b+ PBMCs) for the characterization of disorders (e.g., inflammatory disorders (e.g., systemic lupus erythematosus), hyperproliferative disorders (e.g., cancer)). For example, in some embodiments, the present invention provides systems and methods for detecting the genetic expression profiles for specific genes within PBMCs (e.g., CD11b+ PBMCs) for the characterization of breast cancer, colon cancer, lung cancer, pancreatic cancer, prostate cancer, and/or systemic lupus erythematosus. The present invention is not limited to the detection of particular genes within PBMCs (e.g., CD11b+ PBMCs) for the characterization of particular disorders (see, e.g., FIGS. 5, 7, 8, 9, 10, 11).

The present invention is not limited to a particular type of characterization based upon detected genetic expression profiles for specific genes within PBMCs (e.g., CD11b+ PBMCs). In some embodiments, disorder characterization is accomplished through comparison of genetic expression profiles for specific within CD11b+ PBMCs to established CD11b+ PBMC genetic expression profiles corresponding to different disorder characterizations (e.g., established CD11b+ PBMC genetic expression profile for low risk for developing a particular disorder (e.g., systemic lupus erythematosus, lung cancer, colon cancer, breast cancer, pancreatic cancer, prostate cancer); established CD11b+ PBMC genetic expression profile for medium risk for developing a particular disorder (e.g., systemic lupus erythematosus, lung cancer, colon cancer, breast cancer, pancreatic cancer, prostate cancer); established CD11b+ PBMC genetic expression profile for high risk for developing a particular disorder (e.g., systemic lupus erythematosus, lung cancer, colon cancer, breast cancer, pancreatic cancer, prostate cancer); established CD11b+ PBMC genetic expression profile for high risk for a subject not previously or currently diagnosed with a particular disorder (e.g., systemic lupus erythematosus, lung cancer, colon cancer, breast cancer, pancreatic cancer, prostate cancer); established CD11b+ PBMC genetic expression profile for a person in remission from cancer (e.g., lung cancer, breast cancer, colon cancer, pancreatic cancer, prostate cancer); established CD11b+ PBMC genetic expression profile for a subject diagnosed with a particular disorder (e.g., systemic lupus erythematosus, lung cancer, colon cancer, breast cancer, pancreatic cancer, prostate cancer); established CD11b+ PBMC genetic expression profile for cancer cell proliferation (e.g., lung cancer, colon cancer, breast cancer, pancreatic cancer, prostate cancer) (e.g., prostate epithelial cell proliferation); established CD11b+ PBMC genetic expression profile for cancer cell metastasis (e.g., lung cancer, colon cancer, breast cancer, pancreatic cancer, prostate cancer) (e.g., prostate epithelial cell metastasis). Established threshold levels may be generated from any number of sources, including but not limited to, groups of individuals (e.g., men and/or women, adults and/or children) having a particular disorder (e.g., systemic lupus erythematosus, lung cancer, colon cancer, breast cancer, pancreatic cancer, prostate cancer), groups of individuals (e.g., men and/or women, adults and/or children) not having that particular disorder. For example, regarding prostate cancer, established threshold levels may be generated from any number of sources, including but not limited to, groups of men having prostate cancer, groups of men not having prostate cancer, groups of men having prostate cancer and prostate epithelial cell metastasis, groups of men under 35 years of age, groups of men under 50 years of age, groups of men under 70 years of age, groups of men over 65 years of age, groups of men having prostate cancer and a particular form of treatment, etc. Any number of individuals within a group may be used to generate an established threshold (e.g., 5 individuals, 10 men, 20, 30, 50, 500, 5000, 10, 000, etc.). Threshold levels may be generated with any type or source of bodily sample having PBMCs (e.g., CD11b+ PBMCs) from a subject (e.g., including but not limited to, plasma, serum, whole blood, mucus, and urine).

In some embodiments, samples from a subject are compared to samples from one or more control subjects (e.g., subjects known to have or lack a particular disorder or disease) or from one or more samples taken from the subject at earlier time points.

In some embodiments, disorder status (e.g., stage of disorder, improvement in status of disorder, worsening in status of disorder, no change in status of disorder) is characterized through comparison of genetic expression profiles for specific CD11b+ PBMCs to established CD11b+ PBMC genetic expression profiles corresponding to different disorder statuses (e.g., established CD11b+ PBMC genetic expression profile for particular statuses of particular disorders). For example, experiments conducted during the development of embodiments for the present invention demonstrated CD11b+ PBMC genetic expression profiles for distinguishing advanced prostate cancer and early prostate cancer (e.g., see any one or more of the genes shown in FIG. 5C (e.g., ANXA1, KLF4, NR4A2, FOSB, JUN, VIM, FOS, JUN, PPP1R15A, and SOS1)). In addition, experiments demonstrated CD11b+ PBMC genetic expression profiles for distinguishing active systemic lupus erythematosus and inactive systemic lupus erythematosus (e.g., see any one or more of the genes shown in FIG. 11F (e.g., PTBP1, ZNF274, RHEB, LARP5, KIAA1128, C10orf46, SMAD7, NARG1, FAM123B, CYP4V2)). In addition, in some embodiments, the effect (e.g., improvement, worsening, no change) of a particular treatment (e.g., experimental treatment, traditional treatment, watchful waiting, surgery, pharmaceutical intervention, etc.) is evaluated through comparing pre-treatment and post-treatment genetic expression profiles for specific CD11b+ PBMCs related to particular disorders (e.g., systemic lupus erythematosus, breast cancer, lung cancer, colon cancer, prostate cancer, and pancreatic cancer).

In some embodiments, systems and methods utilizing microarray technologies are used to detect genetic expression profiles for specific genes within PBMCs (e.g., CD11b+ PBMCs) for the characterization of a disorder (e.g., systemic lupus erythematosus, lung cancer, breast cancer, colon cancer, pancreatic cancer, prostate cancer) (e.g., for prostate cancer, altered gene expression in any one or more of the following specific genes: MSRA, ZFAND6, THADA, FYN, RABGAP1L, IMMP2L, RICTOR, JMJD2C, NPTN, and/or VTI1A; see, also, altered gene expression in any one or more of the genes shown in FIG. 5A) (e.g., for distinguishing advanced prostate cancer from control, altered gene expression in any one or more of the following specific genes: ERG, CHIT1, FAM20A, CRISP3, CEACAM6, MS4A1, FLJ22795, GLDN, CEACAM8, and ARG1; see, also, altered gene expression in any one or more of the genes shown in FIG. 5B) (e.g., for distinguishing advanced prostate cancer from early prostate cancer, altered gene expression in any one or more of the following genes: ANXA1, KLF4, NR4A2, FOSB, JUN, VIM, FOS, JUN, PPP1R15A, and SOS1; see, also, altered gene expression in any one or more of the genes shown in FIG. 5C) (e.g., for detecting breast cancer, altered gene expression in any one or more of the following genes: FCRL5, TMEM156, OASL, PPP1R9A, COL4A4, BTLA, FAM110B, TPD52, MGC39900, KIAA0125; see, also, altered gene expression in any one or more of the genes shown in FIG. 7C) (e.g., for detecting lung cancer, altered gene expression in any one or more of the following genes: METTL7B, GATA2, CHRM3, SPRYD5, ENPP3, FLVCR2, SEPT1, NLRC3, PHC2, FAM84B; see, also, altered gene expression in any one or more of the genes shown in FIG. 8C) (e.g., for detecting pancreatic cancer, altered gene expression in any one or more of the following genes: LGR4, C1QC, FAM20A, FMNL2, C1QA, TCF7L2, C1QB, METTL7B, EZR, CACNA2D3; see, also, altered gene expression in any one or more of the genes shown in FIG. 9B) (e.g., for detecting colon cancer, altered gene expression in any one or more of the following genes: FAM20A, FLVCR2, METTL7B, CNTNAP2, WASF1, TCF7L2, ATXN3, ME1, CCR7, GAS6; see, also, altered gene expression in any one or more of the genes shown in FIG. 10B) (e.g., for detecting active systemic lupus erythematosus, altered gene expression in any one or more of the following genes: TOP2A, TRPM7, USP15, UGCG, TTLL5, SSFA2, ZC3HAV1, AFF1, AGGF1, TET2; see, also, altered gene expression in any one or more of the genes shown in FIG. 11B) (e.g., for detecting inactive systemic lupus erythematosus, altered gene expression in any one or more of the following genes: PPBP, SDPR, SPARC, PF4, GNG11, STAT1, PATL1, TBXA2R, PPCSK6, ITGB3; see, also, altered gene expression in any one or more of the genes shown in FIG. 11D) (e.g., for distinguishing active systemic lupus erythematosus from inactive systemic lupus erythematosus, altered gene expression in any one or more of the following genes: PTBP1, ZNF274, RHEB, LARP5, KIAA1128, C10orf46, SMAD7, NARG1, FAM123B, CYP4V2; see, also, altered gene expression in any one or more of the genes shown in FIG. 11F). Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

In some embodiments, systems and methods utilizing genomic amplification technologies are used to detect genetic expression profiles for specific genes within PBMCs (e.g., CD11b+ PBMCs) for the characterization of a particular disorder (e.g., systemic lupus erythematosus, lung cancer, colon cancer, breast cancer, pancreatic cancer, prostate cancer). Genomic DNA and mRNA may be amplified prior to or simultaneous with detection. Illustrative non-limiting examples of nucleic acid amplification techniques include, but are not limited to, polymerase chain reaction (PCR) (e.g., TAQMAN technology), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) may require that RNA be reversed transcribed to DNA prior to amplification (e.g., RT-PCR), whereas other amplification techniques directly amplify RNA (e.g., TMA and NASBA).

In some embodiments, a computer-based analysis program is used to translate the raw data generated by the systems and methods for the detection of genetic expression profiles within PBMCs (e.g., CD11b+ PBMCs) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a PBMC (e.g., CD11b+ PBMC) sample (e.g., a serum sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Once received by the profiling service, the sample is processed and a profile is produced (e.g., genetic expression profile for CD11b+ PBMCs), specific for the diagnostic or prognostic information desired for the subject.

The genetic expression profile data is then prepared in a format suitable for interpretation by a treating clinician. For example, rather than providing raw genetic expression data for specific genes within PBMCs (e.g., CD11b+ PBMCs), the prepared format may represent a diagnosis or risk assessment (e.g., likelihood of a particular disorder prostate cancer being present, likelihood of a particular disorder being at a particular stage) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

In some embodiments, the results are used in a clinical setting to determine a further diagnostic (e.g., the additional of further screening (e.g., PSA or other markers) or diagnostic (e.g., biopsy)) course of action. In other embodiments, the results are used to determine a treatment course of action (e.g., choice of therapies or watchful waiting).

### EXPERIMENTAL

The following example is provided in order to demonstrate and further illustrate certain preferred embodiments and aspects of the present invention and are not to be construed as limiting the scope thereof.

### Example I

This Example describes the materials and methods used for Examples II through VII.

PC-3 xenografts Xenograft tumors were established as previously described (see, e.g., Loberg R D, et al., Neoplasia. 2006 July; 8(7):578-86; herein incorporated by reference in its entirety). Briefly, male athymic mice (5-6 weeks) were injected subcutaneously with 1×10⁵ PC-3 cells in 200 μL Matrigel (BD Biosciences, Inc.). Ten mice were implanted and tumor volumes were calculated by caliper measurement performed weekly to monitor and track tumor growth (tumor volume=L×W×W×0.56). Tumors were harvested once the tumor reached the predefined critical mass of 1000 mm³.

Human samples Blood samples were collected from 22 men with metastatic, castration resistant prostate cancer, 7 women with metastatic breast cancer, 6 men and 1 woman with metastatic colon cancer, 4 men and 7 women with advanced lung cancer, 5 men and 8 women with advanced pancreatic cancer. All cancer patients were eligible for, or were actively receiving. Age matched controls consisted of 15 men and 10 women. Twelve women with a history of breast cancer on hormonal therapy with no evidence of disease for a minimum of two years served as another control group. Seven women with active systemic lupus erythematosus as determined by established clinical criteria were also tested. Cancer patients collected over a several month period, RNA isolated, and samples saved for analysis.

Isolation of CD11b+ peripheral blood mononuclear cells Murine mononuclear cells (PBMCs) were isolated from peripheral blood at two specific timepoints; 1) prior to PC-3 xenograft implantation and 2) 3 weeks post-xenograft implantation. PBMCs were collected in α-MEM with 2% FBS and 5 U/ml heparin (Sigma). Low density cells were isolated by density centrifugation (Histopaque-1083 (Sigma, St. Louis, Mo.) 700×g for 30 min at 25° C.). Positive selection for CD11b⁺ cells were performed using the MACS magnetic bead system (Miltenyi Biotech, Auburn, Calif.) following the manufacturer's protocol. Briefly, 500 μL of peripheral blood were diluted in buffer containing 2 mM EDTA. Cell suspensions were incubated with CD11b+ microbeads for 30 min and then applied to the isolation columns housed in the magnetic field. CD11b− cells were removed by gravity flow and CD11b+ cells were collected for further analysis. Human PBMCs were isolated from peripheral blood by collection in a 7 ml tube containing heparin (purple top). CD11b+ cells were then isolated as above.

Affymetrix cDNA microarray analysis Microarray analysis was performed using the Affymetrix Gene Chip Mouse 430 2.0 arrays or the Affymetrix Human Gene Chip 133 array. RNA was isolated using an RNeasy Micro Kit (Qiagen, Inc.) and 20 ng total RNA was amplified using the Ovation Biotin Labeling system from NuGen, Inc. Amplified total RNA was labeled and processed prior to hybridization. Standard arrays were stained using a fluidics station and scanned for data analysis by a core statistician. Mouse and control samples were run on same date. Systemic lupus erythematosus patients and control samples were run on same date.

Quantitative real time polymerase chain reaction Total RNA was isolated from PBMCs using Trizol (Invitrogen Corp, Carlsbad, Calif.) following the manufacturer's specifications. Purified RNA (5 μg) was converted to cDNA using Super Script II reverse transcriptase (Invitrogen Corp.) following the manufacturer's instructions and used for gene expression analysis by real time PCR using an ABI Prism 7900HT thermocycler. Primers and probes were purchased from Applied Biosystems, Inc. and used with TaqMan® Universal PCR Master Mix, No AmpErase® UNG. GAPDH was used as an internal control to normalize and compare each sample. Cycle conditions for real time PCR were 95° C. (15 sec), 60° C. (1 min), 72° C. (1 min) for 40 cycles. Threshold cycle number for each sample were normalized to GAPDH for that sample and expressed on a log scale relative to GAPDH expression.

Statistics The microarray data was processed using the Affymetrix Microarray Suite version 5.0 software and performed statistical analysis of the expression data set using the Affymetrix MicroDB and Affymetrix DMT software. The Pearson correlation coefficient for individual test samples and the appropriate reference standard was determined using Microsoft Excel vand GraphPad Prism software (GraphPad Software). Principal component analysis (PCA) was performed on all data sets. PCA is an orthogonal linear transformation that transforms the data to a new coordinate system. The greatest variance by any projection of the data lies on the first coordinate (first principal component) and the second greatest variance on the second coordinate, generating the optimum transform for a given data in least square terms. Detailed data analysis and documentation of the sensitivity, reproducibility of the quantitative statistical microarray analysis using Affymetrix technology was performed in collaboration with an Affymetrix Microarray Core Facility (see, e.g., Irizarry, et al., Biostatistics, 4:249-64, 2003; herein incorporated by reference in its entirety).

### Example II

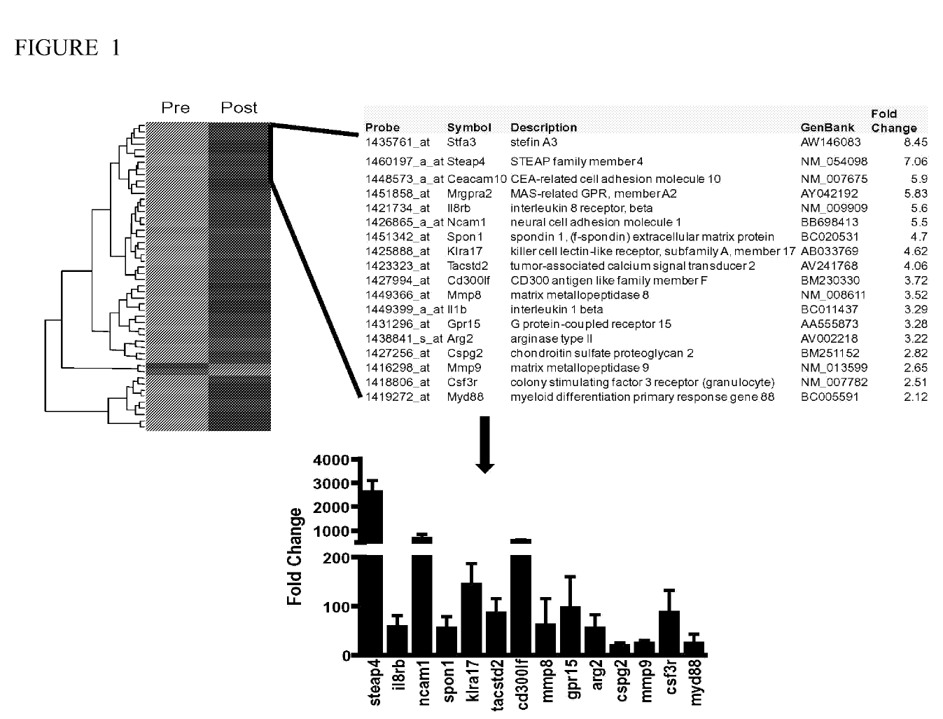

This example demonstrates that PCa changes the genetic profile of CD11b+ PBMCs in mice. To address the hypothesis that the presence of cancer can genotypically alter circulating PBMCs, experiments were conducted to look at the genetic profile of CD11b+ PBMCs from male SCID mice that had a PC-3 xenograft implanted subcutaneously. Blood was collected from 3 mice prior to xenograft implantation and again after the xenografts reached a critical tumor mass (>1000 mm³). RNA was isolated from the two CD11b+PBMC populations applied to the Affymetrix GeneChip® Mouse Genome 430 2.0 Array for comparison (FIG. 1). The data demonstrated that 298 genes were significantly different between mice with prostate cancer versus their control PBMCs prior to xenograft implantation (>2 fold expression; p<0.01). Changes in expression were confirmed by QPCR (FIG. 1). Principal component analysis revealed that the two populations of PBMCs could be differentiated with 100% sensitivity and specificity. The expression profile was indicative of the TAM genotype and indicated that the circulating monocytes were being “educated” prior to tumor infiltration. These genes included several genes expected to be altered as monocytes begin to be educated to a TAM phenotype, including arginase 2, MMP9, and IL8r (FIG. 1).

### Example III

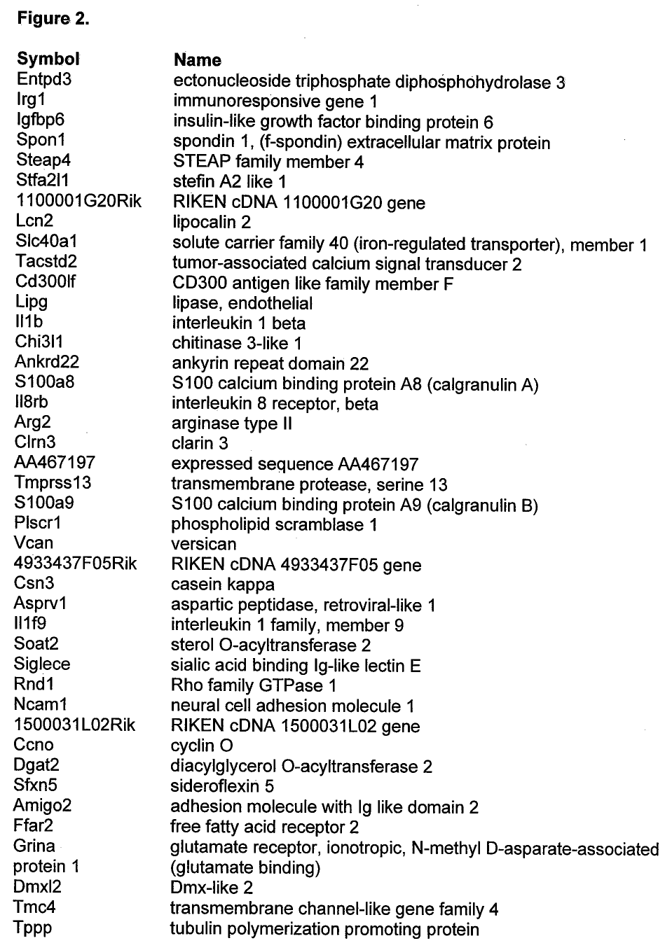

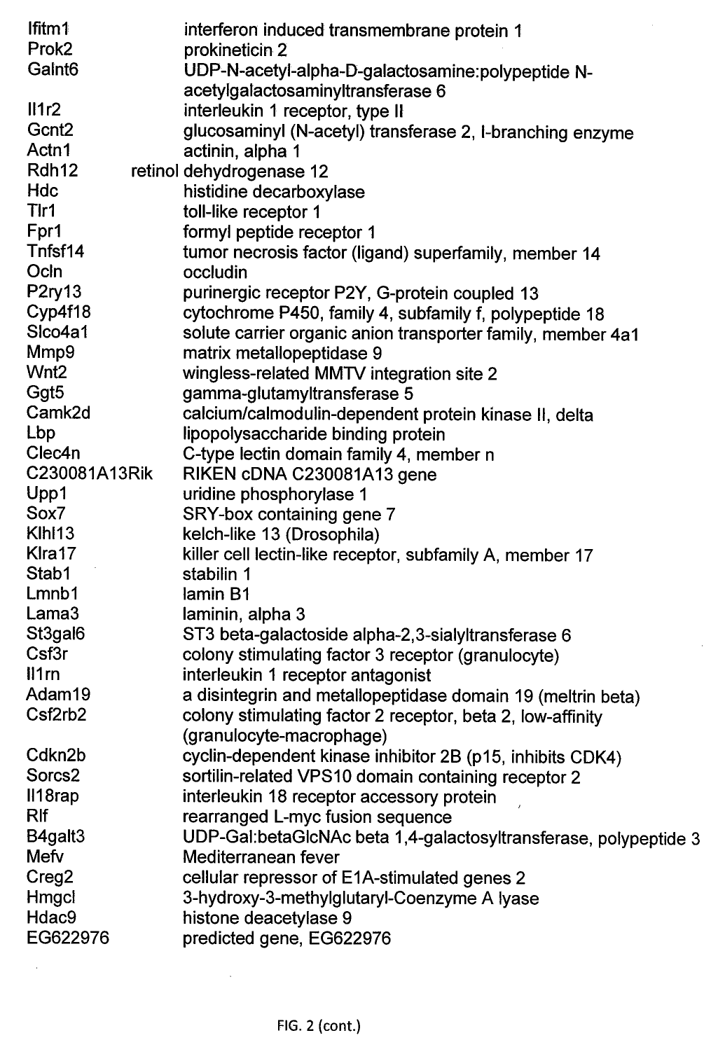

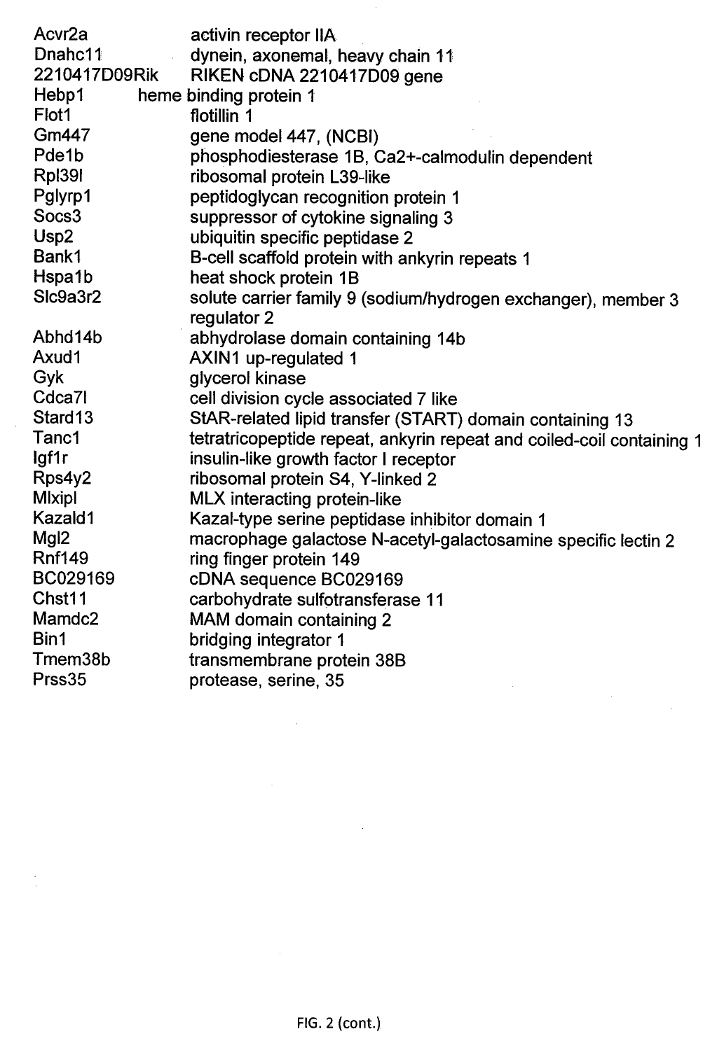

This example demonstrates that the gene expression profile of PBMC CD11b+ cells is similar to tissue derived TAMs. To further delineate the genotype/phenotype of these PBMCs as compared to TAMs, CD11b+ cells were isolated from PC-3 xenograft tumors and subject to array analysis. To make the comparisons between the gene expression of the different populations of cells, Gene Set Enrichment Analysis (GSEA) was utilized as a way to compare two lists of significant genes (see, e.g., Jiang, et al., Bioinformatics, 23:306-313, 2007; Subramanian, et al., Proc. Natl. Acad. Sci., 102(43):15545-15550, 2005; each herein incorporated by reference in their entireties). One comparison was used to come up with a ‘gene set’, which was the set of genes that were found significant in that comparison. We then looked at those genes in the second comparison to see where they appear in a ranked list of the genes (ranking is based on the t-statistic). If the gene set is ranked higher than expected in the second comparison, we then extrapolated that a particular cell type is comparable to another. PBMC CD 11b+ cells demonstrated a significantly similar signature as compared to TAMs (TAM vs cancer present PBMCs, t statistic 49.323, p<0.000001) (FIG. 2).

### Example IV

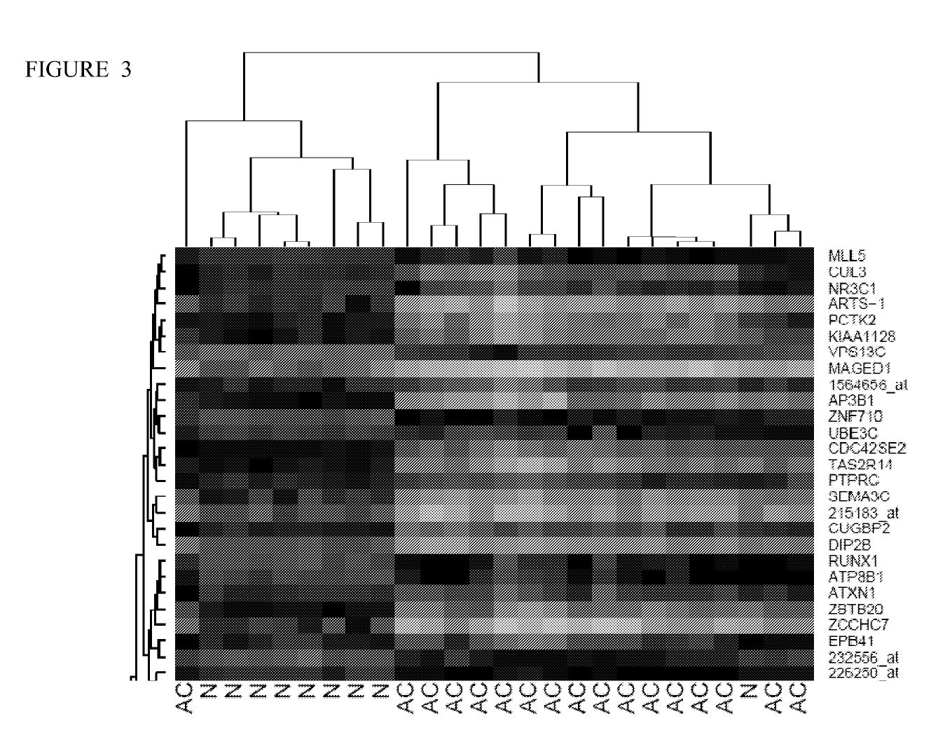

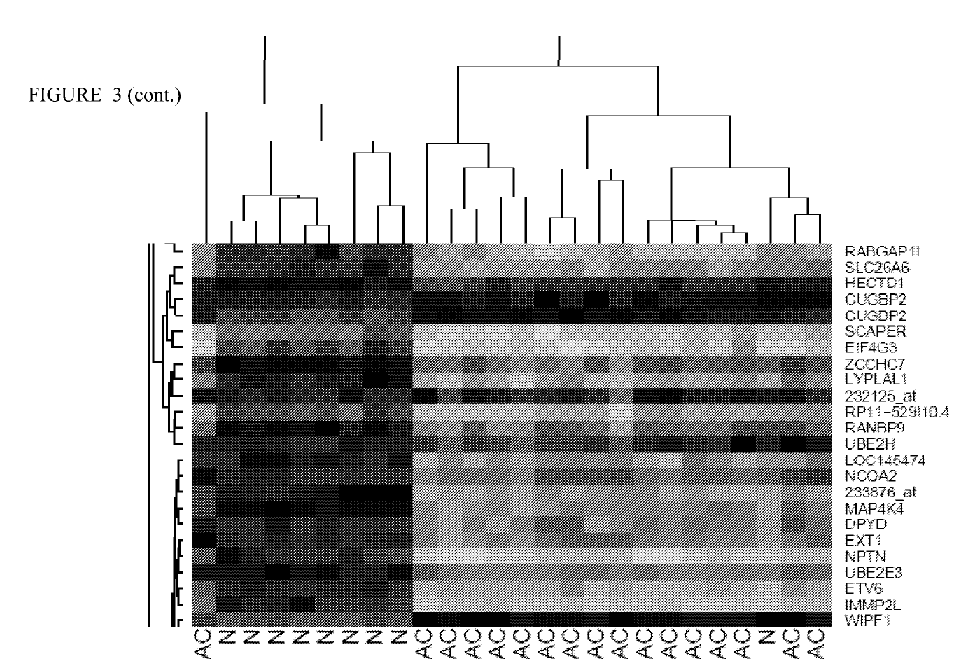

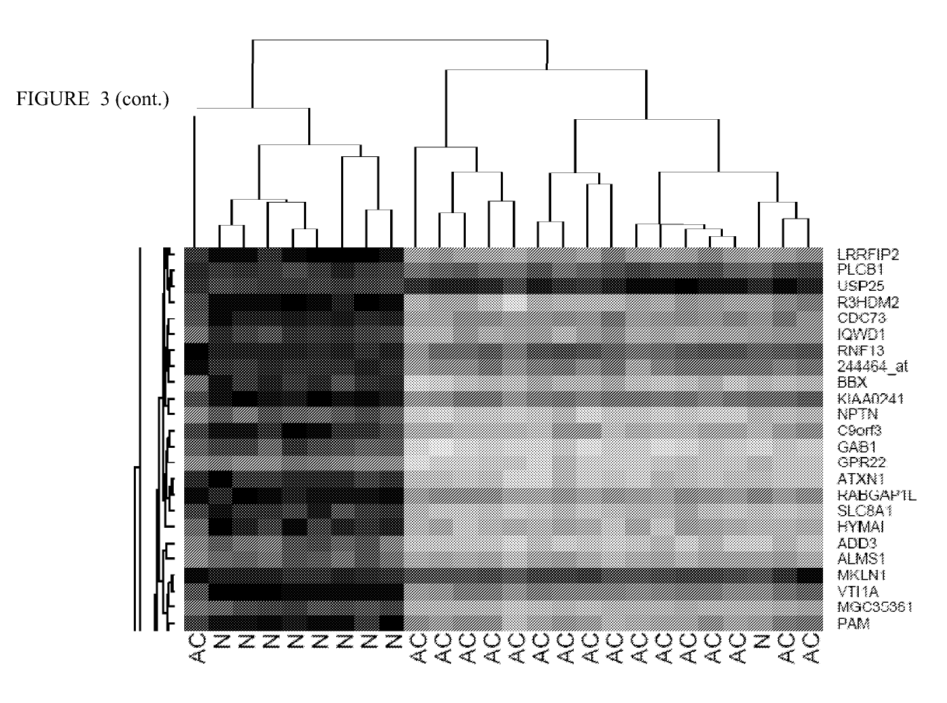

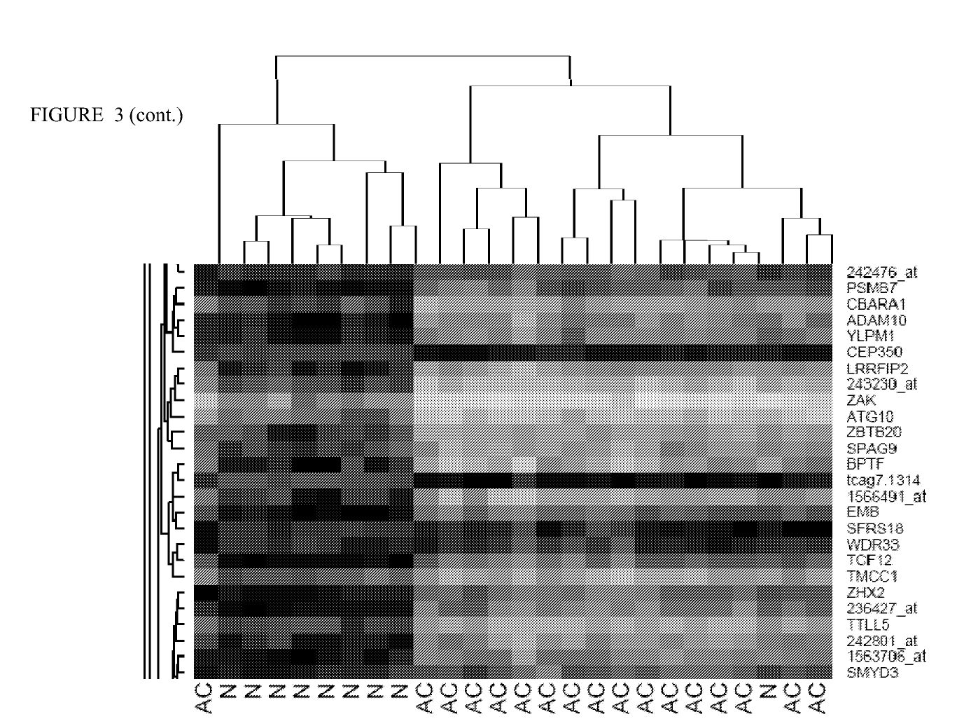

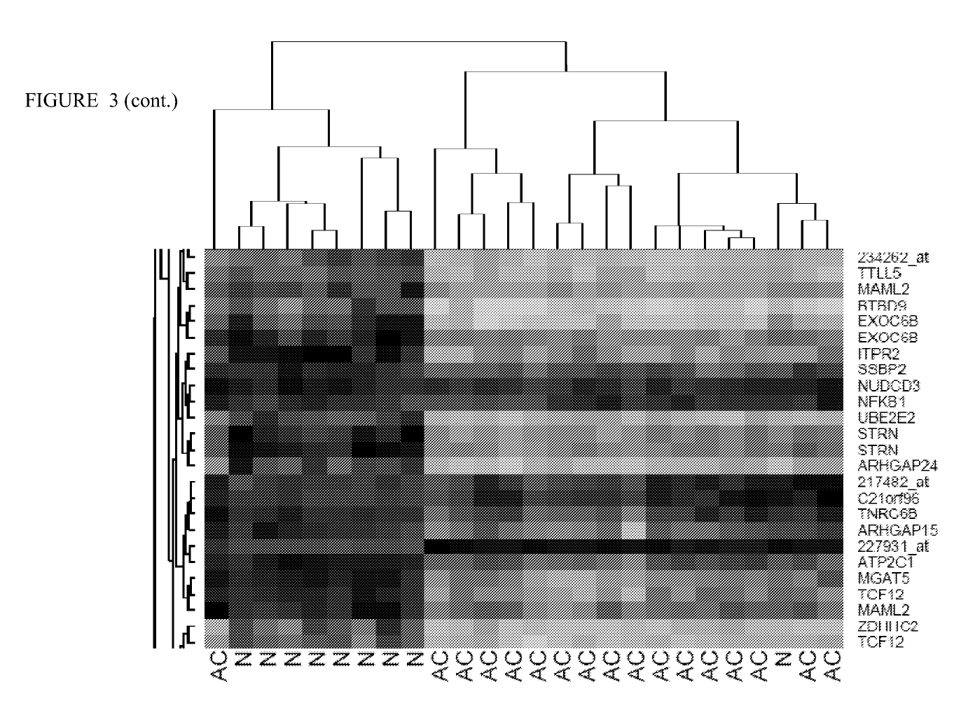

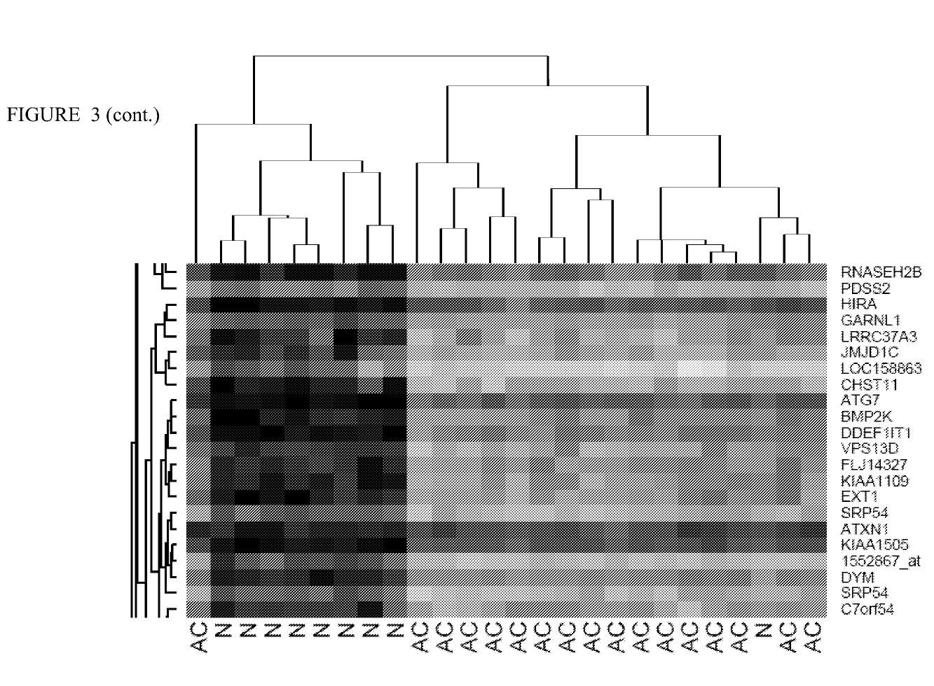

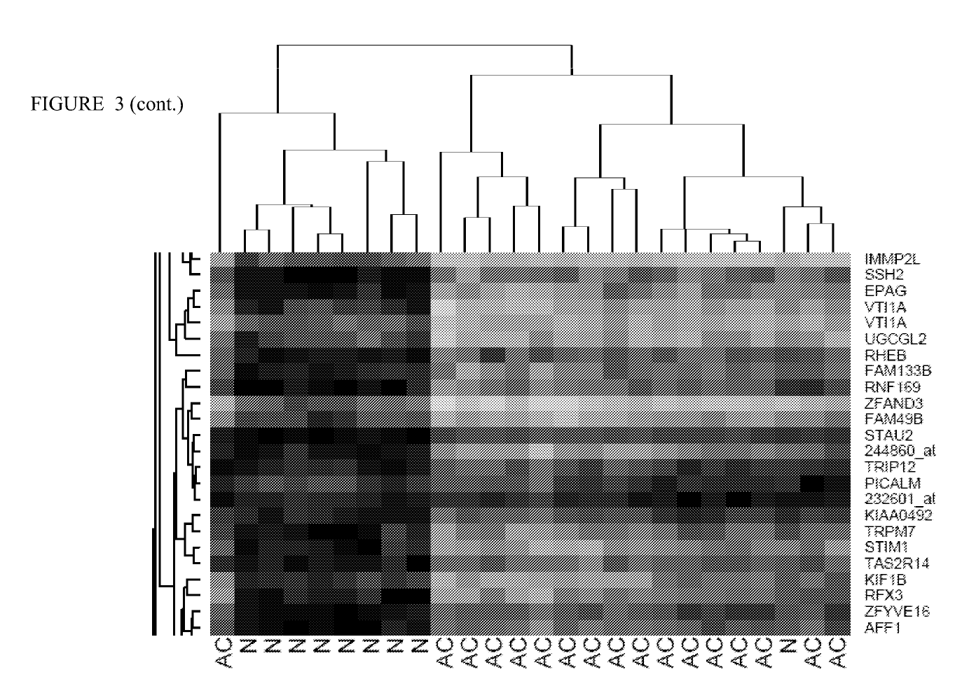

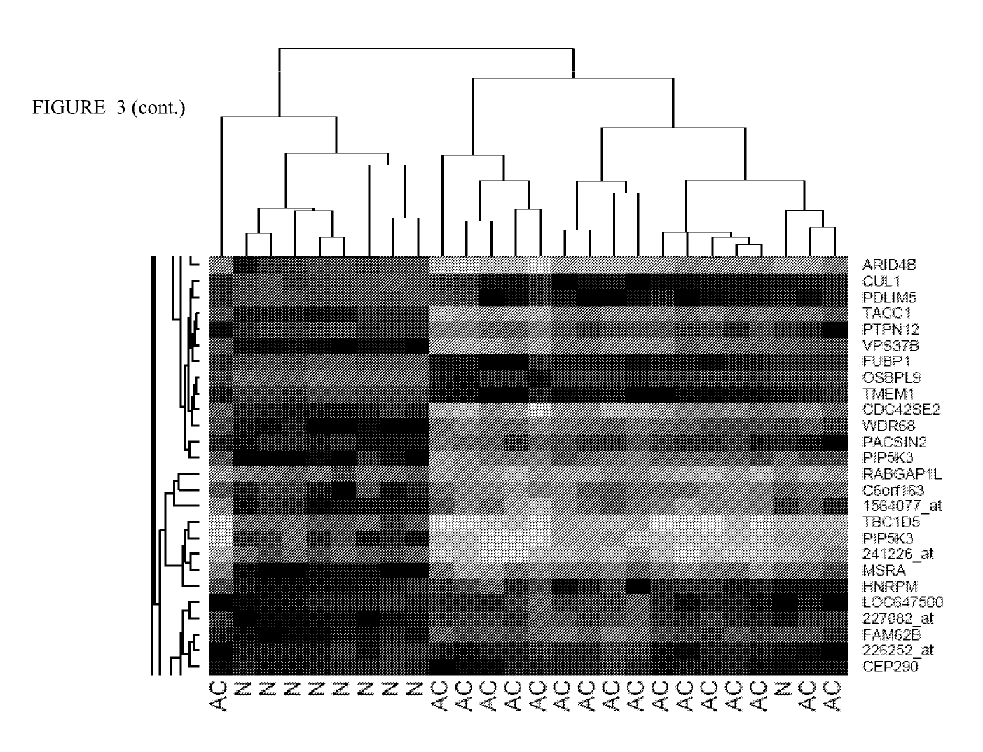

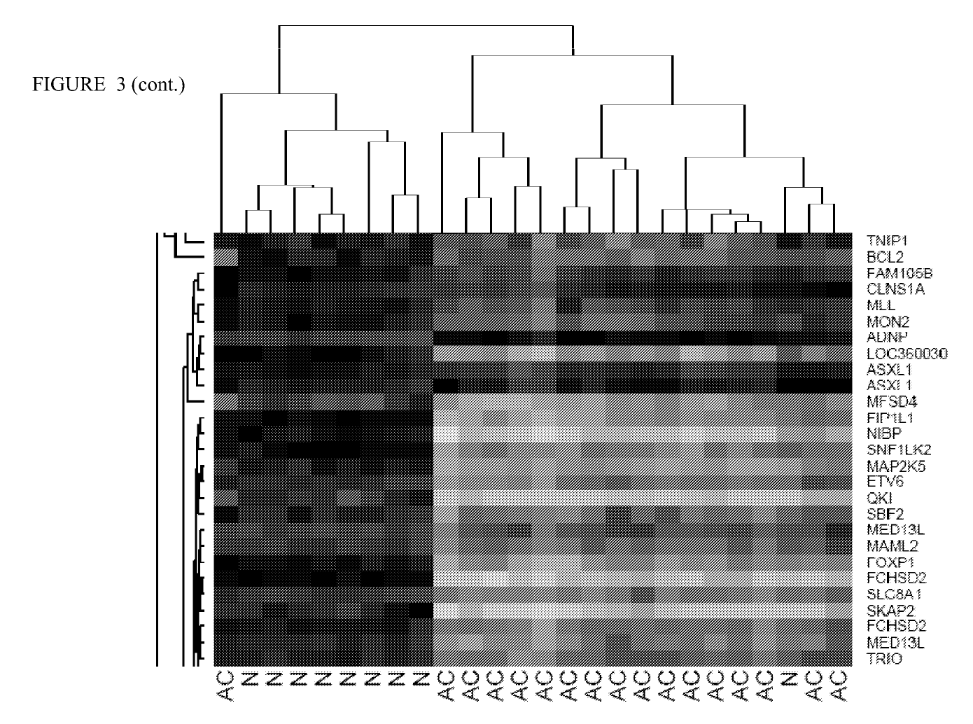

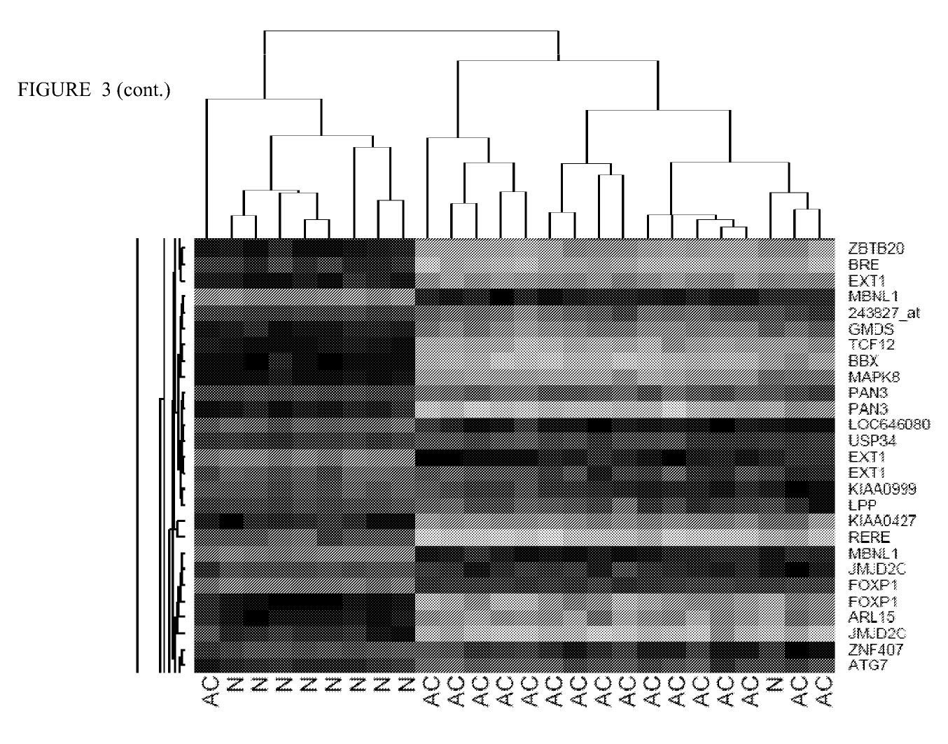

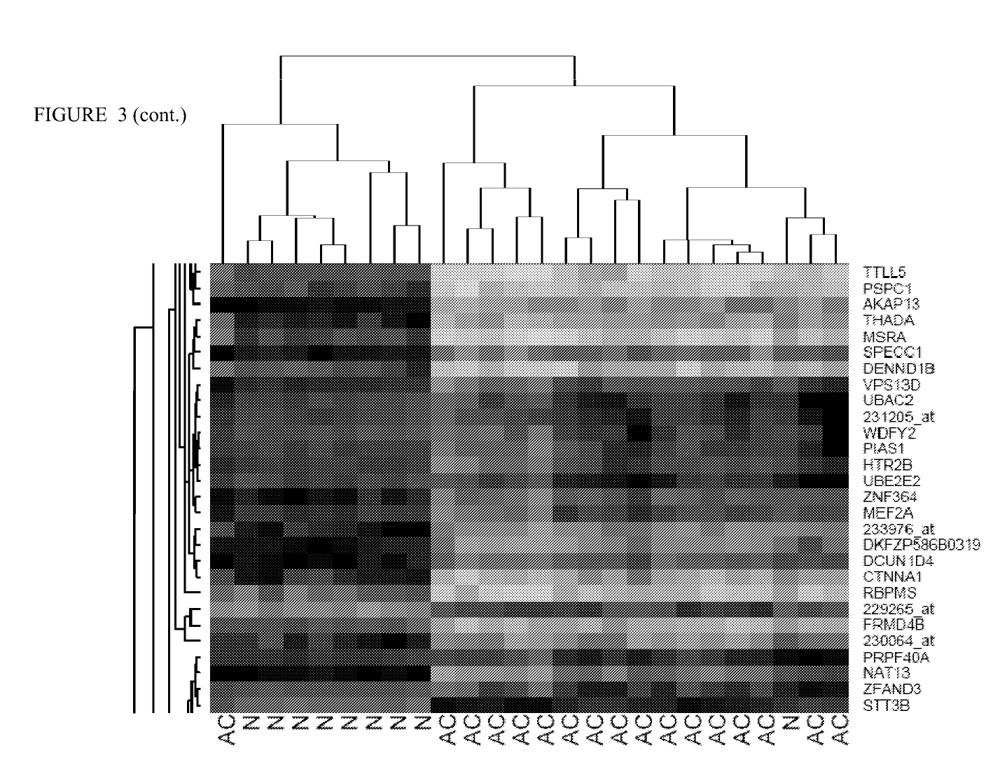

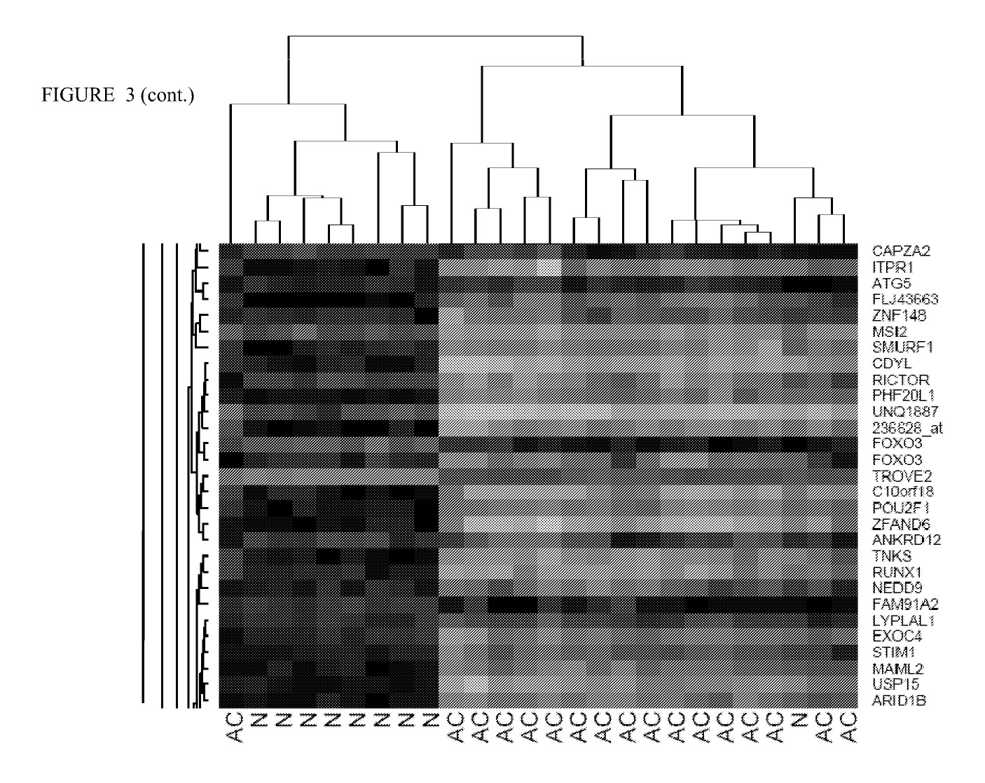

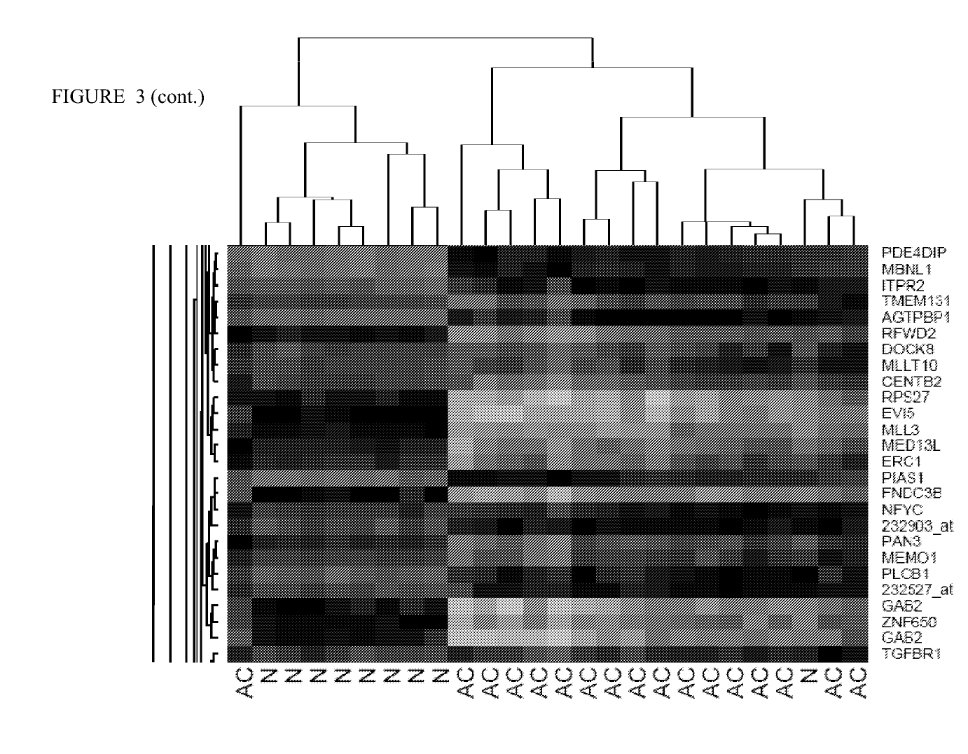

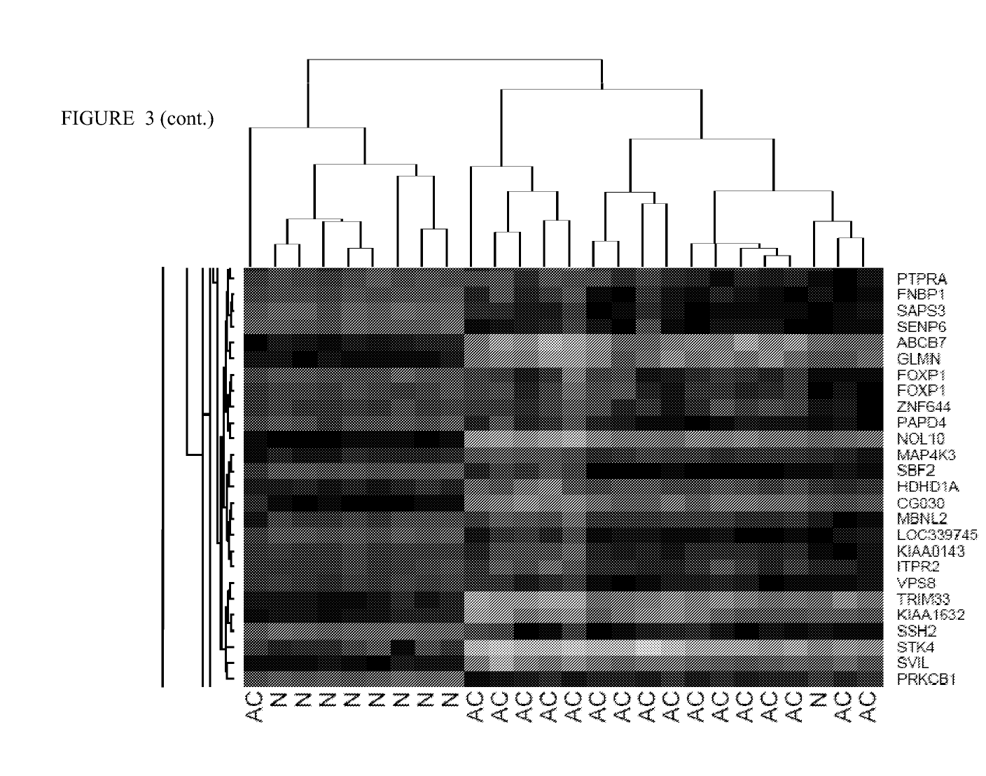

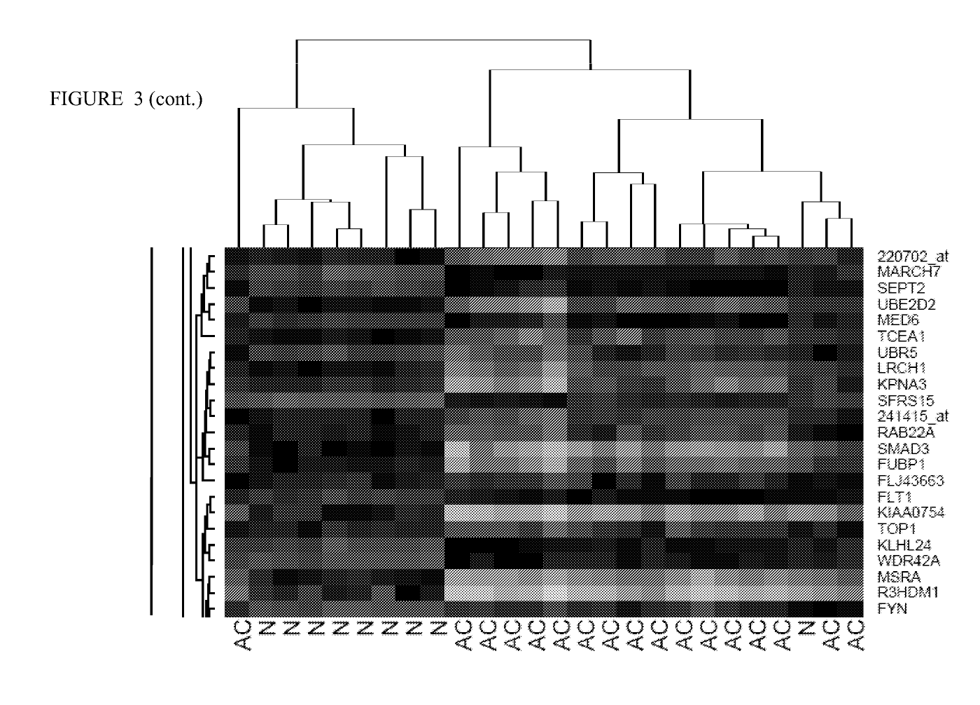

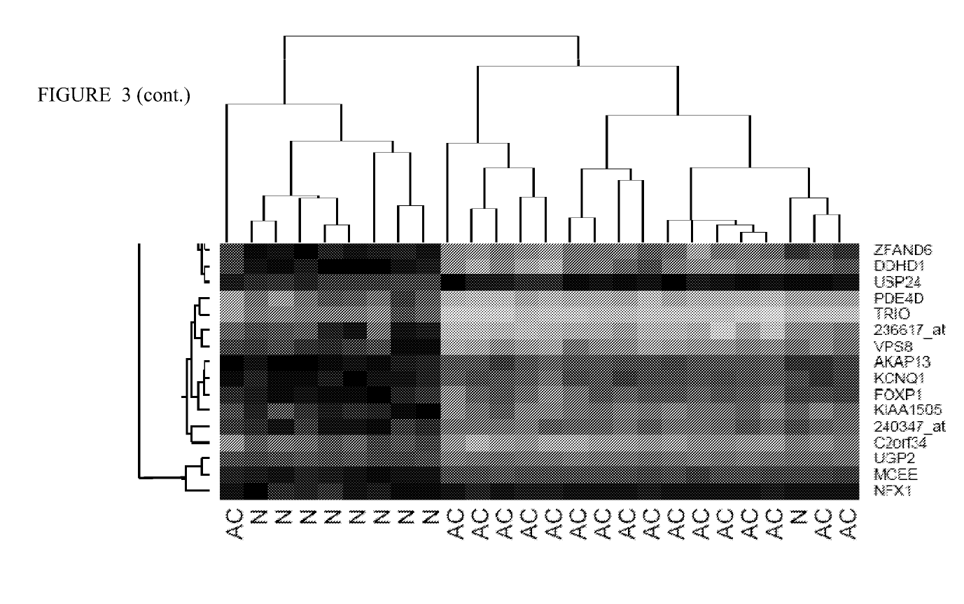

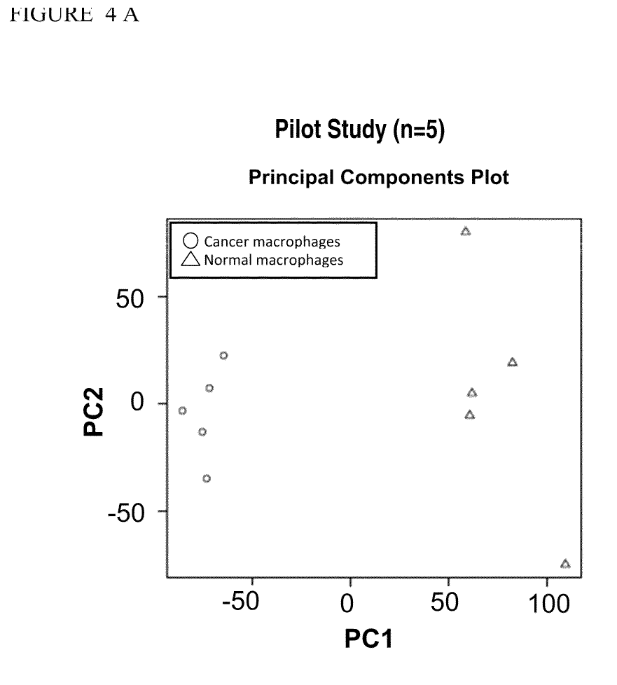

CD11b+ PBMCs were further isolated from prostate cancer patients and defined as active cancer (AC) or stable disease (SD) based on current clinical PSA values and compared to age-matched healthy controls. As shown in FIG. 3, cDNA microarray analysis revealed ˜5,000 genes that were significantly changed in the AC vs. normal controls.

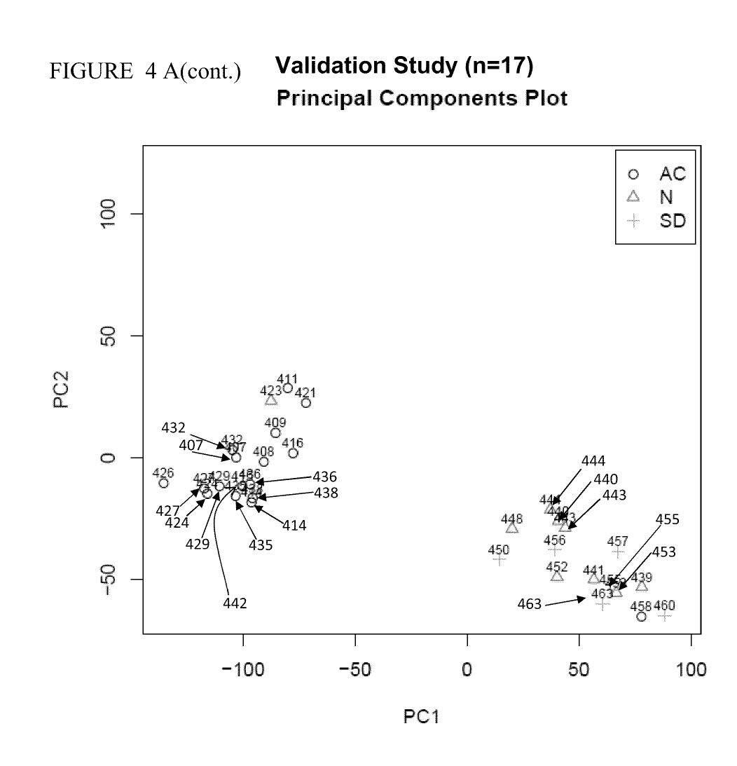

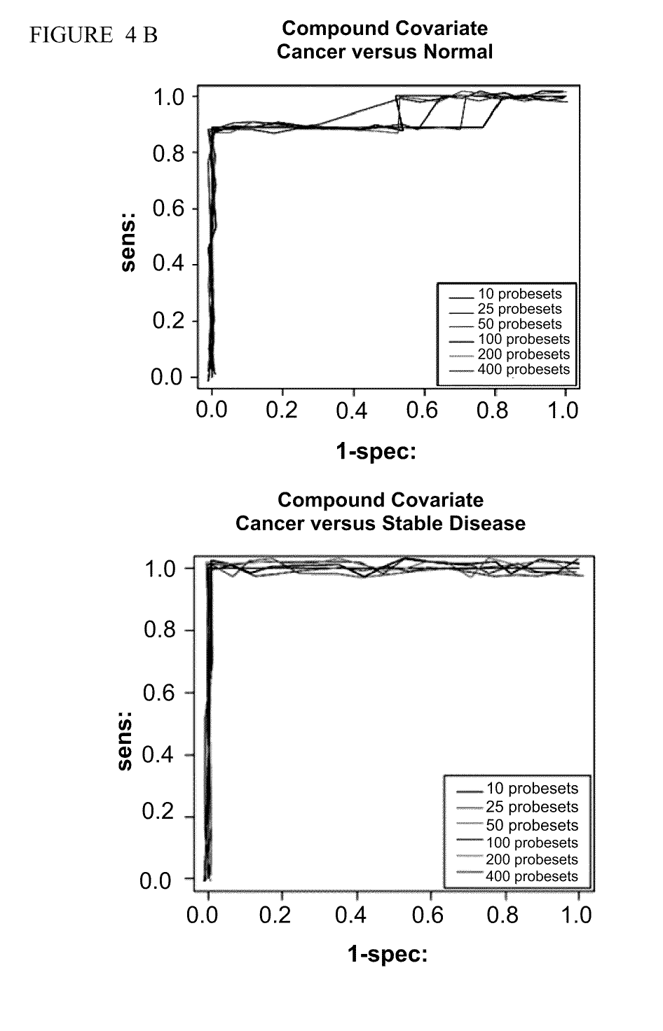

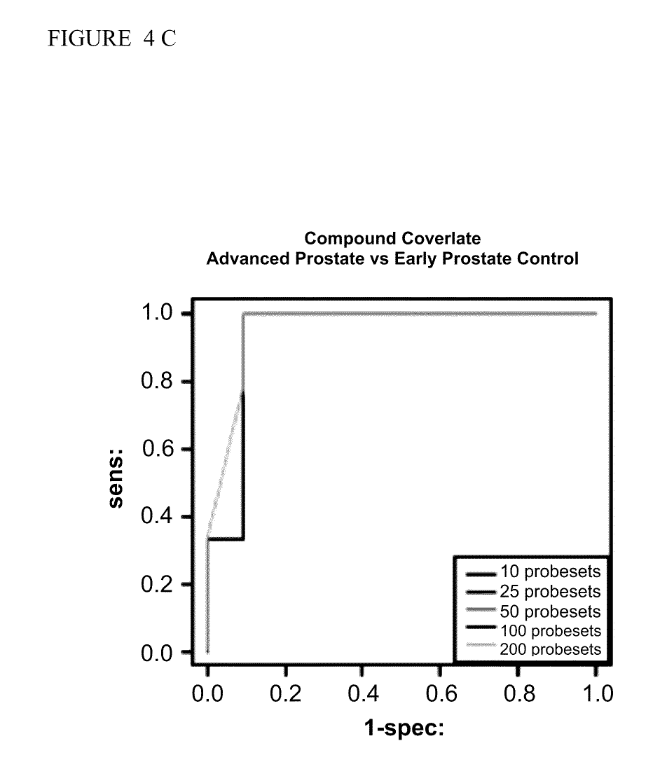

CD11b+ PBMCs from advanced prostate cancer patients with (n=5) compared to aged-matched normal controls (n=5) were next evaluated. All of these patients had osseous metastases and had progressed on castration therapy. The data demonstrated that 902 genes were significantly different between prostate cancer patients and the normal controls (>2 fold expression; p<0.01) (FIG. 4A, FIG. 5A). Principal components analysis revealed separation of cancer patients from controls. This analysis was repeated with a larger group of patients. In the second set of experiments, 17 men with castration resistant prostate cancer and 10 age-matched controls were compared. In addition, 5 men with castration sensitive prostate cancer (men with no evidence of disease, e.g., undetectable PSA, but on castration therapy) were compared. Principal component analysis revealed significant separation of these groups (FIG. 4A). Generation of probe sets using differentially expressed genes generated ROC curves with high sensitivity and specificity (FIG. 4B). As castrated men with no evidence of disease had a similar profile to age-matched controls, the data indicate, for example, that these results were not due to the effect of castration therapy in the patients. These data suggest, for example, that a probe set can be generated that identifies men with prostate cancer. The advanced prostate cancer patients were further compared to another set of controls of 10 age—matched men undergoing prostate cancer biopsy for an elevated PSA who were not diagnosed with cancer (FIG. 4C). These data confirmed that, for example, probe-sets could be generated that differentiated between men with prostate cancer versus those with no clinical evidence of cancer.

Further, a classifier set of 10 genes (see Table 1) able to identify patients having prostate cancer vs. patients not having prostate cancer with 90% specificity and 90% sensitivity by ROC analysis were identified.

TABLE 1
A classifier set of 10 genes able to identify active prostate
cancer vs. normal with 90% specificity and 90% sensitivity
by ROC analysis. Each gene and associated gene product
were shown to be downregulated in active cancer CD11b+ PBMCs
vs. non-cancer CD11b+ PBMCs.

| | |
|---|---|
| MSRA | methionine sulfoxide reductase A |
| ZFAND6 | zinc finger, AN1-type domain 6 |
| THADA | thyroid adenoma associated |
| FYN | FYN oncogene related to SRC, FGR, YES |
| RABGAP1L | RAB GTPase activating protein 1-like |
| IMMP2L | IMP2 inner mitochondrial membrane peptidase-like |
| | (S. cerevisiae) |
| RICTOR | rapamycin-insensitive companion of mTOR |
| JMJD2C | jumonji domain containing 2C |
| NPTN | neuroplastin |
| VTI1A | vesicle transport through interaction with t-SNAREs |
| | homolog 1A (yeast) |

These results demonstrated that the presence of prostate cancer changes the genetic profile of CD11b+ PBMCs and suggests that prostate cancer cells secrete, or stimulate the secretion of, circulating factors that influence the genotype and phenotype of the macrophage precursor cells and represent, for example, a methodology for detecting the presence of prostate cancer as well as monitoring treatment response. These results demonstrated that the presence of prostate cancer changes the genetic profile of CD11b+ PBMCs and suggests that prostate cancer cells secrete, or stimulate the secretion of, circulating factors that influence the genotype and phenotype of the macrophage precursor cells and represent, for example, a methodology for detecting the presence of prostate cancer as well as monitoring treatment response. FIG. 5B shows genes up-regulated and down-regulated in CD11b+ cells isolated from patients with advanced prostate cancer compared to patients not having prostate cancer. FIG. 5C shows genes up-regulated and down-regulated in CD11b+ cells isolated from patients with advanced prostate cancer compared to patients with early prostate cancer.

### Example V

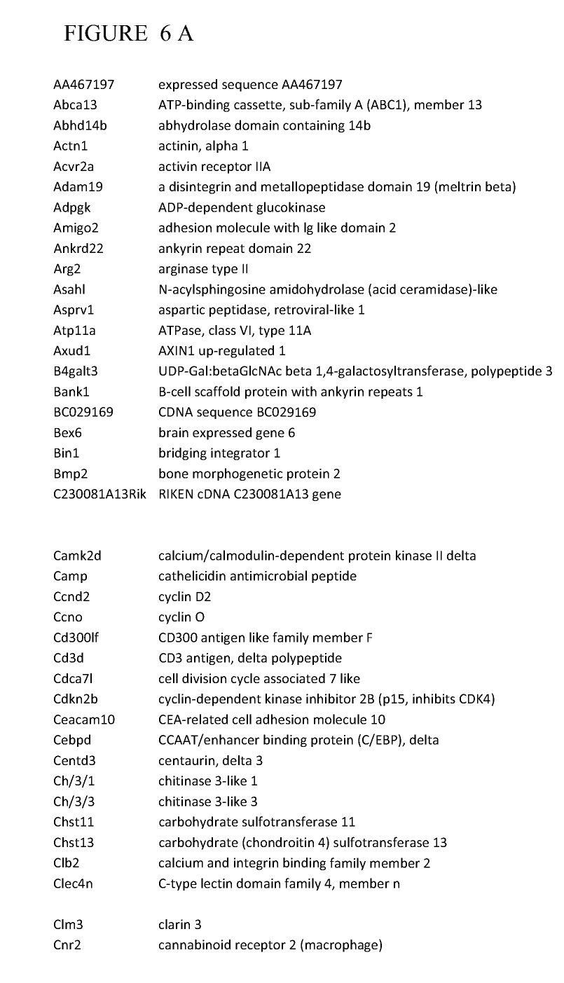

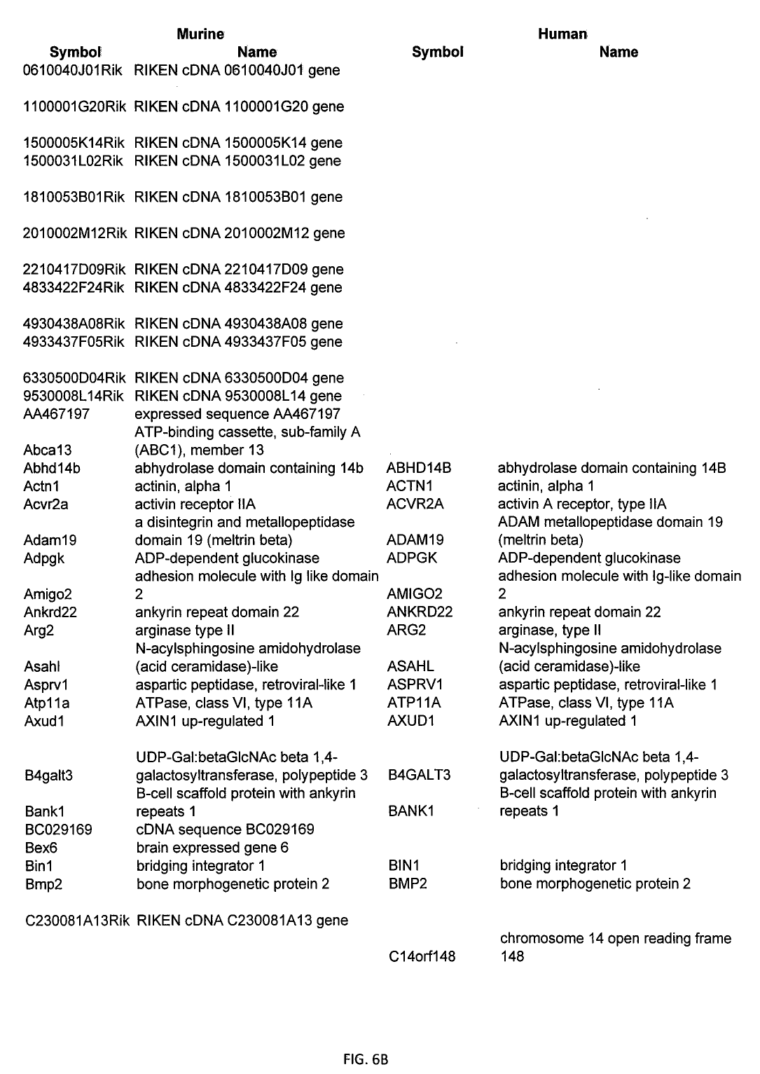

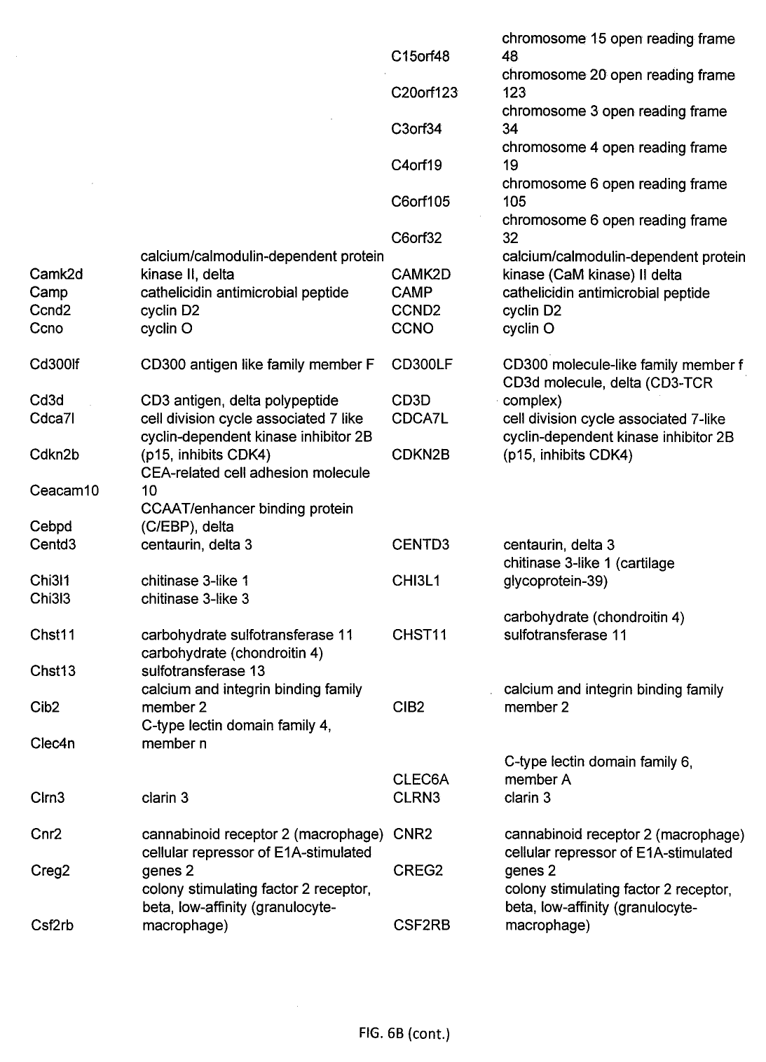

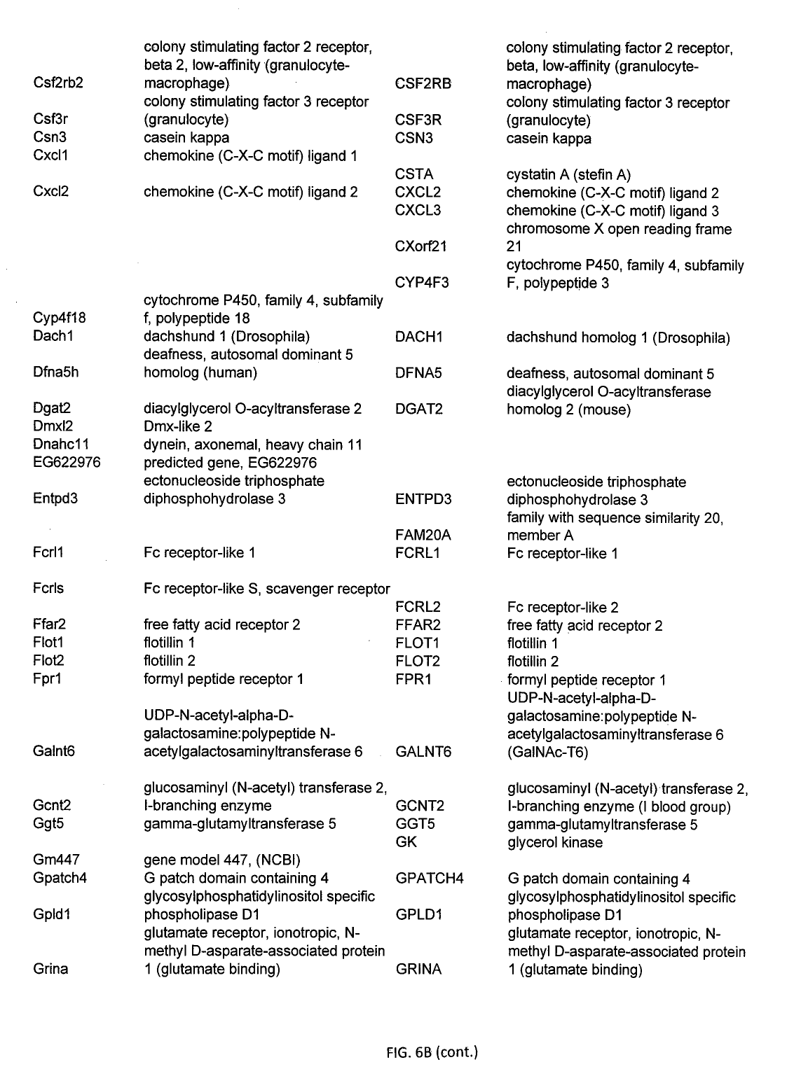

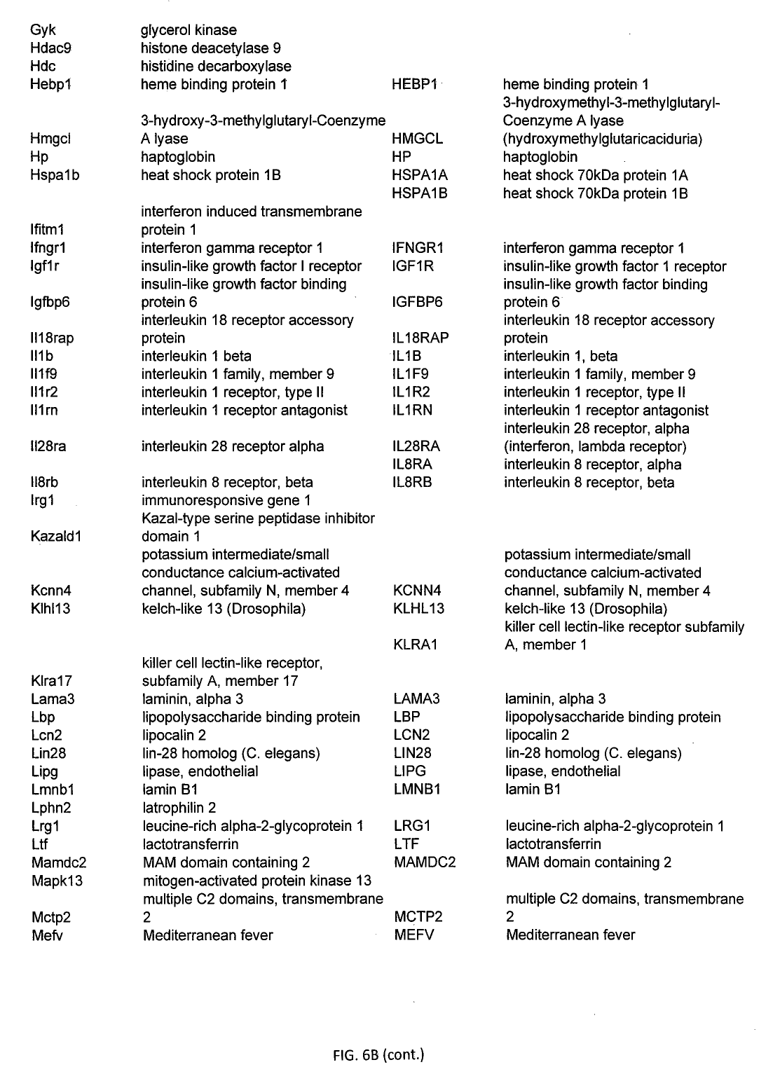

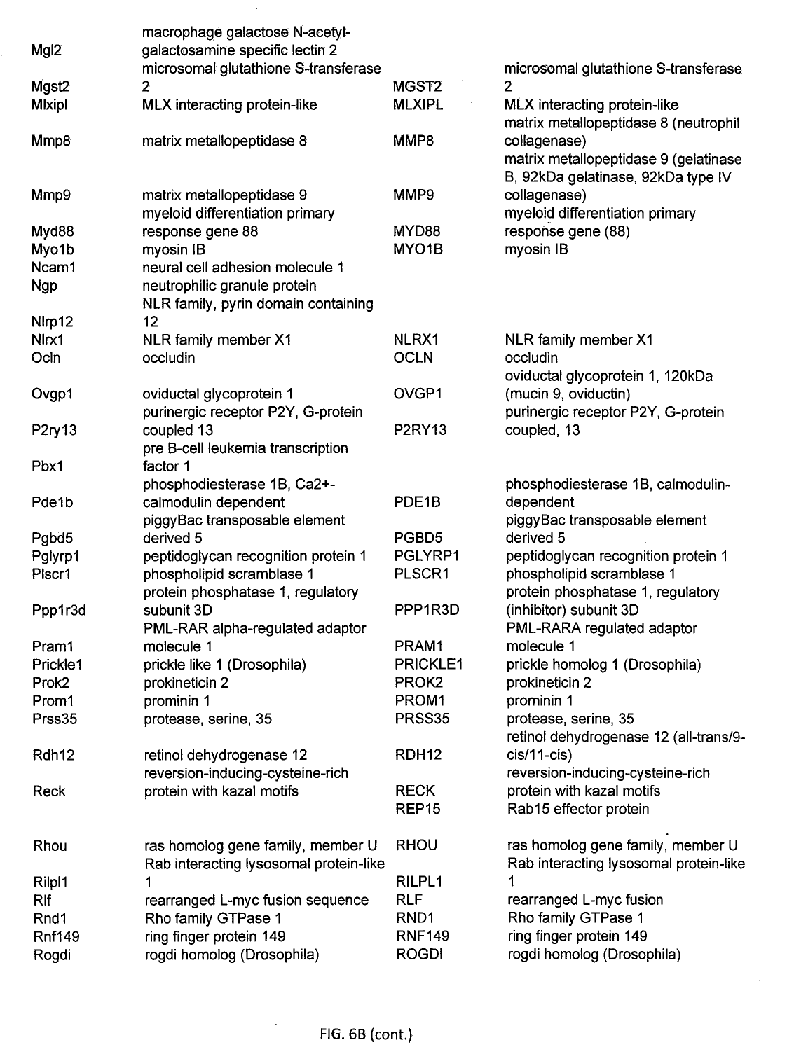

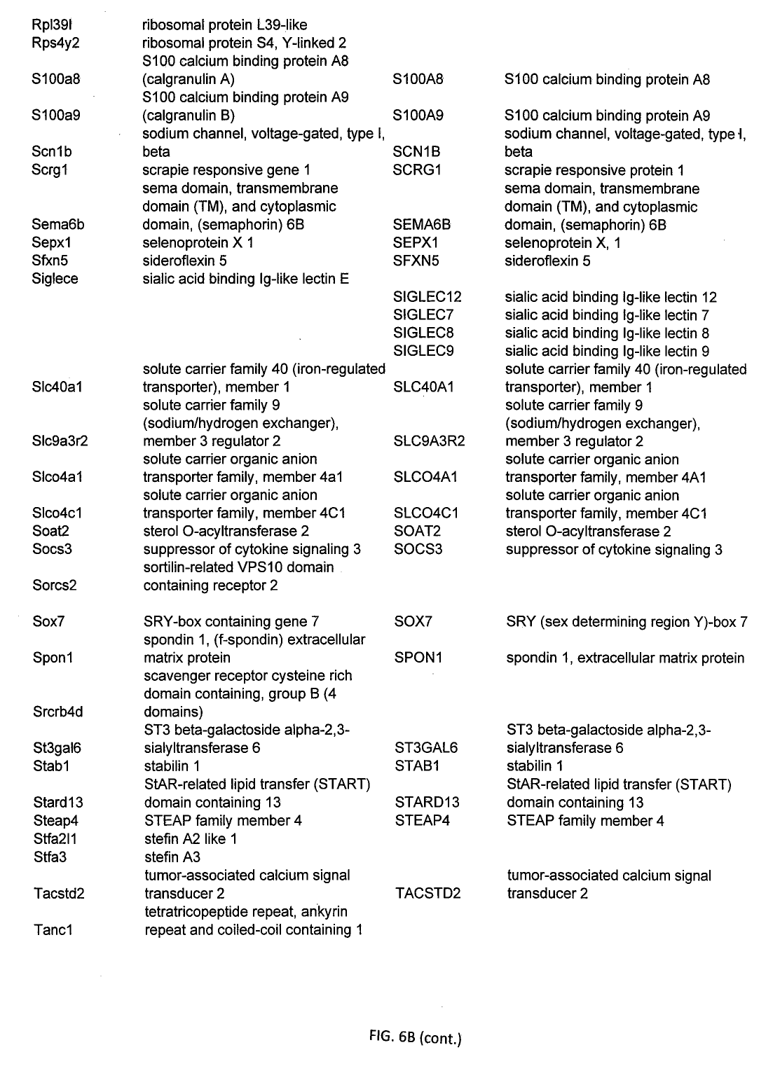

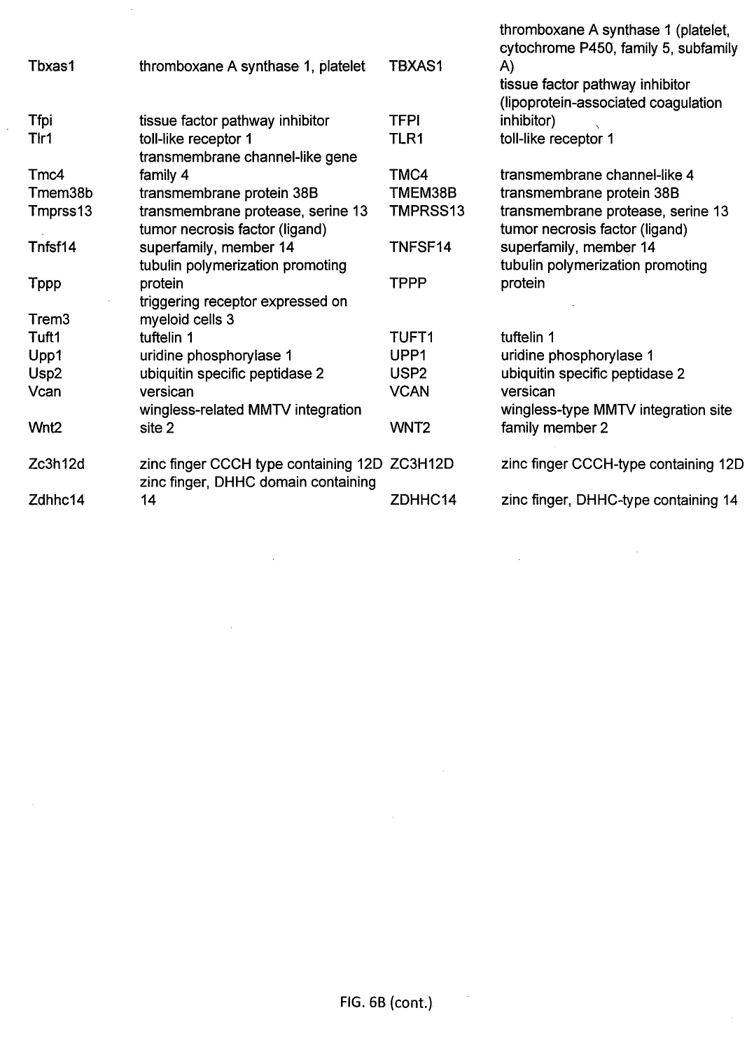

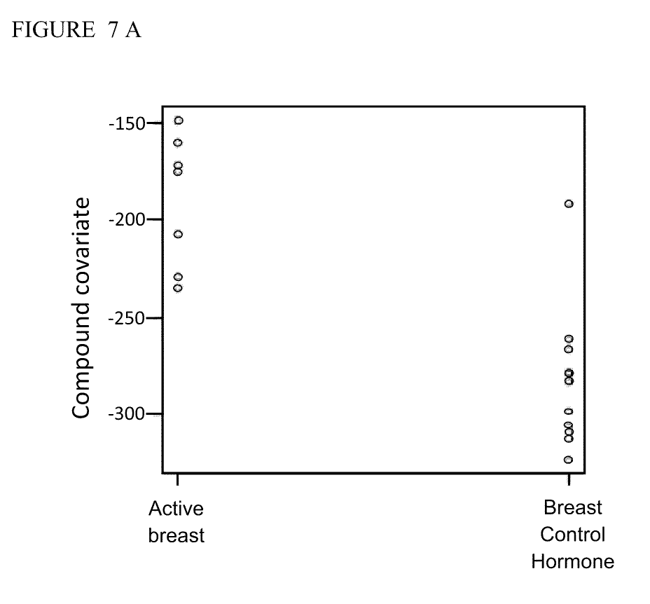

This example demonstrates that genetic changes of CD11b+ cells of mice bearing prostate cancer are similar to genetic changes observed in men with prostate cancer. The in vivo microarray data from the CD11b+ cells was used to compare gene expression changes in the mice bearing prostate cancer xenografts to those from men with advanced prostate cancer. Utilizing GSEA (see, e.g., Jiang, et al., Bioinformatics, 23:306-313, 2007; Subramanian, et al., Proc. Natl. Acad. Sci., 102(43):15545-15550, 2005; each herein incorporated by reference in their entireties) to make comparisons to the human data, mouse gene sets were mapped to human homologs through InParanoid via the Bioconductor homology packages. The mouse Entrez Gene IDs were mapped to InParanoid IDs and then to human Ensembl protein IDs. The Ensembl protein IDs were then mapped to the Entrez Gene IDs. FIGS. 6A and 6B shows mouse and human CD11b+ PBMCs exposed to prostate cancer cells in vivo share similar gene expression profiles.

### Example VI

This example shows that genetic changes of CD11b+ can detect the presence of cancer and unique probe sets can be generated for different cancers. Blood was collected from seven women with metastatic breast cancer. These women all had evidence of metastatic disease and had failed hormonal therapy. Chemotherapy regimens varied among the women. Age matched controls consisted of 10 women with no history of cancer and 12 women with a history of breast cancer on hormonal therapy with no evidence of disease for a minimum of two years. Analysis revealed that CD11b+ cells from women could be differentiated from women without evidence of cancer. Women on hormonal therapy with no evidence of cancer could be differentiated from women with cancer, confirming the data from men with prostate cancer that the data is not the result of differences in hormonal milieu of the host (FIGS. 7A and 7B). FIG. 7C shows genes over-expressed and under-expressed in CD11b+ cells isolated from female patients with active breast cancer compared to females not having breast cancer (e.g., FCRL5, TMEM156, OASL, PPP1R9A, COL4A4, BTLA, FAM110B, TPD52, MGC39900, KIAA0125). Similar analyses were done for lung cancer. A lung cancer specific gene expression set was generated from 3 men and 5 women non-small cell lung cancer as well as 1 man and 2 women with small cell lung cancer. All patients were undergoing or eligible for chemotherapy. Controls were derived from the common set of 10 age-matched men and 10 age-matched women with no history of cancer by verbal report (FIG. 8A). The compound covariate was lung cancer specific. Separation by sex improved the power to differentiate between patients and controls (FIG. 8B). FIG. 8C shows genes over-expressed and under-expressed in CD11b+ cells isolated from patients with lung cancer compared to patients with no lung cancer (e.g., METTL7B, GATA2, CHRM3, SPRYD5, ENPP3, FLVCR2, SEPT1, NLRC3, PHC2, FAM84B). FIG. 8D shows genes over-expressed and under-expressed in CD11b+ cells isolated from female patients with lung cancer compared to female patients with no lung cancer (e.g., NLRC3, CHRM3, KLF8, CXorf57, GATA2, SLC45A3, METTL7B, TRAF5, AKAP12, ARHGAP26). Similar results were found for 5 men and 8 women with advanced pancreatic cancer (FIG. 9A). FIG. 9B shows genes over-expressed and under-expressed in CD11b+ cells isolated from patients with pancreatic cancer compared to patients not having pancreatic cancer (e.g., LGR4, C1QC, FAM20A, FMNL2, C1QA, TCF7L2, C1QB, METTL7B, EZR, CACNA2D3). FIG. 9C shows genes over-expressed and under-expressed in CD11b+ cells isolated from female patients with pancreatic cancer compared to female patients not having pancreatic cancer (e.g., NF1, ZNF224, AKAP13, ZXDB, FCAR, ZNF498, HDAC5, DPP7, SFRS121P1, SNN). Similar results were found for 6 men and 1 woman with metastatic colon cancer (FIG. 10A). FIG. 10B shows genes over-expressed and under-expressed in CD11b+ cells isolated from patients with colon cancer compared to patients not having colon cancer (e.g., FAM20A, FLVCR2, METTL7B, CNTNAP2, WASF1, TCF7L2, ATXN3, ME1, CCR7, GAS6).

### Example VII

This example demonstrates that genetic changes of CD11b+ can detect the presence of active systemic lupus erythematosus and express a signature unique from cancer. Isolated circulating CD11b+ cells from eight women with active systemic lupus erythematosus and seven women with inactive systemic lupus erythematosus were tested as determined by established clinical criteria (see, e.g., J Rheumatol. 2004 October; 31(10):1934-40; herein incorporated by reference in its entirety). Analysis revealed a unique signature that identified these patients from a control pool of ten age and sex matched controls (FIG. 11A). FIG. 11B shows genes over-expressed and under-expressed in CD11b+ cells isolated from patients with active systemic lupus erythematosus compared to patients not having systemic lupus erythematosus (e.g., TOP2A, TRPM7, USP15, UGCG, TTLL5, SSFA2, ZC3HAV1, AFF1, AGGF1, TET2). This signature was also different from the cancer signatures. In addition, patients with inactive systemic lupus erythematosus could also be differentiated from the control population (FIG. 11C). FIG. 11D shows genes over-expressed and under-expressed in CD11b+ cells isolated from patients with inactive systemic lupus erythematosus compared to patients not having systemic lupus erythematosus (e.g., PPBP, SDPR, SPARC, PF4, GNG11, STAT1, PATL1, TBXA2R, PPCSK6, ITGB3). Patients with active systemic lupus erythematosus could also be differentiated from patients with inactive systemic lupus erythematosus (FIG. 11E). FIG. 11F shows genes over-expressed and under-expressed in CD11b+ cells isolated from patients with active systemic lupus erythematosus compared to patients with inactive systemic lupus erythematosus (e.g., PTBP1, ZNF274, RHEB, LARP5, KIAA1128, C10orf46, SMAD7, NARG1, FAM123B, CYP4V2).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for assessing a disorder, comprising: identifying a characteristic associated with CD11b+ peripheral blood mononuclear cells obtained from a patient sample, wherein said characteristic is associated with said disorder.

2. The method of claim 1, wherein said characteristic associated the CD11b+ peripheral blood mononuclear cell is altered gene expression compared to a control sample.

3. The method of claim 1, wherein said disorder is selected from the group consisting of an inflammatory disorder and a hyperproliferative disorder.

4. The method of claim 1, wherein said disorder is systemic lupus erythematosus.

5. The method of claim 3, wherein said hyperproliferative disorder is cancer.

6. The method of claim 5, wherein said cancer is selected from the group consisting of breast cancer, pancreatic cancer, prostate cancer, colon cancer, and lung cancer.

7. The method of claim 2, wherein said disorder is prostate cancer, wherein said altered gene expression is in one or more genes selected from the group consisting of MSRA, ZFAND6, THADA, FYN, RABGAP1L, IMMP2L, RICTOR, JMJD2C, NPTN, and VTI1A.

8. The method of claim 2, wherein said disorder is lung cancer, wherein said altered gene expression is in one or more genes selected from the group consisting of METTL7B, GATA2, CHRM3, SPRYD5, ENPP3, FLVCR2, SEPT1, NLRC3, PHC2, FAM84B.

9. The method of claim 2, wherein said disorder is breast cancer, wherein said altered gene expression is in one or more genes selected from the group consisting of FCRL5, TMEM156, OASL, PPP1R9A, COL4A4, BTLA, FAM110B, TPD52, MGC39900, KIAA0125.

10. The method of claim 2, wherein said disorder is pancreatic cancer, wherein said altered gene expression is in one or more genes selected from the group consisting of LGR4, C1QC, FAM20A, FMNL2, C1QA, TCF7L2, C1QB, METTL7B, EZR, CACNA2D3.

11. The method of claim 2, wherein said disorder is colon cancer, wherein said altered gene expression is in one or more genes selected from the group consisting of FAM20A, FLVCR2, METTL7B, CNTNAP2, WASF1, TCF7L2, ATXN3, ME1, CCR7, GAS6.

12. The method of claim 2, wherein said disorder is systemic lupus erythematosus, wherein said altered gene expression is in one or more genes selected from the group consisting of TOP2A, TRPM7, USP15, UGCG, TTLL5, SSFA2, ZC3HAV1, AFF1, AGGF1, TET2.

13. The method of claim 1, wherein said sample is selected from the group consisting of a biopsy sample, and a blood sample.

14. The method of claim 2, wherein said gene expression is determined using a detection technique selected from the group consisting of microarray analysis, reverse transcriptase PCR, quantitative reverse transcriptase PCR, and hybridization analysis.

15. The method of claim 5, wherein said characteristic is associated with a stage of cancer in said patient.

16. The method of claim 1, further comprising the step of reporting the results of said identifying to a physician.

17. The method of claim 16, further comprising the step of selecting a treatment course of action.

18. The method of claim 16, further comprising the step of administering a medical intervention to said patient.

19. The method of claim 1, wherein a gene expression profile of said CD11b+ peripheral blood mononuclear cells is compared to an established genetic expression profile for CD11b+ peripheral blood mononuclear cells associated with said disorder.
